SEQUENCE LISTING (117 sequences)

SEQ ID NO: 1 - DNA, 1383 bp, Homo sapiens
atggagttggggctgtgctggattttccttgttgctattataaaaggtgtccagtgtcaggtgcagctggtggagtctgggggaggcttggtcaagcctggagggtccctgagactctcctgtgcagcctctggattcaccttcagtgactactacatgagctggatccgccaggctccagggaaggggctggagtggatttcatacattagtggtagtggtagtaccatatactacgcagactctgtgaagggccgattcaccatctccagggacaacgccaagaactcactgtatctgcaaatgaacagcctgagagccgaggacacggccgtgtatcactgtgcgagagccctgggtgggatggacgtctggggccaagggaccacggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 461 aa, Homo sapiens
MELGLCWIFLVAIIKGVQCQVQLVESGGGLVKPGGSLRLSCAASGFTFSDYYMSWIRQAPGKGLEWISYISGSGSTIYYADSVKGRFTISRDNAKNSLYLQMNSLRAEDTAVYHCARALGGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - DNA, 702 bp, Homo sapiens
atgagggtccctgctcagctcctggggctcctgctactctggctccgaggtgccagatgtgacatccagatgacccagtctccatcctccctgtctgcatctgtaggagacagagtcaccatcacttgccgggcaagtcagagcattagcggctttttaaattggtatcagcagaaaccagggaaagcccctaagctcctgatctatgctacatccagtttgcaaagtggggtcccattcaggttcagtggcagtggatctgggacagatttcactctcaccatcagcagtctgcaacctgaagattttgcaacttattactgtcaacagagttacagtgtcccattcactttcggccctgggaccaaagtggatatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 4 - protein, 234 aa, Homo sapiens
MRVPAQLLGLLLLWLRGARCDIQMTQSPSSLSASVGDRVTITCRASQSISGFLNWYQQKPGKAPKLLIYATSSLQSGVPFRFSGSGSGTDFTLTISSLQPEDFATYYCQQSYSVPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1389 bp, Homo sapiens
atggaatttgggctgtgctgggttttccttgttgctattttagaaggtgtccagtgtgaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgctccgggtatagcagcagctggtagggcctactggggccagggaaccctggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - protein, 463 aa, Homo sapiens
MEFGLCWVFLVAILEGVQCEVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAPGIAAAGRAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - DNA, 702 bp, Homo sapiens
atgagggtccctgctcagctcctggggctcctgctactctggctccgaggtgccagatgtgacatccagatgacccagtctccatcctccctgtctgcatctgttggagacagagtcaccatcacttgccggccaagtcaggacattagcagttatttaaattggtatcagcagaaaccagggaaagcccctaagctcctgatctatgctgcatccagtttgcaaagtggggtcccattaaggttcagtggcagtggatctgggacagatttcactctcaccatcagcagtctgcaacctgaagattttgcaacttactactgtcaacagagttacagtaccccattcactttcggccctgggaccaaagtggatatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 8 - protein, 234 aa, Homo sapiens
MRVPAQLLGLLLLWLRGARCDIQMTQSPSSLSASVGDRVTITCRPSQDISSYLNWYQQKPGKAPKLLIYAASSLQSGVPLRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1398 bp, Homo sapiens
atggagtttgggctccgctggatttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggaatgggtctcagctattagtggtcgtggtggtaggacatacttcgcagactccgtgaagggccggttcaccatctccagagacaattccaagaacacgctgtatctgcaaatgaacagcctgagagccgaggacacggccgtatatttctgtgcggtagaaggctatagtgggcgctacggattttttgactactggggccagggaaccctagtcaccgtctcctcagcctccaccaagggcccatcggtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 466 aa, Homo sapiens
MEFGLRWIFLVAILKGVQCEVQLLESGGGLVQPGGSLRLSCAASGFTFSSYAMSWVRQAPGKGLEWVSAISGRGGRTYFADSVKGRFTISRDNSKNTLYLQMNSLRAEDTAVYFCAVEGYSGRYGFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - DNA, 702 bp, Homo sapiens
atggaagccccagctcagcttctcttcctcctgctactctggctcccagataccactggagaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccagtggtatcccagacaggatcagtggcagtgggtctggaacagagttcactctcatcatcagcagcctgcagtctgaagattttgcagtttattactgtcagcagtctaataactggccattcactttcggccctgggaccaaagtggatatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 12 - protein, 234 aa, Homo sapiens
MEAPAQLLFLLLLWLPDTTGEIVMTQSPATLSVSPGERATLSCRASQSVSSNLAWYQQKPGQAPRLLIYGASTRASGIPDRISGSGSGTEFTLIISSLQSEDFAVYYCQQSNNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - skipped

SEQ ID NO: 14 - protein, 460 aa, Homo sapiens
MEFGLSWVFLVAIIKGVQCQVQLVESGGGLVKPGGSLRLSCAASGFTFSDYYMSWIRQAPGKGLEWFSYISSSGSTIYYADSVKGRFTISRDNAKNSLSLQMNSLRAEDTAVYYCARGLTGDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - skipped

SEQ ID NO: 16 - protein, 236 aa, Homo sapiens
MDMRVPAQLLGLLLLWFPGSRCDIQMTQSPSSVSASVGDRVTISCRASQDISGWLAWYQQKPGKAPKLLISATSSLHSGVPSRFSGSGSGTDFTLTISSLQPEDFATYYCQQTNSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - skipped

SEQ ID NO: 18 - protein, 463 aa, Homo sapiens
MEFGLSWVFLVALLRGCQCQVQLVESGGGVVQPGRSLRLSCAASGFTFSSYGMHWVRQAPGKGLEWVAFIWYDGSNKYYADSVKGRFTISRDNSKNTLYLQMNSLRAEDTAVYYCARGYRVYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - skipped

SEQ ID NO: 20 - protein, 236 aa, Homo sapiens
MDMRVPAQLLGLLLLWFPGSRCDIQMTQSPSSVSASVGDRVTITCRASQDISSWLAWYQRKPGKAPKLQIYAASSLESGVPSRFNGSGSGTDFTLSISSLQPEDFATYYCQQTNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - skipped

SEQ ID NO: 22 - protein, 464 aa, Homo sapiens
MEWTWSFLFLVAAATGAHSQVQLVQSGAEVKKPGASVKVSCKASGYTFTSYGISWVRQAPGQGLEWMGWISAYNGNTNYAQKLQDRVTMTTDTSTTTAYMELRSLRSDDTAVYYCARRAYGANFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - skipped

SEQ ID NO: 24 - protein, 240 aa, Homo sapiens
MVLQTQVFISLLLWISGAYGDIVMTQSPDSLAVSLGERATINCKSSQSILFFSNNKNYLAWYRQKPGQPPNLLIYWASTRESGVPDRFSGSGSGTDFTLTISSLQAEDVAVYYCQQYYSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 1380 bp, Homo sapiens
atggagtttgggctgagctgggttttccttgttgctattataaaaggtgtccagtgtcaggtgcagctggtggagtctgggggaggcttggtcaagcctggagggtccctgagactctcctgtgcagcctctggattcaccttcagtgactactatatgagctggatccgccaggctccagggaagggactggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagaggcctaactggggactactggggccagggaaccctggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - protein, 460 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGLTGDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - DNA, 708 bp, Homo sapiens
atggacatgagggtccccgctcagctcctggggctcctgctactctggctccgaggtgccagatgtgacatccagatgacccagtctccatcctccctgtctgcatctgtcggagacagagtcaccatcacttgccggccaagtcagatcattagcagtttattaaattggtatcagcagaaaccagggaaagcccctaagctcctgatccatgctgcatccagtttgcaaagtggggtcccatcaaggttcagtggcagtggatctgggacagatttcactctcaccatcagtagtctgcaacctgaagattttgcaacttactactgtcaacagagttacagtaccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 28 - protein, 236 aa, Homo sapiens
MDMRVPAQLLGLLLLWLRGARCDIQMTQSPSSLSASVGDRVTITCRPSQIISSLLNWYQQKPGKAPKLLIHAASSLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 1398 bp, Homo sapiens
atggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagttttagtatgacctgggtccgccaggctccaggaaaggggctggagtgggtttcatacattagtagtagaagtagtaccatatcctacgcagactctgtgaagggccgattcaccatctccagagacaatgccaagaactcactgtatctgcaaatgaacagcctgagagacgaggacacggctgtgtattactgtgcgagagatcctcttctagcgggagctaccttctttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 466 aa, Homo sapiens
MELGLCWVFLVAILEGVQCEVQLVESGGGLVQPGGSLRLSCAASGFTFSSFSMTWVRQAPGKGLEWVSYISSRSSTISYADSVKGRFTISRDNAKNSLYLQMNSLRDEDTAVYYCARDPLLAGATFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - DNA, 705 bp, Homo sapiens
atggaaaccccagcgcagcttctcttcctcctgctactctggctcccagataccaccggagaatttgtgttgacgcagtctccaggcaccctgtctttgtctccaggggaaagagccaccctctcctgcagggccagtcagagtgttagcagcagttacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 32 - protein, 235 aa, Homo sapiens
METPAQLLFLLLLWLPDTTGEFVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 1383 bp, Homo sapiens
atggagtttgggctgagctgggttttccttgttgctattataaaaggtgtccagtgtcaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaattcactgtatctgcaaatgaacagcctgagagccgaggacacggccgtgtattactgtgcgaggcgtataggaggtatggacgtctggggccaagggaccacggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - protein, 461 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARRIGGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - DNA, 708 bp, Homo sapiens
atggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattagcggctttttaatttggtatcagcagagaccagggaaagcccctaagctcctgatctatgctacatccagtttacaaagtggggtcccatcaaggttcagtggcagtggatctgggacagatttcactctcaccatcagcagtctgcaacctgaagattttgcaacttactactgtcaacagagttacagtaccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 36 - protein, 236 aa, Homo sapiens
MDMRVPAQLLGLLLLWLRGARCDIQMTQSPSSLSASVGDRVTITCRASQSISGFLIWYQQRPGKAPKLLIYATSSLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 1383 bp, Homo sapiens
atggagtttgggctgagctgggttttccttgttgctattataaaaggtgtccagtgtcaggtgcagctggtggagtctgggggaggcttggtcaagcctggagggtccctgagactctcctgtgcagcctctggattcaccttcagtgactactatatgagctggatccgccaggctccagggaagggactggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagaggcctaactggggactactggggccagggaaccctggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 38 - protein, 461 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGLTGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 39-42 - skipped (4 entries)

SEQ ID NO: 43 - DNA, 705 bp, Homo sapiens
atggaaaccccagcgcagcttctcttcctcctgctactctggctcccagataccaccggagaatttgtgttgacgcagtctccaggcaccctgtctttgtctccaggggaaagagccaccctctcctgcagggccagtcagagtgttagcagcagttacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgtagtgtattactgtcagcagtatggtagctcacctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 44 - protein, 235 aa, Homo sapiens
METPAQLLFLLLLWLPDTTGEFVLTQSPGTLSLSPGERATLSCRASQSVSSSYLAWYQQKPGQAPRLLIYGASSRATGIPDRFSGSGSGTDFTLTISRLEPEDFVVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 1386 bp, Homo sapiens
atggagtttgggctgagctgggttttccttgttgctattataaaaggtgtccagtgtcaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaattcactgtatctgcaaatgaacagcctgagagccgaggacacggccgtgtattactgtgcgaggcgtataggaggtatggacgtctggggccaagggaccacggtcaccgtctcctcagcttccaccaagggcccatccgtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 46 - protein, 462 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARRIGGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 47 - DNA, 708 bp, Homo sapiens
atggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattagcggctttttaatttggtatcagcagagaccagggaaagcccctaagctcctgatctatgctacatccagtttacaaagtggggtcccattaaggttcagtggcagtgaatctgggacagatttcactctcaccatcagcagtctgcaacctgaagattttgcaacttactactgtcaacagagttacagtaccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 48 - protein, 236 aa, Homo sapiens
MDMRVPAQLLGLLLLWLRGARCDIQMTQSPSSLSASVGDRVTITCRASQSISGFLIWYQQRPGKAPKLLIYATSSLQSGVPLRFSGSESGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - skipped

SEQ ID NO: 50 - protein, 462 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAIRIGGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 51 - skipped

SEQ ID NO: 52 - protein, 236 aa, Homo sapiens
MDMRVPAQLLGLLLLWLRGARCDIQMTQSPSSLSASVGDRVTITCRASQSISGFLIWYQQKPGKAPKLLIYATSSLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - skipped

SEQ ID NO: 54 - protein, 461 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGLTGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 55 - skipped

SEQ ID NO: 56 - protein, 236 aa, Homo sapiens
MDMRVPAQLLGLLLLWLRGARCDIQMTQSPSSLSASVGDRVTITCRPSQIISSLLNWYQQKPGKAPKLLIYAASSLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - skipped

SEQ ID NO: 58 - protein, 467 aa, Homo sapiens
MELGLCWVFLVAILEGVQCEVQLVESGGGLVQPGGSLRLSCAASGFTFSSFSMTWVRQAPGKGLEWVSYISSRSSTISYADSVKGRFTISRDNAKNSLYLQMNSLRDEDTAVYYCARDPLLAGATFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 59 - skipped

SEQ ID NO: 60 - protein, 235 aa, Homo sapiens
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - skipped

SEQ ID NO: 62 - protein, 466 aa, Homo sapiens; identical to SEQ ID NO: 30

SEQ ID NO: 63-65 - skipped (3 entries)

SEQ ID NO: 66 - protein, 461 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAIRIGGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67-69 - skipped (3 entries)

SEQ ID NO: 70 - protein, 461 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIGGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 71-73 - skipped (3 entries)

SEQ ID NO: 74 - protein, 460 aa, Homo sapiens; identical to SEQ ID NO: 26

SEQ ID NO: 75-77 - skipped (3 entries)

SEQ ID NO: 78 - protein, 461 aa, Homo sapiens; identical to SEQ ID NO: 38

SEQ ID NO: 79-81 - skipped (3 entries)

SEQ ID NO: 82 - protein, 461 aa, Homo sapiens; identical to SEQ ID NO: 54

SEQ ID NO: 83-85 - skipped (3 entries)

SEQ ID NO: 86 - protein, 462 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARRIGGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 87-89 - skipped (3 entries)

SEQ ID NO: 90 - protein, 467 aa, Homo sapiens; identical to SEQ ID NO: 58

SEQ ID NO: 91-93 - skipped (3 entries)

SEQ ID NO: 94 - protein, 467 aa, Homo sapiens
MELGLCWVFLVAILEGVQCEVQLVESGGGLVQPGGSLRLSCAASGFTFSSFSMTWVRQAPGKGLEWVSYISSRSSTISYADSVKGRFTISRDNAKNSLYLQMNSLRDEDTAVYYCARDPLLAGATFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 95-96 - skipped (2 entries)

SEQ ID NO: 97 - DNA, 1413 bp, Homo sapiens
atggagttggggctgagctgggttttccttgttgctattataaaaggtgtccagtgtgaggtgcagctggtggagtctgggggaggcttggtacagcctggggggtccctgagactctcctgtgcagcctctggattcaccttcagtagttttagtatgacctgggtccgccaggctccaggaaaggggctggagtgggtttcatacattagtagtagaagtagtaccatatcctacgcagactctgtgaagggccgattcaccatctccagagacaatgccaagaactcactgtatctgcaaatgaacagcctgagagacgaggacacggctgtgtattactgtgcgagagatcctcttctagcgggagctaccttctttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 98 - protein, 470 aa, Homo sapiens
MELGLCWVFLVAILEGVQCEVQLVESGGGLVQPGGSLRLSCAASGFTFSSFSMTWVRQAPGKGLEWVSYISSRSSTISYADSVKGRFTISRDNAKNSLYLQMNSLRDEDTAVYYCARDPLLAGATFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99-100 - skipped (2 entries)

SEQ ID NO: 101 - DNA, 1395 bp, Homo sapiens
atggagtttgggctgagctgggttttccttgttgctattataaaaggtgtccagtgtcaggtgcagctggtggagtctgggggaggcttggtcaagcctggagggtccctgagactctcctgtgcagcctctggattcaccttcagtgactactatatgagctggatccgccaggctccagggaagggactggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagaggcctaactggggactactggggccagggaaccctggtcaccgtctcctcagcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 102 - protein, 464 aa, Homo sapiens
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGLTGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 104 - protein, 120 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYSGSYYYFDYWGQGTLVTVSSA

SEQ ID NO: 105 - protein, 119 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMSLRAEDTAVYYCARGIAAAGYFDYWGQGTLVTVSSA

SEQ ID NO: 106 - protein, 117 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARALGGMDVWGQGTTVTVSSA

SEQ ID NO: 107 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIK

SEQ ID NO: 108 - protein, 115 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARLTGDYWGQGTLVTVSSA

SEQ ID NO: 109 - protein, 108 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIKR

SEQ ID NO: 110 - protein, 117 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARGYSYFDYWGQGTLVTVSSA

SEQ ID NO: 111 - protein, 118 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCADYGGNYFDYWGQGTLVTVSSA

SEQ ID NO: 112 - protein, 114 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPWTFGQGTKVEIKR

SEQ ID NO: 113 - protein, 116 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCARIVGFDYWGQGTLVTVSSA

SEQ ID NO: 114 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKR

SEQ ID NO: 115 - protein, 114 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAIGMDVWGQGTTVTVSSA

SEQ ID NO: 116 - protein, 115 aa, Homo sapiens; identical to SEQ ID NO: 108

SEQ ID NO: 117 - protein, 108 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKR

(19) United States
(12) Patent Application Publication
(10) Pub. No.: US 2014/0286959 A1
(43) Pub. Date: Sep. 25, 2014

(54) Methods of Treating Inflammatory Disorders Using Anti-M-CSF Antibodies

(71) Applicant: Pfizer Inc., New York, NY (US)

(72) Inventors: Martin Hegen, Brookline, MA (US); Deborah Young, Melrose, MA (US); Heath Guay, Ambler, PA (US); Kyriaki Dunussi-Joannopoulos, Belmont, MA (US); Sudhakar Sridharan, Lansdale, PA (US); Annette Diehl, Collegeville, PA (US); Gail Comer, Phoenixville, PA (US); Margot O'Toole, Newtonville, MA (US); Jean Beebe, Acton, MA (US); Robert Fogel, Warren, NJ (US); Marek Honczarenko, Princeton, NJ (US); David Beidler, Newton, MA (US); Padmalatha S. Reddy, Lexington, MA (US); David J. von Schack, Arlington, MA (US)

(73) Assignee: PFIZER Inc., New York, NY (US)

(21) Appl. No.: 14/356,875

(22) PCT Filed: Nov. 2, 2012
(86) PCT No.: PCT/IB2012/056125

§ 371 Date: May 7, 2014

Related U.S. Application Data
(60) Provisional application No. 61/557,175, filed on Nov. 8, 2011.

(51) Int. Cl. C07K 16/24

(52) U.S. Cl. CPC C07K 16/243
USPC 424/145.1

(57) ABSTRACT

The present invention relates to antibodies and antigen-binding portions thereof that specifically bind to a M-CSF, preferably human M-CSF, and that function to inhibit a M-CSF. The invention also relates to human anti-M-CSF antibodies and antigen-binding portions thereof. The invention also relates to antibodies that are chimeric, bispecific, derivatized, single chain antibodies or portions of fusion proteins. The invention also relates to isolated heavy and light chain immunoglobulins derived from human anti-M-CSF antibodies and nucleic acid molecules encoding such immunoglobulins. The present invention also relates to methods of making human anti-M-CSF antibodies, compositions comprising these antibodies and methods of using the antibodies and compositions for diagnosis and treatment. The invention also provides gene therapy methods using nucleic acid molecules encoding the heavy and/or light immunoglobulin molecules that comprise the human anti-M-CSF antibodies. The invention also relates to transgenic animals and transgenic plants comprising nucleic acid molecules of the present invention.

### RELATED APPLICATIONS

This application claims the benefit of U.S. Provisional Application No. 61/557,175, filed Nov. 8, 2011, the entire contents of which are hereby incorporated by reference.

### BACKGROUND OF THE INVENTION

Macrophage colony stimulating factor (M-CSF) is a member of the family of proteins referred to as colony stimulating factors (CSFs). M-CSF is a secreted or a cell surface glycoprotein comprised of two subunits that are joined by a disulfide bond with a total molecular mass varying from 40 to 90 kD ((Stanley E. R., et al., Mol. Reprod. Dev., 46:4-10 (1997)). Similar to other CSFs, M-CSF is produced by macrophages, monocytes, and human joint tissue cells, such as chondrocytes and synovial fibroblasts, in response to proteins such as interleukin-1 or tumor necrosis factor-alpha. M-CSF stimulates the formation of macrophage colonies from pluripotent hematopoietic progenitor stem cells (Stanley E. R., et al., Mol. Reprod. Dev., 46:4-10 (1997)).

M-CSF typically bind to its receptor, c-fms, in order to exert a biological effect. c-fms contains five extracellular Ig domains, one transmembrane domain, and an intracellular domain with two kinase domains. Upon M-CSF binding to c-fms, the receptor homo-dimerizes and initiates a cascade of signal transduction pathways including the JAK/STAT, PI3K, and ERK pathways.

M-CSF is an important regulator of the function, activation, and survival of monocytes/macrophages. A number of animal models have confirmed the role of M-CSF in various diseases, including rheumatoid arthritis (RA) and cancer. Macrophages comprise key effector cells in RA. The degree of synovial macrophage infiltration in RA has been shown to closely correlate with the extent of underlying joint destruction. M-CSF, endogenously produced in the rheumatoid joint by monocytes/macrophages, fibroblasts, and endothelial cells, acts on cells of the monocyte/macrophage lineage to promote their survival and differentiation into bone destroying osteoclasts, and enhance proinflammatory cellular functions such as cytotoxicity, superoxide production, phagocytosis, chemotaxis and secondary cytokine production. For example, treatment with M-CSF in the rat streptococcus agalactiae sonicate-induced experimental arthritis model lead to enhanced pathology (Abd, A. H., et al., Lymphokine Cytokine Res. 10:43-50 (1991)). Similarly, subcutaneous injections of M-CSF in a murine model of collagen-induced arthritis (CIA), which is a model for RA, resulted in a significant exacerbation of the RA disease symptoms (Campbell I. K., et al., J. Leuk. Biol. 68:144-150 (2000)). Furthermore, MRL/Ipr mice that are highly susceptible to RA and other autoimmune diseases have elevated basal M-CSF serum concentrations (Yui M. A., et al., Am. J. Pathol. 139:255-261 (1991)). The requirement for endogenous M-CSF in maintaining CIA was demonstrated by a significant reduction in the severity of established disease by M-CSF neutralizing mouse monoclonal antibody (Campbell I. K. et al., J. Leuk. Biol. 68:144-150 (2000)).

With respect to cancer, inhibition of colony stimulating factors by antisense oligonucleotides suppresses tumor growth in embryonic and colon tumor xenografts in mice by decelerating macrophage-mediated ECM breakdown (Seyedhossein, A., et al., Cancer Research, 62:5317-5324 (2002)).

M-CSF binding to c-fms and its subsequent activation of monocyte/macrophages is important in a number of disease states. In addition to RA and cancer, the other examples of M-CSF-related disease states include osteoporosis, destructive arthritis, atherogenesis, glomerulonephritis, Kawasaki disease, and HIV-1 infection, in which monocytes/macrophages and related cell types play a role. For instance, osteoclasts are similar to macrophages and are regulated in part by M-CSF. Growth and differentiation signals induced by M-CSF in the initial stages of osteoclast maturation are essential for their subsequent osteoclastic activity in bone.

Osteoclast mediated bone loss, in the form of both focal bone erosions and more diffuse juxta-articular osteoporosis, is a major unsolved problem in RA. The consequences of this bone loss include joint deformities, functional disability, increased risk of bone fractures and increased mortality. M-CSF is uniquely essential for osteoclastogenesis and experimental blockade of this cytokine in animal models of arthritis successfully abrogates joint destruction. Similar destructive pathways are known to operate in other forms of destructive arthritis such as psoriatic arthritis, and could represent venues for similar intervention.

Postmenopausal bone loss results from defective bone remodeling secondary to an uncoupling of bone formation from exuberant osteoclast mediated bone resorption as a consequence of estrogen deficiency. In-vivo neutralization of M-CSF using a blocking antibody has been shown in mice to completely prevent the rise in osteoclast numbers, the increase in bone resorption and the resulting bone loss induced by ovariectomy.

Several lines of evidence point to a central role for M-CSF in atherogenesis, and in proliferative intimal hyperplasia after mechanical trauma to the arterial wall. All the major cell types in atherosclerotic lesions have been shown to express M-CSF, and this is further up-regulated by exposure to oxidized lipoprotein. Blockade of M-CSF signaling with a neutralizing c-fms antibody reduces the accumulation of macrophage-derived foam cells in the aortic root of apolipoprotein E deficient mice maintained on a high fat diet.

In both experimental and human glomerulonephritis, glomerular M-CSF expression has been found to co-localize with local macrophage accumulation, activation and proliferation and correlate with the extent of glomerular injury and proteinuria. Blockade of M-CSF signaling via an antibody directed against its receptor c-fms significantly down-regulates local macrophage accumulation in mice during the renal inflammatory response induced by experimental unilateral ureteric obstruction.

Kawasaki disease (KD) is an acute, febrile, pediatric vasculitis of unknown cause. Its most common and serious complications involve the coronary vasculature in the form of aneurismal dilatation. Serum M-CSF levels are significantly elevated in acute phase Kawasaki's disease, and normalize following treatment with intravenous immunoglobulin. Giant cell arthritis (GCA) is an inflammatory vasculopathy mainly occurring in the elderly in which T cells and macrophages infiltrate the walls of medium and large arteries leading to clinical consequences that include blindness and stroke secondary to arterial occlusion. The active involvement of macrophages in GCA is evidenced by the presence of elevated levels of macrophage derived inflammatory mediators within vascular lesions.

M-CSF has been reported to render human monocyte derived macrophages more susceptible to HIV-1 infection in vitro. In a recent study, M-CSF increased the frequency with which monocyte-derived macrophages became infected, the amount of HIV mRNA expressed per infected cell, and the level of proviral DNA expressed per infected culture.

Given the role of M-CSF in various diseases, a method for inhibiting M-CSF activity is desirable.

There is a critical need for therapeutic anti-M-CSF antibodies.

### SUMMARY OF THE INVENTION

The present invention provides isolated human antibodies or antigen-binding portions thereof that specifically bind human M-CSF and acts as a M-CSF antagonist and compositions comprising said antibody or portion.

The invention also provides for compositions comprising the heavy and/or light chain, the variable regions thereof, or antigen-binding portions thereof an anti-M-CSF antibody, or nucleic acid molecules encoding an antibody, antibody chain or variable region thereof the invention effective in such treatment and a pharmaceutically acceptable carrier. In certain embodiments, the compositions may further comprise another component, such as a therapeutic agent or a diagnostic agent. Diagnostic and therapeutic methods are also provided by the invention. In certain embodiments, the compositions are used in a therapeutically effective amount necessary to treat or prevent a particular disease or condition.

The invention also provides methods for treating or preventing a variety of diseases and conditions such as, but not limited to, lupus, inflammation, cancer, atherogenesis, neurological disorders and cardiac disorders with an effective amount of an anti-M-CSF antibody of the invention, or antigen binding portion thereof, nucleic acids encoding said antibody, or heavy and/or light chain, the variable regions, or antigen-binding portions thereof.

The invention provides isolated cell lines, such as a hybridomas, that produce anti-M-CSF antibodies or antigen-binding portions thereof.

The invention also provides nucleic acid molecules encoding the heavy and/or light chains of anti-M-CSF antibodies, the variable regions thereof, or the antigen-binding portions thereof.

The invention provides vectors and host cells comprising the nucleic acid molecules, as well as methods of recombinantly producing the polypeptides encoded by the nucleic acid molecules.

Non-human transgenic animals or plants that express the heavy and/or light chains, or antigen-binding portions thereof, of anti-M-CSF antibodies are also provided.

The anti-M-CSF antibodies or antigen binding portions thereof, compositions, cell lines, nucleic acid molecules, vectors, host cells, and methods of treating or prevent diseases using the anti-M-CSF antibodies are fully described herein as well as in PCT Application Number PCT/US2004/029390, published as WO 2005/030124, which is incorporated herein by reference in its entirety.

### BRIEF DESCRIPTION OF THE DRAWINGS

FIGS. 1A and 2B are graphs illustrating that the anti-M-CSF antibodies resulted in a dose-related decrease in total monocyte counts in male and female monkeys over time. The monocyte counts were determined by light scatter using an Abbott Diagnostics Inc. Cell Dyn system. Monocyte counts were monitored from 24 hours through 3 weeks after administration of vehicle or antibody 8.10.3 at 0, 0.1, 1 or 5 mg/kg in a dose volume of 3.79 mL/kg over an approximately 5 minute period.

FIG. 1A male monkeys.

FIG. 1B female monkeys.

FIGS. 2A and 2B are graphs illustrating that anti-M-CSF treatment resulted in a reduction in the percentage of CD14+CD16+ monocytes, in male and female monkeys. 0-21 days after administration of vehicle or antibody 8.10.3 at 0, 0.1, 1 or 5 mg/kg in a dose volume of 3.79 mL/kg over an approximately 5 minute period. For each monkey tested, the percentage of monocytes within the CD14+CD16+ subset was determined after each blood draw, on days 1, 3, 7, 14 and 21 after 8.10.3 injection.

FIG. 2A male monkeys.

FIG. 2B female monkeys.

FIGS. 3A and 3B are graphs illustrating that anti-M-CSF treatment resulted in a decrease in the percentage change of total monocytes at all doses of antibody 8.10.3F and antibody 9.14.4I as compared to pre-test levels of monocytes.

FIG. 3A shows data collected from experiments using antibody 8.10.3F.

FIG. 3B shows data collected from experiments using antibody 9.14.4I.

FIG. 4 is a sequence alignment of the predicted amino acid sequences of light and heavy chain variable regions from twenty-six anti-M-CSF antibodies compared with the germline amino acid sequences of the corresponding variable region genes. Differences between the antibody sequences and the germline gene sequences are indicated in bold-faced type. Dashes represent no change from germline. The underlined sequences in each alignment represent, from left to right, the FR1, CDR1, FR2, CDR2, FR3, CDR3 AND FR4 sequences.

FIG. 4A shows an alignment of the predicted amino acid sequence of the light chain variable region for antibody 252 (residues 21-127 of SEQ ID NO: 4) to the germline V_{K}O12, J_{K}3 sequence (SEQ ID NO: 103).

FIG. 4B shows an alignment of the predicted amino acid sequence of the light chain variable region for antibody 88 (residues 21-127 of SEQ ID NO: 8) to the germline V_{K}O12, J_{K}3 sequence (SEQ ID NO: 103).

FIG. 4C shows an alignment of the predicted amino acid sequence of the light chain variable region for antibody 100 (residues 21-127 of SEQ ID NO: 12) to the germline V_{K}L2, J_{K}3 sequence (SEQ ID NO: 107).

FIG. 4D shows an alignment of the predicted amino acid sequence of the light chain variable region for antibody 3.8.3 (residues 23-130 of SEQ ID NO: 16) to the germline V_{K}L5, J_{K}3 sequence (SEQ ID NO: 109).

FIG. 4E shows an alignment of the predicted amino acid sequence of the light chain variable region for antibody 2.7.3 (residues 23-130 of SEQ ID NO: 20) to the germline V_{K}L5, J_{K}4 sequence (SEQ ID NO: 117).

FIG. 4F shows an alignment of the predicted amino acid sequence of the light chain variable region for antibody 1.120.1 (residues 21-134 of SEQ ID NO: 24) to the germline V_{K}B3, J_{K}1 sequence (SEQ ID NO: 112).

FIG. 4G shows an alignment of the predicted amino acid sequence of the heavy chain variable region for antibody 252 (residues 20-136 of SEQ ID NO: 2) to the germline V_{H}3-11, D_{H}7-27 J_{H}6 sequence (SEQ ID NO: 106).

FIG. 4H shows an alignment of the predicted amino acid sequence of the heavy chain variable region for antibody 88 (residues 20-138 of SEQ ID NO: 6) to the germline V_{H}3-7, D_{H}6-13, J_{H}4 sequence (SEQ ID NO: 105).

FIG. 4I shows the alignment of the predicted amino acid sequence of the heavy chain variable region for antibody 100 (residues 20-141 of SEQ ID NO: 10) to the germline V_{H}3-23, D_{H}1-26, J_{H}4 sequence (SEQ ID NO: 104).

FIG. 4J shows an alignment of the predicted amino acid sequence of the heavy chain variable region for antibody 3.8.3 (residues 20-135 of SEQ ID NO: 14) to the germline V_{H}3-11, D_{H}7-27, J_{H}4 sequence (SEQ ID NO: 108).

FIG. 4K shows an alignment of the predicted amino acid sequence of the heavy chain variable region for antibody 2.7.3 (residues 20-137 of SEQ ID NO: 18) to the germline V_{H}3-33, D_{H}1-26, J_{H}4 sequence (SEQ ID NO: 110).

FIG. 4L shows an alignment of the predicted amino acid sequence of the heavy chain variable region for antibody 1.120.1 (residues 20-139 of SEQ ID NO: 22) to the germline V_{H}1-18, D_{H}4-23, J_{H}4 sequence (SEQ ID NO: 111).

FIG. 4M shows an alignment of the predicted amino acid sequence of the light chain variable region for antibody 8.10.3 (residues 21-129 of SEQ ID NO: 44) to the germline V_{K}A27, J_{K}4 sequence (SEQ ID NO: 114).

FIG. 4N shows an alignment of the predicted amino acid sequence of the heavy chain variable region for antibody 8.10.3 (residues 20-141 of SEQ ID NO: 30) to the germline V_{H}3-48. D_{H}1-26, J_{H}4b sequence (SEQ ID NO: 113).

FIG. 4O shows an alignment of the predicted amino acid sequence of the light chain variable region for antibody 9.14.4 (residues 23-130 of SEQ ID NO: 28) to the germline V_{K}O12, J_{K}3 sequence (SEQ ID NO: 103).

FIG. 4P shows an alignment of the predicted amino acid sequence of the heavy chain variable region for antibody 9.14.4 (residues 20-135 of SEQ ID NO: 38) to the germline V_{H}3-11, D_{H}7-27, J_{H}4b sequence (SEQ ID NO: 116).

FIG. 4Q shows an alignment of the predicted amino acid sequence of the light chain variable region for antibody 9.7.2 (residues 23-130 of SEQ ID NO: 48) to the germline V_{K}O12, J_{K}3 sequence (SEQ ID NO: 103).

FIG. 4R shows an alignment of the predicted amino acid sequence of the heavy chain variable region for antibody 9.7.2 (residues 20-136 of SEQ ID NO: 46) to the germline V_{H}3-11, D_{H}6-13, J_{H}6b sequence (SEQ ID NO: 115).

FIG. 4S shows an alignment of the predicted amino acid sequence of the light chain variable region for antibody 9.14.4I (residues 23-130 of SEQ ID NO: 28) to the germline V_{K}O12 J_{K}3 sequence (SEQ ID NO: 103).

FIG. 4T shows an alignment of the predicted amino acid sequence of the heavy chain variable region for antibody 9.14.4I (residues 20-135 of SEQ ID NO: 26) to the germline V_{H}3-11, D_{H}7-27, J_{H}4b sequence (SEQ ID NO: 116).

FIG. 4U shows an alignment of the predicted amino acid sequence of the light chain variable region for antibody 8.10.3F (residues 21-129 of SEQ ID NO: 32) to the germline V_{K}A27, J_{K}4 sequence (SEQ ID NO: 114).

FIG. 4V shows an alignment of the predicted amino acid sequence of the heavy chain variable region for antibody 8.10.3F (residues 20-141 of SEQ ID NO: 30) to the germline V_{H}3-48, D_{H}1-26, J_{H}4b sequence (SEQ ID NO: 113).

FIG. 4W shows an alignment of the predicted amino acid sequence of the light chain variable region for antibody 9.7.2IF (residues 23-130 of SEQ ID NO: 36) to the germline V_{K}O12, J_{K}3 sequence (SEQ ID NO: 103).

FIG. 4X shows an alignment of the predicted amino acid sequence of the heavy chain variable region for antibody 9.7.2IF (residues 20-136 of SEQ ID NO: 34) to the germline V_{H}3-1, D_{H}6-13, J_{H}6b sequence (SEQ ID NO: 115).

FIG. 4Y shows an alignment of the predicted amino acid sequence of the light chain variable region for antibody 9.7.2C-Ser (residues 23-130 of SEQ ID NO: 52) to the germline V_{K}O12, J_{K}3 sequence (SEQ ID NO: 103).

FIG. 4Z shows an alignment of the predicted amino acid sequence of the heavy chain variable region for antibody 9.7.2C-Ser (residues 20-136 of SEQ ID NO: 50) to the germline V_{H}3-11, D_{H}6-13, J_{H}6b sequence (SEQ ID NO: 115).

FIG. 4AA shows an alignment of the predicted amino acid sequence of the light chain variable region for antibody 9.14.4C-Ser (residues 23-130 of SEQ ID NO: 56) to the germline V_{K}O12, J_{K}3 sequence (SEQ ID NO: 103).

FIG. 4BB shows an alignment of the predicted amino acid sequence of the heavy chain variable region for antibody 9.14.4C-Ser (residues 20-135 of SEQ ID NO: 54) to the germline V_{H}3-11, D_{H}7-27, J_{H}4b sequence (SEQ ID NO: 116).

FIG. 4CC shows an alignment of the predicted amino acid sequence of the light chain variable region for antibody 8.10.3C-Ser (residues 21-129 of SEQ ID NO: 60) to the germline V_{K}A27, J_{K}4 sequence (SEQ ID NO: 114).

FIG. 4DD shows an alignment of the predicted amino acid sequence of the heavy chain variable region for antibody 8.10.3C-Ser (residues 20-141 of SEQ ID NO: 58) to the germline V_{H}3-48, D_{H}1-26, J_{H}4b sequence (SEQ ID NO: 113).

FIG. 4EE shows an alignment of the predicted amino acid sequence of the light chain variable region for antibody 8.10.3-CG2 (residues 21-129 of SEQ ID NO: 60) to the germline V_{K}A27, J_{K}4 sequence (SEQ ID NO: 114).

FIG. 4FF shows an alignment of the predicted amino acid sequence of the heavy chain variable region for antibody 8.10.3-CG2 (residues 20-141 of SEQ ID NO: 62) to the germline V_{H}3-48, D_{H}1-26, J_{H}4b sequence (SEQ ID NO: 113).

FIG. 4GG shows an alignment of the predicted amino acid sequence of the light chain variable region for antibody 9.7.2-CG2 (residues 23-130 of SEQ ID NO: 52) to the germline V_{K}O12, J_{K}3 sequence (SEQ ID NO: 103).

FIG. 4HH shows an alignment of the predicted amino acid sequence of the heavy chain variable region for antibody 9.7.2-CG2 (residues 20-136 of SEQ ID NO: 66) to the germline V_{H}3-11, D_{H}6-13, J_{H}6b sequence (SEQ ID NO: 115).

FIG. 4II shows an alignment of the predicted amino acid sequence of the light chain variable region for antibody 9.7.2-CG4 (residues 23-130 of SEQ ID NO: 52) to the germline V_{K}O12, J_{K}3 sequence (SEQ ID NO: 103).

FIG. 4JJ shows an alignment of the predicted amino acid sequence of the heavy chain variable region for antibody 9.7.2-CG4 (residues 20-135 of SEQ ID NO: 70) to the germline V_{H}3-11, D_{H}6-13, J_{H}6b sequence (SEQ ID NO: 115).

FIG. 4KK shows an alignment of the predicted amino acid sequence of the light chain variable region for antibody 9.14.4-CG2 (residues 23-130 of SEQ ID NO: 56) to the germline V_{K}O12. J_{K}3 sequence (SEQ ID NO: 103).

FIG. 4LL shows an alignment of the predicted amino acid sequence of the heavy chain variable region for antibody 9.14.4-CG2 (residues 20-135 of SEQ ID NO: 74) to the germline V_{H}3-11, D_{H}7-27, J_{H}4b sequence (SEQ ID NO: 116).

FIG. 4MM shows an alignment of the predicted amino acid sequence of the light chain variable region for antibody 9.14.4-CG4 (residues 23-130 of SEQ ID NO: 56) to the germline V_{K}O12, J_{K}3 sequence (SEQ ID NO: 103).

FIG. 4NN shows an alignment of the predicted amino acid sequence of the heavy chain variable region for antibody 9.14.4-CG4 (residues 20-135 of SEQ ID NO: 78) to the germline V_{H}3-11, D_{H}7-27, J_{H}4b sequence (SEQ ID NO: 116).

FIG. 4OO shows an alignment of the predicted amino acid sequence of the light chain variable region for antibody 9.14.4-Ser (residues 23-130 of SEQ ID NO: 28) to the germline V_{K}O12, J_{K}3 sequence (SEQ ID NO: 103).

FIG. 4PP shows an alignment of the predicted amino acid sequence of the heavy chain variable region for antibody 9.14.4-Ser (residues 20-135 of SEQ ID NO: 82) to the germline V_{H}3-11, D_{H}7-27, J_{H}4b sequence (SEQ ID NO: 116).

FIG. 4QQ shows an alignment of the predicted amino acid sequence of the light chain variable region for antibody 9.7.2-Ser (residues 23-130 of SEQ ID NO: 48) to the germline V_{K}O12, J_{K}3 sequence (SEQ ID NO: 103).

FIG. 4RR shows an alignment of the predicted amino acid sequence of the heavy chain variable region for antibody 9.7.2-Ser (residues 20-136 of SEQ ID NO: 86) to the germline V_{H}3-11, D_{H}6-13, J_{H}6b sequence (SEQ ID NO: 115).

FIG. 4SS shows an alignment of the predicted amino acid sequence of the light chain variable region for antibody 8.10.3-Ser (residues 21-129 of SEQ ID NO: 44) to the germline V_{K}A27, J_{K}4 sequence (SEQ ID NO: 114).

FIG. 4TT shows an alignment of the predicted amino acid sequence of the heavy chain variable region for antibody 8.10.3-Ser (residues 20-141 of SEQ ID NO: 90) to the germline V_{H}3-48, D_{H}1-26, J_{H}4b sequence (SEQ ID NO: 113).

FIG. 4UU shows an alignment of the predicted amino acid sequence of the light chain variable region for antibody 8.10.3-CG4 (residues 21-129 of SEQ ID NO: 60) to the germline V_{K}A27, J_{K}4 sequence (SEQ ID NO: 114).

FIG. 4W shows an alignment of the predicted amino acid sequence of the heavy chain variable region for antibody 8.10.3-CG4 (residues 20-141 of SEQ ID NO: 94) to the germline V_{H}3-48, D_{H}1-26, J_{H}4b sequence (SEQ ID NO: 113).

FIG. 4WW shows an alignment of the predicted amino acid sequence of the light chain variable region for antibody 9.14.4G1 (residues 23-130 of SEQ ID NO: 28) to the germline V_{K}O12 J_{K}3 sequence (SEQ ID NO: 103).

FIG. 4XX shows an alignment of the predicted amino acid sequence of the heavy chain variable region for antibody 9.14.4G1 (residues 20-135 of SEQ ID NO: 102) to the germline V_{H}3-11, D_{H}7-27, J_{H}4b sequence (SEQ ID NO: 116).

FIG. 4YY shows an alignment of the predicted amino acid sequence of the light chain variable region for antibody 8.10.3FG1 (residues 21-129 of SEQ ID NO:32) to the germline V_{K}A27, J_{K}4 sequence (SEQ ID NO: 114).

FIG. 4ZZ shows an alignment of the predicted amino acid sequence of the heavy chain variable region for antibody 8.10.3FG1 (residues 20-141 of SEQ ID NO: 98) to the germline V_{H}3-48, D_{H}1-26, J_{H}4b sequence (SEQ ID NO: 113).

FIG. 5 is a graph depicting the effect of anti-M-CSF antibody on the development of lymphadenopathy in the murine MRL-Ipr model of lupus. Mice (n=10/group) were dosed with saline (diamond), anti-M-CSF Ab, 5A1 (square), CHOCK IgG1 (triangle) or CTLA-4Ig (X) 3×/wk for 12 weeks and scored for lymphadenopathy as described in the Examples. *Anti-M-CSF treated group is significantly different (p<0.05) from saline. **Anti-M-CSF-treated group is significantly different (p<0.05) from saline and CTLA-4Ig. Anti-M-CSF group was not significantly different from CHOCK IgG1 treated group (p=0.065, weeks 6 through 12).

FIG. 6 is a graph depicting the effect of anti-M-CSF antibody on the development of skin lesions in the murine MRL-Ipr model of lupus. Mice (n=10 per group) were dosed with saline (diamond), anti-M-CSF Ab, 5A1 (square), CHOCK IgG1 (triangle) or CTLA-4Ig (X) 3×/week for 12 weeks and scored for skin lesions as described in the Examples. *Anti-M CSF treated group is significantly different (p<0.05) from saline. **Anti-M CSF-treated group is significantly different (p<0.05) from saline and CTLA-4Ig. Anti-M CSF group was not significantly different from CHOCK IgG1 treated group (p=0.065, weeks 6 through 12).

FIG. 7 is a graph depicting the effect of anti-M-CSF antibody on the development of anti-dsDNA autoantibodies in murine MRL-Lpr model of lupus. Anti-dsDNA antibody titers were determined at 4 time points for mice treated with saline (diamond), CTLA-4Ig (square), anti-M CSF Ab 5A1 (triangle) or CHOCK IgG1 isotype control (X). *CTLA-4Ig is significantly different from saline and anti-M CSF (p<0.05). **Anti-M CSF-treated group is significantly different (p<0.05) from CHOCK IgG1.

FIG. 8 is a graph depicting the effect of anti-M-CSF antibody on glomerular nuclear area and C3 deposition in murine MRL-Ipr model of lupus. Glomerular nuclear area (left) and immunohistochemical staining for C3 deposition (right) was determined microscopically from kidneys harvested at the termination of study for mice treated with saline, CTLA-4Ig, anti-M CSF Ab 5A1, or CHOCK IgG1 isotype control. Data bars represent average mean score and lines are standard error.

FIG. 9 is a graph depicting the effect of anti-M-CSF antibody on the development of proteinuria in the murine NZBWF1/J lupus model. Mice treated with saline (square), CHOCK IgG1 isotype control (triangle) or anti-M CSF antibody (circle), were scored for proteinuria using the following scale: 0=negative; 1=trace; 2=>30 mg/dL; 3=>100 mg/dL; 4=>300 mg/dL; and 5=>2000 mg/dL. FIG. 9A depicts mean proteinuria scores for each group measured biweekly. FIG. 9B depicts individual mouse proteinuria scores at week 10.

FIG. 10 is a graph depicting the effect of anti-M-CSF antibody on the development of anti-dsDNA antibody titres in the murine NZBWF1/J lupus model. Anti-dsDNA antibody levels were determined in mice treated with saline (circle), CHOCK IgG1 isotype control (triangle) or anti-M CSF antibody (diamond) by ELISA as described in the Examples. FIG. 10A depicts the antibody titres at the week 6 time point. FIG. 10B depicts the antibody titres at the week 10 time point.

FIG. 11 is a graph depicting the effect of anti-M-CSF antibody on serum levels of M-CSF in the murine NZBWF1/J lupus model. Serum levels of M-CSF were determined by specific ELISA from serum collected at the termination of the study from saline (circle), isotype control (square) or anti-M CSF (triangle) treated mice.

FIG. 12 is a graph depicting the effect of anti-M-CSF antibody on immune complex deposition and macrophage infiltration in the kidney of NZBWF1/J mice. Bars represent group mean renal immunohistochemical staining scores for mice treated with saline, CHOCK IgG1 control, or anti-M-CSF antibody, and lines indicate standard errors.

FIG. 13 is a graph depicting mean serum antibody 8.10.3F concentration time profiles following administration of single intravenous solution doses to healthy subjects. Upper and lower panels are linear and semi-logarithmic scales, respectively. Legend is antibody dose in mg. Concentrations after 700 hours were either zero (below limit of quantitation) or represented <3 subjects.

FIG. 14 is a graph depicting antibody 8.10.3F Cmax (Upper Panel) and AUC(0-∞) (Lower Panel) values following administration of single intravenous solution doses to healthy subjects. Left panels show observed values; right panels show dose-normalized values. Circles are individual subjects, diamonds are arithmetic means

FIG. 15 is a graph depicting mean antibody 8.10.3F (open squares) and M-CSF (X) concentrations following administration of a single intravenous 100-mg dose to healthy subjects.

FIG. 16 is a graph depicting CD14⁺16⁺ monocyte dose response on Study Day 28 following administration of single intravenous solution doses to healthy subjects.

FIG. 17 is a graph depicting CD14⁺16⁺ time response following administration of a single, 100-mg intravenous solution dose to healthy subjects.

FIG. 18 is a graph depicting mean antibody 8.10.3F (open squares) concentrations and CD14⁺16⁺ monocyte counts (X) following administration of single intravenous 100-mg doses to healthy subjects,

FIG. 19 is a graph depicting mean antibody 8.10.3F concentrations (open squares) and uNTX-1 (X) following administration of a single intravenous 100-mg dose to healthy subjects.

### DETAILED DESCRIPTION OF THE INVENTION

### Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel at al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

The term “polypeptide” encompasses native or artificial proteins, protein fragments and polypeptide analogs of a protein sequence. A polypeptide may be monomeric or polymeric.

The term “isolated protein”, “isolated polypeptide” or “isolated antibody” is a protein, polypeptide or antibody that by virtue of its origin or source of derivation has one to four of the following: (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

Examples of isolated antibodies include an anti-M-CSF antibody that has been affinity purified using M-CSF, an anti-M-CSF antibody that has been synthesized by a hybridoma or other cell line in vitro, and a human anti-M-CSF antibody derived from a transgenic mouse.

A protein or polypeptide is “substantially pure,” “substantially homogeneous”, or “substantially purified” when at least about 60 to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide fragment” as used herein refers to a polypeptide that has an amino-terminal and/or carboxy-terminal deletion, but where the remaining amino acid sequence is identical to the corresponding positions in the naturally-occurring sequence. In some embodiments, fragments are at least 5, 6, 8 or 10 amino acids long. In other embodiments, the fragments are at least 14, at least 20, at least 50, or at least 70, 80, 90, 100, 150 or 200 amino acids long.

The term “polypeptide analog” as used herein refers to a polypeptide that comprises a segment that has substantial identity to a portion of an amino acid sequence and that has at least one of the following properties: (1) specific binding to M-CSF under suitable binding conditions, (2) ability to inhibit M-CSF.

Typically, polypeptide analogs comprise a conservative amino acid substitution (or insertion or deletion) with respect to the normally-occurring sequence. Analogs typically are at least 20 or 25 amino acids long, preferably at least 50, 60, 70, 80, 90, 100, 150 or 200 amino acids long or longer, and can often be as long as a full-length polypeptide.

In certain embodiments, amino acid substitutions of the antibody or antigen-binding portion thereof are those which: (1) reduce susceptibility to proteolysis, (2) reduce susceptibility to oxidation, (3) alter binding affinity for forming protein complexes, or (4) confer or modify other physicochemical or functional properties of such analogs. Analogs can include various muteins of a sequence other than the normally-occurring peptide sequence. For example, single or multiple amino acid substitutions (preferably conservative amino acid substitutions) may be made in the normally-occurring sequence, preferably in the portion of the polypeptide outside the domain(s) forming intermolecular contacts.

A conservative amino acid substitution should not substantially change the structural characteristics of the parent sequence; e.g., a replacement amino acid should not alter the anti-parallel β-sheet that makes up the immunoglobulin binding domain that occurs in the parent sequence, or disrupt other types of secondary structure that characterizes the parent sequence. In general, glycine and proline analogs would not be used in an anti-parallel β-sheet. Examples of art-recognized polypeptide secondary and tertiary structures are described in Proteins, Structures and Molecular Principles (Creighton. Ed. W. H. Freeman and Company, New York (1984)); Introduction to Protein Structure (C. Branden and J. Tooze, eds. Garland Publishing, New York, N.Y. (1991)); and Thornton et al., Nature 354:105 (1991), which are each incorporated herein by reference.

Non-peptide analogs are commonly used in the pharmaceutical industry as drugs with properties analogous to those of the template peptide. These types of non-peptide compound are termed “peptide mimetics” or “peptidomimetics.” Fauchere, J. Adv. Drug Res. 15:29 (1986): Veber and Freidinger, TINS p. 392 (1985); and Evans et al., J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference); for example, by adding internal cysteine residues capable of forming intramolecular disulfide bridges which cyclize the peptide.

An “antibody” refers to an intact antibody or an antigen-binding portion that competes with the intact antibody for specific binding. See generally, Fundamental Immunology, Ch. 7 (Paul, W., ed., 2nd ed. Raven Press, N.Y. (1989)) (incorporated by reference in its entirety for all purposes). Antigen-binding portions may be produced by recombinant DNA techniques or by enzymatic or chemical cleavage of intact antibodies. In some embodiments, antigen-binding portions include Fab, Fab′, F(ab′)₂, Fd, Fv, dAb, and complementarity determining region (CDR) fragments, single-chain antibodies (scFv), chimeric antibodies, diabodies and polypeptides that contain at least a portion of an antibody that is sufficient to confer specific antigen binding to the polypeptide.

From N-terminus to C-terminus, both the mature light and heavy chain variable domains comprise the regions FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987), or Chothia et al., Nature 342:878-883 (1989).

As used herein, an antibody that is referred to by number is the same as a monoclonal antibody that is obtained from the hybridoma of the same number. For example, monoclonal antibody 3.8.3 is the same antibody as one obtained from hybridoma 3.8.3.

As used herein, a Fd fragment means an antibody fragment that consists of the V_{H} and C_{H} 1 domains; an Fv fragment consists of the V_{L} and V_{H} domains of a single arm of an antibody; and a dAb fragment (Ward et al., Nature 341:544-546 (1989)) consists of a V_{H} domain.

In some embodiments, the antibody is a single-chain antibody (scFv) in which a V_{L} and a V_{H} domain are paired to form a monovalent molecule via a synthetic linker that enables them to be made as a single protein chain. (Bird et al., Science 242:423-426 (1988) and Huston et al., Proc. Natl. Acad. Sci. USA 85:5879-5883 (1988).) In some embodiments, the antibodies are diabodies, i.e., are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites. (See e.g., Holliger P. et al., Proc. Natl. Acad. Sci. USA 90:6444-6448 (1993), and Poljak R. J. et al., Structure 2:1121-1123 (1994).) In some embodiments, one or more CDRs from an antibody of the invention may be incorporated into a molecule either covalently or noncovalently to make it an immunoadhesin that specifically binds to M-CSF. In such embodiments, the CDR(s) may be incorporated as part of a larger polypeptide chain, may be covalently linked to another polypeptide chain, or may be incorporated noncovalently.

In embodiments having one or more binding sites, the binding sites may be identical to one another or may be different.

As used herein, the term “human antibody” means any antibody in which the variable and constant domain sequences are human sequences. The term encompasses antibodies with sequences derived from human genes, but which have been changed, e.g. to decrease possible immunogenicity, increase affinity, eliminate cysteines that might cause undesirable folding, etc. The term emcompasses such antibodies produced recombinantly in non-human cells, which might impart glycosylation not typical of human cells. These antibodies may be prepared in a variety of ways, as described below.

The term “chimeric antibody” as used herein means an antibody that comprises regions from two or more different antibodies. In one embodiment, one or more of the CDRs are derived from a human anti-M-CSF antibody. In another embodiment, all of the CDRs are derived from a human anti-M-CSF antibody. In another embodiment, the CDRs from more than one human anti-M-CSF antibodies are combined in a chimeric antibody. For instance, a chimeric antibody may comprise a CDR1 from the light chain of a first human anti-M-CSF antibody, a CDR2 from the light chain of a second human anti-M-CSF antibody and a CDR3 from the light chain of a third human anti-M-CSF antibody, and the CDRs from the heavy chain may be derived from one or more other anti-M-CSF antibodies. Further, the framework regions may be derived from one of the anti-M-CSF antibodies from which one or more of the CDRs are taken or from one or more different human antibodies.

Fragments or analogs of antibodies or immunoglobulin molecules can be readily prepared by those of ordinary skill in the art following the teachings of this specification. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. See Bowie et al., Science 253:164 (1991).

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.). For further descriptions, see Jonsson U. et al., Ann. Biol. Clin. 51:19-26 (1993); Jonsson U. et al., Biotechniques 11:620-627 (1991); Jonsson B. et al., J. Mol. Recognit. 8:125-131 (1995); and Johnsson B. et al., Anal. Biochem. 198:268-277 (1991).

The term “K_{D}” refers to the equilibrium dissociation constant of a particular antibody-antigen interaction.

The term “epitope” includes any protein determinant capable of specific binding to an immunoglobulin or T-cell receptor or otherwise interacting with a molecule. Epitopic determinants generally consist of chemically active surface groupings of molecules such as amino acids or sugar side chains and generally have specific three dimensional structural characteristics, as well as specific charge characteristics. An epitope may be “linear” or “conformational.” In a linear epitope, all of the points of interaction between the protein and the interacting molecule (such as an antibody) occur linearally along the primary amino acid sequence of the protein. In a conformational epitope, the points of interaction occur across amino acid residues on the protein that are separated from one another. An antibody is said to specifically bind an antigen when the dissociation constant is ≦1 mM, preferably ≦100 nM and most preferably ≦10 nM. In certain embodiments, the K_{D} is 1 μM to 500 μM. In other embodiments, the K_{D} is between 500 μM to 1 μM. In other embodiments, the K_{D} is between 1 μM to 100 nM. In other embodiments, the K_{D} is between 100 mM to 10 nM. Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. A high throughout process for “binning” antibodies based upon their cross-competition is described in International Patent Application No. WO 03/48731.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference.

The term “polynucleotide” as referred to herein means a polymeric form of nucleotides of at least 10 bases in length, either ribonucleotides or deoxynucleotides or a modified form of either type of nucleotide. The term includes single and double stranded forms.

The term “isolated polynucleotide” as used herein means a polynucleotide of genomic, cDNA, or synthetic origin or some combination thereof, which by virtue of its origin or source of derivation, the “isolated polynucleotide” has one to three of the following: (1) is not associated with all or a portion of a polynucleotides with which the “isolated polynucleotide” is found in nature, (2) is operably linked to a polynucleotide to which it is not linked in nature, or (3) does not occur in nature as part of a larger sequence.

The term “oligonucleotide” as used herein includes naturally occurring, and modified nucleotides linked together by naturally occurring and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for primers and probes; although oligonucleotides may be double stranded, e.g. for use in the construction of a gene mutant. Oligonucleotides of the invention can be either sense or antisense oligonucleotides.

The term “naturally occurring nucleotides” as used herein includes deoxyribonucleotides and ribonucleotides. The term “modified nucleotides” as used herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al., Nucl. Acids Res. 14:9081 (1986); Stec et al., J. Am. Chem. Soc. 106:6077 (1984); Stein et al., Nucl. Acids Res. 16:3209 (1988); Zon et al., Anti-Cancer Drug Design 6:539 (1991); Zon et al., Oligonucleotides and Analogues: A Practical Approach, pp. 87-108 (F. Eckstein, Ed., Oxford University Press, Oxford England (1991)); U.S. Pat. No. 5,151,510; Uhlmann and Peyman, Chemical Reviews 90:543 (1990), the disclosures of which are hereby incorporated by reference. An oligonucleotide can include a label for detection, if desired.

“Operably linked” sequences include both expression control sequences that are contiguous with the gene of interest and expression control sequences that act in trans or at a distance to control the gene of interest. The term “expression control sequence” as used herein means polynucleotide sequences that are necessary to effect the expression and processing of coding sequences to which they are ligated. Expression control sequences include appropriate transcription initiation, termination, promoter and enhancer sequences: efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, means a nucleic acid molecule capable of transporting another nucleic acid to which it has been linked. In some embodiments, the vector is a plasmid, i.e., a circular double stranded DNA loop into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The term “recombinant host cell” (or simply “host cell”), as used herein, means a cell into which a recombinant expression vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. One example of “high stringency” or “highly stringent” conditions is the incubation of a polynucleotide with another polynucleotide, wherein one polynucleotide may be affixed to a solid surface such as a membrane, in a hybridization buffer of 6×SSPE or SSC, 50% formamide, 5×Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

The term “percent sequence identity” in the context of nucleic acid sequences means the percent of residues when a first contiguous sequence is compared and aligned for maximum correspondence to a second contiguous sequence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183:63-98 (1990); Pearson, Methods Mol. Biol. 132:185-219 (2000); Pearson, Methods Enzymol. 266:227-258 (1996); Pearson, J. Mol. Biol. 276:71-84 (1998); herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleotide sequence encompasses its complement unless otherwise specified. Thus, a reference to a nucleic acid having a particular sequence should be understood to encompass its complementary strand, with its complementary sequence.

The term “percent sequence identity” means a ratio, expressed as a percent of the number of identical residues over the number of residues compared.

The term “substantial similarity” or “substantial sequence similarity,” when referring to a nucleic acid or fragment thereof, means that when optimally aligned with appropriate nucleotide insertions or deletions with another nucleic acid (or its complementary strand), there is nucleotide sequence identity in at least about 85%, preferably at least about 90%, 91%, 92%, 93%, 94%, 95%, 96%, 97%, 98% or 99% of the nucleotide bases, as measured by any well-known algorithm of sequence identity, such as FASTA, BLAST or Gap, as discussed above.

As applied to polypeptides, the term “substantial identity” means that two peptide sequences, when optimally aligned, such as by the programs GAP or BESTFIT using default gap weights, as supplied with the programs, share at least 70%, 75%, 80% or 85% sequence identity, preferably at least 90%, 91%, 92%, 93%, 94% 95%, 96%, 97%, 98% or 99% sequence identity. In certain embodiments, residue positions that are not identical differ by conservative amino acid substitutions. A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity may be adjusted upwards to correct for the conservative nature of the substitution. Means for making this adjustment are well-known to those of skill in the art. See, e.g., Pearson, Methods Mol. Biol. 243:307-31 (1994). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992), herein incorporated by reference. A “moderately conservative” replacement is any change having a nonnegative value in the PAM250 log-likelihood matrix.

Sequence identity for polypeptides, is typically measured using sequence analysis software. Protein analysis software matches sequences using measures of similarity assigned to various substitutions, deletions and other modifications, including conservative amino acid substitutions. For instance, GCG contains programs such as “Gap” and “Bestfit” which can be used with default parameters, as specified with the programs, to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, see GCG Version 6.1. (University of Wisconsin WI) FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters, as supplied with the programs. See, e.g., Altschul et al., J. Mol. Biol. 215:403-410 (1990); Altschul et al., Nucleic Acids Res. 25:3389-402 (1997).

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

Throughout this specification and claims, the word “comprise.” or variations such as “comprises” or “comprising,” will be understood to imply the inclusion of a stated integer or group of integers but not the exclusion of any other integer or group of integers.

### Human Anti-M-CSF Antibodies and Characterization Thereof

In one embodiment, the invention provides humanized anti-M-CSF antibodies. In another embodiment, the invention provides human anti-M-CSF antibodies. In some embodiments, human anti-M-CSF antibodies are produced by immunizing a non-human transgenic animal, e.g., a rodent, whose genome comprises human immunoglobulin genes so that the rodent produces human antibodies.

An anti-M-CSF antibody of the invention can comprise a human kappa or a human lamda light chain or an amino acid sequence derived therefrom. In some embodiments comprising a kappa light chain, the light chain variable domain (V_{L}) is encoded in part by a human V_{K}O12, V_{K}L2, V_{K}L5, V_{K}A27 or V_{K}B3 gene and a J_{K}1. J_{K}2, J_{K}3, or J_{K}4 gene. In particular embodiments of the invention, the light chain variable domain is encoded by V_{K}O12/JK3, V_{K}L2/JK3, V_{K}L5/JK3, V_{K}L5/JK4, V_{K}A27/JK4 or V_{K}B3/JK1 gene.

In some embodiments, the V_{L} of the M-CSF antibody comprises one or more amino acid substitutions relative to the germline amino acid sequence. In some embodiments, the V_{L} of the anti-M-CSF antibody comprises 1, 2, 3, 4, 5, 6, 7, 8, 9, or 10 amino acid substitutions relative to the germline amino acid sequence. In some embodiments, one or more of those substitutions from germline is in the CDR regions of the light chain. In some embodiments, the amino acid substitutions relative to germline are at one or more of the same positions as the substitutions relative to germline in any one or more of the V_{L} of antibodies 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1. For example, the V_{L} of the anti-M-CSF antibody may contain one or more amino acid substitutions compared to germline found in the V_{L} of antibody 88, and other amino acid substitutions compared to germline found in the V_{L} of antibody 252 which utilizes the same V_{K} gene as antibody 88. In some embodiments, the amino acid changes are at one or more of the same positions but involve a different mutation than in the reference antibody.

In some embodiments, amino acid changes relative to germline occur at one or more of the same positions as in any of the V_{L} of antibodies 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, but the changes may represent conservative amino acid substitutions at such position(s) relative to the amino acid in the reference antibody. For example, if a particular position in one of these antibodies is changed relative to germline and is glutamate, one may substitute aspartate at that position. Similarly, if an amino acid substitution compared to germline is serine, one may substitute threonine for serine at that position. Conservative amino acid substitutions are discussed supra.

In some embodiments, the light chain of the human anti-M-CSF antibody comprises the amino acid sequence that is the same as the amino acid sequence of the V_{L} of antibody 252 (SEQ ID NO: 4), 88 (SEQ ID NO: 8), 100 (SEQ ID NO: 12), 3.8.3 (SEQ ID NO: 16), 2.7.3 (SEQ ID NO: 20), 1.120.1 (SEQ ID NO: 24), 9.14.4I (SEQ ID NO: 28), 8.10.3F (SEQ ID NO: 32), 9.7.2IF (SEQ ID NO: 36), 9.1-4.4 (SEQ ID NO: 28), 8.1-0.3 (SEQ ID NO: 44), 9.7.2 (SEQ ID NO: 48), 9.7.2C-Ser (SEQ ID NO: 52), 9.14.4C-Ser (SEQ ID NO: 56), 8.10.3C-Ser (SEQ ID NO: 60), 8.10.3-CG2 (SEQ ID NO: 60), 9.7.2-CG2 (SEQ ID NO: 52), 9.7.2-CG4 (SEQ ID NO: 52), 9.14.4-CG2 (SEQ ID NO: 56), 9.14.4-CG4 (SEQ ID NO: 56), 9.14.4-Ser (SEQ ID NO: 28), 9.7.2-Ser (SEQ ID NO: 48), 8.10.3-Ser (SEQ ID NO: 44), 8.10.3-CG4 (SEQ ID NO: 60) 8.10.3FG1 (SEQ ID NO: 32) or 9.14.4G1 (SEQ ID NO: 28), or said amino acid sequence having up to 1, 2, 3, 4, 5, 6, 7, 8, 9, or 10 conservative amino acid substitutions and/or a total of up to 3 non-conservative amino acid substitutions. In some embodiments, the light chain comprises the amino acid sequence from the beginning of the CDR1 to the end of the CDR3 of any one of the foregoing antibodies.

In some embodiments, the light chain of the anti-M-CSF antibody comprises at least the light chain CDR1, CDR2 or CDR3 of a germline or antibody sequence, as described herein. In another embodiment, the light chain may comprise a CDR1, CDR2 or CDR3 regions of an antibody independently selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, or CDR regions each having less than 4 or less than 3 conservative amino acid substitutions and/or a total of three or fewer non-conservative amino acid substitutions. In other embodiments, the light chain of the anti-M-CSF antibody comprises the light chain CDR1, CDR2 or CDR3, each of which are independently selected from the CDR1, CDR2 and CDR3 regions of an antibody having a light chain variable region comprising the amino acid sequence of the V_{L} region selected from SEQ ID NOS: 4, 8, 12, 16, 20, 24, 28, 32, 36, 44, 48, 52, 56 or 60, or encoded by a nucleic acid molecule encoding the V_{L} region selected from SEQ ID NOS: 3, 7, 11, 27, 31, 35, 43 or 47. The light chain of the anti-M-CSF antibody may comprise the CDR1, CDR2 and CDR3 regions of an antibody comprising the amino acid sequence of the V_{L} region selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1 or SEQ ID NOS: 4, 8, 12, 16, 20, 24, 28, 32, 36, 44, 48, 52, 56 or 60.

In some embodiments, the light chain comprises the CDR1, CDR2 and CDR3 regions of antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, or said CDR regions each having less than 4 or less than 3 conservative amino acid substitutions and/or a total of three or fewer non-conservative amino acid substitutions.

With regard to the heavy chain, in some embodiments, the variable region of the heavy chain amino acid sequence is encoded in part by a human V_{H}3-11, V_{H}3-23, V_{H}3-7, V_{H}1-18, V_{H}3-33, V_{H}3-48 gene and a J_{H}4, J_{H}6, J_{H}4b, or J_{H}6b gene. In a particular embodiment of the invention, the heavy chain variable region is encoded by V_{H}3-11/D_{H}7-27/J_{H}6, V_{H}3-7/D_{H}6-13/J_{H}4, V_{H}3-23/D_{H}-26/J_{H}4, V_{H}3-11/D_{H}7-27/J_{H}4, V_{H}3-33/D_{H}1-26/J_{H}4, V_{H}1-18/D_{H}4-23/J_{H}4, V_{H}3-11/D_{H}7-27/J_{H}4b, V_{H}3-48/D_{H}1-26/J_{H}4b, V_{H}3-11/D_{H}6-13/J_{H}6b, V_{H}3-11/D_{H}7-27/J_{H}4b, V_{H}3-48/D_{H}1-6/J_{H}4b, or V_{H}3-11/D_{H}6-13/J_{H}6b gene. In some embodiments, the V_{H} of the anti-M-CSF antibody contains one or more amino acid substitutions, deletions or insertions (additions) relative to the germline amino acid sequence. In some embodiments, the variable domain of the heavy chain comprises 1, 2, 3, 4, 5, 6, 7, 8, 9, 10, 11, 12, 13, 14, 15, 16, 17, or 18 mutations from the germline amino acid sequence. In some embodiments, the mutation(s) are non-conservative substitutions compared to the germline amino acid sequence. In some embodiments, the mutations are in the CDR regions of the heavy chain. In some embodiments, the amino acid changes are made at one or more of the same positions as the mutations from germline in any one or more of the V_{H} of antibodies 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1. In other embodiments, the amino acid changes are at one or more of the same positions but involve a different mutation than in the reference antibody.

In some embodiments, the heavy chain comprises an amino acid sequence of the variable domain (V_{H}) of antibody 252 (SEQ ID NO: 2), 88 (SEQ ID NO: 6), 100 (SEQ ID NO: 10), 3.8.3 (SEQ ID NO: 14), 2.7.3 (SEQ. ID NO: 18), 1.120.1 (SEQ. ID NO: 22), 9.14.4I (SEQ ID NO: 26), 8.10.3F (SEQ ID NO: 30), 9.7.2IF (SEQ ID NO: 34), 9.1-4.4 (SEQ ID NO: 38), 8.10.3 (SEQ ID NO: 30), 9.7.2 (SEQ ID NO: 46), 9.7.2C-Ser (SEQ ID NO: 50), 9.14.4C-Ser (SEQ ID NO: 54), 8.10.3C-Ser (SEQ ID NO: 58), 8.10.3-CG2 (SEQ ID NO: 62), 9.7.2-CG2 (SEQ ID NO: 66), 9.7.2-CG4 (SEQ ID NO: 70), 9.14.4-CG2 (SEQ ID NO: 74), 9.14.4-CG4 (SEQ ID NO: 78), 9.14.4-Ser (SEQ ID NO: 82), 9.7.2-Ser (SEQ ID NO: 86), 8.10.3-Ser (SEQ ID NO: 90) 8.10.3-CG4 (SEQ ID NO: 94), 8.10.3FG1 (SEQ ID NO: 98) or 9.14.4G1 (SEQ ID NO: 102), or said amino acid sequence having up to 1, 2, 3, 4, 5, 6, 7, 8, 9, or 10 conservative amino acid substitutions and/or a total of up to 3 non-conservative amino acid substitutions. In some embodiments, the heavy chain comprises the amino acid sequence from the beginning of the CDR1 to the end of the CDR3 of any one of the foregoing antibodies.

In some embodiments, the heavy chain comprises the heavy chain CDR1, CDR2 and CDR3 regions of antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, or said CDR regions each having less than 8, less than 6, less than 4, or less than 3 conservative amino acid substitutions and/or a total of three or fewer non-conservative amino acid substitutions.

In some embodiments, the heavy chain comprises a germline or antibody CDR3, as described above, of an antibody sequence as described herein, and may also comprise the CDR1 and CDR2 regions of a germline sequence, or may comprise a CDR1 and CDR2 of an antibody sequence, each of which are independently selected from an antibody comprising a heavy chain of an antibody selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1. In another embodiment, the heavy chain comprises a CDR3 of an antibody sequence as described herein, and may also comprise the CDR1 and CDR2 regions, each of which are independently selected from a CDR1 and CDR2 region of a heavy chain variable region comprising an amino acid sequence of the V_{H} region selected from SEQ ID NOS: 2, 6, 10, 14, 18, 22, 26, 30, 34, 38, 46, 50, 54, 58, 62, 66, 70, 74, 78, 82, 86, 90, 94, 98 or 102, or encoded by a nucleic acid sequence encoding the V_{H} region selected from SEQ ID NOS: 1, 5, 9, 25, 29, 33, 37, 45, 97 or 101. In another embodiment, the antibody comprises a light chain as disclosed above and a heavy chain as disclosed above.

One type of amino acid substitution that may be made is to change one or more cysteines in the antibody, which may be chemically reactive, to another residue, such as, without limitation, alanine or serine. In one embodiment, there is a substitution of a non-canonical cysteine. The substitution can be in a framework region of a variable domain or in the constant domain of an antibody. In another embodiment, the cysteine is in a non-canonical region of the antibody.

Another type of amino acid substitution that may be made is to remove any potential proteolytic sites in the antibody, particularly those that are in a CDR or framework region of a variable domain or in the constant domain of an antibody. Substitution of cysteine residues and removal of proteolytic sites may decrease the risk of any heterogeneity in the antibody product and thus increase its homogeneity. Another type of amino acid substitution is elimination of asparagine-glycine pairs, which form potential deamidation sites, by altering one or both of the residues.

In some embodiments, the C-terminal lysine of the heavy chain of the anti-M-CSF antibody of the invention is not present (Lewis D. A., et al., Anal. Chem., 66(5): 585-95 (1994)). In various embodiments of the invention, the heavy and light chains of the anti-M-CSF antibodies may optionally include a signal sequence.

In one aspect, the invention relates to human anti-M-CSF monoclonal antibodies and the cell lines engineered to produce them. Table 1A lists the sequence identifiers (SEQ ID NOS) of the nucleic acids that encode the variable region of the heavy and light chains and the corresponding predicted amino acid sequences for the monoclonal antibodies: 252, 88, 100, 3.8.3, 2.7.3, 1.120.1, 9.14.4I, 8.10.3F, 9.7.2IF, 9.14.4, 8.10.3 and 9.7.2. Additional variant antibodies 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4 8.10.3FG1 or 9.14.4G1 could be made by methods known to one skilled in the art. Table 1B lists the percent amino acid identity of the heavy chain, light chains, and both heavy and light chains of the antibodies 252, 88, 100, 3.8.3, 2.7.3, 1.120.1, 9.7.2, 9.14.4, 8.10.3, 8.10.3C-Ser, 8.10.3-CG2, 8.10.3-Ser, 8.10.3-CG4, and 8.10.3FG1 as compared to the heavy chain, light chains, and heavy and light chains, respectively of antibody 8.10.3F.

In another aspect, the invention relates to anti-M-CSF monoclonal antibodies that are substantially similar to the monoclonal antibody 8.10.3F, wherein the amino acid sequence of the heavy chain, or the light chain, or both the heavy chain and light chain of the antibodies share at least about 70%, 75%, 80%, 85%, 90%, 91%, 92%, 93%, 94%, 95%, 96%, 97%, 98% or 99% identity with the heavy chain, or light chain, or both the heavy chain and light chain of the antibody 8.10.3F respectively.

TABLE 1A
HUMAN ANTI-M-CSF ANTIBODIES

| SEQUENCE IDENTIFIER | |
|---|---|
| (SEQ ID NOS:) | |
| Full Length | |
| Heavy | Light |

| MAb | DNA | Protein | DNA | Protein |
|---|---|---|---|---|
| 252 | 1 | 2 | 3 | 4 |
| 88 | 5 | 6 | 7 | 8 |
| 100 | 9 | 10 | 11 | 12 |
| 3.8.3 | | 14 | | 16 |
| 2.7.3 | | 18 | | 20 |
| 1.120.1 | | 22 | | 24 |
| 9.14.4I | 25 | 26 | 27 | 28 |
| 9.14.4 | 37 | 38 | 27 | 28 |
| 9.14.4C-Ser | | 54 | | 56 |
| 9.14.4-CG2 | | 74 | | 56 |
| 9.14.4-CG4 | | 78 | | 56 |
| 9.14.4-Ser | | 82 | 27 | 28 |
| 9.14.4-G1 | 101 | 102 | 27 | 28 |
| 8.10.3F | 29 | 30 | 31 | 32 |
| 8.10.3 | 29 | 30 | 43 | 44 |
| 8.10.3C-Ser | | 58 | | 60 |
| 8.10.3-CG2 | | 62 | | 60 |
| 8.10.3-Ser | | 90 | 43 | 44 |
| 8.10.3-CG4 | | 94 | | 60 |
| 8.10.3FG1 | 97 | 98 | 31 | 32 |
| 9.7.2IF | 33 | 34 | 35 | 36 |
| 9.7.2 | 45 | 46 | 47 | 48 |
| 9.7.2C-Ser | | 50 | | 52 |
| 9.7.2-CG2 | | 66 | | 52 |
| 9.7.2-CG4 | | 70 | | 52 |
| 9.7.2-Ser | | 86 | 47 | 48 |

TABLE 1B

| | Percentage Identity with Heavy Chain, Light Chain, or | | |
|---|---|---|---|
| | Both Heavy and Light Chains of Antibody 8.10.3F | | |
| | | | Heavy & Light |
| MAb | Heavy Chain | Light Chain | Chain Combined |
| 8.10.3-CG2 | 100 | 99 | 99 |
| 8.10.3-CG4 | 94 | 99 | 96 |
| 8.10.3-Ser | 95 | 99 | 96 |
| 8.10.3C-Ser | 95 | 99 | 96 |
| 8.10.3FG1 | 94 | 100 | 96 |
| 8.10.3 | 100 | 99 | 99 |
| 1.120.1 | 87 | 85 | 86 |
| 100 | 94 | 90 | 93 |
| 2.7.3 | 89 | 84 | 87 |
| 252 | 95 | 84 | 91 |
| 3.8.3 | 96 | 83 | 92 |
| 88 | 95 | 84 | 91 |
| 9.14.4 | 91 | 83 | 88 |
| 9.7.2 | 90 | 83 | 88 |

Class and Subclass of Anti-M-CSF antibodies

The class and subclass of anti-M-CSF antibodies may be determined by any method known in the art. In general, the class and subclass of an antibody may be determined using antibodies that are specific for a particular class and subclass of antibody. Such antibodies are commercially available. The class and subclass can be determined by ELISA, or Western Blot as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

In some embodiments, the anti-M-CSF antibody is a monoclonal antibody. The anti-M-CSF antibody can be an IgG, an IgM, an IgE, an IgA, or an IgD molecule. In preferred embodiments, the anti-M-CSF antibody is an IgG and is an IgG1, IgG2, IgG3 or IgG4 subclass. In other preferred embodiments, the antibody is subclass IgG2 or IgG4. In another preferred embodiment, the antibody is subclass IgG1.

### Species and Molecular Selectivity

In another aspect of the invention, the anti-M-CSF antibodies demonstrate both species and molecule selectivity. In some embodiments, the anti-M-CSF antibody binds to human, cynomologus monkey and mouse M-CSF. Following the teachings of the specification, one may determine the species selectivity for the anti-M-CSF antibody using methods well known in the art. For instance, one may determine the species selectivity using Western blot, FACS, ELISA, RIA, a cell proliferation assay, or a M-CSF receptor binding assay. In a preferred embodiment, one may determine the species selectivity using a cell proliferation assay or ELISA.

In another embodiment, the anti-M-CSF antibody has a selectivity for M-CSF that is at least 100 times greater than its selectivity for GM-/G-CSF. In some embodiments, the anti-M-CSF antibody does not exhibit any appreciable specific binding to any other protein other than M-CSF. One can determine the selectivity of the anti-M-CSF antibody for M-CSF using methods well known in the art following the teachings of the specification. For instance one can determine the selectivity using Western blot, FACS, ELISA, or RIA.

### Identification of M-CSF Epitopes Recognized by Anti-M-CSF Antibodies

The invention provides a human anti-M-CSF monoclonal antibody that binds to M-CSF and competes with, cross-competes with and/or binds the same epitope and/or binds to M-CSF with the same K_{D} as (a) an antibody selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1; (b) an antibody that comprises a heavy chain variable region having an amino acid sequence of SEQ ID NO: 2, 6, 10, 14, 18, 22, 26, 30, 34, 38, 46, 50, 54, 58, 62, 66, 70, 74, 78, 82, 86, 90, 94, 98 or 102; (c) an antibody that comprises a light chain variable region having an amino acid sequence of SEQ ID NO: 4, 8, 12, 16, 20, 24, 28, 32, 36, 44, 48, 52, 56 or 60; (d) an antibody that comprises both a heavy chain variable region as defined in (b) and a light chain variable region as defined in (c).

One can determine whether an antibody binds to the same epitope, competes for binding with, cross competes for binding with or has the same K_{D} an anti-M-CSF antibody by using methods known in the art. In one embodiment, one allows the anti-M-CSF antibody of the invention to bind to M-CSF under saturating conditions and then measures the ability of the test antibody to bind to M-CSF. If the test antibody is able to bind to M-CSF at the same time as the anti-M-CSF antibody, then the test antibody binds to a different epitope as the anti-M-CSF antibody. However, if the test antibody is not able to bind to M-CSF at the same time, then the test antibody binds to the same epitope, an overlapping epitope, or an epitope that is in close proximity to the epitope bound by the human anti-M-CSF antibody. This experiment can be performed using ELISA, RIA, or FACS. In a preferred embodiment, the experiment is performed using BIACORE™.

### Binding Affinity of Anti-M-CSF Antibodies to M-CSF

In some embodiments of the invention, the anti-M-CSF antibodies bind to M-CSF with high affinity. In some embodiments, the anti-M-CSF antibody binds to M-CSF with a K_{D} of 1×10⁻⁷ M or less. In other preferred embodiments, the antibody binds to M-CSF with a K_{D} of 1×10⁻⁸ M, 1×10⁻⁹ M, 1×10⁻¹⁰M, 1×10⁻¹¹ M, 1×10⁻¹²M or less. In certain embodiments, the K_{D} is 1 pM to 500 pM. In other embodiments, the K_{D} is between 500 pM to 1 μM. In other embodiments, the K_{D} is between 1 μM to 100 nM. In other embodiments, the K_{D} is between 100 mM to 10 nM. In an even more preferred embodiment, the antibody binds to M-CSF with substantially the same K_{D} as an antibody selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1. In another preferred embodiment, the antibody binds to M-CSF with substantially the same K_{D} as an antibody that comprises a CDR2 of a light chain, and/or a CDR3 of a heavy chain from an antibody selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1. In still another preferred embodiment, the antibody binds to M-CSF with substantially the same K_{D} as an antibody that comprises a heavy chain variable region having an amino acid sequence of SEQ ID NO: 2, 6, 10, 14, 18, 22, 26, 30, 34, 38, 46, 50, 54, 58, 62, 66, 70, 74, 78, 82, 86, 90, 94, 98 or 102, or that comprises a light chain variable region having an amino acid sequence of SEQ ID NO: 4, 8, 12, 16, 20, 24, 28, 32, 36, 44, 48, 52, 56 or 60. In another preferred embodiment, the antibody binds to M-CSF with substantially the same K_{D} as an antibody that comprises a CDR2, and may optionally comprise a CDR1 and/or CDR3, of a light chain variable region having an amino acid sequence of the V_{L} region of SEQ ID NO: 4, 8, 12, 16, 20, 24, 28, 32, 36, 44, 48, 52, 56 or 60, or that comprises a CDR3, and may optionally comprise a CDR1 and/or CDR2, of a heavy chain variable region having an amino acid sequence of the V_{H} region of SEQ ID NO: 2, 6, 10, 14, 18, 22, 26, 30, 34, 38, 46, 50, 54, 58, 62, 66, 70, 74, 78, 82, 86, 90, 94, 98 or 102.

In some embodiments, the anti-M-CSF antibody has a low dissociation rate. In some embodiments, the anti-M-CSF antibody has an k_{off} of 2.0×10⁻⁴ s⁻¹ or lower. In other preferred embodiments, the antibody binds to M-CSF with a k_{off} of 2.0×10⁻⁵ or a k_{off} 2.0×10⁻⁶ s⁻¹ or lower. In some embodiments, the k_{off} is substantially the same as an antibody described herein, such as an antibody selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1. In some embodiments, the antibody binds to M-CSF with substantially the same k_{off} as an antibody that comprises (a) a CDR3, and may optionally comprise a CDR1 and/or CDR2, of a heavy chain of an antibody selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1; or (b) a CDR2, and may optionally comprise a CDR1 and/or CDR3, of a light chain from an antibody selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1. In some embodiments, the antibody binds to M-CSF with substantially the same k_{off} as an antibody that comprises a heavy chain variable region having an amino acid sequence of SEQ ID NO: 2, 6, 10, 14, 18, 22, 26, 30, 34, 38, 46, 50, 54, 58, 62, 66, 70, 74, 78, 82, 86, 90, 94, 98 or 102; or that comprises a light chain variable region having an amino acid sequence of SEQ ID NO: 4, 8, 12, 16, 20, 24, 28, 32, 36, 44, 48, 52, 56 or 60; In another preferred embodiment, the antibody binds to M-CSF with substantially the same k_{off} as an antibody that comprises a CDR2, and may optionally comprise a CDR1 and/or CDR3, of a light chain variable region having an amino acid sequence of SEQ ID NO: 4, 8, 12, 16, 20, 24, 28, 32, 36, 44, 48, 52, 56 or 60; or a CDR3, and may optionally comprise a CDR1 and/or CDR2, of a heavy chain variable region having an amino acid sequence of SEQ ID NO: 2, 6, 10, 14, 18, 22, 26, 30, 34, 38, 46, 50, 54, 58, 62, 66, 70, 74, 78, 82, 86, 90, 94, 98 or 102.

The binding affinity and dissociation rate of an anti-M-CSF antibody to a M-CSF can be determined by methods known in the art. The binding affinity can be measured by competitive ELISAs, RIAs, surface plasmon resonance (e.g., by using BIACORE™ technology). The dissociation rate can be measured by surface plasmon resonance. Preferably, the binding affinity and dissociation rate is measured by surface plasmon resonance. More preferably, the binding affinity and dissociation rate are measured using BIACORE™ technology. Example VI exemplifies a method for determining affinity constants of anti-M-CSF monoclonal antibodies by BIACORE™ technology.

### Inhibition of M-CSF Activity by Anti-M-CSF Antibody

Inhibition of M-CSF Binding to c-fms

In another embodiment, the invention provides an anti-M-CSF antibody that inhibits the binding of a M-CSF to c-fms receptor and blocks or prevents activation of c-fms. In an preferred embodiment, the M-CSF is human. In another preferred embodiment, the anti-M-CSF antibody is a human antibody. The IC₅₀ can be measured by ELISA, RIA, and cell based assays such as a cell proliferation assay, a whole blood monocyte shape change assay, or a receptor binding inhibition assay. In one embodiment, the antibody or portion thereof inhibits cell proliferation with an IC₅₀ of no more than 8.0×10⁻⁷ M, preferably no more than 3×10⁻⁷ M, or more preferably no more than 8×10⁻⁸ M as measured by a cell proliferation assay. In another embodiment, the IC₅₀ as measured by a monocyte shape change assay is no more than 2×10⁻⁶ M, preferably no more than 9.0×10⁻⁷ M, or more preferably no more than 9×10⁻⁸ M. In another preferred embodiment, the ICs as measured by a receptor binding assay is no more than 2×10⁻⁶ M, preferably no more than 8.0×10⁻⁷ M, or more preferably no more than 7.0×10⁻⁸ M. Examples III, IV, and V exemplify various types of assays.

In another aspect anti-M-CSF antibodies of the invention inhibit monocyte/macrophage cell proliferation in response to a M-CSF by at least 20%, more preferably 40%, 45%, 50%, 55%, 60%, 65%, 70%, 80%, 85%, 90%, 95% or 100% compared to the proliferation of cell in the absence of antibody.

### Methods of Producing Antibodies and Antibody Producing Cell Lines

### Immunization

In some embodiments, human antibodies are produced by immunizing a non-human animal comprising in its genome some or all of human immunoglobulin heavy chain and light chain loci with a M-CSF antigen. In a preferred embodiment, the non-human animal is a XENOMOUSE™ animal (Abgenix Inc., Fremont, Calif.). Another non-human animal that may be used is a transgenic mouse produced by Medarex (Medarex, Inc., Princeton, N.J.).

XENOMOUSE™ mice are engineered mouse strains that comprise large fragments of human immunoglobulin heavy chain and light chain loci and are deficient in mouse antibody production. See, e.g., Green et al., Nature Genetics 7:13-21 (1994) and U.S. Pat. Nos. 5,916,771, 5,939,598, 5,985,615, 5,998,209, 6,075,181, 6,091,001, 6,114,598, 6,130,364, 6,162,963 and 6,150,584. See also WO 91/10741, WO 94/02602, WO 96/34096, WO 96/33735, WO 98/16654, WO 98/24893, WO 98/50433, WO 99/45031, WO 99/53049, WO 00/09560, and WO 00/037504.

In another aspect, the invention provides a method for making anti-M-CSF antibodies from non-human, non-mouse animals by immunizing non-human transgenic animals that comprise human immunoglobulin loci with a M-CSF antigen. One can produce such animals using the methods described in the above-cited documents. The methods disclosed in these documents can be modified as described in U.S. Pat. No. 5,994,619. U.S. Pat. No. 5,994,619 describes methods for producing novel cultural inner cell mass (CICM) cells and cell lines, derived from pigs and cows, and transgenic CICM cells into which heterologous DNA has been inserted. CICM transgenic cells can be used to produce cloned transgenic embryos, fetuses, and offspring. The '619 patent also describes the methods of producing the transgenic animals, that are capable of transmitting the heterologous DNA to their progeny. In preferred embodiments, the non-human animals are rats, sheep, pigs, goats, cattle or horses.

XENOMOUSE™ mice produce an adult-like human repertoire of fully human antibodies and generate antigen-specific human antibodies. In some embodiments, the XENOMOUSE™ mice contain approximately 80% of the human antibody V gene repertoire through introduction of megabase sized, germline configuration yeast artificial chromosome (YAC) fragments of the human heavy chain loci and kappa light chain loci. In other embodiments, XENOMOUSE™ mice further contain approximately all of the lambda light chain locus. See Mendez et al., Nature Genetics 15:146-156 (1997), Green and Jakobovits, J. Exp. Med. 188:483-495 (1998), and WO 98/24893, the disclosures of which are hereby incorporated by reference.

In some embodiments, the non-human animal comprising human immunoglobulin genes are animals that have a human immunoglobulin “minilocus”. In the minilocus approach, an exogenous Ig locus is mimicked through the inclusion of individual genes from the Ig locus. Thus, one or more V_{H} genes, one or more D_{H} genes, one or more J_{H} genes, a mu constant domain, and a second constant domain (preferably a gamma constant domain) are formed into a construct for insertion into an animal. This approach is described, inter alia, in U.S. Pat. Nos. 5,545,807, 5,545,806, 5,569,825, 5,625,126, 5,633,425, 5,661,016, 5,770,429, 5,789,650, 5,814,318, 5,591,669, 5,612,205, 5,721,367, 5,789,215, and 5,643,763, hereby incorporated by reference.

In another aspect, the invention provides a method for making humanized anti-M-CSF antibodies. In some embodiments, non-human animals are immunized with a M-CSF antigen as described below under conditions that permit antibody production. Antibody-producing cells are isolated from the animals, fused with myelomas to produce hybridomas, and nucleic acids encoding the heavy and light chains of an anti-M-CSF antibody of interest are isolated. These nucleic acids are subsequently engineered using techniques known to those of skill in the art and as described further below to reduce the amount of non-human sequence, i.e., to humanize the antibody to reduce the immune response in humans

In some embodiments, the M-CSF antigen is isolated and/or purified M-CSF. In a preferred embodiment, the M-CSF antigen is human M-CSF. In some embodiments, the M-CSF antigen is a fragment of M-CSF. In some embodiments, the M-CSF fragment is the extracellular domain of M-CSF. In some embodiments, the M-CSF fragment comprises at least one epitope of M-CSF. In other embodiments, the M-CSF antigen is a cell that expresses or overexpresses M-CSF or an immunogenic fragment thereof on its surface. In some embodiments, the M-CSF antigen is a M-CSF fusion protein. M-CSF can be purified from natural sources using known techniques. Recombinant M-CSF is commercially available.

Immunization of animals can be by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane, supra, and U.S. Pat. No. 5,994,619. In a preferred embodiment, the M-CSF antigen is administered with an adjuvant to stimulate the immune response. Exemplary adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks. Example I exemplifies a method for producing anti-M-CSF monoclonal antibodies in XENOMOUSE™ mice.

### Production of Antibodies and Antibody-Producing Cell Lines

After immunization of an animal with a M-CSF antigen, antibodies and/or antibody-producing cells can be obtained from the animal. In some embodiments, anti-M-CSF antibody-containing serum is obtained from the animal by bleeding or sacrificing the animal. The serum may be used as it is obtained from the animal, an immunoglobulin fraction may be obtained from the serum, or the anti-M-CSF antibodies may be purified from the serum.

In some embodiments, antibody-producing immortalized cell lines are prepared from cells isolated from the immunized animal. After immunization, the animal is sacrificed and lymph node and/or splenic B cells are immortalized. Methods of immortalizing cells include, but are not limited to, transfecting them with oncogenes, infecting them with an oncogenic virus, cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Immortalized cells are screened using M-CSF, a portion thereof, or a cell expressing M-CSF. In a preferred embodiment, the initial screening is performed using an enzyme-linked immunoassay (ELISA) or a radioimmunoassay. An example of ELISA screening is provided in WO 00/37504, incorporated herein by reference.

Anti-M-CSF antibody-producing cells, e.g. hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, the immunized animal is a non-human animal that expresses human immunoglobulin genes and the splenic B cells are fused to a myeloma cell line from the same species as the non-human animal. In a more preferred embodiment, the immunized animal is a XENOMOUSE™ animal and the myeloma cell line is a non-secretory mouse myeloma. In an even more preferred embodiment, the myeloma cell line is P3-X63-AG8-653. See, e.g., Example I.

Thus, in one embodiment, the invention provides methods of producing a cell line that produces a human monoclonal antibody or a fragment thereof directed to M-CSF comprising (a) immunizing a non-human transgenic animal described herein with M-CSF, a portion of M-CSF or a cell or tissue expressing M-CSF; (b) allowing the transgenic animal to mount an immune response to M-CSF; (c) isolating B lymphocytes from a transgenic animal; (d) immortalizing the B lymphocytes: (e) creating individual monoclonal populations of the immortalized B lymphocytes; and (f) screening the immortalized B lymphocytes to identify an antibody directed to M-CSF.

In another aspect, the invention provides hybridomas that produce an human anti-M-CSF antibody. In a preferred embodiment, the hybridomas are mouse hybridomas, as described above. In other embodiments, the hybridomas are produced in a non-human, non-mouse species such as rats, sheep, pigs, goats, cattle or horses. In another embodiment, the hybridomas are human hybridomas.

In another preferred embodiment, a transgenic animal is immunized with M-CSF, primary cells, e.g., spleen or peripheral blood cells, are isolated from an immunized transgenic animal and individual cells producing antibodies specific for the desired antigen are identified. Polyadenylated mRNA from each individual cell is isolated and reverse transcription polymerase chain reaction (RT-PCR) is performed using sense primers that anneal to variable region sequences, e.g., degenerate primers that recognize most or all of the FR1 regions of human heavy and light chain variable region genes and antisense primers that anneal to constant or joining region sequences. cDNAs of the heavy and light chain variable regions are then cloned and expressed in any suitable host cell, e.g., a myeloma cell, as chimeric antibodies with respective immunoglobulin constant regions, such as the heavy chain and κ or λ constant domains. See Babcook, J. S. et al., Proc. Natl. Acad. Sci. USA 93:7843-48, 1996, herein incorporated by reference. Anti M-CSF antibodies may then be identified and isolated as described herein.

In another embodiment, phage display techniques can be used to provide libraries containing a repertoire of antibodies with varying affinities for M-CSF. For production of such repertoires, it is unnecessary to immortalize the B cells from the immunized animal. Rather, the primary B cells can be used directly as a source of DNA. The mixture of cDNAs obtained from B cell, e.g., derived from spleens, is used to prepare an expression library, for example, a phage display library transfected into E. coli. The resulting cells are tested for immunoreactivity to M-CSF. Techniques for the identification of high affinity human antibodies from such libraries are described by Griffiths et al., EMBO J., 13:3245-3260 (1994); Nissim et al., ibid, pp. 692-698 and by Griffiths et al., ibid, 12:725-734. Ultimately, clones from the library are identified which produce binding affinities of a desired magnitude for the antigen and the DNA encoding the product responsible for such binding is recovered and manipulated for standard recombinant expression. Phage display libraries may also be constructed using previously manipulated nucleotide sequences and screened in a similar fashion. In general, the cDNAs encoding heavy and light chains are independently supplied or linked to form Fv analogs for production in the phage library.

The phage library is then screened for the antibodies with the highest affinities for M-CSF and the genetic material recovered from the appropriate clone. Further rounds of screening can increase affinity of the original antibody isolated.

In another aspect, the invention provides hybridomas that produce an human anti-M-CSF antibody. In a preferred embodiment, the hybridomas are mouse hybridomas, as described above. In other embodiments, the hybridomas are produced in a non-human, non-mouse species such as rats, sheep, pigs, goats, cattle or horses. In another embodiment, the hybridomas are human hybridomas.

### Nucleic Acids, Vectors, Host Cells, and Recombinant Methods of Making Antibodies

### Nucleic Acids

The present invention also encompasses nucleic acid molecules encoding anti-M-CSF antibodies. In some embodiments, different nucleic acid molecules encode a heavy chain and a light chain of an anti-M-CSF immunoglobulin. In other embodiments, the same nucleic acid molecule encodes a heavy chain an a light chain of an anti-M-CSF immunoglobulin. In one embodiment, the nucleic acid encodes a M-CSF antibody of the invention.

In some embodiments, the nucleic acid molecule encoding the variable domain of the light chain comprises a human V_{K}L5, O12, L2, B3, A27 gene and a JK1, JK2, JK3, or JK4 gene.

In some embodiments, the nucleic acid molecule encoding the light chain, encodes an amino acid sequence comprising 1, 2, 3, 4, 5, 6, 7, 8, 9, or 10 mutations from the germline amino acid sequence. In some embodiments, the nucleic acid molecule comprises a nucleotide sequence that encodes a V_{L} amino acid sequence comprising 1, 2, 3, 4, 5, 6, 7, 8, 9, or 10 non-conservative amino acid substitutions and/or 1, 2, or 3 non-conservative substitutions compared to germline sequence. Substitutions may be in the CDR regions, the framework regions, or in the constant domain.

In some embodiments, the nucleic acid molecule encodes a V_{L} amino acid sequence comprising one or more variants compared to germline sequence that are identical to the variations found in the V_{L} of one of the antibodies 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1.

In some embodiments, the nucleic acid molecule encodes at least three amino acid mutations compared to the germline sequence found in the V_{L} of one of the antibodies 252, 88, 100, 3.8.3, 2.7.3, 1.120.1, 9.14.4, 8.10.3, or 9.7.2.

In some embodiments, the nucleic acid molecule comprises a nucleotide sequence that encodes the V₁ amino acid sequence of monoclonal antibody 252 (SEQ ID NO: 4), 88 (SEQ ID NO: 8), 100 (SEQ ID NO: 12), 3.8.3 (SEQ ID NO: 16), 2.7.3 (SEQ ID NO: 20), 1.120.1 (SEQ ID NO: 24), 9.14.4I (SEQ ID NO: 28), 8.10.3F (SEQ ID NO: 32), 9.7.2IF (SEQ ID NO: 36), 9.1-4.4 (SEQ ID NO: 28), 8.1-0.3 (SEQ ID NO: 44), 9.7.2 (SEQ ID NO: 48), 9.7.2C-Ser (SEQ ID NO: 52), 9.14.4C-Ser (SEQ ID NO: 56), 8.10.3C-Ser (SEQ ID NO: 60), 8.10.3-CG2 (SEQ ID NO: 60), 9.7.2-CG2 (SEQ ID NO: 52), 9.7.2-CG4 (SEQ ID NO: 52), 9.14.4-CG2 (SEQ ID NO: 56), 9.14.4-CG4 (SEQ ID NO: 56), 9.14.4-Ser (SEQ ID NO: 28), 9.7.2-Ser (SEQ ID NO: 48), 8.10.3-Ser (SEQ ID NO: 44), 8.10.3-CG4 (SEQ ID NO: 60) 8.10.3FG1 (SEQ ID NO: 32) or 9.14.4G1 (SEQ ID NO: 28), or a portion thereof. In some embodiments, said portion comprises at least the CDR2 region. In some embodiments, the nucleic acid encodes the amino acid sequence of the light chain CDRs of said antibody. In some embodiments, said portion is a contiguous portion comprising CDR1-CDR3.

In some embodiments, the nucleic acid molecule comprises a nucleotide sequence that encodes the light chain amino acid sequence of one of SEQ ID NOS: 4, 8, 12, 16, 20, 24, 28, 32, 36, 44, 48, 52, 56 or 60. In some preferred embodiments, the nucleic acid molecule comprises the light chain nucleotide sequence of SEQ ID NOS: 3, 7, 11, 27, 31, 35, 43 or 47, or a portion thereof.

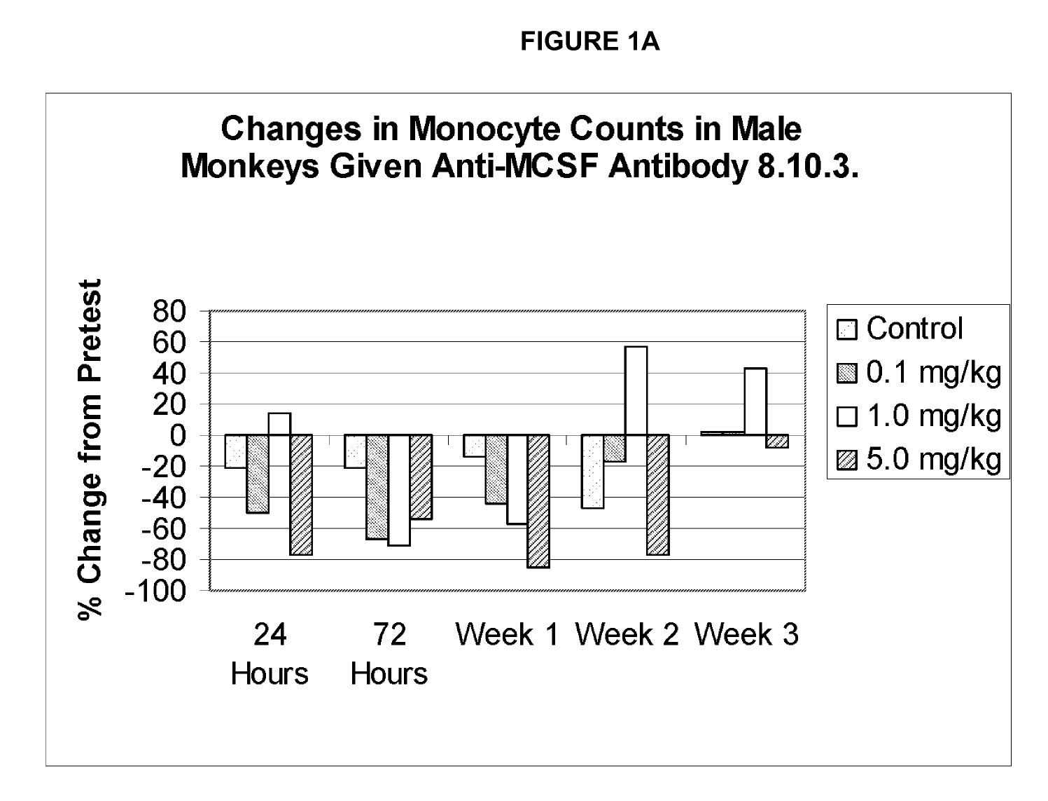

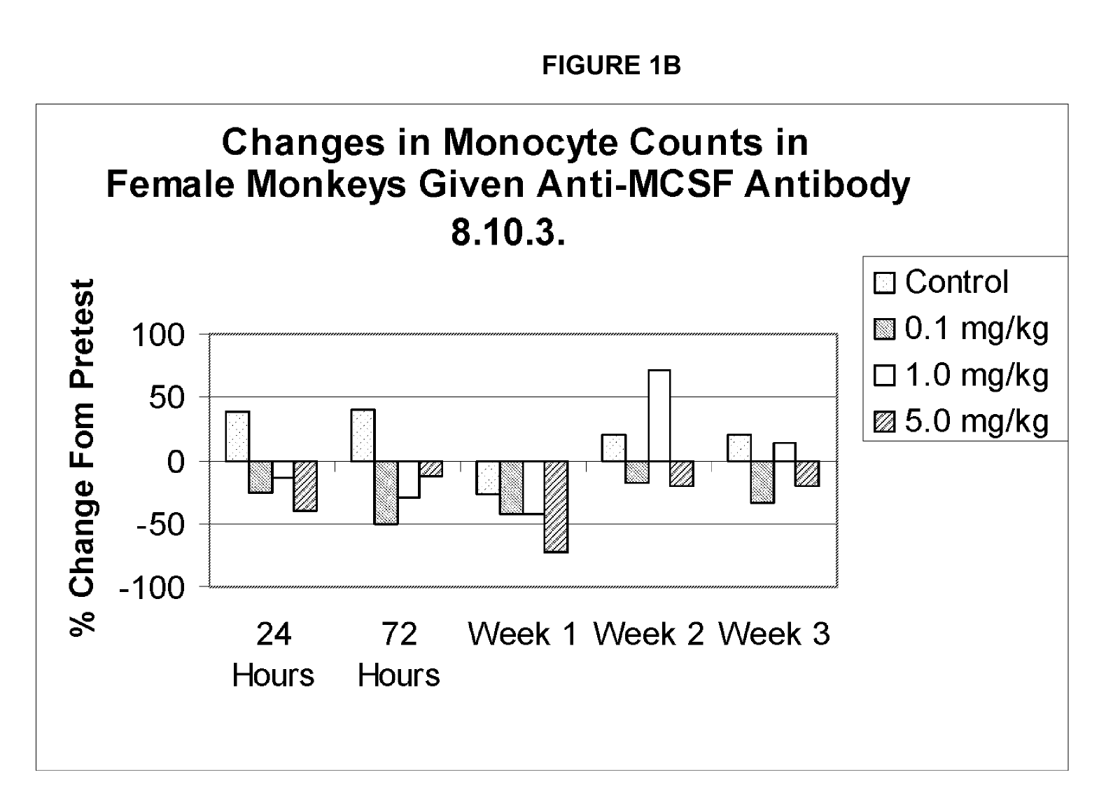

In some embodiments, the nucleic acid molecule encodes a V_{L} amino acid sequence that is at least 70%, 75%, 80%, 85%, 90%, 91%, 92%, 93%, 94%, 95%, 96%, 97%, 98% or 99% identical to a V_{L} amino acid sequence shown in FIG. 1 or to a V_{L} amino acid sequences of any one of antibodies 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, or an amino acid sequence of any one of SEQ ID NOS: 4, 8, 12, 16, 20, 24, 28, 32, 36, 44, 48, 52, 56 or 60. Nucleic acid molecules of the invention include nucleic acids that hybridize under highly stringent conditions, such as those described above, to a nucleic acid sequence encoding the light chain amino acid sequence of SEQ ID NOS: 4, 8, 12, 16, 20, 24, 28, 32, 36, 44, 48, 52, 56 or 60, or that has the light chain nucleic acid sequence of SEQ ID NOS: 3, 7, 11, 27, 31, 35, 43 or 47.

In another embodiment, the nucleic acid encodes a full-length light chain of an antibody selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, or a light chain comprising the amino acid sequence of SEQ ID NOS: 4, 8, 12, 16, 20, 24, 28, 32, 36, 44, 48, 52, 56 or 60 and a constant region of a light chain, or a light chain comprising a mutation. Further, the nucleic acid may comprise the light chain nucleotide sequence of SEQ ID NOS: 3, 7, 11, 27, 31, 35, 43 or 47 and the nucleotide sequence encoding a constant region of a light chain, or a nucleic acid molecule encoding a light chain comprise a mutation.

In another preferred embodiment, the nucleic acid molecule encodes the variable domain of the heavy chain (V_{H}) that comprises a human V_{H} 1-18, 3-33, 3-11, 3-23, 3-48, or 3-7 gene sequence or a sequence derived therefrom. In various embodiments, the nucleic acid molecule comprises a human V_{H} 1-18 gene, a D_{H}4-23 gene and a human J_{H}4 gene; a human V_{H} 3-33 gene, a human D_{H}1-26 gene and a human J_{H}4 gene; a human V_{H} 3-11 gene, a human D_{H}7-27 gene and a human J_{H}4 gene; a human V_{H} 3-11 gene, a human D_{H} 7-27 gene and a human J_{H}6 gene; a human V_{H} 3-23 gene, a human D_{H}1-26 gene and a human J_{H}4 gene; a human V_{H} 3-7 gene, a human D_{H}6-13 gene and a human J_{H}4 gene; a human V_{H}3-11 gene, a human D_{H}7-27 gene, and a human J_{H}4b gene; a human V_{H}3-48 gene, a human D_{H}1-26 gene, and a human J_{H}4b gene; a human V_{H}3-11 gene, a human D_{H}6-13 gene, and a human J_{H}6b gene, or a sequence derived from the human genes.

In some embodiments, the nucleic acid molecule encodes an amino acid sequence comprising 1, 2, 3, 4, 5, 6, 7, 8, 9, 10, 11, 12, 13, 14, 15, 16, 17 or 18 mutations compared to the germline amino acid sequence of the human V, D or J genes. In some embodiments, said mutations are in the V_{H} region. In some embodiments, said mutations are in the CDR regions.

In some embodiments, the nucleic acid molecule encodes one or more amino acid mutations compared to the germline sequence that are identical to amino acid mutations found in the V_{H} of monoclonal antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1. In some embodiments, the nucleic acid encodes at least three amino acid mutations compared to the germline sequences that are identical to at least three amino acid mutations found in one of the above-listed monoclonal antibodies.

In some embodiments, the nucleic acid molecule comprises a nucleotide sequence that encodes at least a portion of the V_{H} amino acid sequence of antibody 252 (SEQ ID NO: 4), 88 (SEQ ID NO: 8), 100 (SEQ ID NO: 12), 3.8.3 (SEQ ID NO: 16), 2.7.3 (SEQ ID NO: 20), 1.120.1 (SEQ ID NO: 24), 9.14.4I (SEQ ID NO: 28), 8.10.3F (SEQ ID NO: 32), 9.7.2IF (SEQ ID NO: 36), 9.1-4.4 (SEQ ID NO: 28), 8.1-0.3 (SEQ ID NO: 44), 9.7.2 (SEQ ID NO: 48), 9.7.2C-Ser (SEQ ID NO: 52), 9.14.4C-Ser (SEQ ID NO: 56), 8.10.3C-Ser (SEQ ID NO: 60), 8.10.3-CG2 (SEQ ID NO: 60), 9.7.2-CG2 (SEQ ID NO: 52), 9.7.2-CG4 (SEQ ID NO: 52), 9.14.4-CG2 (SEQ ID NO: 56), 9.14.4-CG4 (SEQ ID NO: 56), 9.14.4-Ser (SEQ ID NO: 28), 9.7.2-Ser (SEQ ID NO: 48), 8.10.3-Ser (SEQ ID NO: 44), 8.10.3-CG4 (SEQ ID NO: 60) 8.10.3FG1 (SEQ ID NO: 32) or 9.14.4G1 (SEQ ID NO: 28), or said sequence having conservative amino acid mutations and/or a total of three or fewer non-conservative amino acid substitutions. In various embodiments the sequence encodes one or more CDR regions, preferably a CDR3 region, all three CDR regions, a contiguous portion including CDR1-CDR3, or the entire V_{H} region.

In some embodiments, the nucleic acid molecule comprises a heavy chain nucleotide sequence that encodes the amino acid sequence of one of SEQ ID NOS: 2, 6, 10, 14, 18, 22, 26, 30, 34, 38, 46, 50, 54, 58, 62, 66, 70, 74, 78, 82, 86, 90, 94, 98 or 102. In some preferred embodiments, the nucleic acid molecule comprises at least a portion of the heavy chain nucleotide sequence of SEQ ID NO: 1, 5, 9, 25, 29, 33, 37, 45, 97 or 101. In some embodiments, said portion encodes the V_{H} region, a CDR3 region, all three CDR regions, or a contiguous region including CDR1-CDR3.

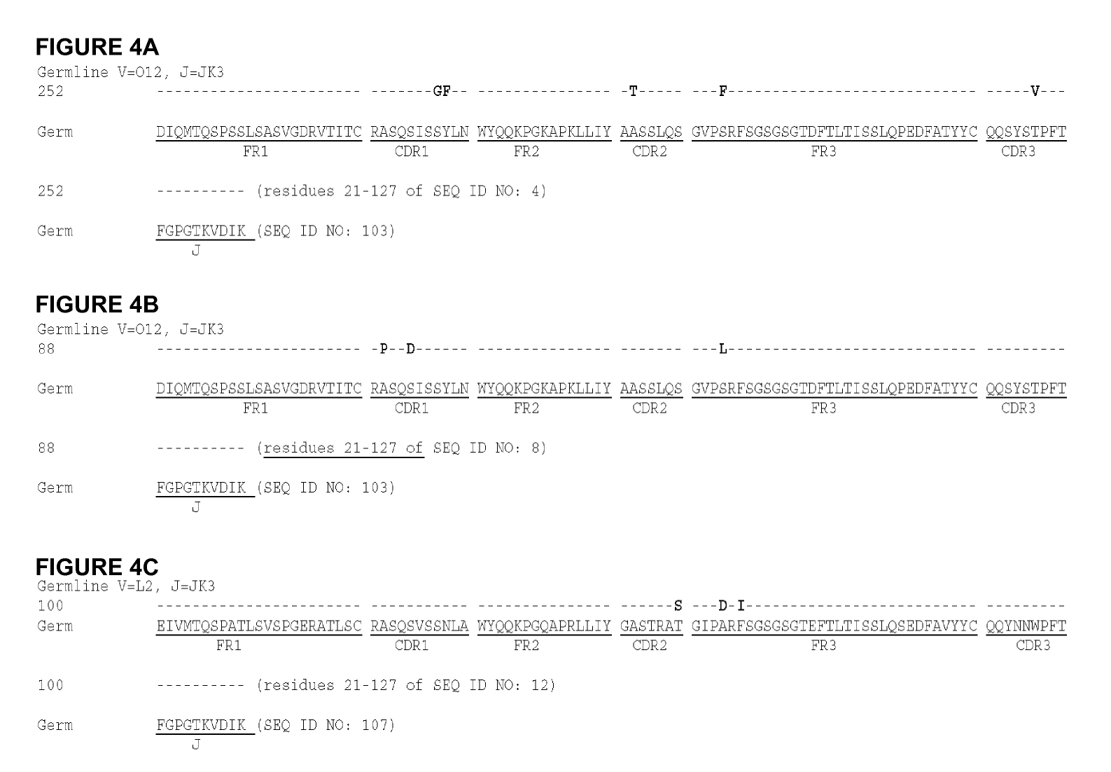

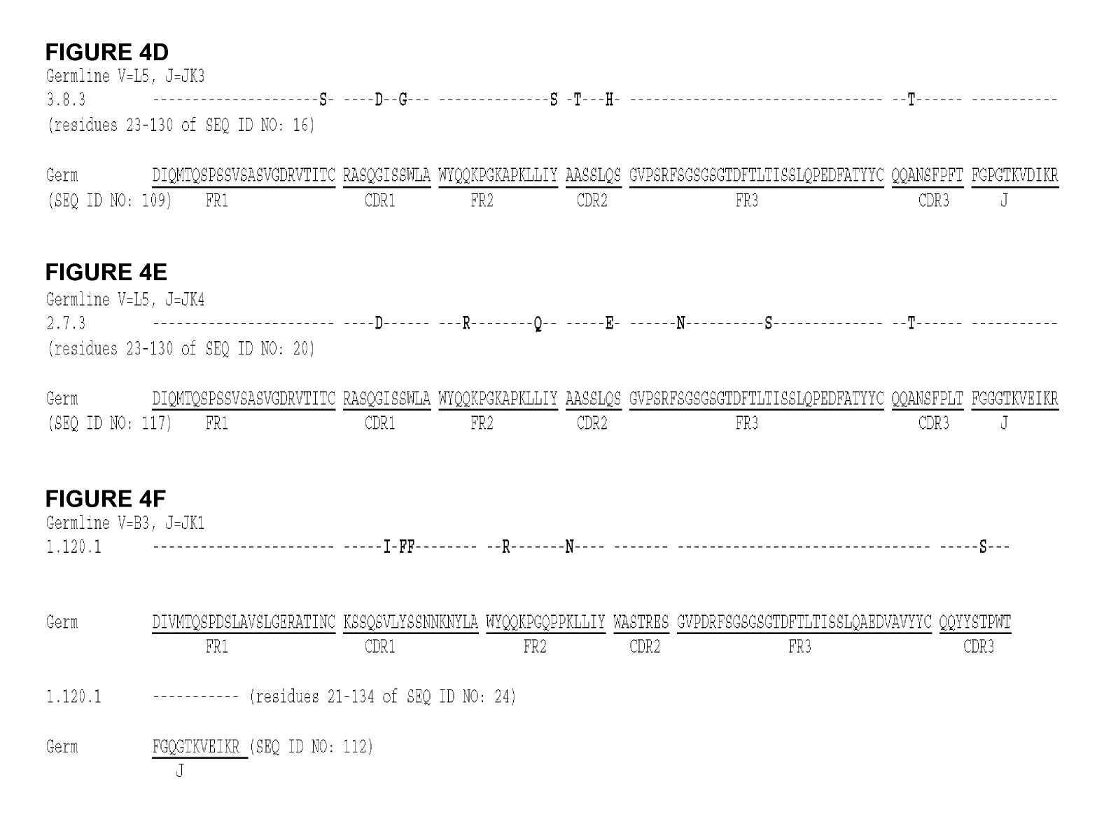

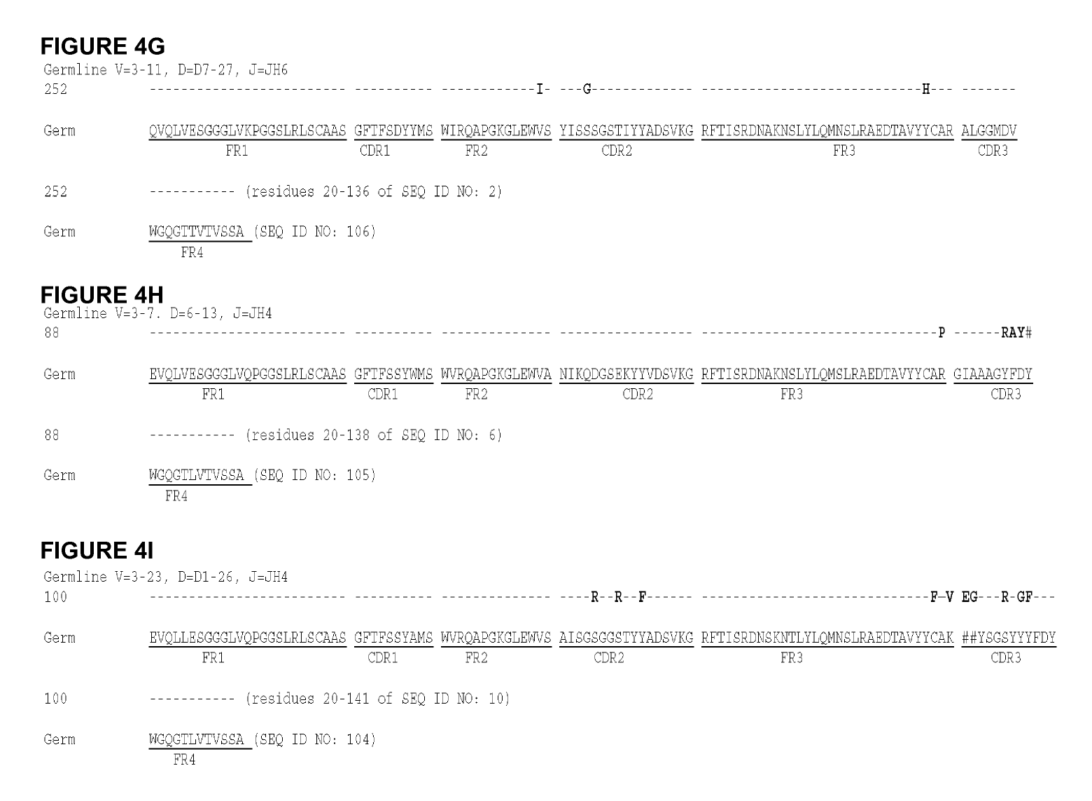

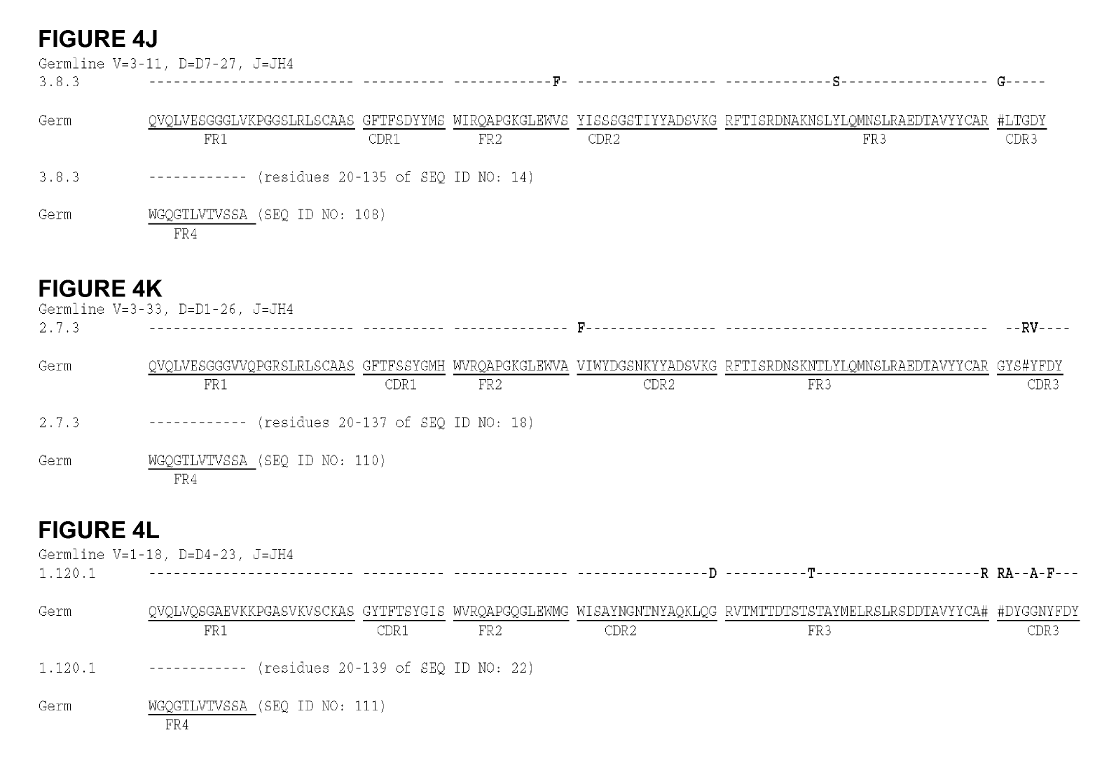

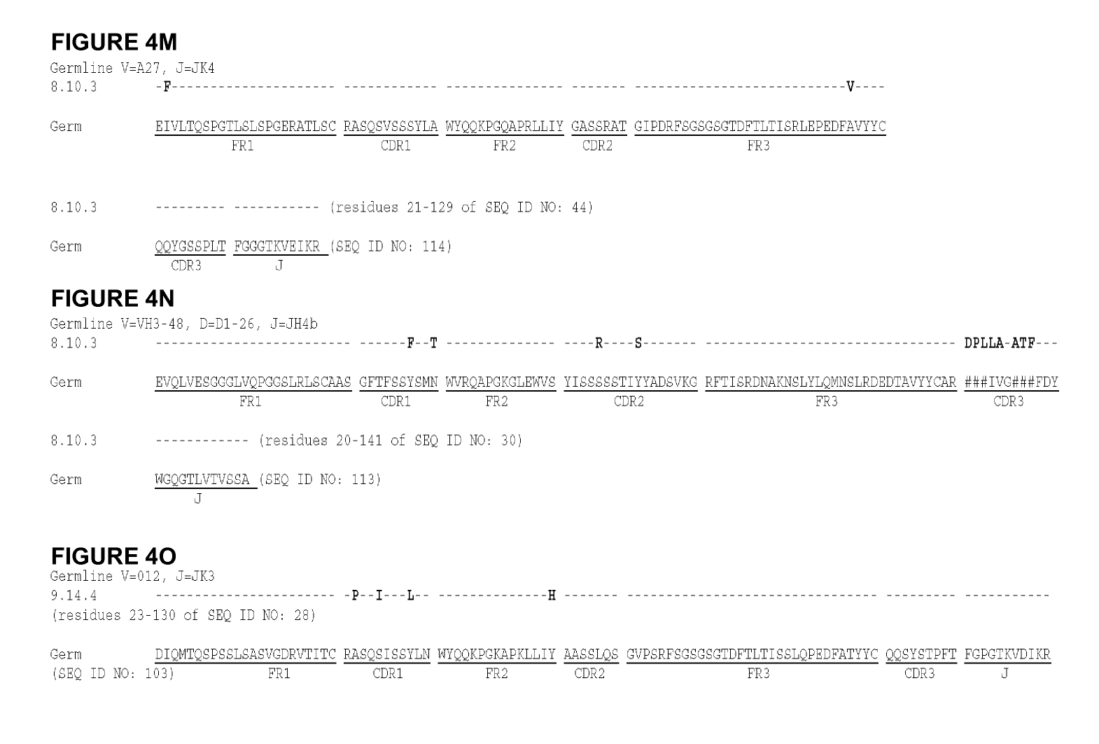

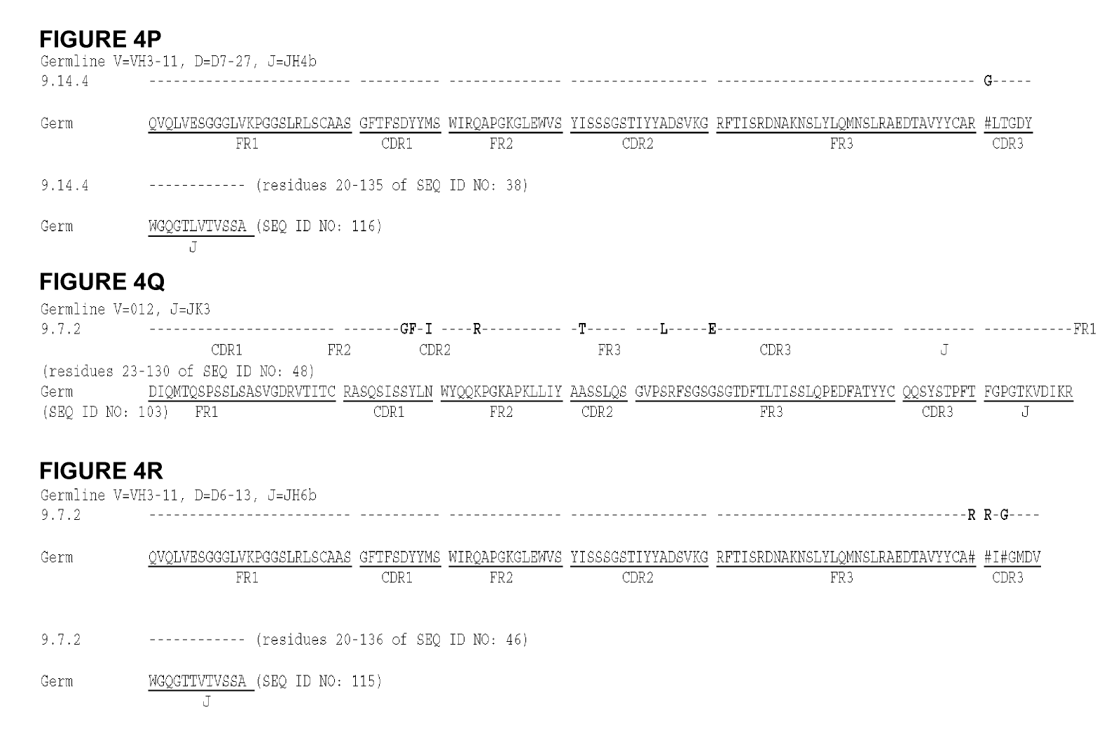

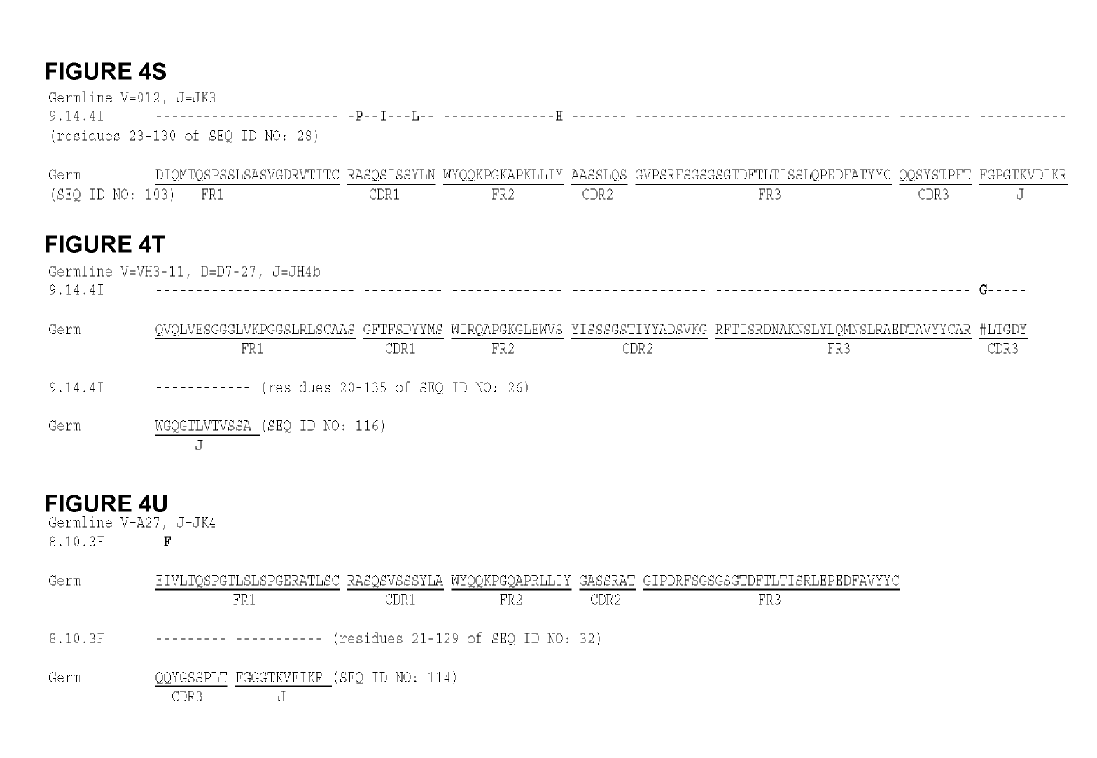

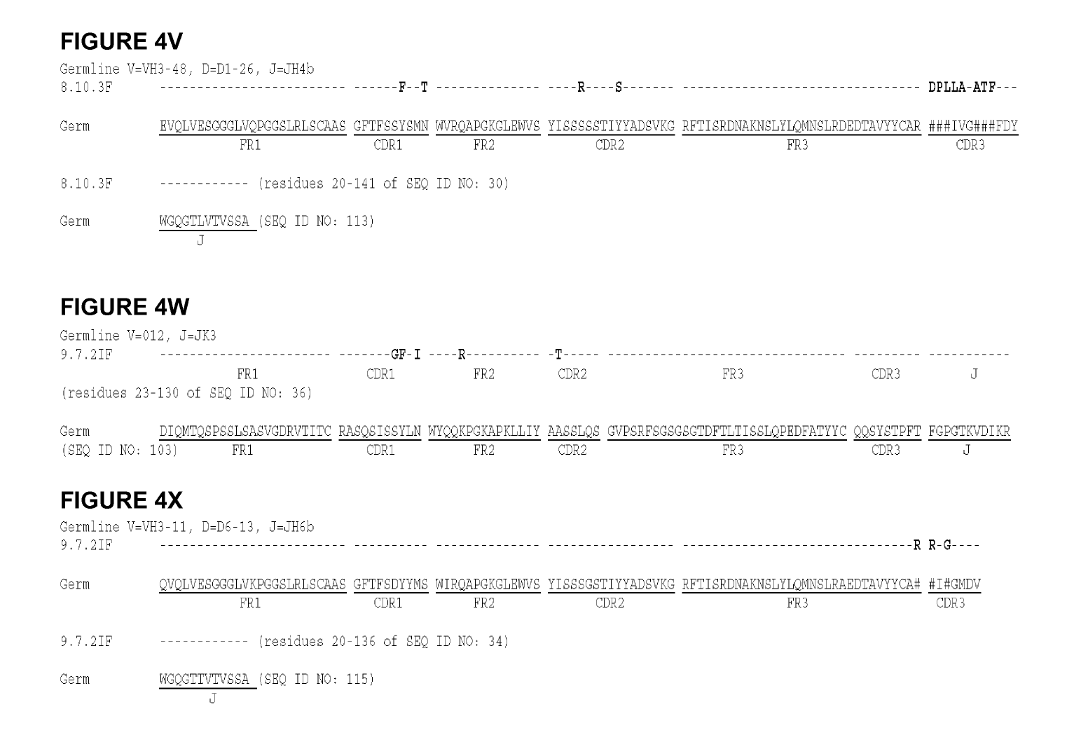

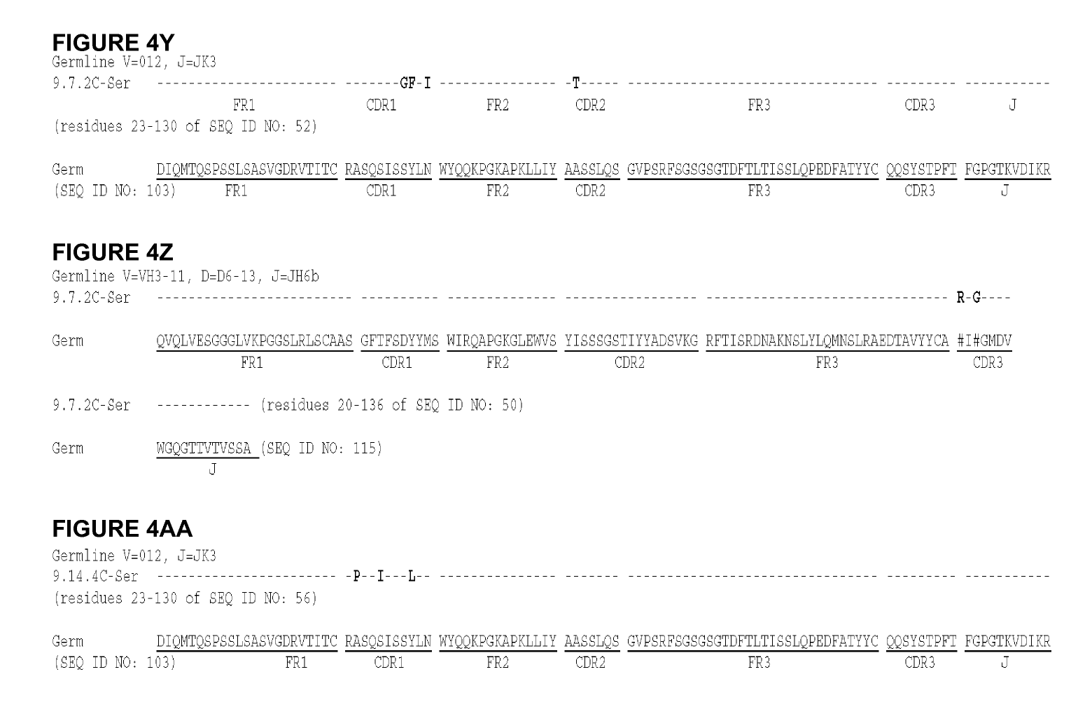

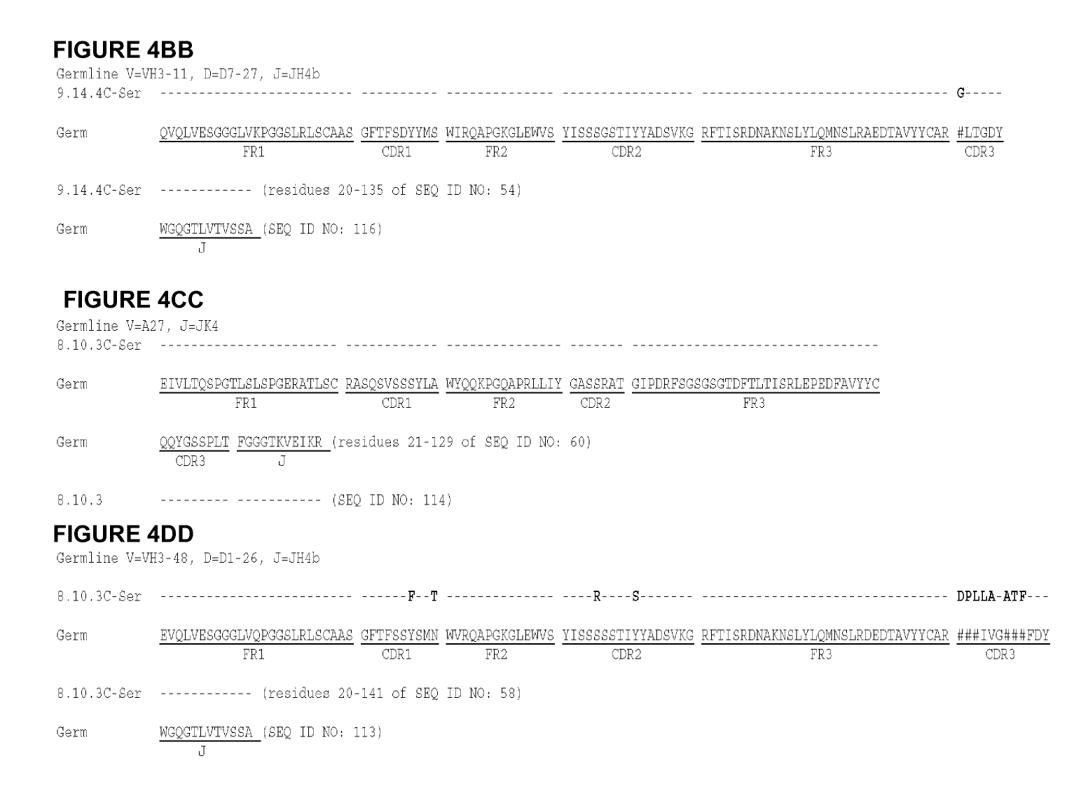

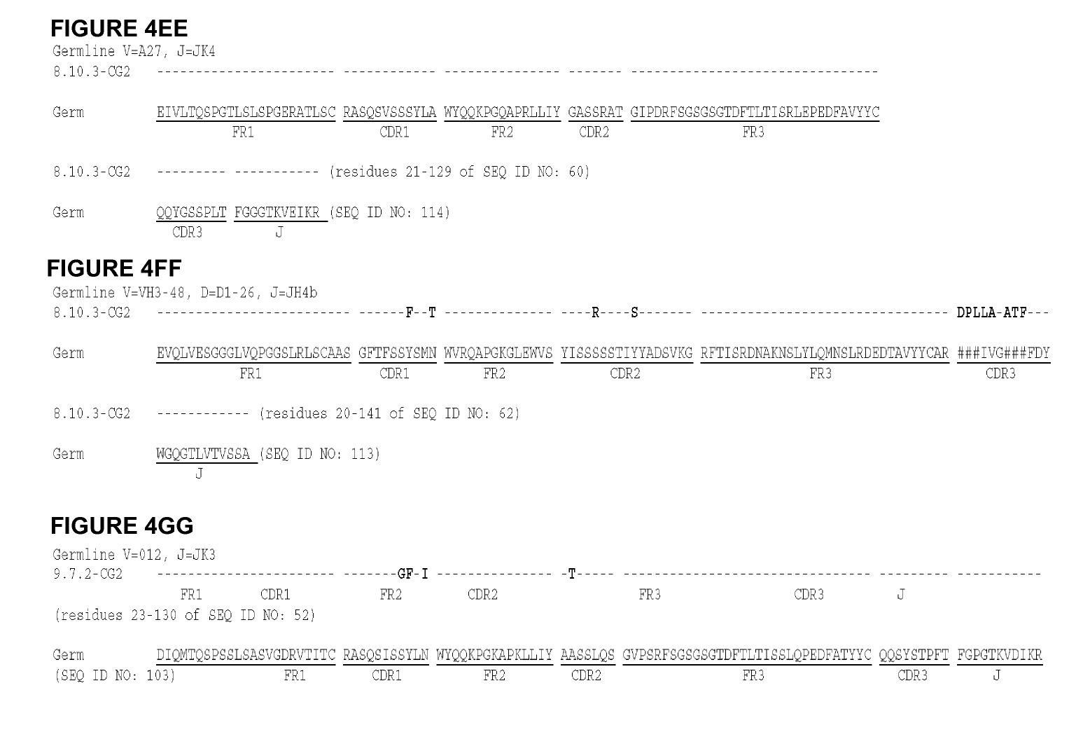

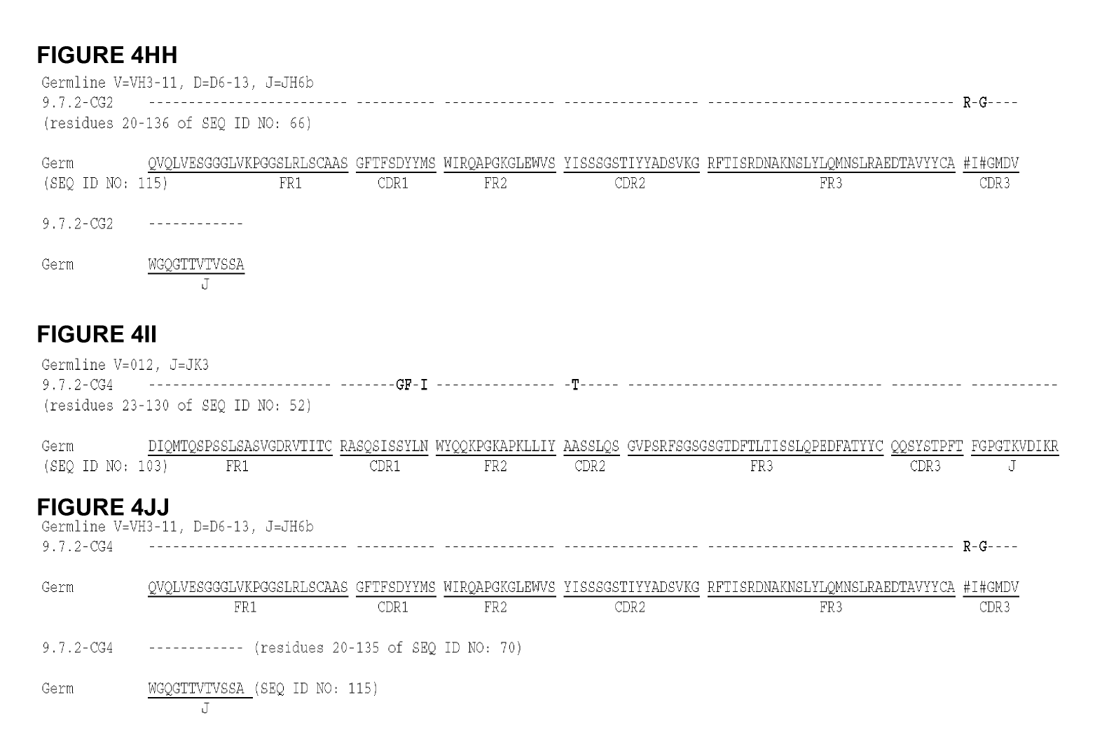

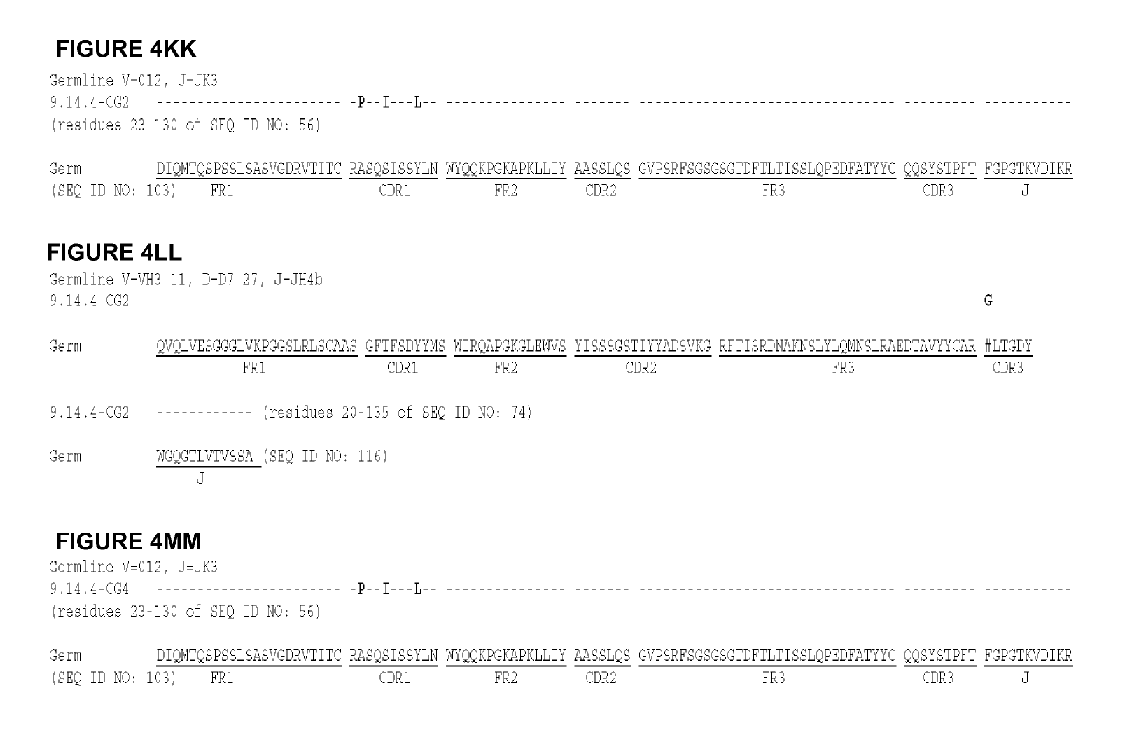

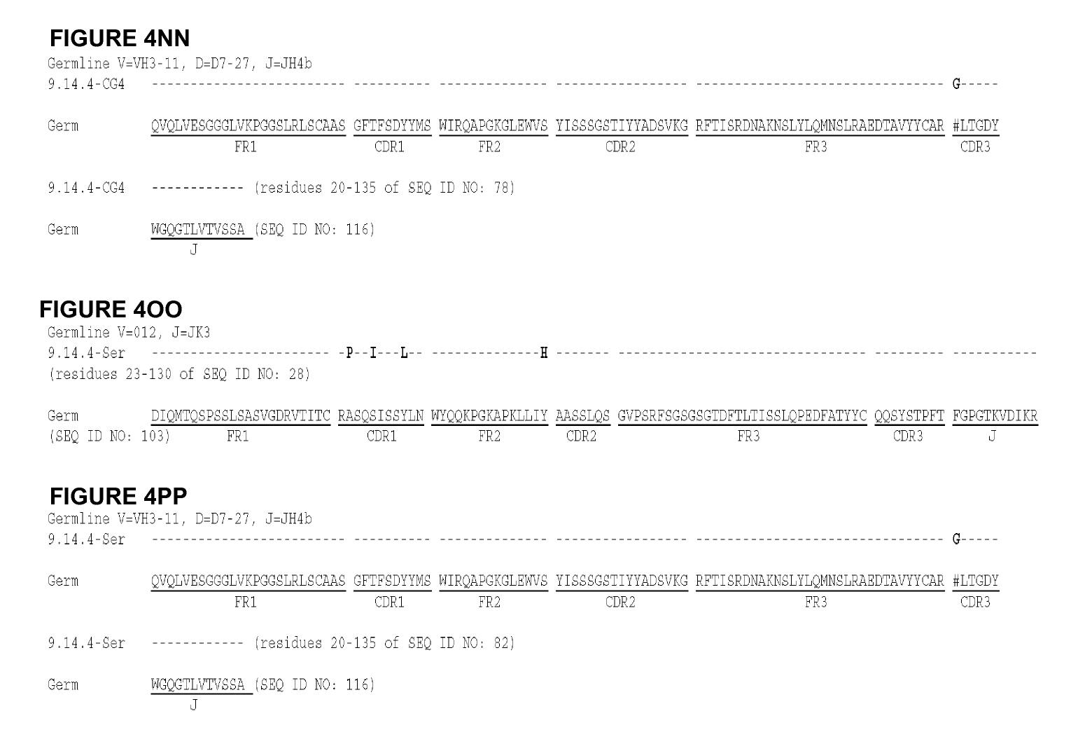

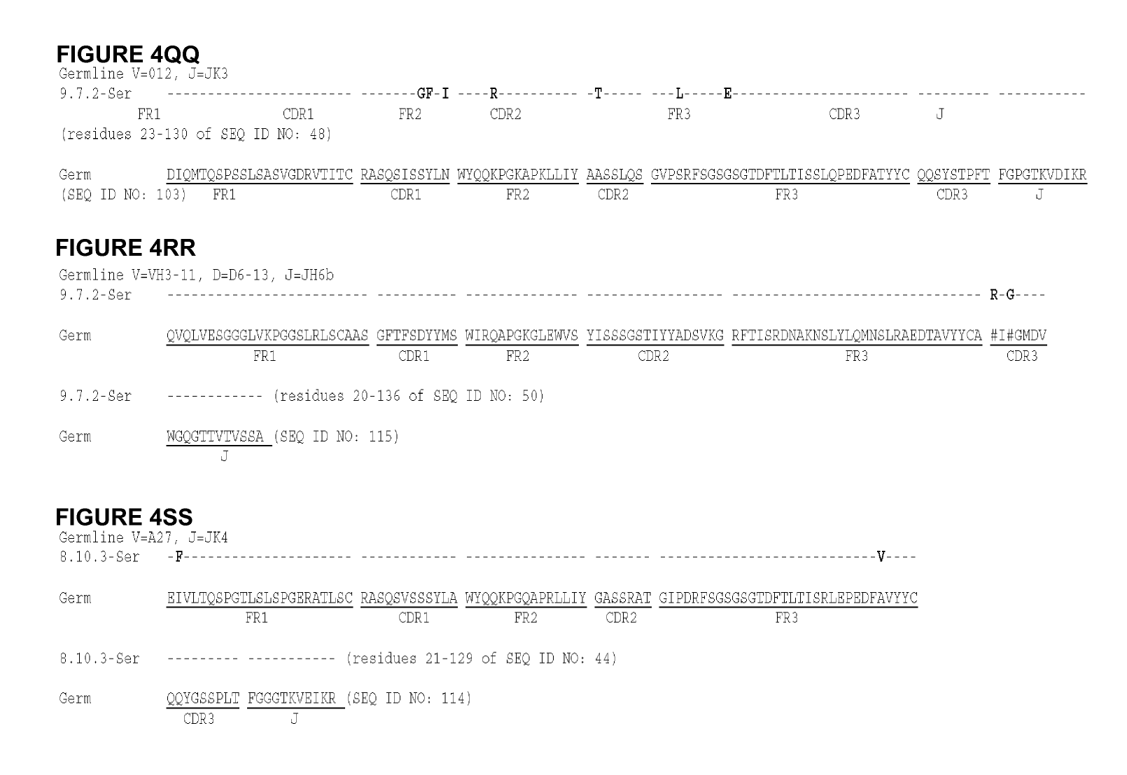

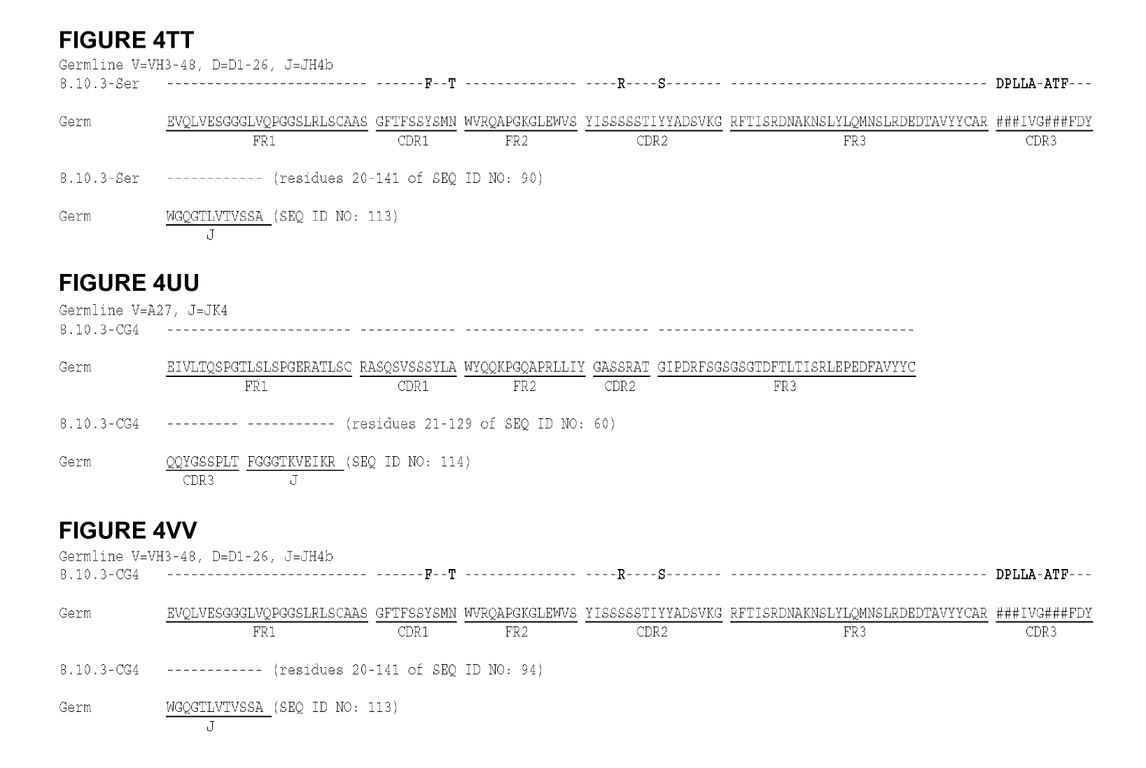

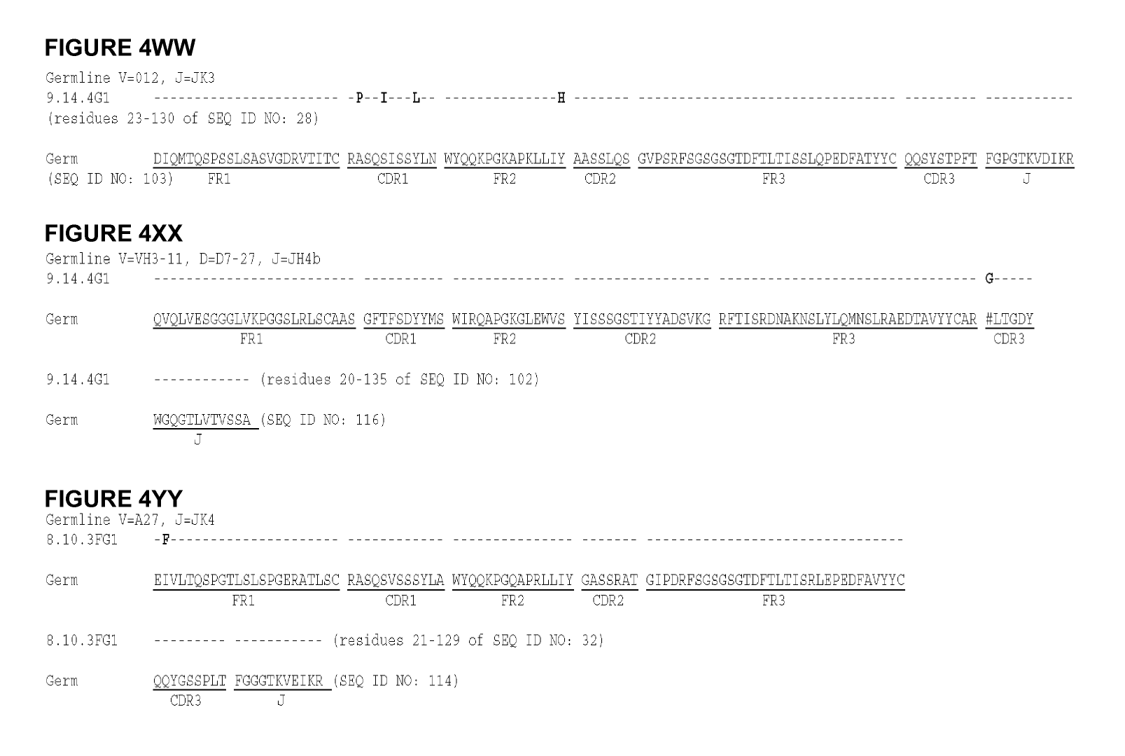

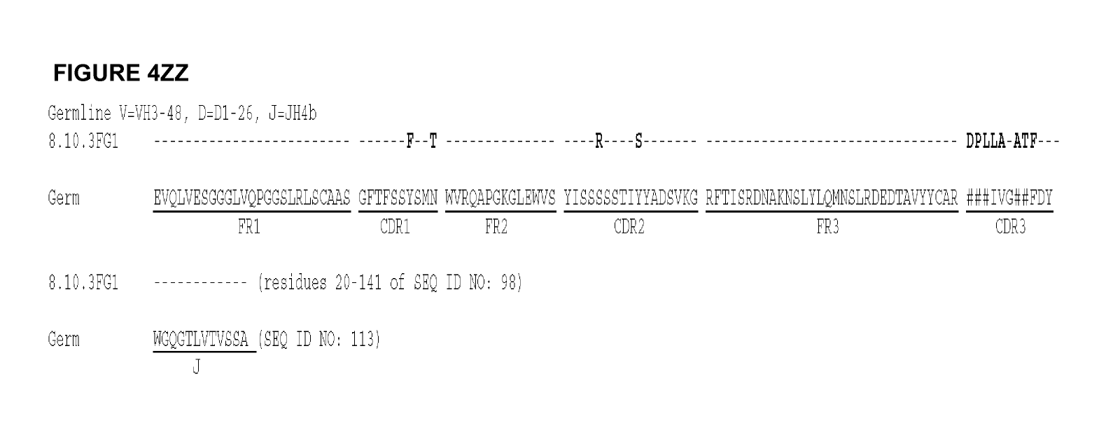

In some embodiments, the nucleic acid molecule encodes a V_{H} amino acid sequence that is at least 70%, 75%, 80%, 85%, 90%, 91%, 92%, 93%, 94%, 95%, 96%, 97%, 98% or 99% identical to the V_{H} amino acid sequences shown in FIG. 4 or to a V_{H} amino acid sequence of any one of SEQ ID NOS: 2, 6, 10, 14, 18, 22, 26, 30, 34, 38, 46, 50, 54, 58, 62, 66, 70, 74, 78, 82, 86, 90, 94, 98 or 102. Nucleic acid molecules of the invention include nucleic acids that hybridize under highly stringent conditions, such as those described above, to a nucleotide sequence encoding the heavy chain amino acid sequence of SEQ ID NOS: 2, 6, 10, 14, 18, 22, 26, 30, 34, 38, 46, 50, 54, 58, 62, 66, 70, 74, 78, 82, 86, 90, 94, 98 or 102 or that has the nucleotide sequence of SEQ ID NOS: 1, 5, 9, 25, 29, 33, 37, 45, 97 or 101.

In another embodiment, the nucleic acid encodes a full-length heavy chain of an antibody selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, or a heavy chain having the amino acid sequence of SEQ ID NOS: 2, 6, 10, 14, 18, 22, 26, 30, 34, 38, 46, 50, 54, 58, 62, 66, 70, 74, 78, 82, 86, 90, 94, 98 or 102 and a constant region of a heavy chain, or a heavy chain comprising a mutation. Further, the nucleic acid may comprise the heavy chain nucleotide sequence of SEQ ID NOS: 1, 5, 9, 25, 29, 33, 37, 45, 97 or 101 and a nucleotide sequence encoding a constant region of a light chain, or a nucleic acid molecule encoding a heavy chain comprising a mutation.

A nucleic acid molecule encoding the heavy or entire light chain of an anti-M-CSF antibody or portions thereof can be isolated from any source that produces such antibody. In various embodiments, the nucleic acid molecules are isolated from a B cell isolated from an animal immunized with M-CSF or from an immortalized cell derived from such a B cell that expresses an anti-M-CSF antibody. Methods of isolating mRNA encoding an antibody are well-known in the art. See, e.g., Sambrook et al. The mRNA may be used to produce cDNA for use in the polymerase chain reaction (PCR) or cDNA cloning of antibody genes. In a preferred embodiment, the nucleic acid molecule is isolated from a hybridoma that has as one of its fusion partners a human immunoglobulin-producing cell from a non-human transgenic animal. In an even more preferred embodiment, the human immunoglobulin producing cell is isolated from a XENOMOUSE™ animal. In another embodiment, the human immunoglobulin-producing cell is from a non-human, non-mouse transgenic animal, as described above. In another embodiment, the nucleic acid is isolated from a non-human, non-transgenic animal. The nucleic acid molecules isolated from a non-human, non-transgenic animal may be used, e.g., for humanized antibodies.

In some embodiments, a nucleic acid encoding a heavy chain of an anti-M-CSF antibody of the invention can comprise a nucleotide sequence encoding a V_{H} domain of the invention joined in-frame to a nucleotide sequence encoding a heavy chain constant domain from any source. Similarly, a nucleic acid molecule encoding a light chain of an anti-M-CSF antibody of the invention can comprise a nucleotide sequence encoding a V_{L} domain of the invention joined in-frame to a nucleotide sequence encoding a light chain constant domain from any source.

In a further aspect of the invention, nucleic acid molecules encoding the variable domain of the heavy (V_{H}) and light (V_{L}) chains are “converted” to full-length antibody genes. In one embodiment, nucleic acid molecules encoding the V_{H} or V_{L} domains are converted to full-length antibody genes by insertion into an expression vector already encoding heavy chain constant (C_{H}) or light chain (C_{L}) constant domains, respectively, such that the V_{H} segment is operatively linked to the C_{H} segment(s) within the vector, and the V_{L} segment is operatively linked to the C_{L} segment within the vector. In another embodiment, nucleic acid molecules encoding the V_{H} and/or V_{L} domains are converted into full-length antibody genes by linking, e.g., ligating, a nucleic acid molecule encoding a V_{H} and/or V_{L} domains to a nucleic acid molecule encoding a C_{H} and/or C_{L} domain using standard molecular biological techniques. Nucleic acid sequences of human heavy and light chain immunoglobulin constant domain genes are known in the art. See, e.g., Kabat et al., Sequences of Proteins of Immunological Interest, 5th Ed., NIH Publ. No. 91-3242, 1991. Nucleic acid molecules encoding the full-length heavy and/or light chains may then be expressed from a cell into which they have been introduced and the anti-M-CSF antibody isolated.

The nucleic acid molecules may be used to recombinantly express large quantities of anti-M-CSF antibodies. The nucleic acid molecules also may be used to produce chimeric antibodies, bispecific antibodies, single chain antibodies, immunoadhesins, diabodies, mutated antibodies and antibody derivatives, as described further below. If the nucleic acid molecules are derived from a non-human, non-transgenic animal, the nucleic acid molecules may be used for antibody humanization, also as described below.

In another embodiment, a nucleic acid molecule of the invention is used as a probe or PCR primer for a specific antibody sequence. For instance, the nucleic acid can be used as a probe in diagnostic methods or as a PCR primer to amplify regions of DNA that could be used, inter alia, to isolate additional nucleic acid molecules encoding variable domains of anti-M-CSF antibodies. In some embodiments, the nucleic acid molecules are oligonucleotides. In some embodiments, the oligonucleotides are from highly variable regions of the heavy and light chains of the antibody of interest. In some embodiments, the oligonucleotides encode all or a part of one or more of the CDRs of antibody 252, 88, 100, 3.8.3, 2.7.3, or 1.120.1, or variants thereof described herein.

### Vectors

The invention provides vectors comprising nucleic acid molecules that encode the heavy chain of an anti-M-CSF antibody of the invention or an antigen-binding portion thereof. The invention also provides vectors comprising nucleic acid molecules that encode the light chain of such antibodies or antigen-binding portion thereof. The invention further provides vectors comprising nucleic acid molecules encoding fusion proteins, modified antibodies, antibody fragments, and probes thereof.

In some embodiments, the anti-M-CSF antibodies, or antigen-binding portions of the invention are expressed by inserting DNAs encoding partial or full-length light and heavy chains, obtained as described above, into expression vectors such that the genes are operatively linked to necessary expression control sequences such as transcriptional and transnational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. The antibody gene is ligated into a vector such that transcriptional and transnational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors. In a preferred embodiment, both genes are inserted into the same expression vector. The antibody genes are inserted into the expression vector by standard methods (e.g., ligation of complementary restriction sites on the antibody gene fragment and vector, or blunt end ligation if no restriction sites are present).

A convenient vector is one that encodes a functionally complete human C_{H} or C_{L} immunoglobulin sequence, with appropriate restriction sites engineered so that any V_{H} or V_{L} sequence can easily be inserted and expressed, as described above. In such vectors, splicing usually occurs between the splice donor site in the inserted J region and the splice acceptor site preceding the human C domain, and also at the splice regions that occur within the human C_{H} exons. Polyadenylation and transcription termination occur at native chromosomal sites downstream of the coding regions. The recombinant expression vector also can encode a signal peptide that facilitates secretion of the antibody chain from a host cell. The antibody chain gene may be cloned into the vector such that the signal peptide is linked in-frame to the amino terminus of the immunoglobulin chain. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. No. 5,168,062, U.S. Pat. No. 4,510,245 and U.S. Pat. No. 4,968,615. Methods for expressing antibodies in plants, including a description of promoters and vectors, as well as transformation of plants is known in the art. See, e.g., U.S. Pat. No. 6,517,529, herein incorporated by reference. Methods of expressing polypeptides in bacterial cells or fungal cells, e.g., yeast cells, are also well known in the art.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification), the neomycin resistance gene (for G418 selection), and the glutamate synthetase gene.

### Non-Hybridoma Host Cells and Methods of Recombinantly Producing Protein

Nucleic acid molecules encoding anti-M-CSF antibodies and vectors comprising these nucleic acid molecules can be used for transfection of a suitable mammalian, plant, bacterial or yeast host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455 (which patents are hereby incorporated herein by reference). Methods of transforming plant cells are well known in the art, including, e.g., Agrobacterium-mediated transformation, biolistic transformation, direct injection, electroporation and viral transformation. Methods of transforming bacterial and yeast cells are also well known in the art.

Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Cell lines of particular preference are selected through determining which cell lines have high expression levels. Other cell lines that may be used are insect cell lines, such as Sf9 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Plant host cells include, e.g., Nicotiana, Arabidopsis, duckweed, corn, wheat, potato, etc. Bacterial host cells include E. coli and Streptomyces species. Yeast host cells include Schizosaccharomyces pombe, Saccharomyces cerevisiae and Pichia pastoris.

Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

It is possible that antibodies expressed by different cell lines or in transgenic animals will have different glycosylation from each other. However, all antibodies encoded by the nucleic acid molecules provided herein, or comprising the amino acid sequences provided herein are part of the instant invention, regardless of the glycosylation state or pattern or modification of the antibodies.

### Transgenic Animals and Plants

Anti-M-CSF antibodies of the invention also can be produced transgenically through the generation of a mammal or plant that is transgenic for the immunoglobulin heavy and light chain sequences of interest and production of the antibody in a recoverable form therefrom. In connection with the transgenic production in mammals, anti-M-CSF antibodies can be produced in, and recovered from, the milk of goats, cows, or other mammals. See, e.g., U.S. Pat. Nos. 5,827,690, 5,756,687, 5,750,172, and 5,741,957. In some embodiments, non-human transgenic animals that comprise human immunoglobulin loci are immunized with M-CSF or an immunogenic portion thereof, as described above. Methods for making antibodies in plants, yeast or fungi/algae are described, e.g., in U.S. Pat. No. 6,046,037 and U.S. Pat. No. 5,959,177.

In some embodiments, non-human transgenic animals or plants are produced by introducing one or more nucleic acid molecules encoding an anti-M-CSF antibody of the invention into the animal or plant by standard transgenic techniques. See Hogan and U.S. Pat. No. 6,417,429, supra. The transgenic cells used for making the transgenic animal can be embryonic stem cells or somatic cells. The transgenic non-human organisms can be chimeric, nonchimeric heterozygotes, and nonchimeric homozygotes. See, e.g., Hogan et al., Manipulating the Mouse Embryo: A Laboratory Manual 2ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999). In some embodiments, the transgenic non-human animals have a targeted disruption and replacement by a targeting construct that encodes a heavy chain and/or a light chain of interest. In a preferred embodiment, the transgenic animals comprise and express nucleic acid molecules encoding heavy and light chains that specifically bind to M-CSF, preferably human M-CSF. In some embodiments, the transgenic animals comprise nucleic acid molecules encoding a modified antibody such as a single-chain antibody, a chimeric antibody or a humanized antibody. The anti-M-CSF antibodies may be made in any transgenic animal. In a preferred embodiment, the non-human animals are mice, rats, sheep, pigs, goats, cattle or horses. The non-human transgenic animal expresses said encoded polypeptides in blood, milk, urine, saliva, tears, mucus and other bodily fluids.

### Phage Display Libraries

The invention provides a method for producing an anti-M-CSF antibody or antigen-binding portion thereof comprising the steps of synthesizing a library of human antibodies on phage, screening the library with M-CSF or a portion thereof, isolating phage that bind M-CSF, and obtaining the antibody from the phage. By way of example, one method for preparing the library of antibodies for use in phage display techniques comprises the steps of immunizing a non-human animal comprising human immunoglobulin loci with M-CSF or an antigenic portion thereof to create an immune response, extracting antibody producing cells from the immunized animal; isolating RNA from the extracted cells, reverse transcribing the RNA to produce cDNA, amplifying the cDNA using a primer, and inserting the cDNA into a phage display vector such that antibodies are expressed on the phage. Recombinant anti-M-CSF antibodies of the invention may be obtained in this way.

Recombinant anti-M-CSF human antibodies of the invention can be isolated by screening a recombinant combinatorial antibody library. Preferably the library is a scFv phage display library, generated using human V_{L} and V_{H} cDNAs prepared from mRNA isolated from B cells. Methodologies for preparing and screening such libraries are known in the art. There are commercially available kits for generating phage display libraries (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology 9:1370-1372 (1991); Hay et al., Hum. Antibod. Hybridomas 3:81-85 (1992); Huse et al., Science 246:1275-1281 (1989); McCafferty et al., Nature 348:552-554 (1990); Griffiths et al., EMBO J. 12:725-734 (1993); Hawkins et al., J. Mol. Biol. 226:889-896 (1992); Clackson et al., Nature 352:624-628 (1991); Gram et al., Proc. Natl. Acad. Sci. USA 89:3576-3580 (1992); Garrad et al., Bio/Technology 9:1373-1377 (1991); Hoogenboom et al., Nuc. Acid Res. 19:4133-4137 (1991); and Barbas at al., Proc. Natl. Acad. Sci. USA 88:7978-7982 (1991).

In one embodiment, to isolate a human anti-M-CSF antibodies with the desired characteristics, a human anti-M-CSF antibody as described herein is first used to select human heavy and light chain sequences having similar binding activity toward M-CSF, using the epitope imprinting methods described in PCT Publication No. WO 93/06213. The antibody libraries used in this method are preferably scFv libraries prepared and screened as described in PCT Publication No. WO 92/01047, McCafferty et al., Nature 348:552-554 (1990); and Griffiths et al., EMBO J. 12:725-734 (1993). The scFv antibody libraries preferably are screened using human M-CSF as the antigen.

Once initial human V_{L} and V_{H} domains are selected, “mix and match” experiments are performed, in which different pairs of the initially selected V_{L} and V_{H} segments are screened for M-CSF binding to select preferred V_{L}/V_{H} pair combinations. Additionally, to further improve the quality of the antibody, the V_{L} and V_{H} segments of the preferred V_{L}/V_{H} pair(s) can be randomly mutated, preferably within the CDR3 region of V_{H} and/or V_{L}, in a process analogous to the in vivo somatic mutation process responsible for affinity maturation of antibodies during a natural immune response. This in vitro affinity maturation can be accomplished by amplifying V_{H} and V_{L} domains using PCR primers complimentary to the V_{H} CDR3 or V_{L} CDR3, respectively, which primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be re-screened for binding to M-CSF.

Following screening and isolation of an anti-M-CSF antibody of the invention from a recombinant immunoglobulin display library, nucleic acids encoding the selected antibody can be recovered from the display package (e.g., from the phage genome) and subcloned into other expression vectors by standard recombinant DNA techniques. If desired, the nucleic acid can further be manipulated to create other antibody forms of the invention, as described below. To express a recombinant human antibody isolated by screening of a combinatorial library, the DNA encoding the antibody is cloned into a recombinant expression vector and introduced into a mammalian host cells, as described above.

### Class Switching

Another aspect of the invention provides a method for converting the class or subclass of an anti-M-CSF antibody to another class or subclass. In some embodiments, a nucleic acid molecule encoding a V_{L} or V_{H} that does not include any nucleic acid sequences encoding C_{L} or C_{H} is isolated using methods well-known in the art. The nucleic acid molecule then is operatively linked to a nucleic acid sequence encoding a C_{L} or C_{H} from a desired immunoglobulin class or subclass. This can be achieved using a vector or nucleic acid molecule that comprises a C_{L} or C_{H} chain, as described above. For example, an anti-M-CSF antibody that was originally IgM can be class switched to an IgG. Further, the class switching may be used to convert one IgG subclass to another, e.g., from IgG1 to IgG2. Another method for producing an antibody of the invention comprising a desired isotype comprises the steps of isolating a nucleic acid encoding a heavy chain of an anti-M-CSF antibody and a nucleic acid encoding a light chain of an anti-M-CSF antibody, isolating the sequence encoding the V_{H} region, ligating the V_{H} sequence to a sequence encoding a heavy chain constant domain of the desired isotype, expressing the light chain gene and the heavy chain construct in a cell, and collecting the anti-M-CSF antibody with the desired isotype.

In some embodiments, anti-M-CSF antibodies of the invention have the serine at position 228 (according to the EU-numbering convention) of the heavy chain changed to a proline. Accordingly, the CPSC sub-sequence in the F_{C} region of IgG4 becomes CPPC, which is the sub-sequence in IgG1. (Aalberse, R. C. and Schuurman, J., Immunology, 105:9-19 (2002)). For example, the serine at residue 243 SEQ ID NO: 46 (which corresponds to reside 228 in the EU-numbering convention) would become proline. Similarly, the serine at residue 242 of SEQ ID NO: 38 (which corresponds to reside 228 in the EU-numbering convention) would become proline. In some embodiments, the framework region of the IgG4 antibody can be back-mutated to the germline framework sequence. Some embodiments comprise both the back-mutates framework region and the serine to proline change in the F_{C} region. See, e.g., SEQ ID NO: 54 (antibody 9.14.4C-Ser) and SEQ ID NO: 58 (antibody 8.10.3C-Ser) in Table 1A.

### Deimmunized Antibodies

Another way of producing antibodies with reduced immunogenicity is the deimmunization of antibodies. In another aspect of the invention, the antibody may be deimmunized using the techniques described in, e.g., PCT Publication Nos. WO98/52976 and WO00/34317 (which incorporated herein by reference in their entirety).

### Mutated Antibodies

In another embodiment, the nucleic acid molecules, vectors and host cells may be used to make mutated anti-M-CSF antibodies. The antibodies may be mutated in the variable domains of the heavy and/or light chains, e.g., to alter a binding property of the antibody. For example, a mutation may be made in one or more of the CDR regions to increase or decrease the K_{D} of the antibody for M-CSF, to increase or decrease k_{off}, or to alter the binding specificity of the antibody. Techniques in site-directed mutagenesis are well-known in the art. See, e.g., Sambrook et al. and Ausubel et al., supra. In a preferred embodiment, mutations are made at an amino acid residue that is known to be changed compared to germline in a variable domain of an anti-M-CSF antibody. In another embodiment, one or more mutations are made at an amino acid residue that is known to be changed compared to the germline in a CDR region or framework region of a variable domain, or in a constant domain of a monoclonal antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2,9,7,2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1. In another embodiment, one or more mutations are made at an amino acid residue that is known to be changed compared to the germline in a CDR region or framework region of a variable domain of a heavy chain amino acid sequence selected from SEQ ID NOS: 2, 6, 10, 14, 18, 22, 26, 30, 34, 38, 46, 50, 54, 58, 62, 66, 70, 74, 78, 82, 86, 90, 94, 98 or 102, or whose heavy chain nucleotide sequence is presented in SEQ ID NOS: 1, 5, 9, 25, 29, 33, 37, 45, 97 or 101. In another embodiment, one or more mutations are made at an amino acid residue that is known to be changed compared to the germline in a CDR region or framework region of a variable domain of a light chain amino acid sequence selected from SEQ ID NOS: 4, 8, 12, 16, 20, 24, 28, 32, 36, 44, 48, 52, 56 or 60, or whose light chain nucleotide sequence is presented in SEQ ID NOS: 3, 7, 11, 27, 31, 35, 43 or 47.

In one embodiment, the framework region is mutated so that the resulting framework region(s) have the amino acid sequence of the corresponding germline gene. A mutation may be made in a framework region or constant domain to increase the half-life of the anti-M-CSF antibody. See, e.g., PCT Publication No. WO 00/09560, herein incorporated by reference. A mutation in a framework region or constant domain also can be made to alter the immunogenicity of the antibody, to provide a site for covalent or non-covalent binding to another molecule, or to alter such properties as complement fixation, FcR binding and antibody-dependent cell-mediated cytotoxicity (ADCC). According to the invention, a single antibody may have mutations in any one or more of the CDRs or framework regions of the variable domain or in the constant domain.

In some embodiments, there are from 1 to 8 including any number in between, amino acid mutations in either the V_{H} or V_{L} domains of the mutated anti-M-CSF antibody compared to the anti-M-CSF antibody prior to mutation. In any of the above, the mutations may occur in one or more CDR regions. Further, any of the mutations can be conservative amino acid substitutions. In some embodiments, there are no more than 5, 4, 3, 2, or 1 amino acid changes in the constant domains.

### Modified Antibodies

In another embodiment, a fusion antibody or immunoadhesin may be made that comprises all or a portion of an anti-M-CSF antibody of the invention linked to another polypeptide. In a preferred embodiment, only the variable domains of the anti-M-CSF antibody are linked to the polypeptide. In another preferred embodiment, the V_{H} domain of an anti-M-CSF antibody is linked to a first polypeptide, while the V_{L} domain of an anti-M-CSF antibody is linked to a second polypeptide that associates with the first polypeptide in a manner such that the V_{H} and V_{L} domains can interact with one another to form an antibody binding site. In another preferred embodiment, the V_{H} domain is separated from the V_{L} domain by a linker such that the V_{H} and V_{L} domains can interact with one another (see below under Single Chain Antibodies). The V_{H}-linker-V_{L} antibody is then linked to the polypeptide of interest. The fusion antibody is useful for directing a polypeptide to a M-CSF-expressing cell or tissue. The polypeptide may be a therapeutic agent, such as a toxin, growth factor or other regulatory protein, or may be a diagnostic agent, such as an enzyme that may be easily visualized, such as horseradish peroxidase. In addition, fusion antibodies can be created in which two (or more) single-chain antibodies are linked to one another. This is useful if one wants to create a divalent or polyvalent antibody on a single polypeptide chain, or if one wants to create a bispecific antibody.

To create a single chain antibody, (scFv) the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker. See, e.g., Bird et al., Science 242:423-426 (1988); Huston et al., Proc. Natl. Acad. Sci. USA 85:5879-5883 (1988); McCafferty et al., Nature 348:552-554 (1990). The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to M-CSF and to another molecule.

In other embodiments, other modified antibodies may be prepared using anti-M-CSF antibody-encoding nucleic acid molecules. For instance, “Kappa bodies” (III et al., Protein Eng. 10: 949-57 (1997)), “Minibodies” (Martin et al., EMBO J. 13: 5303-9 (1994)), “Diabodies” (Holliger et al., Proc. Natl. Acad. Sci. USA 90: 6444-6448 (1993)), or “Janusins” (Traunecker et al., EMBO J. 10:3655-3659 (1991) and Traunecker et al., Int. J. Cancer (Suppl.) 7:51-52 (1992)) may be prepared using standard molecular biological techniques following the teachings of the specification.

Bispecific antibodies or antigen-binding fragments can be produced by a variety of methods including fusion of hybridomas or linking of Fab′ fragments. See, e.g., Songsivilai & Lachmann, Clin. Exp. Immunol. 79: 315-321 (1990), Kostelny et al., J. Immunol. 148:1547-1553 (1992). In addition, bispecific antibodies may be formed as “diabodies” or “Janusins.” In some embodiments, the bispecific antibody binds to two different epitopes of M-CSF. In some embodiments, the bispecific antibody has a first heavy chain and a first light chain from monoclonal antibody 252, 88, 100, 3.8.3, 2.7.3, 1.120.1, 9.14.4I, 8.10.3F, 9.7.2IF, 9.14.4, 8.10.3, or 9.7.2 and an additional antibody heavy chain and light chain. In some embodiments, the additional light chain and heavy chain also are from one of the above-identified monoclonal antibodies, but are different from the first heavy and light chains.

In some embodiments, the modified antibodies described above are prepared using one or more of the variable domains or CDR regions from a human anti-M-CSF monoclonal antibody provided herein, from an amino acid sequence of said monoclonal antibody, or from a heavy chain or light chain encoded by a nucleic acid sequence encoding said monoclonal antibody.

### Derivatized and Labeled Antibodies

An anti-M-CSF antibody or antigen-binding portion of the invention can be derivatized or linked to another molecule (e.g., another peptide or protein). In general, the antibodies or portion thereof is derivatized such that the M-CSF binding is not affected adversely by the derivatization or labeling. Accordingly, the antibodies and antibody portions of the invention are intended to include both intact and modified forms of the human anti-M-CSF antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antigen-binding portion of the invention may be derivatized include fluorescent compounds, including fluorescein, fluorescein isothiocyanate, rhodamine, 5-dimethylamine-1-naphthalenesulfonyl chloride, phycoerythrin, lanthanide phosphors and the like. An antibody can also be labeled with enzymes that are useful for detection, such as horseradish peroxidase, β-galactosidase, luciferase, alkaline phosphatase, glucose oxidase and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a colored reaction product, which is detectable. An antibody can also be labeled with biotin, and detected through indirect measurement of avidin or streptavidin binding. An antibody can also be labeled with a predetermined polypeptide epitope recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

An anti-M-CSF antibody can also be labeled with a radiolabeled amino acid. The radiolabeled anti-M-CSF antibody can be used for both diagnostic and therapeutic purposes. For instance, the radiolabeled anti-M-CSF antibody can be used to detect M-CSF-expressing tumors by x-ray or other diagnostic techniques. Further, the radiolabeled anti-M-CSF antibody can be used therapeutically as a toxin for cancerous cells or tumors. Examples of labels for polypeptides include, but are not limited to, the following radioisotopes or radionuclides—³H, ¹⁴C, ¹⁵N, ³⁵S, ⁹⁰Y, ⁹⁹Tc, ¹¹¹In, ¹²⁵I, and ¹³¹I.

An anti-M-CSF antibody can also be derivatized with a chemical group such as polyethylene glycol (PEG), a methyl or ethyl group, or a carbohydrate group. These groups are useful to improve the biological characteristics of the antibody, e.g., to increase serum half-life or to increase tissue binding.

### Pharmaceutical Compositions and Kits

The invention also relates to compositions comprising a human anti-M-CSF antagonist antibody for the treatment of subjects in need of treatment for rheumatoid arthritis, osteoporosis, or atherosclerosis. In some embodiments, the subject of treatment is a human. In other embodiments, the subject is a veterinary subject. Hyperproliferative disorders where monocytes play a role that may be treated by an antagonist anti-M-CSF antibody of the invention can involve any tissue or organ and include but are not limited to brain, lung, squamous cell, bladder, gastric, pancreatic, breast, head, neck, liver, renal, ovarian, prostate, colorectal, esophageal, gynecological, nasopharynx, or thyroid cancers, melanomas, lymphomas, leukemias or multiple myelomas. In particular, human antagonist anti-M-CSF antibodies of the invention are useful to treat or prevent carcinomas of the breast, prostate, colon and lung.

This invention also encompasses compositions for the treatment of a condition selected from the group consisting of lupus, including systemic lupus erythematosus, lupus nephritis, and cutaneous lupus, arthritis, psoriatic arthritis, Reiter's syndrome, gout, traumatic arthritis, rubella arthritis and acute synovitis, rheumatoid arthritis, rheumatoid spondylitis, ankylosing spondylitis, osteoarthritis, gouty arthritis and other arthritic conditions, sepsis, septic shock, endotoxic shock, gram negative sepsis, toxic shock syndrome, Alzheimer's disease, stroke, neurotrauma, asthma, adult respiratory distress syndrome, cerebral malaria, chronic pulmonary inflammatory disease, silicosis, pulmonary sarcoidosis, bone resorption disease, osteoporosis, restenosis, cardiac and renal reperfusion injury, thrombosis, glomerularonephritis, diabetes, graft vs. host reaction, allograft rejection, inflammatory bowel disease, Crohn's disease, ulcerative colitis, multiple sclerosis, muscle degeneration, eczema, contact dermatitis, psoriasis, sunburn, or conjunctivitis in a mammal, including a human, comprising an amount of a human anti-M-CSF monoclonal antibody of the invention effective in such treatment and a pharmaceutically acceptable carrier.

Treatment may involve administration of one or more antagonist anti-M-CSF monoclonal antibodies of the invention, or antigen-binding fragments thereof, alone or with a pharmaceutically acceptable carrier. As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Anti-M-CSF antibodies of the invention and compositions comprising them, can be administered in combination with one or more other therapeutic, diagnostic or prophylactic agents. Additional therapeutic agents include other anti-neoplastic, anti-tumor, anti-angiogenic or chemotherapeutic agents. Such additional agents may be included in the same composition or administered separately. In some embodiments, one or more inhibitory anti-M-CSF antibodies of the invention can be used as a vaccine or as adjuvants to a vaccine.

The compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection. In another embodiment, the invention includes a method of treating a subject in need thereof with an antibody or an antigen-binding portion thereof that specifically binds to M-CSF comprising the steps of: (a) administering an effective amount of an isolated nucleic acid molecule encoding the heavy chain or the antigen-binding portion thereof, an isolated nucleic acid molecule encoding the light chain or the antigen-binding portion thereof, or both the nucleic acid molecules encoding the light chain and the heavy chain or antigen-binding portions thereof; and (b) expressing the nucleic acid molecule.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the anti-M-CSF antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies of the present invention can be administered by a variety of methods known in the art, although for many therapeutic applications, the preferred route/mode of administration is subcutaneous, intramuscular, or intravenous infusion. As will be appreciated by the skilled artisan, the route and/or mode of administration will vary depending upon the desired results.

In certain embodiments, the antibody compositions active compound may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc. New York, 1978).

In certain embodiments, an anti-M-CSF antibody of the invention can be orally administered, for example, with an inert diluent or an assimilable edible carrier. The compound (and other ingredients, if desired) can also be enclosed in a hard or soft shell gelatin capsule, compressed into tablets, or incorporated directly into the subject's diet. For oral therapeutic administration, the anti-M-CSF antibodies can be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Additional active compounds also can be incorporated into the compositions. In certain embodiments, an anti-M-CSF antibody of the invention is co-formulated with and/or co-administered with one or more additional therapeutic agents. These agents include antibodies that bind other targets, antineoplastic agents, antitumor agents, chemotherapeutic agents, peptide analogues that inhibit M-CSF, soluble c-fms that can bind M-CSF, one or more chemical agents that inhibit M-CSF, anti-inflammatory agents, anti-coagulants, agents that lower blood pressure (i.e, angiotensin-converting enzyme (ACE) inhibitors). Such combination therapies may require lower dosages of the anti-M-CSF antibody as well as the co-administered agents, thus avoiding possible toxicities or complications associated with the various monotherapies.

Inhibitory anti-M-CSF antibodies of the invention and compositions comprising them also may be administered in combination with other therapeutic regimens, in particular in combination with radiation treatment for cancer. The compounds of the present invention may also be used in combination with anticancer agents such as endostatin and angiostatin or cytotoxic drugs such as adriamycin, daunomycin, cis-platinum, etoposide, taxol, taxotere and alkaloids, such as vincristine, farnesyl transferase inhibitors, VEGF inhibitors, and antimetabolites such as methotrexate.

The compounds of the invention may also be used in combination with antiviral agents such as Viracept, AZT, aciclovir and famciclovir, and antisepsis compounds such as Valant.

The compositions of the invention may include a “therapeutically effective amount” or a “prophylactically effective amount” of an antibody or antigen-binding portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier The specification for the dosage unit forms of the invention are dictated by and directly dependent on (a) the unique characteristics of the anti-M-CSF antibody or portion and the particular therapeutic or prophylactic effect to be achieved, and (b) the limitations inherent in the art of compounding such an antibody for the treatment of sensitivity in individuals.

An exemplary, non-limiting range for a therapeutically or prophylactically effective amount of an antibody or antibody portion of the invention is 0.025 to 50 mg/kg, more preferably 0.1 to 50 mg/kg, more preferably 0.1-25, 0.1 to 10 or 0.1 to 3 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Another aspect of the present invention provides kits comprising an anti-M-CSF antibody or antigen-binding portion of the invention or a composition comprising such an antibody or portion. A kit may include, in addition to the antibody or composition, diagnostic or therapeutic agents. A kit also can include instructions for use in a diagnostic or therapeutic method. In a preferred embodiment, the kit includes the antibody or a composition comprising it and a diagnostic agent that can be used in a method described below. In another preferred embodiment, the kit includes the antibody or a composition comprising it and one or more therapeutic agents that can be used in a method described below. One embodiment of the invention is a kit comprising a container, instructions on the administration of an anti-M-CSF antibody to a human suffering from an inflammatory disease, or instructions for measuring the number of CD14+CD16+ monocytes in a biological sample and an anti-M-CSF antibody.

This invention also relates to compositions for inhibiting abnormal cell growth in a mammal comprising an amount of an antibody of the invention in combination with an amount of a chemotherapeutic agent, wherein the amounts of the compound, salt, solvate, or prodrug, and of the chemotherapeutic agent are together effective in inhibiting abnormal cell growth. Many chemotherapeutic agents are known in the art. In some embodiments, the chemotherapeutic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, e.g. anti-androgens, and anti-angiogenesis agents.

Anti-angiogenic agents, such as MMP-2 (matrix-metalloproteinase 2) inhibitors. MMP-9 (matrix-metalloproteinase 9) inhibitors, and COX-II (cyclooxygenase II) inhibitors, can be used in conjunction with an anti-M-CSF antibody of the invention. Examples of useful COX-II inhibitors include CELEBREX™ (celecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP inhibitors are those that do not demonstrate arthralgia. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, RS 13-0830, and the compounds recited in the following list: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methyl-benzyloxy)-benzenesulfonyl]-3-hydroxy-3-methyl-piperidine-2-carboxylic acid hydroxyamide; 3-[[4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of said compounds.

A compound comprising a human anti-M-CSF monoclonal antibody of the invention can also be used with signal transduction inhibitors, such as agents that can inhibit EGF-R (epidermal growth factor receptor) responses, such as EGF-R antibodies, EGF antibodies, and molecules that are EGF-R inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN™ (Genentech, Inc.). EGF-R inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies C225 and anti-EGFR 22Mab (ImClone Systems Incorporated), ABX-EGF (Abgenix/Cell Genesys), EMD-7200 (Merck KgaA), EMD-5590 (Merck KgaA), MDX-447/H-477 (Medarex Inc. and Merck KgaA), and the compounds ZD-1834, ZD-1838 and ZD-1839 (AstraZeneca), PKI-166 (Novartis), PKI-166/CGP-75166 (Novartis), PTK 787 (Novartis), CP 701 (Cephalon), leflunomide (Pharmacia/Sugen), CI-1033 (Warner Lambert Parke Davis), CI-1033/PD 183,805 (Warner Lambert Parke Davis), CL-387,785 (Wyeth-Ayerst), BBR-1611 (Boehringer Mannheim GmbH/Roche). Naamidine A (Bristol Myers Squibb), RC-3940-II (Pharmacia), BIBX-1382 (Boehringer Ingelheim), OLX-103 (Merck & Co.), VRCTC-310 (Ventech Research), EGF fusion toxin (Seragen Inc.), DAB-389 (Seragen/Lilgand), ZM-252808 (Imperial Cancer Research Fund), RG-50864 (INSERM), LFM-A12 (Parker Hughes Cancer Center), WHI-P97 (Parker Hughes Cancer Center), GW-282974 (Glaxo), KT-8391 (Kyowa Hakko) and EGF-R Vaccine (York Medical/Centro de Immunologia Molecular (CIM)). These and other EGF-R-inhibiting agents can be used in the present invention.

VEGF inhibitors, for example SU-5416 and SU-6668 (Sugen Inc.), AVASTIN™ (Genentech), SH-268 (Schering), and NX-1838 (NeXstar) can also be combined with the compound of the present invention. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998). WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc.); anti-VEGF monoclonal antibody of Genentech, Inc.; and angiozyme, a synthetic ribozyme from Ribozyme and Chiron. These and other VEGF inhibitors can be used in the present invention as described herein. ErbB2 receptor inhibitors, such as GW-282974 (Glaxo Wellcome plc), and the monoclonal antibodies AR-209 (Aronex Pharmaceuticals Inc.) and 2B-1 (Chiron), can furthermore be combined with the compound of the invention, for example those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are all hereby incorporated herein in their entireties by reference. ErbB2 receptor inhibitors useful in the present invention are also described in U.S. Pat. No. 6,465,449 (issued Oct. 15, 2002), and in U.S. Pat. No. 6,284,764 (issued Sep. 4, 2001), both of which are incorporated in their entireties herein by reference. The erbB2 receptor inhibitor compounds and substance described in the aforementioned PCT applications, U.S. patents, and U.S. provisional applications, as well as other compounds and substances that inhibit the erbB2 receptor, can be used with the compound of the present invention in accordance with the present invention.

Anti-survival agents include anti-IGF-IR antibodies and anti-integrin agents, such as anti-integrin antibodies.

Anti-inflammatory agents can be used in conjunction with an anti-M-CSF antibody of the invention. For the treatment of rheumatoid arthritis, the human anti-M-CSF antibodies of the invention may be combined with agents such as TNF-∀ inhibitors such as TNF drugs (such as REMICADE™, CDP-870 and HUMIRA™) and TNF receptor immunoglobulin molecules (such as ENBREL™), IL-1 inhibitors, receptor antagonists or soluble IL-1ra (e.g. Kineret or ICE inhibitors), COX-2 inhibitors (such as celecoxib, rofecoxib, valdecoxib and etoricoxib), metalloprotease inhibitors (preferably MMP-13 selective inhibitors), p2×7 inhibitors, ∀2δ ligands (such as NEUROTIN™ AND PREGABALIN™), low dose methotrexate, leflunomide, hydroxychloroquine, d-penicillamine, auranofin or parenteral or oral gold. The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc.

Anti-coagulant agents can be used in conjunction with an anti-M-CSF antibody of the invention. Examples of anti-coagulant agents include, but are not limited to, warfarin (COUMADIN™), heparin, and enoxaparin (LOVENOX™).

The human anti-M-CSF antibodies of the present invention may also be used in combination with cardiovascular agents such as calcium channel blockers, lipid lowering agents such as statins, fibrates, beta-blockers, Ace inhibitors, Angiotensin-2 receptor antagonists and platelet aggregation inhibitors. The compounds of the present invention may also be used in combination with CNS agents such as antidepressants (such as sertraline), anti-Parkinsonian drugs (such as deprenyl, L-dopa, REQUIP™, MIRAPEX™, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, ∀2δ LIGANDS (such NEUROTIN™ and PREGABALIN™) inhibitors, COX-2 inhibitors, propentofylline or metrifonate.

The human anti-M-CSF antibodies of the present invention may also be used in combination with osteoporosis agents such as roloxifene, droloxifene, lasofoxifene or fosomax and immunosuppressant agents such as FK-506 and rapamycin.

### Diagnostic Methods of Use

In another aspect, the invention provides diagnostic methods. The anti-M-CSF antibodies can be used to detect M-CSF in a biological sample in vitro or in vivo. In one embodiment, the invention provides a method for diagnosing the presence or location of a M-CSF-expressing tumor in a subject in need thereof, comprising the steps of injecting the antibody into the subject, determining the expression of M-CSF in the subject by localizing where the antibody has bound, comparing the expression in the subject with that of a normal reference subject or standard, and diagnosing the presence or location of the tumor.

The anti-M-CSF antibodies can be used in a conventional immunoassay, including, without limitation, an ELISA, an RIA, FACS, tissue immunohistochemistry, Western blot or immunoprecipitation. The anti-M-CSF antibodies of the invention can be used to detect M-CSF from humans. In another embodiment, the anti-M-CSF antibodies can be used to detect M-CSF from primates such as cynomologus monkey, rhesus monkeys, chimpanzees or apes. The invention provides a method for detecting M-CSF in a biological sample comprising contacting a biological sample with an anti-M-CSF antibody of the invention and detecting the bound antibody. In one embodiment, the anti-M-CSF antibody is directly labeled with a detectable label. In another embodiment, the anti-M-CSF antibody (the first antibody) is unlabeled and a second antibody or other molecule that can bind the anti-M-CSF antibody is labeled. As is well known to one of skill in the art, a second antibody is chosen that is able to specifically bind the particular species and class of the first antibody. For example, if the anti-M-CSF antibody is a human IgG, then the secondary antibody could be an anti-human-IgG. Other molecules that can bind to antibodies include, without limitation, Protein A and Protein G, both of which are available commercially, e.g., from Pierce Chemical Co.

Suitable labels for the antibody or secondary antibody have been disclosed supra, and include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

In other embodiments, M-CSF can be assayed in a biological sample by a competition immunoassay utilizing M-CSF standards labeled with a detectable substance and an unlabeled anti-M-CSF antibody. In this assay, the biological sample, the labeled M-CSF standards and the anti-M-CSF antibody are combined and the amount of labeled M-CSF standard bound to the unlabeled antibody is determined. The amount of M-CSF in the biological sample is inversely proportional to the amount of labeled M-CSF standard bound to the anti-M-CSF antibody.

One can use the immunoassays disclosed above for a number of purposes. For example, the anti-M-CSF antibodies can be used to detect M-CSF in cells or on the surface of cells in cell culture, or secreted into the tissue culture medium. The anti-M-CSF antibodies can be used to determine the amount of M-CSF on the surface of cells or secreted into the tissue culture medium that have been treated with various compounds. This method can be used to identify compounds that are useful to inhibit or activate M-CSF expression or secretion. According to this method, one sample of cells is treated with a test compound for a period of time while another sample is left untreated. If the total level of M-CSF is to be measured, the cells are lysed and the total M-CSF level is measured using one of the immunoassays described above. The total level of M-CSF in the treated versus the untreated cells is compared to determine the effect of the test compound.

An immunoassay for measuring total M-CSF levels is an ELISA or Western blot. If the cell surface level of M-CSF is to be measured, the cells are not lysed, and the M-CSF cell surface levels can be measured using one of the immunoassays described above. An immunoassay for determining cell surface levels of M-CSF can include the steps of labeling the cell surface proteins with a detectable label, such as biotin or ¹²⁵I, immunoprecipitating the M-CSF with an anti-M-CSF antibody and then detecting the labeled M-CSF. Another immunoassay for determining the localization of M-CSF, e.g., cell surface levels, can be immunohistochemistry. Methods such as ELISA, RIA, Western blot, immunohistochemistry, cell surface labeling of integral membrane proteins and immunoprecipitation are well known in the art. See, e.g., Harlow and Lane, supra. In addition, the immunoassays can be scaled up for high throughput screening in order to test a large number of compounds for inhibition or activation of M-CSF.

Another example of an immunoassay for measuring secreted M-CSF levels can be an antigen capture assay, ELISA, immunohistochemistry assay, Western blot and the like using antibodies of the invention. If secreted M-CSF is to be measured, cell culture media or body fluid, such as blood serum, urine, or synovial fluid, can be assayed for secreted M-CSF and/or cells can be lysed to release produced, but not yet secreted M-CSF. An immunoassay for determining secreted levels of M-CSF includes the steps of labeling the secreted proteins with a detectable label, such as biotin or ¹²⁵I, immunoprecipitating the M-CSF with an anti-M-CSF antibody and then detecting the labeled M-CSF. Another immunoassay for determining secreted levels of M-CSF can include the steps of (a) pre-binding anti-M-CSF antibodies to the surface of a microtiter plate; (b) adding tissue culture cell media or body fluid containing the secreted M-CSF to the wells of the microtiter plate to bind to the anti-M-CSF antibodies; (c) adding an antibody that will detect the anti-M-CSF antibody, e.g., anti-M-CSF labeled with digoxigenin that binds to an epitope of M-CSF different from the anti-M-CSF antibody of step (a); (d) adding an antibody to digoxigenin conjugated to peroxidase; and (e) adding a peroxidase substrate that will yield a colored reaction product that can be quantitated to determine the level of secreted M-CSF in tissue culture cell media or a body fluid sample. Methods such as ELISA, RIA, Western blot, immunohistochemistry, and antigen capture assay are well known in the art. See, e.g., Harlow and Lane, supra. In addition, the immunoassays can be scaled up for high throughput screening in order to test a large number of compounds for inhibition or activation of M-CSF.

The anti-M-CSF antibodies of the invention can also be used to determine the levels of cell surface M-CSF in a tissue or in cells derived from the tissue. In some embodiments, the tissue is from a diseased tissue. In some embodiments, the tissue can be a tumor or a biopsy thereof. In some embodiments of the method, a tissue or a biopsy thereof can be excised from a patient. The tissue or biopsy can then be used in an immunoassay to determine, e.g., total M-CSF levels, cell surface levels of M-CSF, or localization of M-CSF by the methods discussed above.

The method can comprise the steps of administering a detectably labeled anti-M-CSF antibody or a composition comprising them to a patient in need of such a diagnostic test and subjecting the patient to imaging analysis to determine the location of the M-CSF-expressing tissues. Imaging analysis is well known in the medical art, and includes, without limitation, x-ray analysis, magnetic resonance imaging (MRI) or computed tomography (CE). The antibody can be labeled with any agent suitable for in vivo imaging, for example a contrast agent, such as barium, which can be used for x-ray analysis, or a magnetic contrast agent, such as a gadolinium chelate, which can be used for MRI or CE. Other labeling agents include, without limitation, radioisotopes, such as ⁹⁹Tc. In another embodiment, the anti-M-CSF antibody will be unlabeled and will be imaged by administering a second antibody or other molecule that is detectable and that can bind the anti-M-CSF antibody. In an embodiment, a biopsy is obtained from the patient to determine whether the tissue of interest expresses M-CSF.

The anti-M-CSF antibodies of the invention can also be used to determine the secreted levels of M-CSF in a body fluid such as blood serum, urine, or synovial fluid derived from a tissue. In some embodiments, the body fluid is from a diseased tissue. In some embodiments, the body fluid is from a tumor or a biopsy thereof. In some embodiments of the method, body fluid is removed from a patient. The body fluid is then used in an immunoassay to determine secreted M-CSF levels by the methods discussed above. One embodiment of the invention is a method of assaying for the activity of a M-CSF antagonist comprising: administering a M-CSF antagonist to a primate or human subject and measuring the number of CD14+CD16+ monocytes in a biological sample.

### Therapeutic Methods of Use

In another embodiment, the invention provides a method for inhibiting M-CSF activity by administering an anti-M-CSF antibody to a patient in need thereof. Any of the types of antibodies described herein may be used therapeutically. In a preferred embodiment, the anti-M-CSF antibody is a human, chimeric or humanized antibody. In another preferred embodiment, the M-CSF is human and the patient is a human patient. Alternatively, the patient may be a mammal that expresses a M-CSF that the anti-M-CSF antibody cross-reacts with. The antibody may be administered to a non-human mammal expressing a M-CSF with which the antibody cross-reacts (i.e. a primate) for veterinary purposes or as an animal model of human disease. Such animal models may be useful for evaluating the therapeutic efficacy of antibodies of this invention.

As used herein, the term “a disorder in which M-CSF activity is detrimental” is intended to include diseases and other disorders in which the presence of high levels of M-CSF in a subject suffering from the disorder has been shown to be or is suspected of being either responsible for the pathophysiology of the disorder or a factor that contributes to a worsening of the disorder. Such disorders may be evidenced, for example, by an increase in the levels of M-CSF secreted and/or on the cell surface or increased tyrosine autophosphorylation of c-fms in the affected cells or tissues of a subject suffering from the disorder. The increase in M-CSF levels may be detected, for example, using an anti-M-CSF antibody as described above.

In one embodiment, an anti-M-CSF antibody may be administered to a patient who has a c-fms-expressing tumor or a tumor that secretes M-CSF and/or that expresses M-CSF on its cell surface. Preferably, the tumor expresses a level of c-fms or M-CSF that is higher than a normal tissue. The tumor may be a solid tumor or may be a non-solid tumor, such as a lymphoma. In a more preferred embodiment, an anti-M-CSF antibody may be administered to a patient who has a c-fms-expressing tumor, a M-CSF-expressing tumor, or a tumor that secretes M-CSF that is cancerous. Further, the tumor may be cancerous. In an even more preferred embodiment, the tumor is a cancer of lung, breast, prostate or colon. In another preferred embodiment, the anti-M-CSF antibody administered to a patient results in M-CSF no longer bound to the c-fms receptor. In a highly preferred embodiment, the method causes the tumor not to increase in weight or volume or to decrease in weight or volume. In another embodiment, the method causes c-fms on tumor cells to not be bound by M-CSF. In another embodiment, the method causes M-CSF on tumor cells to not be bound to c-fms. In another embodiment, the method causes secreted M-CSF of the tumor cells to not be bound to c-fms. In a preferred embodiment, the antibody is selected from 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, or comprises a heavy chain, light chain or antigen binding region thereof. In another embodiment, the antibody is an anti-M-CSF antibody that has a heavy chain, a light chain, or both a heavy chain and light chain, that is or are 90%, 91%, 92,%, 93%, 94%, 95%, 96%, 96%, 97%, 98%, or 99% identical to the heavy chain, light chain, or both the heavy and light chain of antibody 8.10.3F respectively.

In another preferred embodiment, an anti-M-CSF antibody may be administered to a patient who expresses inappropriately high levels of M-CSF. It is known in the art that high-level expression of M-CSF can lead to a variety of common cancers. In one embodiment, said method relates to the treatment of cancer such as brain, squamous cell, bladder, gastric, pancreatic, breast, head, neck, esophageal, prostate, colorectal, lung, renal, kidney, ovarian, gynecological or thyroid cancer. Patients that can be treated with a compounds of the invention according to the methods of this invention include, for example, patients that have been diagnosed as having lung cancer, bone cancer, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors (e.g., sarcomas, carcinomas or lymphomas that are cancers of body tissues other than blood, bone marrow or the lymphatic system), solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas). In a more preferred embodiment, the anti-M-CSF antibody is administered to a patient with breast cancer, prostate cancer, lung cancer or colon cancer. In an even more preferred embodiment, the method causes the cancer to stop proliferating abnormally, or not to increase in weight or volume or to decrease in weight or volume.

In another preferred embodiment, an anti-M-CSF antibody may be administered to a patient who expresses inappropriately high levels of M-CSF, which can lead to a variety of inflammatory or immune disorders. Such disorders include, but are not limited to, lupus, including SLE, lupus nephritis, and cutaneous lupus, arthritis, psoriatic arthritis, Reiter's syndrome, gout, traumatic arthritis, rubella arthritis and acute synovitis, rheumatoid arthritis, rheumatoid spondylitis, ankylosing spondylitis, osteoarthritis, gouty arthritis and other arthritic conditions, sepsis, septic shock, endotoxic shock, gram negative sepsis, toxic shock syndrome, Alzheimer's disease, stroke, neurotrauma, asthma, adult respiratory distress syndrome, cerebral malaria, chronic pulmonary inflammatory disease, silicosis, pulmonary sarcoidosis, bone resorption disease, osteoporosis, restenosis, cardiac and renal reperfusion injury, thrombosis, glomerularonephritis, diabetes, graft vs. host reaction, allograft rejection, inflammatory bowel disease, Crohn's disease, ulcerative colitis, multiple sclerosis, muscle degeneration, eczema, contact dermatitis, psoriasis, sunburn, and conjunctivitis.

The antibody may be administered once, but more preferably is administered multiple times. For example, the antibody may be administered from three times daily to once every six months or longer. The administering may be on a schedule such as three times daily, twice daily, once daily, once every two days, once every three days, once weekly, once every two weeks, once every month, once every two months, once every three months and once every six months. The antibody may also be administered continuously via a minipump. The antibody may be administered via an oral, mucosal, buccal, intranasal, inhalable, intravenous, subcutaneous, intramuscular, parenteral, intratumor or topical route. The antibody may be administered at the site of the tumor or inflamed body part, into the tumor or inflamed body part, or at a site distant from the site of the tumor or inflamed body part. The antibody may be administered once, at least twice or for at least the period of time until the condition is treated, palliated or cured. The antibody generally will be administered for as long as the tumor is present provided that the antibody causes the tumor or cancer to stop growing or to decrease in weight or volume or until the inflamed body part is healed. The antibody will generally be administered as part of a pharmaceutical composition as described supra. The dosage of antibody will generally be in the range of 0.1-100 mg/kg, more preferably 0.5-50 mg/kg, more preferably 1-20 mg/kg, and even more preferably 1-10 mg/kg. The serum concentration of the antibody may be measured by any method known in the art.

In another aspect, efficacy of the anti-M-CSF antibody in treating or preventing various disorders and diseases may be determined by examining patients for changes in symptoms, various biomarkers, tissue histology, and physiological conditions, that are associated with the various disorders and diseases. Such symptoms, biomarkers, tissue histology and conditions are within the skill of a person skilled in the art to determine. For example, efficacy of the anti-M-CSF antibody in treating or preventing lupus in patients may include analyzing or observing changes in lupus related symptoms such as skin lesions, proteinuria, lymphadenopathy, serum M-CSF levels, anti-dsDNA antibody levels, and changes in kidney pathology such as by examining macrophage infiltration, inflammatory infiltrates, proteinaceous casts, size of glomerular tufts, glomerular IgG deposits, and C3 deposits. Changes in monocyte populations, such as CD14+CD16+ monocytes, and changes in osteoclast markers, such as uNTX-1 can also be examined. Biomarkers for systemic lupus, cutaneous lupus, and lupus nephritis may include erythrocyte sedimentation rate (ESR), C-reactive protein (CRP), complement (C3/C4), Ig levels (IgA, IgM, IgG), antinuclear antibodies (ANA), extractable nuclear antigen (ENA), and anti-dsDNA antibodies.

In addition to pharmacodynamic biomarker data, proof of principle biomarkers may include markers for the M-CSF pathway, proinflammatory cytokines/chemokines, immune cell subsets (B cell, T cell, and DC), and other disease-related markers from whole blood, serum, urine, and tissue samples taken throughout clinical studies.

For example, an exploratory serum biomarker panel can be used to examine changes in serum levels of cytokines, chemokines, and additional serum proteins related to M-CSF, monocyte, macrophage, and lupus (systemic, nephritis, and cutaneous) activity. Serum biomarkers may be examined by standard immunoassay techniques that are well known to persons skilled in the art. A panel of cytokine/chemokine serum biomarkers may include, for example: IFN-gamma, IFN-alpha, IL-12, TNF-alpha, IL-2, IL-4, IL-5, IL-13, IL-15, IL-6, IL-10, TGF-beta, IL-1-alpha, IL-1-beta, IL-21, IL-22, IL-23, IL-17A, IL-17F, CXCL10, CXCL9, CCL2, CCL19, RANTES, CXCL11. CCL7, CCL3, CXCL13, CCL8, CXCL8, CD40L, soluble TNFR, and soluble IL-1 receptor antagonist. A panel related to M-CSF, monocyte, and macrophage activity include: M-CSF, GM-CSF, RANKL, soluble CD14, and neopterin. A subset of serum biomarkers related to cutaneous lupus, systemic lupus may include: E-selectin, BAFF, soluble CD27, soluble CD154, and CCL17.

Urine from treated patients can be examined for changes in a panel of urine biomarkers that may include, among others: M-CSF, MCP-1, IL-6, IL-10, CCL3, and RANTES.

To evaluate changes in the circulating immune cell repertoire with anti-M-CSF antibody exposure, fluorescence activated cell sorting (FACS) of whole blood from treated human subjects may also be examined. Immune cell subsets can be defined by established surface marker expression patterns. For example, a B cell subset panel may include evaluation of total B cells, naïve B cells, memory B cells (non-switched and class-switched), plasma cells, double negative B cells, and IgD naïve B cells. A transitional B cell panel may include transitional B cells, immature B cells, and germinal center B cells. A T cell subset panel may delineate total T cells, NK T cells, NK cells, naïve T cells, memory T cells, central memory T cells, and effector memory T cells. A dendritic cell panel may examine myeloid dendritic cells as well as plasmacytoid dendritic cells. Such cell subsets are within the skill of a person skilled in the art to determine and detect.

In addition, changes in RNA expression with anti-M-CSF antibody exposure may be examined. RNA may be isolated from whole blood and tissue biopsy samples from treated patients. Gene expression can be quantitated from isolated RNA, for example, via standard Taqman RT-qPCR TLDA panel assay techniques. For example, Table 1C lists a panel of target genes that are associated with M-CSF pathway engagement, cytokine/chemokine activity, and disease-related gene expression. RNA expression of one or more of the genes listed in Table 1C may be examined to determine efficacy of treatment.

For example, lesional and nonlesional skin biopsies from cutaneous lupus patients may be evaluated for M-CSF-dependent changes by immunohistochemistry (IHC) and RNA profiling. Cell populations to be examined by IHC include macrophage, dendritic cell (pDC and mDC) and T cell populations. Additional IHC staining for biomarkers listed in the serum biomarker panel are under consideration. Expression analysis of RNA isolated from skin biopsies may examine one or more of the genes listed in Table 1C.

TABLE 1C

| GENE, GeneBook | Entrez Gene ID |
|---|---|
| granulocyte colony stimulating factor | 0 |
| alpha 2M receptor expression | 2 |
| ACACA | 31 |
| ACACB | 32 |
| ACP5 | 54 |
| AKT1 | 207 |
| Akt2 expression | 208 |
| ALAS2 | 212 |
| ALOX15 | 246 |
| Amd1 | 262 |
| ANPEP | 290 |
| BIRC5 | 332 |
| APOC1 | 341 |
| APOE | 348 |
| FAS | 355 |
| FASLG | 356 |
| ARAF | 369 |
| ARF6 WT | 382 |
| ARG1 | 383 |
| ARHGAP5 | 394 |
| BAX | 581 |
| cyclin D1 | 595 |
| BCL2 | 596 |
| BCL2L1 | 598 |
| high level CSF-1 | 629 |
| bone morphogenetic protein-2-induced colony | 650 |
| stimulating factor-1 | |
| CA2 | 760 |
| OPGL/CSF-1 treatment | 796 |
| CALCR | 799 |
| CASP3 | 836 |
| CSF-1 | 841 |
| caspase 9 | 842 |
| Runx1 | 861 |
| CBL | 867 |
| CCND2 | 894 |
| CSF-1 | 920 |
| CD14 | 929 |
| CD34 positive hematopoietic progenitor cells | 947 |
| CD36 | 948 |
| CD44 | 960 |
| CD47 | 961 |
| CD68 | 968 |
| CD97 | 976 |
| CDH5 | 1003 |
| CDK4 | 1019 |
| CDKN1A | 1026 |
| CDKN2D | 1032 |
| CCR5 | 1234 |
| COL3A1 | 1281 |
| CP | 1356 |
| CPE | 1363 |
| CREB1 | 1385 |
| CREB-binding protein | 1387 |
| CRP | 1401 |
| MAPK14 | 1432 |
| CSF-1 | 1435 |
| macrophages CSF-1 anti-sense | 1435 |
| oligodeoxynucleotide | |
| Anti-CSF I antibody | 1435 |
| Anti-CSF-1 Fab | 1435 |
| c-fms gene product | 1436 |
| CSF-1 receptor expressing BT20 breast cancer cell | 1436 |
| line | |
| c-fms proto-oncogene | 1436 |
| CSF-1 receptor mRNA | 1436 |
| mouse c-fms | 1436 |
| CSF1R | 1436 |
| granulocyte-macrophage colony-stimulating factor | 1437 |
| (GM-CSF)-stimulated BAC1.2F5 macrophages | |
| GM-CSF | 1437 |
| CSF2RA | 1438 |
| granulocyte colony stimulating factor | 1440 |
| c-Src kinase activity | 1445 |
| CTSK | 1513 |
| CYP11A1 | 1583 |
| CYP17A1 | 1586 |
| DCK | 1633 |
| DNMT1 | 1786 |
| DUSP1 | 1843 |
| epidermal growth factor (EGF) | 1950 |
| EGR1 | 1958 |
| EGR2 | 1959 |
| EGR3 | 1960 |
| CSF-1-induced TCF/SAP-1 modification | 2002 |
| SLC29A1 | 2030 |
| erythropoietin (EPO) | 2056 |
| ErbB2 | 2064 |
| endogenous ets-2 | 2114 |
| colony-stimulating factor 1 | 2114 |
| ETS2 | 2114 |
| ETV3 | 2117 |
| plasma prothrombin fragment F1 | 2147 |
| F2R | 2149 |
| F2RL1 | 2150 |
| F2RL2 | 2151 |
| FABP7 | 2173 |
| PTK2B | 2185 |
| FASN | 2194 |
| FCER1G | 2207 |
| FCGR2A | 2212 |
| FCGR2B | 2213 |
| FCGR3A | 2214 |
| FLT3 | 2322 |
| FN1 | 2335 |
| c-fos | 2353 |
| FYN | 2534 |
| GAPDH | 2597 |
| growth factor independent-1 | 2672 |
| CXCR3 | 2833 |
| several other GRB2-associated proteins | 2885 |
| HCK | 3055 |
| HDAC1 | 3065 |
| HDAC2 | 3066 |
| HLX1 | 3142 |
| HNRNPH1 | 3187 |
| HRAS | 3265 |
| HSD17B1 | 3292 |
| HSPA5 | 3309 |
| HTR5A | 3361 |
| IRF8 | 3394 |
| IFNB1 | 3456 |
| IFN-gamma | 3458 |
| purified IFN-gamma | 3458 |
| IFN-gamma | 3458 |
| IFNGR1 | 3459 |
| IGF-1 | 3479 |
| IGF1R | 3480 |
| IGFBP2 | 3485 |
| IGF binding protein-3 | 3486 |
| IKK beta | 3551 |
| IL-1 alpha | 3552 |
| IL-1 beta | 3553 |
| murine interleukin-1 receptor antagonist | 3557 |
| IL-2-receptor | 3558 |
| interleukin-2 (IL-2) | 3558 |
| IL-3 | 3562 |
| IL-4 | 3565 |
| IL-6 | 3569 |
| IL6ST | 3572 |
| IL-7 enhanced macrophage colony-stimulating | 3574 |
| factor (CSF-1)-induced colony formation | |
| IL-8 | 3576 |
| IL8 | 3576 |
| IL10 level | 3586 |
| IL10RA | 3587 |
| Il11ra2 | 3590 |
| IL12A | 3592 |
| IL12B | 3593 |
| IL18 | 3606 |
| CSF-1 | 3630 |
| INPP5D | 3635 |
| INPPL1 | 3636 |
| IRF7 | 3665 |
| ITGA5 | 3678 |
| ITGAM | 3684 |
| ITGAV | 3685 |
| all CD11c(high) DC subsets | 3687 |
| ITGB3 | 3690 |
| ITGB5 | 3693 |
| jun | 3725 |
| JUNB | 3726 |
| KCNA3 | 3738 |
| KCNJ2 | 3759 |
| LHB | 3972 |
| TNF-beta | 4049 |
| LYN | 4067 |
| Smad 4 increased CSF-1 transcription | 4089 |
| MAP3K3 | 4215 |
| MMP2 | 4313 |
| MMP9 | 4318 |
| MMP12 | 4321 |
| MMP16 | 4325 |
| MSR1 | 4481 |
| chimeric c-Myc transactivators | 4609 |
| NFATC1 | 4772 |
| NF-kappaB site | 4790 |
| NFKB2 | 4791 |
| CSF-1 antibody | 4843 |
| NPY1R | 4886 |
| NT5E | 4907 |
| chimeric colony-stimulating factor-1/TrkB-receptor | 4915 |
| colony-stimulating factor-1 | 4982 |
| OPRD1 | 4985 |
| CSF-1-mediated activation | 4988 |
| SERPINE1 | 5054 |
| PAI-2 | 5055 |
| PAK2 | 5062 |
| PDE6A | 5145 |
| PDGFA | 5154 |
| PDGFB receptor | 5155 |
| PENK | 5179 |
| PIK3CA | 5290 |
| PIK3CB | 5291 |
| PIK3CD | 5293 |
| PI3Kgamma(-/ | 5294 |
| PIK3R2 | 5296 |
| phospholipase A2 activity | 5319 |
| PLA2G4A | 5321 |
| uPA promoter activation | 5328 |
| uPA receptor | 5328 |
| PLCG2 | 5336 |
| PLD2 | 5338 |
| PPARG | 5468 |
| PRKACA | 5566 |
| PRKCA | 5578 |
| MAPK1 | 5594 |
| MAPK3 | 5595 |
| ERK5/BMK1 | 5598 |
| MAPK8 | 5599 |
| MAPK9 | 5601 |
| MAP2K1 | 5604 |
| MAP2K2 | 5605 |
| augmented PTH-induced increases | 5741 |
| anti-CSF-1 | 5741 |
| anti-CSF-1 | 5744 |
| anti-CSF-1 | 5745 |
| PTK2 | 5747 |
| SHP-1 | 5777 |
| PTPN6 | 5777 |
| anti-CD148 monoclonal antibody | 5795 |
| PTPRO | 5800 |
| RAC1 | 5879 |
| not dominant-negative c-Raf-1 | 5894 |
| RBP1 | 5947 |
| RPS6KB2 | 6199 |
| CCL3 | 6348 |
| CCL5 | 6352 |
| CCL13 | 6357 |
| CX3CL1 | 6376 |
| CSF-1 | 6446 |
| SHC1 | 6464 |
| INI1/hSNF5/BAF47 | 6598 |
| SPI1 | 6688 |
| SRC-1 | 6714 |
| STAT1 | 6772 |
| STAT3 | 6774 |
| STX3 | 6809 |
| Syk Piceatannol | 6850 |
| Substance P augmentation | 6863 |
| mCSF-1 | 6868 |
| TGF-beta1 | 7040 |
| TGF-beta 2 | 7042 |
| Tpo | 7066 |
| TLR1 | 7096 |
| TLR2 | 7097 |
| TLR5 | 7100 |
| 12-0-tetradecanoyl-phorbol-13-acetate (TPA)- | 7112 |
| induced down-regulation | |
| TNF-alpha | 7124 |
| TNFAIP3 | 7128 |
| TNFRSF1A | 7132 |
| TNFRSF1B | 7133 |
| TOP2A | 7153 |
| TP53 | 7157 |
| Tpo | 7173 |
| Tpt1 | 7178 |
| HSP90B1 | 7184 |
| TRAF2 | 7186 |
| vascular cell adhesion molecule-1 (VCAM-1) | 7412 |
| vascular endothelial growth factor (VEGF)-A | 7422 |
| production | |
| WAS | 7454 |
| CNBP binding | 7555 |
| CXCR4 | 7852 |
| GPR68 | 8111 |
| SHP-1 | 8431 |
| OPGL | 8600 |
| RANKL | 8600 |
| SRC-1 | 8648 |
| SOCS1 | 8651 |
| MARCO | 8685 |
| RIPK1 | 8737 |
| TNFRSF11A | 8792 |
| SKAP2 | 8935 |
| CH25H | 9023 |
| DOK2 | 9046 |
| MAYP | 9050 |
| IL1RL1 | 9173 |
| CD163 | 9332 |
| GRAP2 | 9402 |
| ITM2B | 9445 |
| GDF15 | 9518 |
| CLOCK | 9575 |
| IKBKE | 9641 |
| HCK (p) | 9846 |
| MAFB | 9935 |
| Abi1 | 10006 |
| IL18BP | 10068 |
| STX6 | 10228 |
| RAMP2 | 10266 |
| SIGMAR1 | 10280 |
| TRAIP | 10293 |
| SIRPB1 | 10326 |
| TLR6 | 10333 |
| KHDRBS1 | 10657 |
| SLC7A9 | 11136 |
| TRIM35 | 23087 |
| CSF-1-induced TCF/SAP-1 modification | 23415 |
| STX12 | 23673 |
| phospholipase A2 activity | 26279 |
| NKIRAS1 | 28512 |
| RHOD | 29984 |
| TLR9 | 54106 |
| HPP-CFC-1 | 55997 |
| CXCR7 | 57007 |
| SLAP-2 | 84174 |
| ADSSL1 | 122622 |
| OSCAR | 126014 |
| GAB3 | 139716 |
| 192162 | 192162 |
| MPEG1 | 219972 |
| ANXA8 | 1653145 |
| CCR2 | 729230 |
| Hsd3b | 3283/3284 |
| BCLXL | |
| Fcris | |

In another aspect, the anti-M-CSF antibody may be co-administered with other therapeutic agents, such as anti-inflammatory agents, anti-coagulant agents, agents that will lower or reduce blood pressure, anti-neoplastic drugs or molecules, to a patient who is in need of treatment. In one aspect, the invention relates to a method for the treatment of the hyperproliferative disorder in a mammal comprising administering to said mammal a therapeutically effective amount of a compound of the invention in combination with an anti-tumor agent selected from the group consisting of, but not limited to, mitotic inhibitors, alkylating agents, anti-metabolites, intercalating agents, growth factor inhibitors, cell cycle inhibitors, enzymes, topoisomerase inhibitors, biological response modifiers, anti-hormones, kinase inhibitors, matrix metalloprotease inhibitors, genetic therapeutics and anti-androgens. In a more preferred embodiment, the antibody may be administered with an antineoplastic agent, such as adriamycin or taxol. In another preferred embodiment, the antibody or combination therapy is administered along with radiotherapy, chemotherapy, photodynamic therapy, surgery or other immunotherapy. In yet another preferred embodiment, the antibody will be administered with another antibody. For example, the anti-M-CSF antibody may be administered with an antibody or other agent that is known to inhibit tumor or cancer cell proliferation, e.g., an antibody or agent that inhibits erbB2 receptor, EGF-R, CD20 or VEGF.

Co-administration of the antibody with an additional therapeutic agent (combination therapy) encompasses administering a pharmaceutical composition comprising the anti-M-CSF antibody and the additional therapeutic agent and administering two or more separate pharmaceutical compositions, one comprising the anti-M-CSF antibody and the other(s) comprising the additional therapeutic agent(s). Further, although co-administration or combination therapy generally means that the antibody and additional therapeutic agents are administered at the same time as one another, it also encompasses instances in which the antibody and additional therapeutic agents are administered at different times. For instance, the antibody may be administered once every three days, while the additional therapeutic agent is administered once daily. Alternatively, the antibody may be administered prior to or subsequent to treatment of the disorder with the additional therapeutic agent. Similarly, administration of the anti-M-CSF antibody may be administered prior to or subsequent to other therapy, such as radiotherapy, chemotherapy, photodynamic therapy, surgery or other immunotherapy

The antibody and one or more additional therapeutic agents (the combination therapy) may be administered once, twice or at least the period of time until the condition is treated, palliated or cured. Preferably, the combination therapy is administered multiple times. The combination therapy may be administered from three times daily to once every six months. The administering may be on a schedule such as three times daily, twice daily, once daily, once every two days, once every three days, once weekly, once every two weeks, once every month, once every two months, once every three months and once every six months, or may be administered continuously via a minipump. The combination therapy may be administered via an oral, mucosal, buccal, intranasal, inhalable, intravenous, subcutaneous, intramuscular, parenteral, intratumor or topical route. The combination therapy may be administered at a site distant from the site of the tumor. The combination therapy generally will be administered for as long as the tumor is present provided that the antibody causes the tumor or cancer to stop growing or to decrease in weight or volume.

In a still further embodiment, the anti-M-CSF antibody is labeled with a radiolabel, an immunotoxin or a toxin, or is a fusion protein comprising a toxic peptide. The anti-M-CSF antibody or anti-M-CSF antibody fusion protein directs the radiolabel, immunotoxin, toxin or toxic peptide to the M-CSF-expressing cell. In a preferred embodiment, the radiolabel, immunotoxin, toxin or toxic peptide is internalized after the anti-M-CSF antibody binds to the M-CSF on the surface of the target cell.

In another aspect, the anti-M-CSF antibody may be used to treat noncancerous states in which high levels of M-CSF and/or M-CSF have been associated with the noncancerous state or disease. In one embodiment, the method comprises the step of administering an anti-M-CSF antibody to a patient who has a noncancerous pathological state caused or exacerbated by high levels of M-CSF and/or M-CSF levels or activity. In a more preferred embodiment, the anti-M-CSF antibody slows the progress of the noncancerous pathological state. In a more preferred embodiment, the anti-M-CSF antibody stops or reverses, at least in part, the noncancerous pathological state.

### Gene Therapy

The nucleic acid molecules of the instant invention can be administered to a patient in need thereof via gene therapy. The therapy may be either in vivo or ex vivo. In a preferred embodiment, nucleic acid molecules encoding both a heavy chain and a light chain are administered to a patient. In a more preferred embodiment, the nucleic acid molecules are administered such that they are stably integrated into chromosomes of B cells because these cells are specialized for producing antibodies. In a preferred embodiment, precursor B cells are transfected or infected ex vivo and re-transplanted into a patient in need thereof. In another embodiment, precursor B cells or other cells are infected in vivo using a virus known to infect the cell type of interest. Typical vectors used for gene therapy include liposomes, plasmids and viral vectors. Exemplary viral vectors are retroviruses, adenoviruses and adeno-associated viruses. After infection either in vivo or ex vivo, levels of antibody expression can be monitored by taking a sample from the treated patient and using any immunoassay known in the art or discussed herein.

In a preferred embodiment, the gene therapy method comprises the steps of administering an isolated nucleic acid molecule encoding the heavy chain or an antigen-binding portion thereof of an anti-M-CSF antibody and expressing the nucleic acid molecule. In another embodiment, the gene therapy method comprises the steps of administering an isolated nucleic acid molecule encoding the light chain or an antigen-binding portion thereof of an anti-M-CSF antibody and expressing the nucleic acid molecule. In a more preferred method, the gene therapy method comprises the steps of administering of an isolated nucleic acid molecule encoding the heavy chain or an antigen-binding portion thereof and an isolated nucleic acid molecule encoding the light chain or the antigen-binding portion thereof of an anti-M-CSF antibody of the invention and expressing the nucleic acid molecules. The gene therapy method may also comprise the step of administering another anti-cancer agent, such as taxol or adriamycin.

In order that this invention may be better understood, the following examples are set forth. These examples are for purposes of illustration only and are not to be construed as limiting the scope of the invention in any manner.

### Example I

### Generation of Cell Lines Producing Anti-M-CSF Antibody

Antibodies of the invention were prepared, selected, and assayed as follows:

### Immunization and Hybridoma Generation

Eight to ten week old XENOMOUSE™ mice were immunized intraperitoneally or in their hind footpads with human M-CSF (10 μg/dose/mouse). This dose was repeated five to seven times over a three to eight week period. Four days before fusion, the mice were given a final injection of human M-CSF in PBS. The spleen and lymph node lymphocytes from immunized mice were fused with the non-secretory myeloma P3-X63-Ag8.653 cell line, and the fused cells were subjected to HAT selection as previously described (Galfre and Milstein, Methods Enzymol. 73:3-46, 1981). A panel of hybridomas all secreting M-CSF specific human IgG2 and IgG4 antibodies was recovered. Antibodies also were generated using XENOMAX™ technology as described in Babcook, J. S. et al., Proc. Natl. Acad. Sci. USA 93:7843-48, 1996. Nine cell lines engineered to produce antibodies of the invention were selected for further study and designated 252, 88, 100, 3.8.3, 2.7.3, 1.120.1, 9.14.4, 8.10.3 and 9.7.2. The hybridomas were deposited under terms in accordance with the Budapest Treaty with the American Type Culture Collection (ATCC), University Blvd., Manassas, Va. 20110-2209 on Aug. 8, 2003. The hybridomas have been assigned the following accession numbers:

| | |
|---|---|
| Hybridoma 3.8.3 (LN 15891) | PTA-5390 |
| Hybridoma 2.7.3 (LW 15892) | PTA-5391 |
| Hybridoma 1.120.1 (LN 15893) | PTA-5392 |
| Hybridoma 9.7.2 (LN 15894) | PTA-5393 |
| Hybridoma 9.14.4 (LN 15895) | PTA-5394 |
| Hybridoma 8.10.3 (LN 15896) | PTA-5395 |
| Hybridoma 88-gamma (UC 25489) | PTA-5396 |
| Hybridoma 88-kappa (UC 25490) | PTA-5397 |
| Hybridoma 100-gamma (UC 25491) | PTA-5398 |
| Hybridoma 100-kappa (UC 25492) | PTA-5399 |
| Hybridoma 252-gamma (UC 25493) | PTA-5400 |
| Hybridoma 252-kappa (UC 25494) | PTA-5401 |

### Example II

### Gene Utilization Analysis

DNA encoding the heavy and light chains of monoclonal antibodies 252, 88, 100, 3.8.3, 2.7.3, 1.120.1, 9.14.4, 8.10.3 and 9.7.2 was cloned from the respective hybridoma cell lines and the DNA sequences were determined by methods known to one skilled in the art. Additionally, DNA from the hybridoma cell lines 9.14.4, 8.10.3 and 9.7.2 was mutated at specific framework regions in the variable domain and/or isotype-switched to obtain, for example, 9.14.4I, 8.10.3F, and 9.7.2IF, respectively. From nucleic acid sequence and predicted amino acid sequence of the antibodies, the identity of the gene usage for each antibody chain was determined (“VBASE”). Table 2 sets forth the gene utilization of selected antibodies in accordance with the invention:

TABLE 2
Heavy and Light Chain Gene Utilization

| | Heavy Chain | | | | Kappa Light Chain | | |
|---|---|---|---|---|---|---|---|
| Clone | SEQ ID NO: | V_{H} | D_{H} | J_{H} | SEQ ID NO: | V_{κ} | J_{κ} |
| 252 | 1, 2 | 3-11 | 7-27 | 6 | 3, 4 | O12 | 3 |
| 88 | 5, 6 | 3-7 | 6-13 | 4 | 7, 8 | O12 | 3 |
| 100 | 9, 10 | 3-23 | 1-26 | 4 | 11, 12 | L2 | 3 |
| 3.8.3 | 14 | 3-11 | 7-27 | 4 | 16 | L5 | 3 |
| 2.7.3 | 18 | 3-33 | 1-26 | 4 | 20 | L5 | 4 |
| 1.120.1 | 22 | 1-18 | 4-23 | 4 | 24 | B3 | 1 |
| 9.14.4I | 25, 26 | 3-11 | 7-27 | 4b | 27, 28 | O12 | 3 |
| 8.10.3F | 29, 36 | 3-48 | 1-26 | 4b | 31, 32 | A27 | 4 |
| 9.7.2IF | 33, 34 | 3-11 | 6-13 | 6b | 35, 36 | O12 | 3 |
| 9.14.4 | 37, 38 | 3-11 | 7-27 | 4b | 27, 28 | O12 | 3 |
| 8.10.3 | 29, 30 | 3-48 | 1-26 | 4b | 43, 44 | A27 | 4 |
| 9.7.2 | 45, 46 | 3-11 | 6-13 | 6b | 47, 48 | O12 | 3 |
| 8.10.3FG1 | 97, 98 | 3-48 | 1-26 | 4b | 31, 32 | A27 | 4 |
| 9.14.4G1 | 101, 102 | 3-11 | 7-27 | 4b | 27, 28 | O12 | 3 |
| 9.14.4C-Ser | 54 | 3-11 | 7-27 | 4b | 56 | O12 | 3 |
| 9.14.4-CG2 | 74 | 3-11 | 7-27 | 4b | 56 | O12 | 3 |
| 9.14.4-CG4 | 78 | 3-11 | 7-27 | 4b | 56 | O12 | 3 |
| 8.10.3C-Ser | 58 | 3-48 | 1-26 | 4b | 60 | A27 | 4 |
| 8.10.3-CG2 | 62 | 3-48 | 1-26 | 4b | 60 | A27 | 4 |
| 8.10.3-CG4 | 94 | 3-48 | 1-26 | 4b | 60 | A27 | 4 |
| 8.10.3-Ser | 90 | 3-48 | 1-26 | 4b | 43, 44 | A27 | 4 |
| 9.7.2C-Ser | 50 | 3-11 | 6-13 | 6b | 52 | O12 | 3 |
| 9.7.2-CG2 | 66 | 3-11 | 6-13 | 6b | 52 | O12 | 3 |
| 9.7.2-CG4 | 70 | 3-11 | 6-13 | 6b | 52 | O12 | 3 |
| 9.7.2-Ser | 86 | 3-11 | 6-13 | 6b | 47, 48 | O12 | 3 |
| 9.14.4-Ser | 82 | 3-11 | 7-27 | 4b | 27, 28 | O12 | 3 |

Mutagenesis of specific residues of the heavy and light chains was carried out by designing primers and using the QuickChange Site Directed Mutagenesis Kit from Stratagene, according to the manufacturer's instructions. Mutations were confirmed by automated sequencing, and mutagenized inserts were subcloned into expression vectors. The expression vectors were transfected into HEK293 cells to produce enough of the antibodies for characterization.

### Example III

### M-CSF Mouse Monocytic Cell Proliferation Assay

In vitro assays were conducted to measure M-CSF-dependent mouse monocytic cell proliferation in the presence of anti-M-CSF antibodies to determine the degree of inhibition by anti-M-CSF antibodies.

Mouse monocytic cells, M-NFS-60 cells, from American Type Culture Collection (ATCC) (Manassas, Va.), were obtained and maintained in RPMI-1640 medium containing 2 mM L-glutamine (ATCC), 10% heat inactivated fetal bovine serum (FBS) (Invitrogen, Carlsbad, Calif.), 0.05 mM 2-mercaptoethanol (Sigma, St. Louis Mo.) (assay medium), with 15 ng/ml human M-CSF. M-NSF-60 cells were split to 5×10⁴ for next day use or to 2.5×10⁴ for use in 2 days. Prior to use in the assay, the cells were washed three times with RPMI-1640, counted and the volume adjusted with assay medium to yield 2×10⁵ cells/ml. All conditions were conducted in triplicate in 96-well treated tissue culture plates (Corning, Corning, N.Y.). To each well 50 μl of the washed cells, either 100 pM or 1000 pM M-CSF in a volume of 25 μl and test or control antibody at various concentrations in a volume of 25 μl in acetate buffer (140 mM sodium chloride, 20 mM sodium acetate, and 0.2 mg/ml polysorbate 80, pH 5.5) to a final volume of 100 μl was added. Antibodies of the invention were tested alone and with human M-CFS. The plates were incubated for 24 hours (hrs) at 37° C. with 5% CO₂.

After 24 hrs, 10 μl/well of 0.5 μCi ³H-thymidine (Amersham Biosciences, Piscataway, N.J.) was added and pulsed with the cells for 3 hrs. To detect the amount of incorporated thymidine, the cells were harvested onto pre-wet unifilter GFIC filterplates (Packard, Meriden, Conn.) and washed 10 times with water. The plates were allowed to dry overnight Bottom seals were added to the filterplates. Next, 45 μl Microscint 20 (Packard, Meriden, Conn.) per well was added. After a top seal was added, the plates were counted in a Trilux microbeta counter (Wallac, Norton, Ohio).

These experiments demonstrate that anti-M-CSF antibodies of the invention inhibit mouse monocytic cell proliferation in response to M-CSF. Further, by using various concentrations of antibodies, the IC₅₀ for inhibition of mouse nonocytic cell proliferation was determined for antibodies 252, 88, 100, 3.8.3, 2.7.3, 1.120.1, 9.14.4I, 8.10.3F, 9.7.2IF, 9.14.4, 8.10.3, and 9.7.2 (Cell Proliferation Assay, Table 3a and Table 3b).

TABLE 3a

| Antibody | 252 | 88 | 100 | 3.8.3 | 2.7.3 | 1.120.1 |
|---|---|---|---|---|---|---|
| M-CSF Mouse | 1.86 × 10⁻¹⁰ | 2.31 × 10⁻¹⁰ | 7.44 × 10⁻¹⁰ | 7.3 × 10⁻¹¹ | 1.96 × 10⁻¹⁰ | 1.99 × 10⁻¹⁰ |
| Monocytic Cell | | | | | | |
| Proliferation Assay | | | | | | |
| [IC₅₀, M] | | | | | | |
| Human Whole Blood | 8.67 × 10⁻¹⁰ | 5.80 × 10⁻¹⁰ | 1.53 × 10⁻¹⁰ | 8.6 × 10⁻¹¹ | 7.15 × 10⁻¹⁰ | 8.85 × 10⁻¹⁰ |
| Monocyte Activation | | | | | | |
| Assay | | | | | | |
| [IC₅₀, M] | | | | | | |
| Receptor Binding | 7.47 × 10⁻¹⁰ | 4.45 × 10⁻¹⁰ | 1.252 × 10⁻⁹ | 7.0 × 10⁻¹¹ | 3.08 × 10⁻¹⁰ | 1.57 × 10⁻¹⁰ |
| Inhibition Assay | | | | | | |
| [IC₅₀, M] | | | | | | |

TABLE 3b

| Antibody | 9.14.4I | 8.10.3F | 9.7.2IF | 9.14.4 | 8.10.3 | 9.7.2 |
|---|---|---|---|---|---|---|
| M-CSF Mouse | 2.02 × 10⁻¹⁰ | 4.13 × 10⁻¹⁰ | 7.37 × 10⁻¹⁰ | 2.02 × 10⁻¹⁰ | 4.13 × 10⁻¹⁰ | 7.37 × 10⁻¹⁰ |
| Monocytic Cell | | | | | | |
| Proliferation Assay | | | | | | |
| [IC₅₀, M] | | | | | | |
| Human Whole | 2.49 × 10⁻¹⁰ | 4.46 × 10⁻¹⁰ | 1.125 × 10⁻⁹ | 6.48 × 10⁻¹⁰ | 2.8 × 10⁻¹⁰ | 1.98 × 10⁻¹⁰ |
| Blood Monocyte | | | | | | |
| Activation Assay | | | | | | |
| [IC₅₀, M] | | | | | | |
| Receptor Binding | 2.97 × 10⁻¹⁰ | 9.8 × 10⁻¹¹ | 5.29 × 10⁻¹⁰ | 4.1 × 10⁻¹¹ | 1.5 × 10⁻⁹ | 6 × 10⁻¹² |
| Inhibition Assay | | | | | | |
| [IC₅₀, M] | | | | | | |

### Example IV

### Human Whole Blood Monocyte Activation Assay

In vitro assays were conducted to measure M-CSF dependent monocyte shape changes in the presence of anti-M-CSF antibodies to determine if the anti-M-CSF antibodies were capable of inhibiting whole blood monocyte activation and their degree of inhibition of monocyte shape changes.

In individual wells of a 96-well tissue culture plate, 6 μl of 1.7 nM anti-M-CSF and 94 μl of whole human blood for a final concentration of 102 pM anti-M-CSF antibody were mixed. The plates were incubated at 37° C. in a CO₂ tissue culture incubator. Next, the plates were removed from the Incubator. To each well, 100 μl of a fixative solution (0.5% formalin in phosphate buffered saline without MgCl₂ or CaCl₂) was added and the plates were incubated for 10 minutes at room temperature. For each sample, 180 μl from each well and 1 ml of Red Cell Lysis Buffer were mixed. The tubes were vortexed for 2 seconds. Next, the samples were incubated at 37° C. for 5 minutes in a shaking water bath to lyse the red blood cells, but to leave monocytes intact. Immediately following this incubation, the samples were read on a fluorescence-activated cell scanning (FACS) machine (BD Beckman FACS) and data was analyzed using FACS Station Software Version 3.4.

These experiments demonstrate that anti-M-CSF antibodies of the invention inhibit monocyte shape changes compared to control samples. Using the monocyte shape change assay, the IC₅₀ was determined for antibodies 252, 88, 100, 3.8.3, 2.7.3, 1.120.1, 9.14.4I, 8.10.3F, 9.7.2IF, 9.14.4, 8.10.3, and 9.7.2 (Human Whole Blood Monocyte Activation, Table 3a and Table 3b).

### Example V

### c-fms Receptor Binding Inhibition Assay

In vitro assays were conducted to measure M-CSF binding to c-fms receptor in the presence of anti-M-CSF antibodies to determine if the anti-M-CSF antibodies were capable of inhibiting M-CSF binding to c-fms receptor and their degree of inhibition.

NIH-3T3 cells transfected with human c-fms or M-NSF-60 cells maintained in Dulbecco's phosphate buffered saline without magnesium or calcium were washed. NIH-3T3 cells were removed from tissue culture plates with 5 mM ethylene-diamine-tetra-acetate (EDTA), pH 7.4. The NIH-3T3 cells were returned to the tissue culture incubator for 1-2 minutes and the flask(s) were tapped to loosen the cells. The NIH-3T3 cells and the M-NSF-60 cells were transferred to 50 ml tubes and washed twice with reaction buffer (1×RPMI without sodium bicarbonate containing 50 mM N-2-Hydroxyethylpiperazine-N′-2-ethanesulfonic acid (HEPES), pH 7.4). Next, the NIH-3T3 cells were resuspended in reaction buffer for a final concentration of 1.5×10⁵ cell/ml. The M-NSF-60 cells were resuspended in a reaction buffer for a final concentration of 2.5×10⁶ cells/ml.

For the assay, 9 μl of a sterile 0.4 M sucrose solution, 100 μl of ¹²⁵I-M-CSF (Amersham, IMQ7228v) at a final concentration of 200 pM in RPMI-1640 containing 50 mM HEPES (pH 7.4), 0.2% bovine serum albumin, and 100 μl of unlabeled M-CSF at a final concentration of 200 nM were mixed in a binding tube. Next, 50 μl/tube of increasing concentrations of a test antibody was added. In order to determine non-specific binding of the antibodies, we included samples to which we also added 200 nM M-CSF. To control tubes, we did not add antibody. Next, 15,000 NIH-3T3 cells or 250,000 M-NSF-60 cells were added per tube. All tubes were incubated at room temperature for 3 hrs and subjected to centrifugation at 10,000 rpm for 2 min. The tips of the tubes containing the cell pellets were cut off and the amount of M-CSF bound to the cells was determined using a Packard Cobra II Gamma counter. The specific binding was determined by subtracting non-specific binding from total binding. All assays were performed in duplicate. The binding data was analyzed using the computer program, Graph Pad Prism 2.01.

These experiments demonstrate that anti-M-CSF antibodies of the invention inhibit the binding of M-CSF to c-fms receptor compared to control samples. Further, by using various concentrations of antibodies, the IC₅₀ for inhibition of receptor binding was determined for antibodies 252, 88, 100, 3.8.3, 2.7.3, 1.120.1, 9.14.4I, 8.10.3F, 9.7.2IF, 9.14.4, 8.10.3, and 9.7.2 (Receptor Binding Inhibition Assay, Table 3a and Table 3b).

### Example VI

### Determination of Affinity Constants (K_{D}) of Anti-M-CSF Monoclonal Antibodies by BIACORE™

Affinity measures of purified antibodies were performed by surface plasmon resonance using the BIACORE™ 3000 instrument, following the manufacturer's protocols.

For antibodies 3.8.3, 2.7.3 and 1.120.1, the experiments were performed in a BIACORE™ 3000 instrument at 25° C. in Dulbecco's phosphate buffered saline containing 0.0005% Tween-20. Protein concentrations were obtained from sedimentation velocity experiments or by measuring the wavelength of the sample at 280 nm using theoretical extinction coefficients derived from amino acid sequences. For experiments measuring the binding of antibody to immobilized antigens, M-CSF was immobilized on a B1 chip by standard direct amine coupling procedures. Antibody samples were prepared at 0.69 μM for 3.8.3, 2.7.3 and 1.120.1. These samples were diluted 3-fold serially to 8.5 nM or 2.8 nM for roughly a 100-fold range in concentrations. For each concentration, the samples were injected in duplicate at 5 μl/min flow for 4 min. The dissociation was monitored for 2000 seconds. The data were fit globally to a simple 1:1 binding model using BIACORE™ Biaevaluation software. In all cases, this method was used to obtain k_{off} and it was found that this data set compared well to data obtained from global fit of association and dissociation data.

For antibodies 252, 88 and 100, the experiments were performed in a BIACORE™ 3000 instrument at 25° C. in HBS-EP Buffer (0.01M HEPES, pH 7.4, 0.15 M NaCl, 3 mM EDTA, 0.005% Surfactant P20). For experiments measuring the binding of antibody to immobilized antigens, a M-CSF was immobilized on a CM5 Research Grade Sensor chip by standard direct amine coupling procedures. Antibody samples were prepared at 12.5 nM for antibodies 252 and 100 and at 25.0 nM for antibody 88. These samples were two-fold serially diluted to 0.78 nM for roughly a 15-30 fold range in concentrations. For each concentration, the samples were injected in duplicate in random order at 30 μl/min flow for 3 min. The dissociation was monitored for 300 sec. The data were fit globally to a simple 1:1 binding model using BIACORE™ Biaevaluation software. In all cases, this method was used to obtain k_{off} and it was found that this data set compared well to data obtained from global fit of association and dissociation data.

Table 4 shows results for antibodies 252, 88, 100, 3.8.3, 2.7.3 and 1.120.1.

TABLE 4

| | 252 | 88 | 100 | 3.8.3 | 2.7.3 | 1.120.1 |
|---|---|---|---|---|---|---|
| K_{D} (M) | 1.33 × 10⁻¹¹ | 1.33 × 10⁻⁹ | 2.0 × 10⁻¹¹ | 4.0 × 10⁻¹⁰ | 4.7 × 10⁻⁹ | 5.4 × 10⁻⁹ |
| k_{off} (1/s) | 1.03 × 10⁻⁶ | 7.3 × 10⁻⁵ | 1.7 × 10⁻⁵ | | | |

### Example VII

### Production of 8.10.3 Antibodies from 8.10.3 Hybridoma Cells

Antibody 8.10.3 was produced in 3 L sparged spinners. The 3 L sparged spinner flask is a glass vessel where cultures are mixed with an impeller controlled by a magnetic platform. The spinner is connected to gas lines to provide 5% CO₂ and air. 8.10.3 hybridoma cells were initially thawed into T-25 cell culture flasks. The cells were progressively expanded until there was a sufficient number of cells to seed the sparged spinners.

Two 3 L sparged spinner flasks were seeded with 8.10.3 hybridoma cells in Hybridoma Serum-Free Medium with the additions noted on Table 5, for the two sparged flasks. The concentrations for Ultra low IgG serum (Gibco cat#16250-078), L-glutamine (JRH Biosciences cat#59202-500M), Non-Essential Amino Acids (Gibco cat#11140-050), Peptone (Difco cat#211693), glucose (In-house stock prepared from JT Baker cat#1920-07), and Anti-foam C (Sigma cat.#A-8011) are given at their final concentrations in the media. The balance of the volume in each reactor is Hybridoma Serum-Free Medium.

TABLE 5
Conditions for Growing Hybridoma 8.10.3
in two 3L sparged spinners.

| Conditions | Spinner 1 | Spinner 2 |
|---|---|---|
| Seeding density (1 × 10⁶ cells/ml) | 0.16 ml | 0.16 ml |
| Hybridoma Serum-Free Medium (Gibco cat# | Balance | Balance |
| 12045-076) | | |
| Ultra low IgG serum (Gibco cat# 16250-078) | 5% | 5% |
| L-glutamine (JRH Biosciences cat# 59202-500M) | 8 mmol/L | 8 mmol/L |
| Non-Essential Amino Acids (Gibco cat# 11140- | 1% | 1% |
| 050) | | |
| Peptone (Difco cat# 211693) | 1 g/L | 1 g/L |
| 2M glucose (In-house stock prepared from JT | 8 g/L | 8 g/L |
| Baker cat# 1920-07) | | |
| Anti-foam C (Sigma cat.# A-8011) | 1 ml/L | 1 ml/L |

The cultures were grown for 15 days and were harvested when the viability was below 20%. Viability was determined by trypan blue exclusion method with an automated cell counter (Cedex, Innovatis). Harvesting was accomplished by centrifugation and subsequent filtration. Clarified supernatant was obtained after centrifugation for 15 minutes at 7000 rpm and subsequent filtration with a sterile 0.22 μm 4″ Opticap Millipore filter (cat#KVSCO4HB3) into a 10 L sterile TC-Tech bag (cat #P/N 12420 Bag Style CC-10-112420). The filtrate was then purified in the following example.

### Example VIII

### Purification of an Anti-M-CSF Antibody

A Protein A column (Amersham Pharmacia) was prepped by washing with 3 column volumes of 8M Urea, followed by an equilibration wash with 20 mM Tris (pH 8). The final filtrate from Example VII was spiked with 2% v/v of 1M Tris pH 8.3 and 0.02% NaN₃ before being loaded onto the Protein A column via gravity-drip mode. After load was complete, the resin was washed with 5 column volumes of 20 mM Tris (pH 8), followed by 5 column volumes of the elution buffer (0.1 M Glycine pH 3.0). Any precipitation was noted, and then a 10% v/v spike of 1M Tris pH 8.3 was added to the eluted antibody. The eluted protein was then dialyzed into 100 fold the volume amount of eluted material of dialysis buffer (140 mM NaCl/20 mM Sodium Acetate pH 5.5). Following dialysis, the antibody was sterile filtered with a 0.22 μm filter and stored until further use.

### Example IX

### Monkey Treatment and Monocyte Counts

One male and one female cynomolgus monkey per dosage group were intravenously administered vehicle or antibody 8.10.3 (produced as describe in Examples VII and VIII) at 0, 0.1, 1, or 5 mg/kg in a dose volume of 3.79 mL/kg over an approximately 5 minute period. Blood samples for clinical laboratory analysis were collected at 24 and 72 hours postdose and weekly for 3 weeks. The monocyte counts were determined by light scatter using an Abbott Diagnostics Inc. Cell Dyn system (Abbott Park, Illinois).

A dose-related decrease (˜25% to 85%) in total monocytes at all doses (FIGS. 1A and 1B) was observed. Monocyte counts at the 0.1 and 1 mg/kg appeared to rebound to near control levels by week 2, while monocyte counts at 5 mg/kg were still decreased at 3 weeks.

### CD14+CD16+ Monocyte Subset Analysis

Primate whole blood was drawn into Vacutainer tubes containing sodium heparin. 0.2 ml of each blood sample was added to a 15 ml conical polypropylene centrifuge tube containing 10 ml of red blood cell lysis buffer (Sigma), and incubated in a 37° C. water bath for 15 minutes. The tubes were then centrifuged in a Sorvall RT7 centrifuge for 5 minutes at 1,200 rpm. The supernatant was aspirated, the pellet resuspended in 10 ml of 4° C. FACS buffer (Hanks' Balanced Salt Solution/2% FBS/0.02% sodium azide), and the tube centrifuged again for 5 minutes at 1,200 rpm. The supernatant was aspirated and the pellet resuspended in an antibody cocktail consisting of 80 μl 4° C. FACS buffer, 10 μl FITC-conjugated anti-human CD14 monoclonal antibody (BD Biosciences, San Diego, Calif.), 0.5 μl Cy5-PE-conjugated anti-human CD16 monoclonal antibody (BD Biosciences, San Diego, Calif.), and 10 μl PE-conjugated anti-human CD89 monoclonal antibody (BD Biosciences, San Diego, Calif.). The cell suspension was incubated on ice for 20 minutes, after which 10 ml of 4° C. FACS buffer was added and the cells centrifuged as before. The supernatant was aspirated, and the cell pellet resuspended in 400 μl FACS buffer and the cells analyzed on a FACSCaliber flow cytometer (BD Biosciences, San Jose, Calif.). Data for 30,000 cells were collected from each sample.

The monocyte population was identified by a combination of forward angle light scatter and orthogonal light scatter. Cells within the monocyte gate were further analyzed for expression of CD14 and CD16. Two distinct population of monocytes were observed, one expressing high levels of CD14 with little or no CD16 expression (CD14++CD16−) and the other expressing lower levels of CD14, but high levels of CD16 (CD14+CD16+), similar to the two monocyte subsets previously described in human peripheral blood (Ziegler-Heitbrock H. W., Immunology Today 17:424-428 (1996)). For each primate tested, the percentage of monocytes within the CD14+CD16+ subset was determined after each blood draw, on days 1, 3, 7, 14, and 21 after 8.10.3 injection.

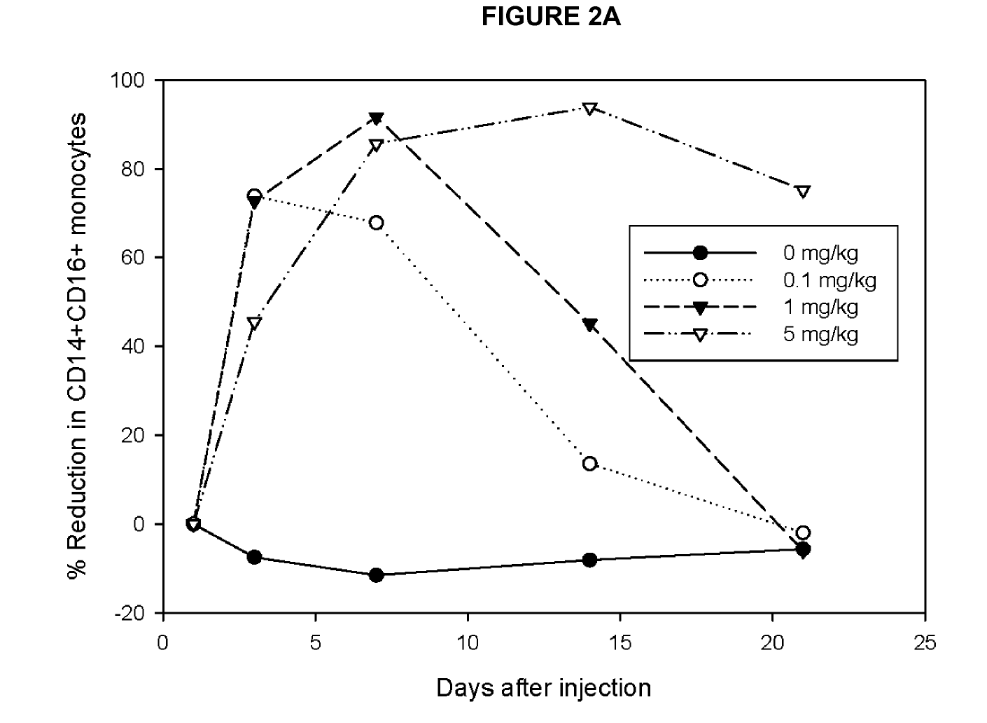

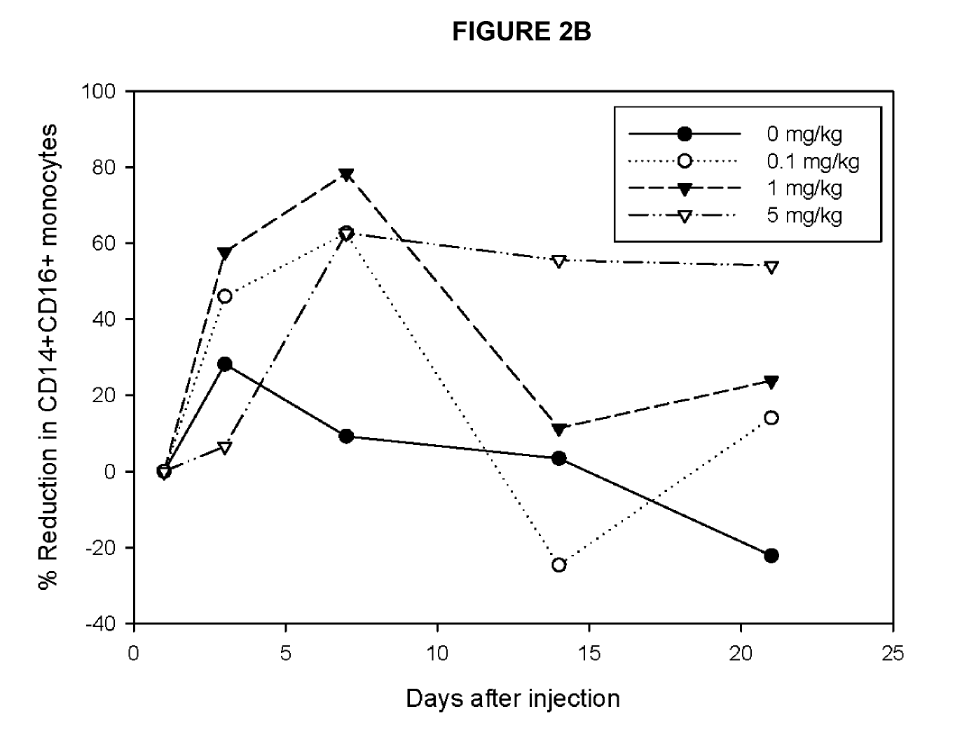

In general, 8.10.3 treatment resulted in a reduction in the percentage of CD14+CD16+ monocytes (see FIGS. 2A and 2B). Monkeys not receiving 8.10.3 Antibody demonstrated relatively stable CD14+CD16+ monocyte levels. CD14+CD16+ monocytes have been termed “proinflammatory” because they produce higher levels of TNF-α and other inflammatory cytokines (Frankenberger, M. T., et al., Blood 87:373-377 (1996)). It has also been reported that the differentiation of monocytes from the conventional CD14++CD16− phenotype to the proinflammatory phenotype is dependent on M-CSF (Saleh M. N., et al., Blood 85: 2910-2917 (1995)).

### Example X

### Monkey Treatment and Monocyte Counts

Three male cynomolgus monkeys per dosage group were intravenously administered vehicle (20 mM Sodium acetate, pH 5.5, 140 mM NaCl), purified antibody 8.10.3F, or purified antibody 9.14.4I at 0, 1, or 5 mg/kg in a dose volume of 3.79 mL/kg over an approximately 5 minute period. The monkeys were 4 to 9 years of age and weighed 6 to 10 kg. Blood samples for clinical laboratory analysis were collected at 2, 4, 8, 15, 23, and 29 days. Monocyte counts were determined by light scatter using an Abbott Diagnostics Inc. Cell Dyn system (Abbott Park, Illinois).

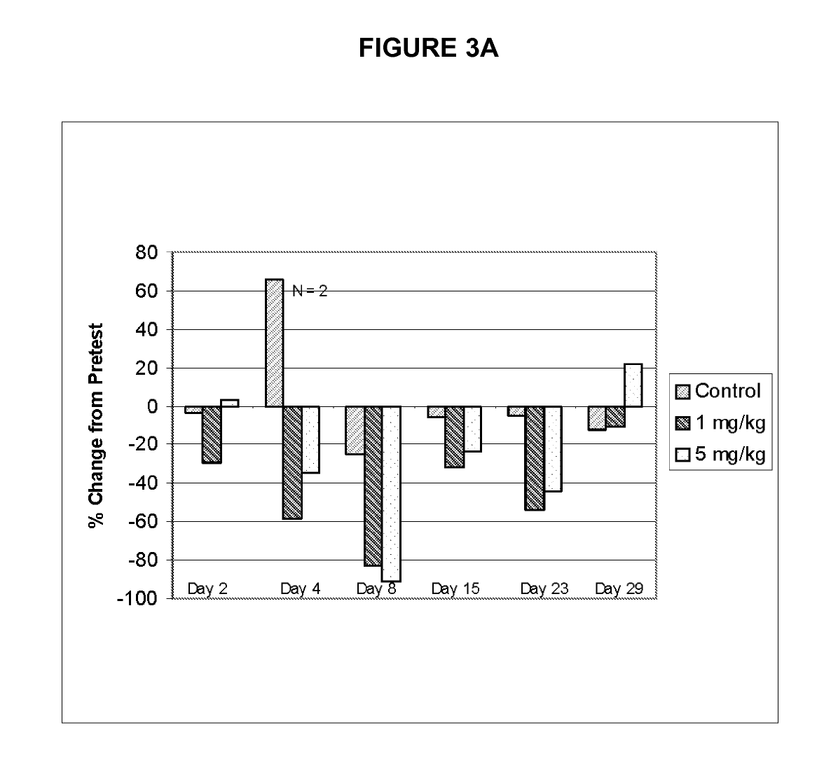

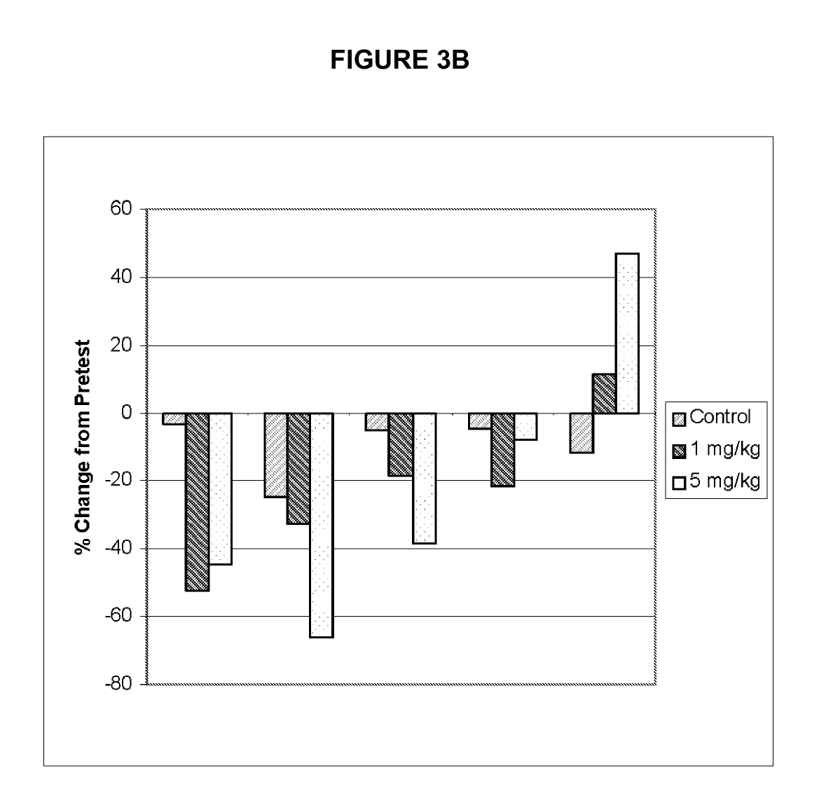

A decrease in the percentage change in total monocytes at all doses of antibody 8.10.3F and antibody 9.14.4I as compared to pre-test levels of monocytes (FIGS. 3A and 3B) was observed (see e.g., day 4, 8, 15, and 23 in FIGS. 3A and 3B).

### Example XI

### Effect of Anti-M-CSF Antibody in Murine Lupus Models

In this example, the effects of M-CSF neutralization by the rat anti-murine M-CSF antibody on the generation of lupus-like disease was tested in 2 separate murine models: the MRL-Fas^{Ipr} and the NZBWF1/J. The effects of the anti-M-CSF antibody were also compared with murine CTLA-4Ig, which has been previously shown to reduce disease in MRL-Fas^{Ipr} mice.

Several candidates for treatment of human disease have been evaluated in murine models of SLE which exhibit many of the same features as human disease (Perry et al, J Biomed Biotechnol. 2011: 271694. Epub 2011 Feb. 14).

MRL-Fas^{Ipr} mice spontaneously develop symptoms resembling those observed in human lupus, including high titers of circulating anti-double-stranded DNA (dsDNA) serum autoantibodies, IgG deposits in the glomeruli, and, in severe disease, proteinuria, lymphadenopathy, and skin lesions. The development of lymphadenopathy is due to an accumulation of double negative (CD4− CD8−) and B220+ T-cells (Watson et al, Journal of Experimental Medicine. 176(6):1645-56 (1992)). Cytotoxic T-cell lymphocyte antigen-4 (CTLA-4) is involved in regulating T lymphocyte activation, and the presence of serum CTLA-4Ig is associated with decreased expansion of autoreactive B lymphocytes, decreased numbers of CD4+ T lymphocytes, and a beneficial effect in a mouse model of SLE (Mihara et al, J Clin Invest. 106(1):91-101 (2002)). MRL-Fas^{Ipr} mice deficient in M-CSF are protected from nephritis (Lenda et al. J. Immunol. 173(7):4644-4754 (2004)).

The NZBWF1/J model is the oldest classical model of lupus generated by the F1 hybrid between the NZB and NZW strains which develops a severe lupus-like phenotype comparable to that of lupus patients including lymphadenopathy, splenomegaly, elevated serum antinuclear autoantibodies including anti-dsDNA IgG. Immune complex-mediated glomerulonephritis becomes apparent at 5-6 months of age, leading to kidney failure and death at 10-12 months (Mihara et al.). As in human SLE, disease in the NZBWF1 is strongly biased in favor of females (Theofilopoulos & Dixon, Adv Immunol. 37:269-390 (1985)).

### Materials and Methods

The antibodies and control proteins used in this example are summarized in Table 6.

TABLE 6

| Protein^{a} | Activity |
|---|---|
| Rat anti-mouse M-CSF Ab 5A1 | Neutralizing antibody for mouse M-CSF |
| CHOCK IgG1 | Rat isotype control immunoglobulin |
| CTLA-4Ig | Positive control for reducing lupus |
| | disease |

CHOCK = Chinese hamster ovary cells expressing CK-1;
CTLA = cytotoxic T lymphocyte-associated antigen: Ig = immunoglobulin;
M-CSF = macrophage colony stimulating factor.
^{a}Proteins were manufactured at Pfizer.

Female MRL-Fas^{Ipr} and NZBWF1/J (Jackson Laboratory, Bar Harbor, Me.) were housed in the pathogen-free animal facility at Pfizer. Mice were used according to protocols approved by the Pfizer Animal Care and Use Committee.

### SLE Studies

The effect of M-CSF neutralization on the development of lupus-like disease was examined using both the MRL-Fas^{Ipr} and NZBWF1/J models of SLE. Ten week old female MRL-Fas^{Ipr} or 26 week old female NZBWF1/J mice were treated 3 times per week intraperitoneally (IP) for 10 weeks with saline, 10 mg/kg of 5A1 anti-M-CSF. CHOCK IgG1 isotype control antibody or CTLA-41 g (MRL model only). MRL-Fas^{Ipr} mice were examined bi-weekly for proteinuria, skin lesions and lymphadenopathy, and NZBWF1/J mice were examined bi-weekly for proteinuria. Proteinuria was measured using Albustix (Bayer, Tarrytown, N.Y.) and scored on a scale of 0-5 where 0=none; 1=trace; 2=30 mg/dL; 3=100 mg/dL; 4=300 mg/dL; and 5=≧2000 mg/dL. Lymph nodes were palpated and lymphadenopathy was scored on a scale of 0-3 where 0=none; 1=small; 2=moderate, at two different sites; 3=large, at three or more different sites. Skin lesions were assessed by gross pathology and scored on a scale of 0-3 where 0=none; 1=small (face, ears); 2=moderate (<2 cm face, ears and back); 3=severe (>2 cm face, ears and back). Serum in both models was also collected bi-weekly and examined for anti-dsDNA IgG serum antibodies by enzyme immunosorbent assay (ELISA). At the conclusion of the study, brain, lung and kidney were collected in either 10% non-buffered formalin for pathology or frozen in optimal cutting temperature (OCT) compound for immunohistochemistry.

### Anti-dsDNA Serum Antibody and M-CSF ELISA

Anti-dsDNA IgG serum antibodies were measured by ELISA. Briefly, Immulon 1B plates (Thermolab Systems, Billerica, Mass.) were UV irradiated over night and then coated with 2 μg/mL calf thymus DNA (Sigma Aldrich, St. Louis, Mo.) for 1 hour at room temperature. Plates were blocked with phosphate buffered saline (PBS) plus 1% bovine serum albumin (BSA), diluted serum samples were added (starting at 1:100 dilution), and bound antibody was detected with horseradish peroxidase (HRP)-conjugated goat antibodies directed against mouse IgG antibody (Southern Biotech, Birmingham, Ala.). Plates were developed using 3,3′,5,5′-tetramethylbenzidine (TMB; KPL, Gaithersburg, Md.) and reactions were stopped with 2N sulfuric acid. Absorbancies were read at 450 nm using a SpectraMax Plus 384 microplate reader with SoftMax Pro software (Molecular Devices, Sunnyvale, Calif.). Arbitrary units of antibody were determined using standard positive control serum pooled from diseased MRL-Fas^{Ipr} or NZBWF1/J mice. Serum levels of M-CSF were determined by an anti-murine M-CSF kit (R & D Systems, Minneapolis, Minn.; Cat.# MC00) as specified by the manufacturer.

### Histology

In the MRL-Ipr study, kidneys were examined for pathology and Ig and C3 deposits. Formalin-fixed specimens of right and left kidneys were submitted for hematoxylin and eosin (H&E) and periodic acid-Schiff (PAS; with hematoxylin counterstain)-staining. Specimens of right and left kidneys were also frozen in liquid nitrogen and were stained immunohistochemically for C3, IgG, and IgM. All tissue sections were examined by a board certified veterinary pathologist. F4/80 stained macrophages were examined by immunohistochemistry.

### Results

A study was set up in 8 week old MRL-Ipr mice to test the effect of blocking M-CSF with a neutralizing antibody, 5A1 (see, for example, Campbell I. K., et al., J. Leuk. Biol. 68:144-150 (2000) and ATCC Number CRL-2702). Mice were treated with 400 μg antibody (10 mg/kg) intraperitoneally (IP), daily for 12 weeks. Control mice were treated with the same dose of an isotype control antibody, CHOCK IgG1 (see, for example, ATCC Number HB-9421) or saline. Mice were also treated with murine CTLA-4Ig (extracellular domain of murine CTLA4 fused to murine IgG2a containing effector function null mutation) as a positive control. Mice were examined for anti-dsDNA serum antibody, proteinuria, skin lesions and lymphadenopathy bi-weekly. The study design is outlined in Table 7.

TABLE 7
Study Design for Testing the Efficacy of Anti-M-CSF Antibody in
MRL^{lpr} Lupus Model

| | | | |
|---|---|---|---|
| | | | Animals examined |
| Treatment | Number | IP Dose | microscopically^{a} |

| | | | |
|---|---|---|---|
| Saline | 10 | 100 μL | 1-10 |
| | | 3x/week | |
| CTLA-4Ig | 10 | 10 mg/kg, | 11-20 |
| | | 3x/week | |
| Rat anti-M-CSF Ab | 10 | 400 μg | 31-40 |
| 5A1 | | 3x/week | |
| Rat CHOCK IgG1 | 10 | 400 μg | 41-40 |
| control Ab | | 3x/week | |

^{a}One H&E-stained, one PAS-stained, one C3-immunostained, one IgG-immunostained, one IgM immunostained, and one control antibody-immunostained slide were examined for each animal.

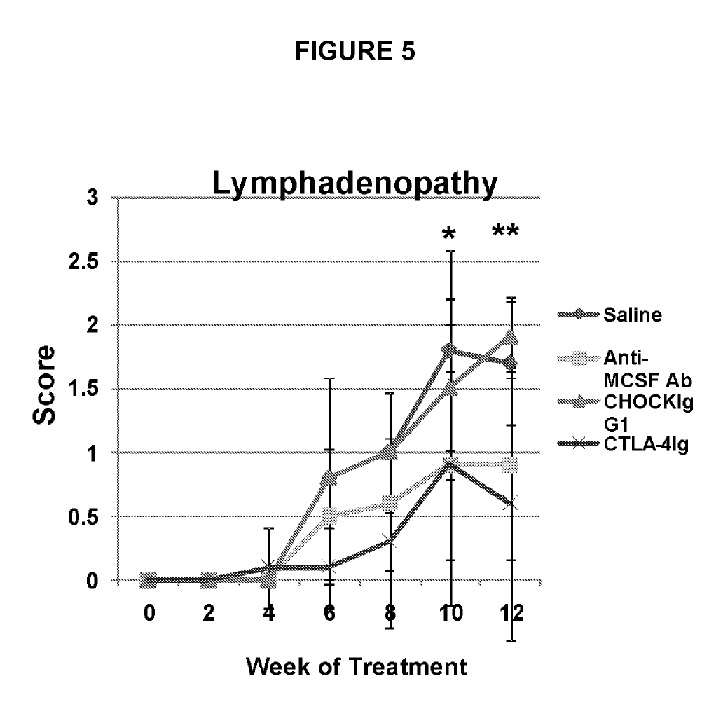

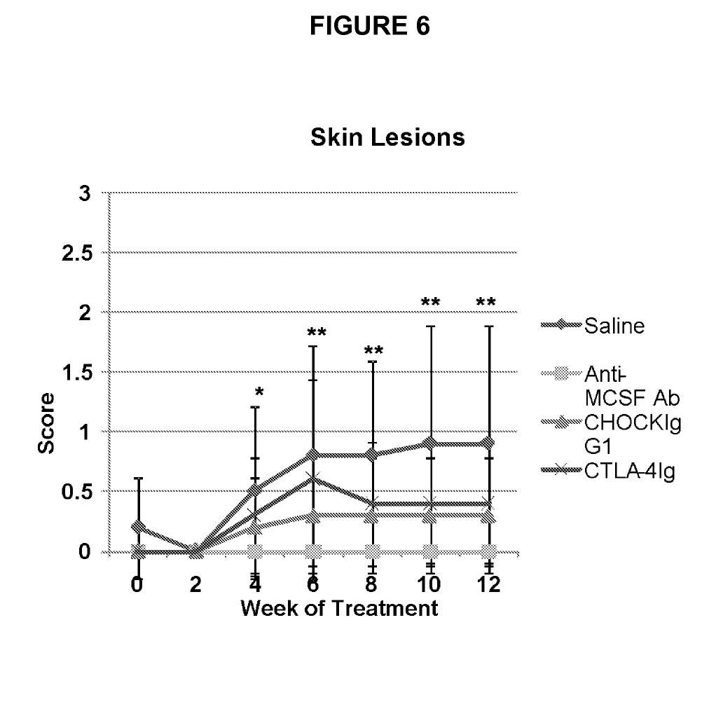

As shown in FIG. 5, treatments with the positive control protein CTLA-4Ig and anti-M-CSF antibody significantly reduced the severity of lymphadenopathy in MRL-Ipr mice. FIG. 6 shows that treatment with anti-M-CSF antibody also significantly reduced the severity of the skin lesions that developed in this model. Interestingly, treatment with anti-M-CSF prevented the development of skin lesions in MRL-Ipr mice, in contrast to treatment with CTLA-4Ig, which reduced the severity of skin lesions in MRL-Ipr mice.

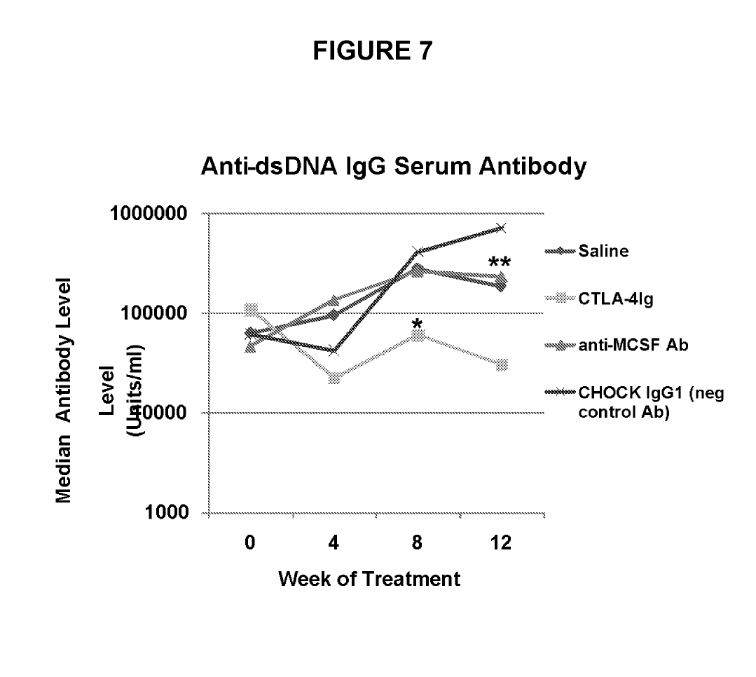

As shown in FIG. 7, mice also developed less anti-dsDNA antibodies at the 12-week time point in this study when compared with the isotype control treated mice; whereas, treatment with CTLA-4Ig was very effective in reducing the development of anti-dsDNA antibodies at the 4 time-points tested.

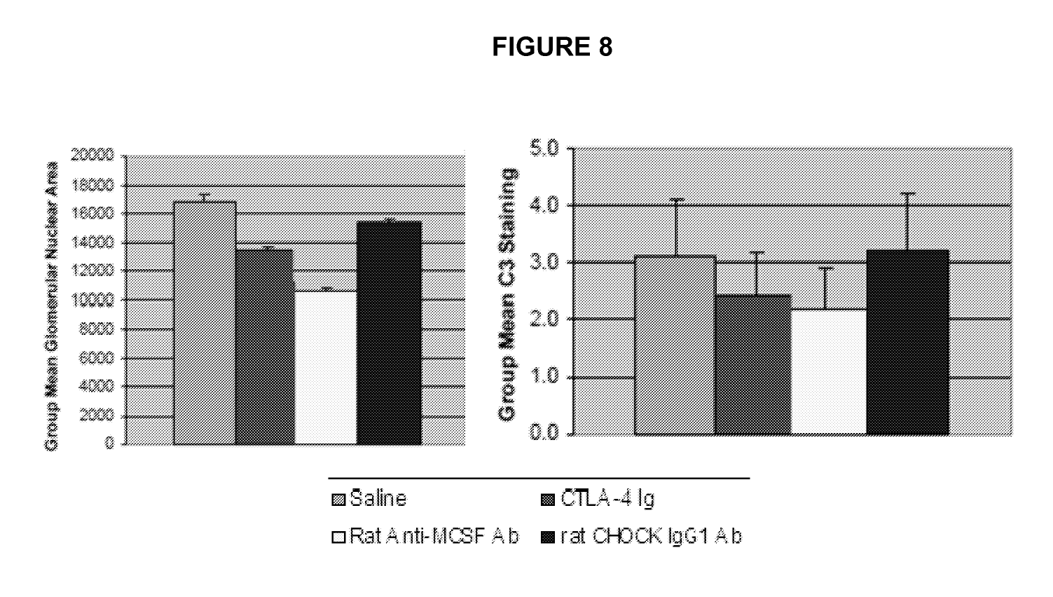

In this study, mice did not develop significant levels of proteinuria; therefore, kidney function was not assessed. Microscopically, only administration of CTLA-4Ig was associated with beneficial effects on the severity of inflammatory infiltrates and proteinaceous casts, the size of glomerular tufts, glomerular cellularity, and the incidence and severity of glomerular IgG deposits. As shown in FIG. 8, administration of rat anti-M-CSF antibody was associated with lower glomerular cellularity, and with a slightly lower group mean immunohistochemical staining score for C3 compared with administration of saline or isotype control. However, this was not associated with any other clear beneficial effects on any other parameters measured.

A subsequent study evaluated the efficacy of anti-M-CSF antibody 5A1 in ameliorating lupus nephritis disease in the NZBWF1/J model. Twenty-six week old mice were treated with 400 μg (10 mg/kg) of either control Ig or anti-M-CSF or saline, 3 times per week IP for 10 weeks. Mice were scored bi-weekly for proteinuria, weight gain/loss, and anti-dsDNA titers. Kidneys were harvested after 10 weeks of dosing and examined for pathology and immune complex deposition. The study design is described in Table 8.

TABLE 8
Study Design for Testing Efficacy of Anti-M-CSF Antibody in the
NZBWF1/J Lupus Model

| | | | Animals Examined | Animals Examined |
|---|---|---|---|---|
| | | Dose | Microscopically: | Microscopically: |
| Treatment | Number | (IP) | Kidneys^{a} | Kidneys^{b} |
| Saline | 10 | 100 μL | 46, 47, 48, 49, 50, | 46, 47, 48, 49, 50, |
| | | 3x/week | 51, 52, 53, 54, 55 | 51, 52, 53, 54, 55 |
| Rat anti- | 10 | 400 μg | 57, 58, 59, 60, 61, | 57, 58, 60, 61, 62, |
| M-CSF | | 3x/week | 62, 63, 64, 65 | 63, 64, 65 |
| Ab 5A1 | | | | |
| Rat | 10 | 400 μg | 66, 67, 69, 70, 71, | 66, 67, 69, 70, 71. |
| CHOCK | | 3x/week | 72, 73, 74, 75 | 72, 73, 74, 75 |
| IgG1 | | | | |
| control Ab | | | | |

^{a}One H&E and one PAS stained tissue section and two F4/80 immunostained tissue sections were examined from each animal.
^{b}Four immunostained tissue sections for IgG, IgM and C3 were examined from each animal

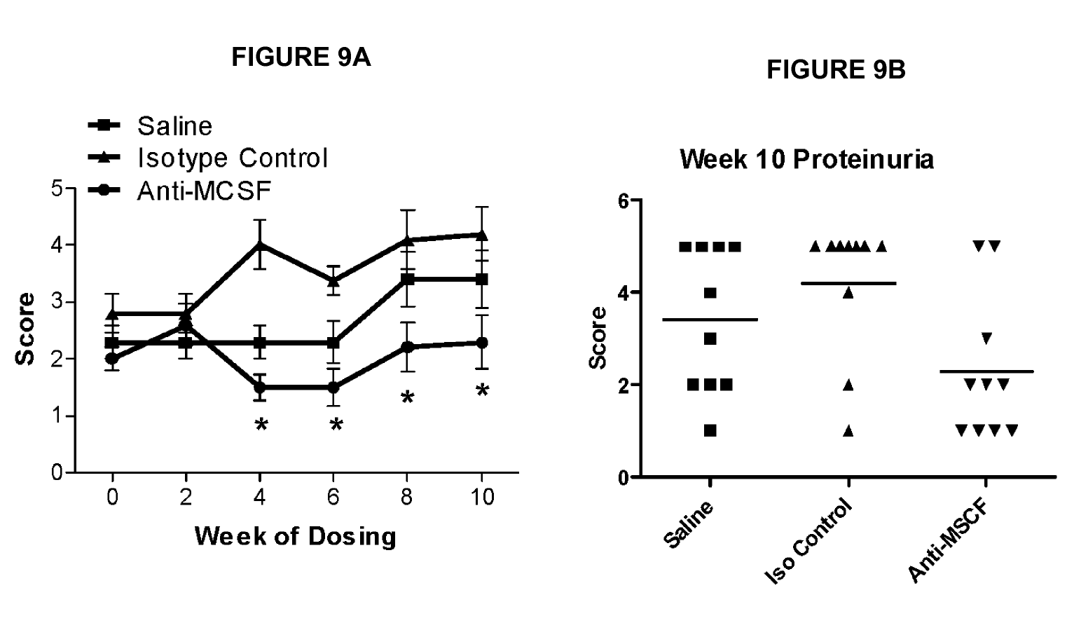

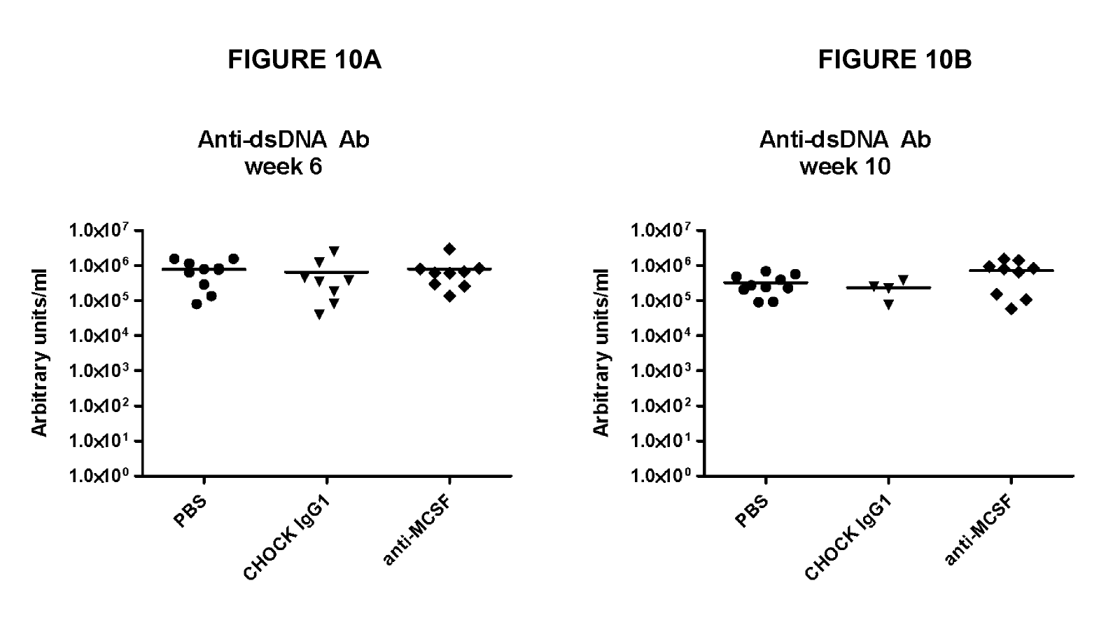

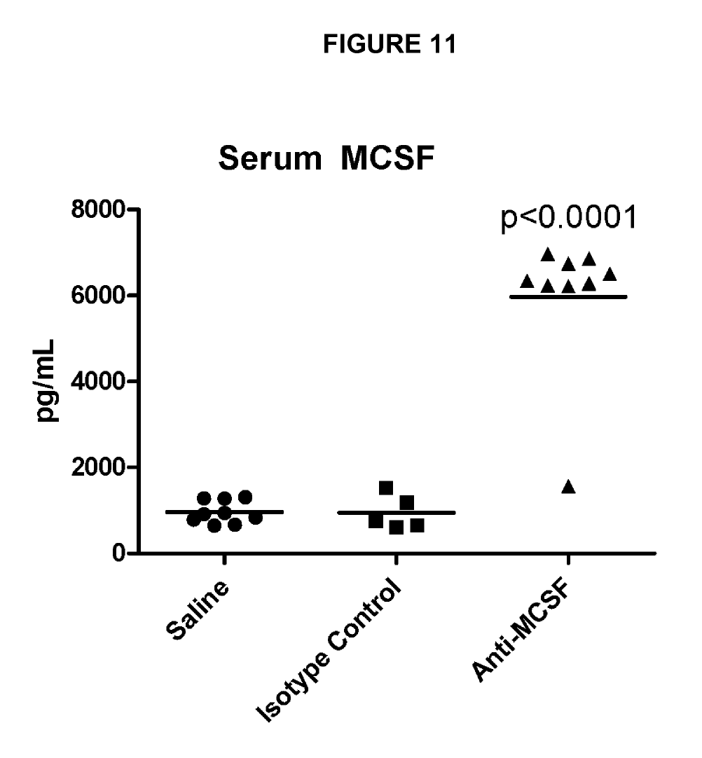

As shown in FIG. 9, anti-M-CSF treatment had a significant effect on the development of proteinuria when compared with the isotype control starting at 4 weeks after dosing until the end of the study in the NZBWF1/J lupus model. Treatment with antibody to M-CSF however did not affect the development of anti-dsDNA autoantibodies that contribute to immune complex deposition and kidney damage in this model. As shown in FIG. 10, anti-dsDNA antibody levels were similar in saline, isotype control, and anti-M-CSF treated mice at the 6 and 10 week time points after dosing. FIG. 11 shows that treatment with M-CSF antibody raises the serum level of M-CSF as determined by ELISA, indicating that the antibody is reacting with its target and sequesters M-CSF in the serum.

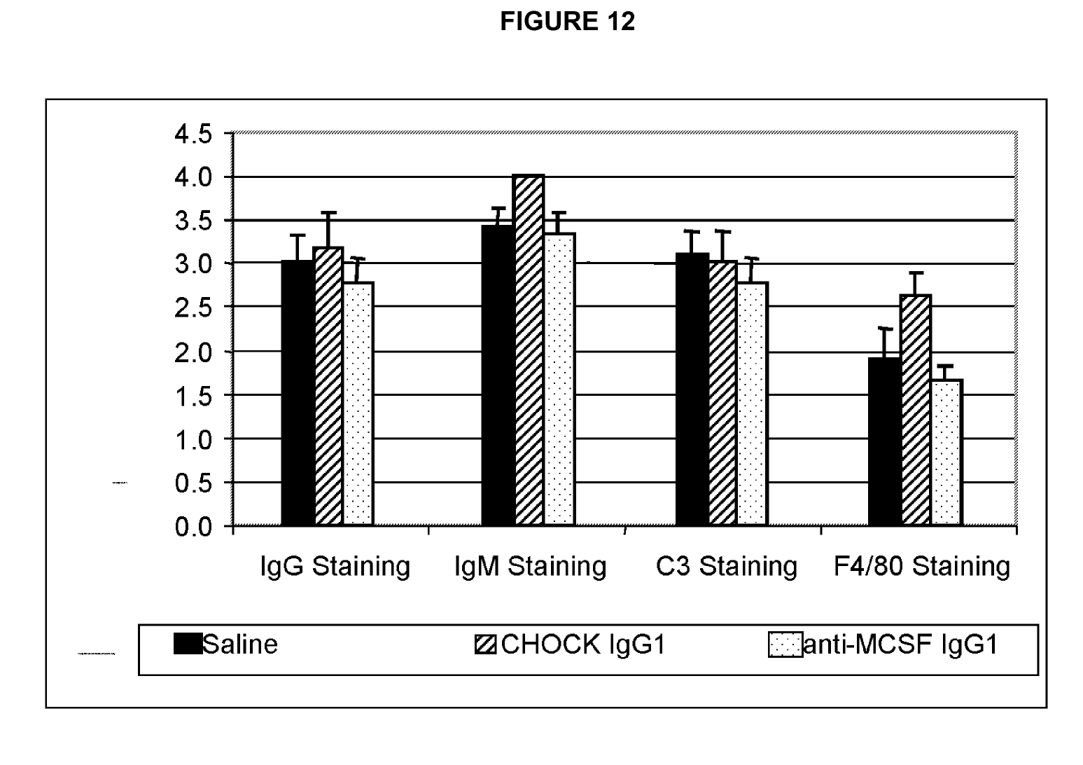

Microscopic examination of kidneys collected at the termination of the study and stained for immune deposits showed similar levels of IgG, IgM and C3 staining in all 3 treated groups, shown in FIG. 12. Interestingly, kidneys obtained from mice treated with anti-M-CSF showed a slight reduction in staining for macrophages (F4/80 positive a transmembrane protein expressed by mature macrophages.) when compared with the isotype control treated mice. Renal F4/80 staining for macrophages is present in interstitial cells in the outer medulla and the inner cortex in all mice. Staining is present in fewer cells in these locations in mice administered saline or rat anti-M-CSF compared with mice administered CHOCK IgG1 isotype control antibody suggesting that M-CSF blockade had an effect on macrophage infiltration in the kidney in this model. Anti-M-CSF treatment also appeared to reduce renal proteinaceous casts and tubular basophilia and degeneration in kidney of NZBWF1/J mice. The findings are less severe in animals administered saline or anti-M-CSF compared with mice administered CHOCK IgG1 isotype control.

### Example XII

### Study Design

This was a randomized, double-blind (sponsor unblinded), placebo-controlled, dose-escalating, parallel-group study in 6 sequential cohorts investigating the safety and tolerability of 6 single dose levels of human monoclonal anti-M-CSF antibody 8.10.3F, administered as an intravenous (IV) formulation, in healthy adult volunteers.

### Subject Disposition

A total of 48 subjects were enrolled in the study. Subjects in Cohorts 1 through 5 received doses of 3, 10, 30, 100, or 300 mg antibody 8.10.3F or placebo; these subjects were confined to the CRU (clinical research unit) for 3 days after dosing, were discharged, and returned to the CRU for scheduled assessments. Subjects in Cohort 6 were treated with 100 mg antibody 8.10.3F or placebo and were confined to the CRU for 21 days after dosing, were discharged, and returned to the CRU for scheduled assessments. At each dose level, 6 subjects were treated with single doses of antibody 8.10.3F and 2 with single doses of placebo. All enrolled subjects completed the study.

### Pharmacokinetic Evaluations

### A) Serum for Analysis of Antibody 8.10.3F

Samples for analysis of antibody 8.10.3F were collected pre-dose and at protocol-specified times post-dose through Day 28, as well as each additional outpatient visit after Day 28. Three mL of venous blood was collected into appropriately labeled tubes containing no additive, centrifuged within 40 minutes of collection, and the serum was stored frozen at approximately −70 degrees Celsius within 60 minutes of collection.

Serum samples were analyzed for antibody 8.10.3F at PPD Development (2244 Dabney Road, Richmond, Va. 23230, USA) using a validated analytical assay. Antibody 8.10.3F samples were assayed using a validated, sensitive and specific Enzyme-Linked Immunosorbent Assay (ELISA). The serum specimens were stored at approximately −70° C. until assay, and samples were assayed within 439 days of established matrix stability data. Sample concentrations were determined by interpolation from a calibration standard curve (over the range of 35.0 ng/mL to 1600 ng/mL) that had been fit using a 4-parameter logistic equation. Those samples with concentrations above the upper limit of quantification (1600 ng/mL) were adequately diluted into calibration range. The LLOQ (lower limit of quantification) for antibody 8.10.3F was 35.0 ng/mL. Clinical specimens with serum antibody 8.10.3F concentrations below the LLOQ are reported as <35.0 ng/mL.

The between-day assay accuracy, expressed as the ratio (%) of the estimated to the theoretical Quality Control (QC) concentrations, ranged from −7.81% to 2.22% for low, medium, high and diluted QC samples. Assay precision, expressed as the between-day coefficients of variation (CV %) of the estimated concentrations of QC samples was less than 10.0% for low (75.0 ng/mL), medium (320 ng/mL), high (1200 ng/mL) and diluted (1200 ng/mL, 16000 ng/mL and 160000 ng/mL) concentrations.

### B) Calculation of Pharmacokinetic Parameters

Antibody 8.10.3F PK parameter values were calculated for each subject for each treatment using noncompartmental analysis of serum concentration-time data. The study design did not include PK sampling during the IV infusion. Given the anticipated long terminal t1/2 of antibody 8.10.3F, area under the concentration-time curve (AUC) during the infusion was expected to be minimal relative to the total AUC; therefore no attempt was made to estimate concentrations during or at the end of the infusion.

Samples below the LLOQ were included as zero. Actual sample collection times were used for the PK analysis. Pharmacokinetic parameter values were calculated using WinNonlin version 4.0.1.

### Pharmacodynamic Evaluations

### A) Whole Blood for Analysis of CD14⁺16⁺ Monocytes

Samples for analysis of CD14⁺16⁺ monocytes were collected pre-dose and on Days 2, 4, 7, 14, and 28, as well as at each additional outpatient visit after Day 28. Three mL of venous blood was collected into each of 2 tubes; one containing lithium heparin, and the other containing potassium EDTA. Samples were analyzed within 2 hours of collection. Whole blood samples were analyzed for the percent monocyte subpopulations (CD14 and CD16) in human peripheral blood at Pfizer Drug Safety Research & Development (DSRD), PGRD, Ann Arbor, Mich. using a validated, sensitive and specific flow cytometry assay (Becton-Dickinson FACSCalibur Flow Cytometry). Monocyte subpopulations were differentiated based on the ability of cells to scatter light and emit fluorescent signals.

For the assay performance characteristics for overall sample analysis, BD CaliBRITE beads were used to adjust instrument settings, to set fluorescence compensation and to evaluate instrument sensitivity before each run of the monocyte subsetting-CD14/16 assay.

### B) Serum for Bone Specific Alkaline Phosphatase (BSAP)

Samples for analysis of BSAP were collected pre-dose and on Days 2, 4, 7, 14, and 28, as well as at each additional outpatient visit after Day 28. Five mL of venous blood was collected into a tube containing no additive, centrifuged within 40 minutes, and approximately equal volumes of serum were transferred into 2 tubes and stored frozen at approximately −70° C. within 60 minutes of collection.

Samples were analyzed for BSAP concentration at Pacific Biometrics Inc. (PBI) (220 West Harrison Street, Seattle, Wash. 98119, US) using a validated analytical assay. BSAP samples were assayed using a validated, sensitive and specific Enzyme Immunoassay (EIA). The performance of the method during validation was documented. The serum specimens were stored at approximately −70° C. until assay, and samples were assayed within 365 days of established stability data generated during validation. Sample concentrations were determined by interpolation from a calibration standard curve that had been fit using a quadratic fitting equation. Those samples with concentrations above the ULOQ (140 U/L) were adequately diluted into calibration range. The assay sensitivity expressed as the observed limit of detection (LOD) for BSAP was 0.4 U/L. Clinical specimens with serum BSAP concentrations below the LOD are reported as below limit of detection.

The assay performance characteristics were evaluated by QC. Three levels of QC were placed on each run. The run acceptance criteria were: 2 out of 3 QC results must be within 2.0 standard deviation index (SDI), and the third must be within 2.5 SDI. For all four sample analysis runs, the mean run SDI ranged from −1.1 to 0.8 for QC samples.

### C) Urine for Analysis of NTX-1

Samples for analysis of urinary NTX-1 were collected pre-dose and on Days 2, 4, 7, 14, and 28, as well as at each additional outpatient visit after Day 28. The second morning void urine was collected in a clean container. At each timepoint, a 5-mL aliquot was collected for NTX-1. Two additional 5-mL aliquots were collected for exploratory biomarkers as scheduled. Samples were frozen at approximately −70° C.

Urine samples were analyzed for urinary N-telopeptide of cross-linked collagen I (uNTX-1) concentration at Pacific Biometrics Inc. (PBI) (220 West Harrison Street, Seattle, Wash. 98119, USA) using validated analytical assays, uNTX-1 samples were assayed using a validated, sensitive and specific ELISA for NTX-1 and a validated kinetic Jaffe for urine creatinine.

The urine specimens were stored at approximately −70° C. until assay, and samples were assayed within 365 days of established stability data generated during validation. Sample NTX-1 concentrations were determined by interpolation from a calibration standard curve that has been fit using a 4-parameter fitting equation. Urine creatinine values were determined from a linear calibration. Those samples with NTX-1 concentrations above the ULOQ (3000 nM equivalents per liter) were adequately diluted into calibration range. The NTX-1 assay sensitivity expressed as the LLOQ for NTX-1 was 44.0 nM equivalents per liter. Clinical specimens with NTX-1 concentrations below the LLOQ are reported as below limit of detection.

NTX-1 assay values were standardized to an equivalent amount of bone collagen, and are expressed in nanomoles bone collagen equivalents per liter (nmol BCE/L). uNTX-1 assay values are corrected for urinary dilution by urinary creatinine analysis and expressed in nanomole bone collagen equivalents per liter (nM BCE) per millimole creatinine per liter (mM creatinine).

The assay performance characteristics were evaluated by quality controls (QC). Three levels of QC were placed on each run. The run acceptance criteria were: 2 out of 3° C. results must be within 2.0 standard deviation index (SDI), and the third must be within 2.5 SDI. For all four sample analysis runs, the mean run SDI ranged from −0.2 to 0.8 for NTX-1 QC samples, and from −0.1 to 0.2 for urine creatinine QC samples.

### D) K₂EDTA Plasma for Analysis of Total Human M-CSF

K₂EDTA plasma samples collected for exploratory biomarkers were used for analysis of total M-CSF. Samples were collected pre-dose and on Days 2, 7, and 28, as well as at each additional outpatient visit after Day 28. A 10 mL venous blood sample was obtained in a blood collection tube containing no potassium EDTA (K₂EDTA) as an anticoagulant. After centrifugation, the plasma was stored frozen at approximately −70° C. ELISA assay kit (DMC00) from R & D Systems, Inc. (614 McKinley Place NE, Minneapolis 55413). The quantitative sandwich enzyme immunoassay technique was employed in this assay and the assay procedures and critical reagents were followed the kit insert. The K₂EDTA plasma specimens were stored at approximately −70° C. until assay. Sample concentrations are determined by interpolation from a calibration standard curve (over the range of 31.2 pg/mL to 2000 pg/mL) that has been fit using a 4-parameter logistic equation. Those samples with concentrations above the upper limit of quantification (2000 pg/mL) were adequately diluted into calibration range. The lower limit of quantification (LLOQ) for M-CSF was 31.2 pg/mL. Clinical specimens with serum M-CSF concentrations below the LLOQ are reported as <31.2 μg/mL.

K₂EDTA plasma samples were analyzed for M-CSF concentration at Pfizer Discovery-Molecular Pharmacology, PGRD, Ann Arbor, Mich. using a commercially available

### Results

### A) Serum Antibody 8.10.3F Pharmacokinetics

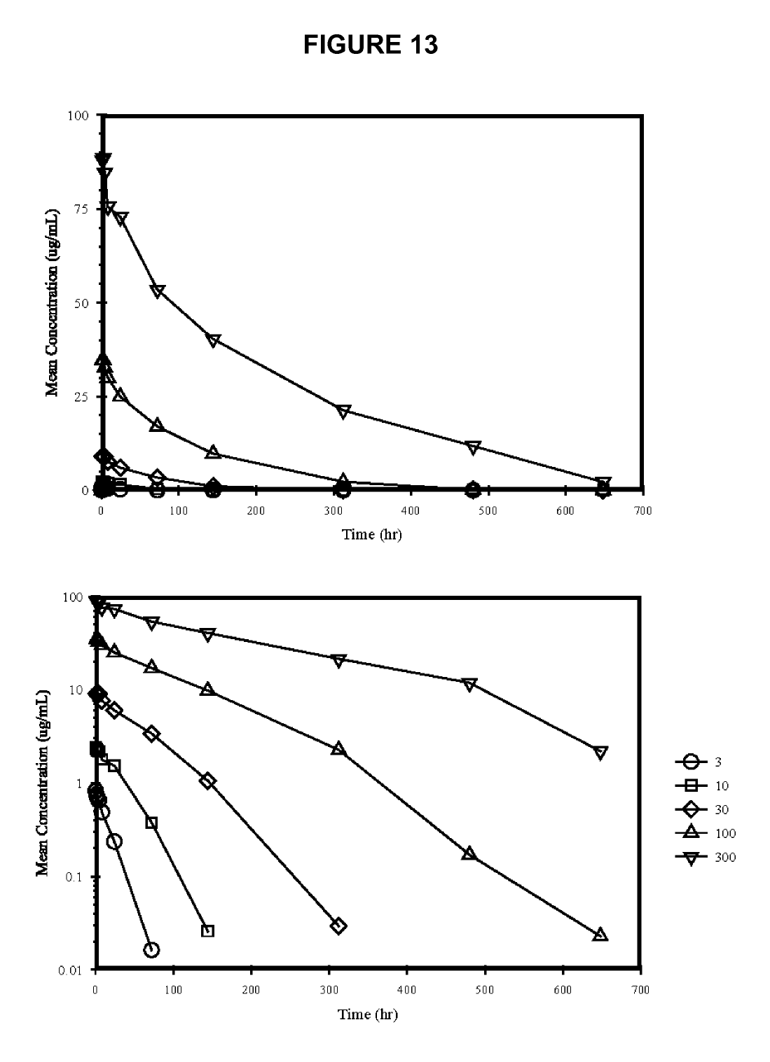

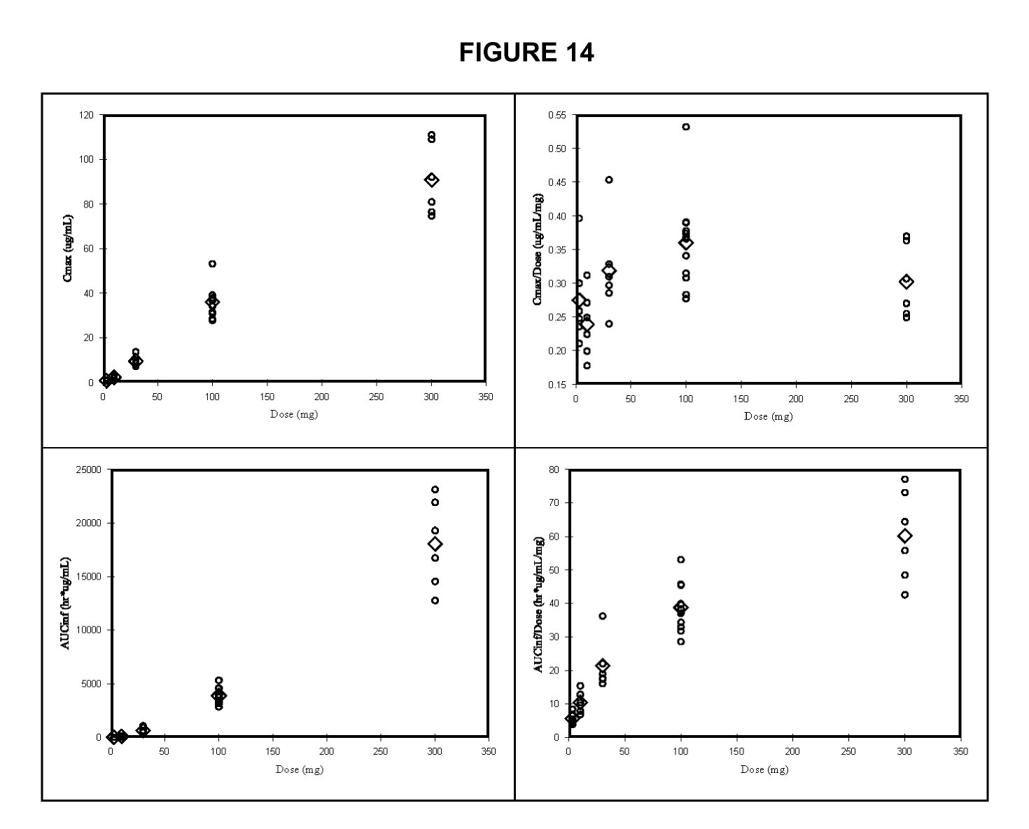

Table 9 and Table 10 contain summaries of serum antibody 8.10.3F pharmacokinetic parameters following administration of a single IV dose in healthy patients. Mean serum concentration-time profiles following administration of each dose are depicted in FIG. 13. Plots of Cmax and AUC values vs. Antibody 8.10.3F dose are shown in FIG. 14.

Following a single infusion of antibody 8.10.3F administered over 1 hour to healthy adult volunteers, Cmax increased in a dose-proportional manner. However, the extent of systemic exposure, AUC(0-∞), increased in a greater than dose-proportional manner over the dose range of 3-300 mg. Because of the nonlinearity of the system, the apparent terminal t (used to calculate AUC[0-∞]) was not felt to be a meaningful measure of the duration of exposure. Instead, the study day at which antibody 8.10.3F concentrations were below the LLOQ for all subjects was determined by inspection of the concentration-time tables. Serum antibody 8.10.3F concentrations were below the LLOQ following the 3, 10, 30, 100, and 300 mg doses by Days 7, 14, 28, 56, and 84, respectively.

TABLE 9
Summary of Serum Antibody 8.10.3F Pharmacokinetic
Parameter Values Following Administration of Single Intravenous
Solution Doses to Healthy Subjects

| Parameter, | Parameter Summary Statistics^{a} by Antibody 8.10.3F Dose | | | | |
|---|---|---|---|---|---|
| Units | 3 mg | 10 mg | 30 mg | 100 mg^{b} | 300 mg |
| N | 6 | 6 | 6 | 12 | 6 |
| Cmax, | 0.825 (24) | 2.39 (21) | 9.57 (23) | 36.0 (19) | 90.7 |
| μg/mL | | | | | (18) |
| tmax, hr | 0.25 (0.25-0.28) | 0.76 (0.25-4.0) | 2.0 (1.3-4.0) | 1.3 (1.3-2.0) | 3.1 (1.8-4.0) |
| AUC (0-tlqc). | 13.6 (49) | 97.7 (33) | 601 (40) | 3870 (18) | 17800 |
| μg * hr/mL | | | | | (22) |
| AUC (0-∞), | 16.9 (31) | 105 (30) | 640 (36) | 3870 (18) | 18100 |
| μg * hr/mL | | | | | (23) |
| T-BLQ^{c} | 7 | 14 | 21 | 56 | 84 |
| CL, mL/min | 3.18 (27) | 1.72 (29) | 0.842 (25) | 0.442 (17) | 0.290 |
| | | | | | (24) |
| Vd, mL/min | 4.20 (23) | 3.63 (26) | 3.21 (24) | 2.33 (22) | 2.92 |
| | | | | | (15) |
| Duration of | 7 | 14 | 21 | 56 | 84 |
| exposure | | | | | |
| (Days)^{c} | | | | | |

^{a}Median (range) for tmax; arithmetic mean (% CV) for all other parameters
^{b}Outpatient and inpatient cohorts combined.
^{c}Study Day on which serum concentrations were below the lower limit of quantitation (LLQ = 0.035 μg/ml) in all subjects
N = Number of subjects

TABLE 10
Mean Serum Antibody 8.10.3F Concentrations (ng/mL)
Following Administration of Single Intravenous Solution Doses to
Healthy Subjects

| | Study Day | | | | | |
|---|---|---|---|---|---|---|
| | 1 | 2 | 7 | 28^{a} | 56^{a} | 84 |
| Dose | 3 mg | | | | | |
| N | 6 | 6 | 6 | | | |
| Mean | 0.00 | 235.83 | 0.00 | | | |
| Median | 0.00 | 224.50 | 0.00 | | | |
| STD | 0.00 | 53.16 | 0.00 | | | |
| Dose | 10 mg | | | | | |
| N | 6 | 6 | 6 | 6 | | |
| Mean | 0.00 | 1531.67 | 25.88^{b} | 0.00 | | |
| Median | 0.00 | 1495.00 | 18.30^{b} | 0.00 | | |
| STD | 0.00 | 386.13 | 32.03 | 0.00 | | |
| Dose | 30 mg | | | | | |
| N | 6 | 6 | 6 | 6 | | |
| Mean | 0.00 | 5991.67 | 1057.83 | 0.00 | | |
| Median | 0.00 | 5480.00 | 803.50 | 0.00 | | |
| STD | 0.00 | 1597.09 | 717.84 | 0.00 | | |
| Dose | 100 mg | | | | | |
| N | 12 | 12 | 12 | 12 | 12 | |
| Mean | 0.00 | 24958.33 | 9751.67 | 22.93^{b} | 0.00 | |
| Median | 0.00 | 25400.00 | 9810.00 | 0.00 | 0.00 | |
| STD | 0.00 | 4170.78 | 2216.52 | 28.90 | 0.00 | |
| Dose | 300 mg | | | | | |
| N | 6 | 6 | 6 | 6 | 6 | 6 |
| Mean | 0.00 | 72800.00 | 40333.33 | 2476.00 | 11.82^{b} | 0.00 |
| Median | 0.00 | 75850.00 | 42300.00 | 1614.50 | 0.00 | 0.00 |
| STD | 0.00 | 13733.17 | 8571.74 | 2266.54 | 28.94 | 0.00 |

^{a}Data is summarized by nominal Study Day: Day 28 for the 300 mg dose group combines data collected on Day 28 for 5 of 6 Subjects with those collected on Day 29 for the other subject. Similarly, Day 56 for the 300 mg dose group combines data from 4 or 6 subjects collected on Day 56 with data from the other 2 of 6 subjects collected on Day 58..
^{b}Mean and/or median values below the limit of quantitation reflect inclusion of samples reported as BLQ (<35 ng/mL) as 0 in calculations

### B) Total Serum M-CSF

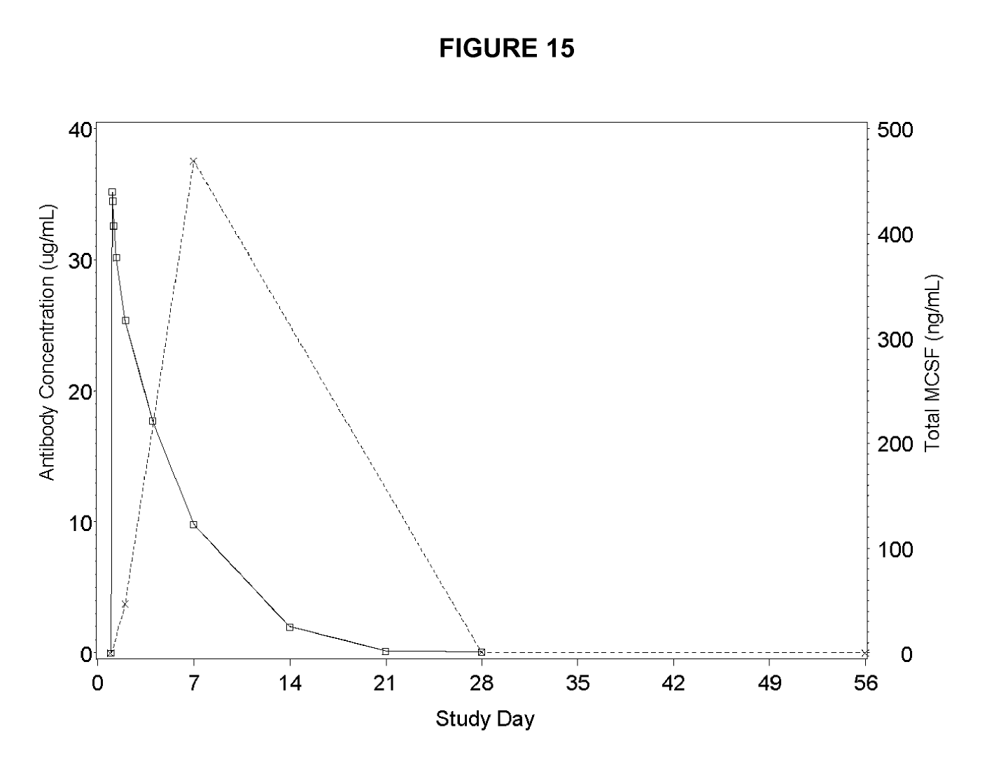

Total (free and antibody 8.10.3F bound) M-CSF concentrations are presented in Table 11 by dose and Study Day and illustrated for the 100-mg dose in FIG. 15.

In the placebo treatment group, mean M-CSF concentration was 0.21 ng/mL at baseline and did not change substantially (range 0.21-0.28 ng/mL) following the administration of placebo to healthy adult volunteers, up to Day 84 (Table 11). Following administration of antibody 8.10.3F, the maximum mean M-CSF concentration increased with increasing dose. Peak concentrations of total M-CSF were attained on Day 2 or 7. Using the equilibrium dissociation constant for antibody 8.10.3F (K_{D} 2.8×10⁻¹⁰ M) and the concentrations of M-CSF and of antibody 8.10.3F to determine ratios of free and bound ligand, it was calculated that during the time that antibody 8.10.3F was detectable, 100% of the measured M-CSF ligand was bound to antibody 8.10.3F. Although the sampling frequency for M-CSF was not as frequent as it was for antibody 8.10.3F, M-CSF concentrations declined in parallel with antibody 8.10.3F, suggesting that the temporary increase in M-CSF was subsequently cleared as the antibody-antigen complex.

TABLE 11
Mean M-CSF Concentrations (ng/mL) Following
Administration of Single Intravenous Solution Doses to Healthy
Subjects

| | Study Day | | | | | |
|---|---|---|---|---|---|---|
| | 1 | 2 | 7 | 28 | 56a | 84 |
| Dose | 0 mg | | | | | |
| N | 12 | 12 | 12 | 10 | 6 | 2 |
| Mean | 0.21 | 0.24 | 0.28 | 0.23 | 0.22 | 0.21 |
| Median | 0.21 | 0.20 | 0.19 | 0.19 | 0.22 | 0.21 |
| STD | 0.04 | 0.07 | 0.19 | 0.10 | 0.03 | 0.01 |
| Dose | 3 mg | | | | | |
| N | 6 | 6 | 6 | | | |
| Mean | 0.20 | 4.26 | 0.24 | | | |
| Median | 0.20 | 4.43 | 0.24 | | | |
| SDT | 0.02 | 1.16 | 0.04 | | | |
| Dose | 10 mg | | | | | |
| N | 6 | 6 | 6 | 6 | | |
| Mean | 0.18 | 21.07 | 0.49 | 0.21 | | |
| Median | 0.9 | 18.37 | 0.40 | 0.21 | | |
| STD | 0.01 | 6.18 | 0.25 | 0.02 | | |
| Dose | 30 mg | | | | | |
| N | 6 | 6 | 6 | 6 | | |
| Mean | 0.18 | 34.28 | 56.10 | 0.35 | | |
| Median | 0.18 | 33.30 | 34.45 | 0.25 | | |
| STD | 0.02 | 7.28 | 57.48 | 0.19 | | |
| Dose | 100 mg | | | | | |
| N | 12 | 12 | 12 | 12 | 12 | |
| Mean | 0.20 | 42.75 | 429.33 | 0.54 | 0.26 | |
| Median | 0.20 | 46.84 | 468.84 | 0.46 | 0.25 | |
| STD | 0.03 | 14.15 | 104.64 | 0.19 | 0.03 | |
| Dose | 300 mg | | | | | |
| N | 6 | 6 | 6 | 6 | 6 | 6 |
| Mean | 0.21 | 22.89 | 482.74 | 247.85 | 0.35 | 0.24 |
| Median | 0.21 | 23.93 | 391.79 | 256.58 | 0.32 | 0.24 |
| STD | 0.03 | 5.37 | 176.28 | 181.54 | 0.08 | 0.03 |

^{a}Day 56 for the 100 mg dose combines measurements taken on Day 56 in Cohort 4 and Day 52 in Cohort 6.
Primary data stored in ePharm as artifact number 1236578.

### C) CD14⁺16⁺ Monocytes

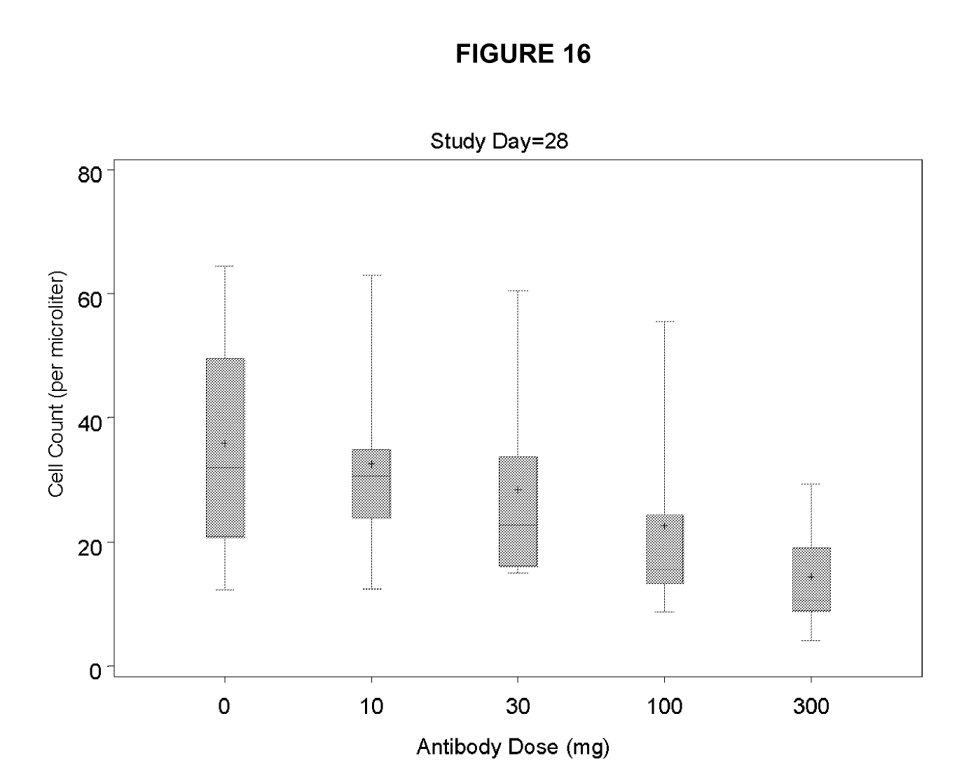

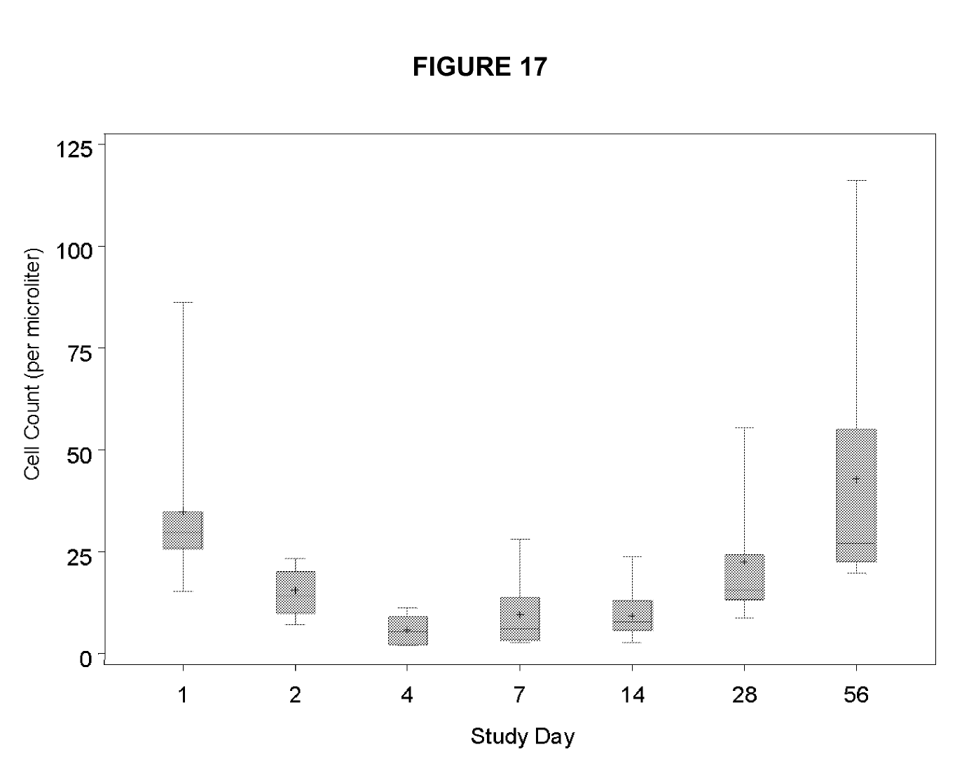

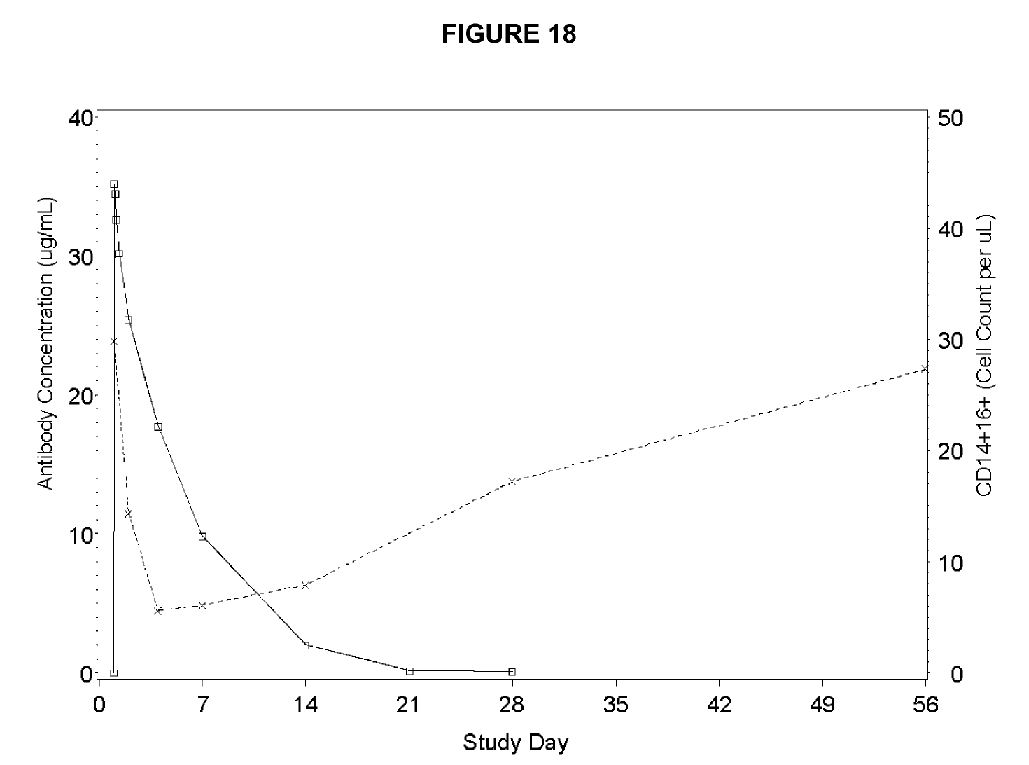

Dose response for CD14⁺16⁺ monocyte count (the sum of CD14^{bright CD}16⁺ and CD14^{dim}CD16⁺) on Study Day 28 is presented in FIG. 16. Mean CD14⁺16⁺ monocyte cell counts are presented in Table 12 by dose and Study Day and illustrated for the 100-mg dose in FIG. 17 and FIG. 18. The data in FIG. 17 on Day 56 combines measurements made on Day 56 for Cohort 4 and Day 52 for Cohort 6. All figures presenting CD14⁺16⁺ cell count data exclude a single observation that was considered an outlier. Subject 1031 (100-mg) on Study Day 28 had a reported value of 179.0 cells/mcl.

Treatment with antibody 8.10.3F caused a rapid decline in absolute numbers of peripheral circulating CD14⁺CD16⁺ monocytes with a nadir ranging from approximately 60 to 80% suppression on Day 4 for all doses. Increasing doses maintained suppression of CD14⁺CD16⁺ monocytes for increasing duration. Absolute numbers of CD14⁺CD16⁺ monocytes returned to baseline within 14 days for the 3 and 10 mg doses, within 28 days for the 30 mg dose, and within 56 days for the 100 and 300 mg dose.

TABLE 12
CD14+16+ Monocyte Counts (cells/μL) Following
Administration of Single Intravenous Solution Doses to Healthy
Subjects

| | Study Day | | | | | | | |
|---|---|---|---|---|---|---|---|---|
| | 1 | 2 | 4 | 7 | 14 | 28 | 56 | 84 |
| Dose | 0 mg | | | | | | | |
| N | 11 | 12 | 12 | 12 | 12 | 10 | 5 | 2 |
| Mean | 32.72 | 34.19 | 37.53 | 40.62 | 37.42 | 35.91 | 36.66 | 39.95 |
| Median | 26.6 | 30.50 | 36.00 | 36.70 | 37.50 | 32.00 | 41.20 | 39.95 |
| STD | 16.55 | 18.16 | 16.03 | 18.14 | 14.99 | 17.46 | 13.29 | 15.20 |
| Dose | 3 mg | | | | | | | |
| N | 6 | 6 | 6 | 6 | 6 | | | |
| Mean | 27.70 | 23.05 | 12.32 | 22.22 | 40.95 | | | |
| Median | 25.80 | 16.25 | 10.90 | 19.70 | 42.40 | | | |
| STD | 11.17 | 18.72 | 6.10 | 11.19 | 15.17 | | | |
| Dose | 10 mg | | | | | | | |
| N | 6 | 6 | 6 | 6 | 6 | 6 | | |
| Mean | 19.88 | 13.82 | 8.82 | 14.90 | 37.50 | 32.58 | | |
| Median | 17.60 | 10.70 | 8.50 | 17.05 | 29.30 | 30.60 | | |
| STD | 9.99 | 9.37 | 7.11 | 9.43 | 30.59 | 16.95 | | |
| Dose | 30 mg | | | | | | | |
| N | 6 | 6 | 6 | 6 | 6 | 6 | | |
| Mean | 23.90 | 13.72 | 3.92 | 5.03 | 8.52 | 28.47 | | |
| Median | 19.00 | 12.05 | 4.25 | 4.80 | 8.75 | 22.75 | | |
| STD | 7.78 | 8.11 | 1.95 | 3.21 | 2.54 | 17.39 | | |
| Dose | 100 mg | | | | | | | |
| N | 12 | 12 | 12 | 12 | 11 | 12 | 12 | |
| Mean | 35.61 | 14.98 | 5.55 | 8.93 | 9.37 | 37.43 | 41.89 | |
| Median | 29.85 | 14.00 | 5.05 | 5.60 | 7.90 | 18.10 | 30.25 | |
| STD | 18.66 | 5.58 | 3.50 | 7.73 | 5.75 | 52.98 | 27.64 | |
| Dose | 300 mg | | | | | | | |
| N | 6 | 6 | 6 | 6 | 6 | 6 | 6 | 6 |
| Mean | 36.35 | 10.92 | 6.42 | 11.00 | 12.05 | 15.73 | 38.52 | 35.77 |
| Median | 34.80 | 8.95 | 6.25 | 7.75 | 9.55 | 14.85 | 29.80 | 27.35 |
| STD | 17.41 | 5.82 | 3.18 | 10.54 | 8.34 | 9.48 | 23.51 | 20.84 |

D) BSAP and uNTX-1

Treatment with antibody 8.10.3F was associated with dose and time dependent decrease in uNTX-1, a marker of the bone resorptive activity of osteoclasts. The rate of decrease of uNTX-1 was slower than for CD14⁺CD16⁺ monocytes and recovery from suppression was more protracted. In the 30, 100, and 300 mg dose groups, uNTX-1 declined to a minimum of 64.4, 60.5, and 39.9% of baseline values, which occurred on Days 7, 14, and 28, respectively. On Day 28, mean uNTX-1 was 78.0, 69.4, and 39.3% for the same respective dose groups. uNTX-1 returned to baseline by approximately Day 56 for doses ≦100 mg.

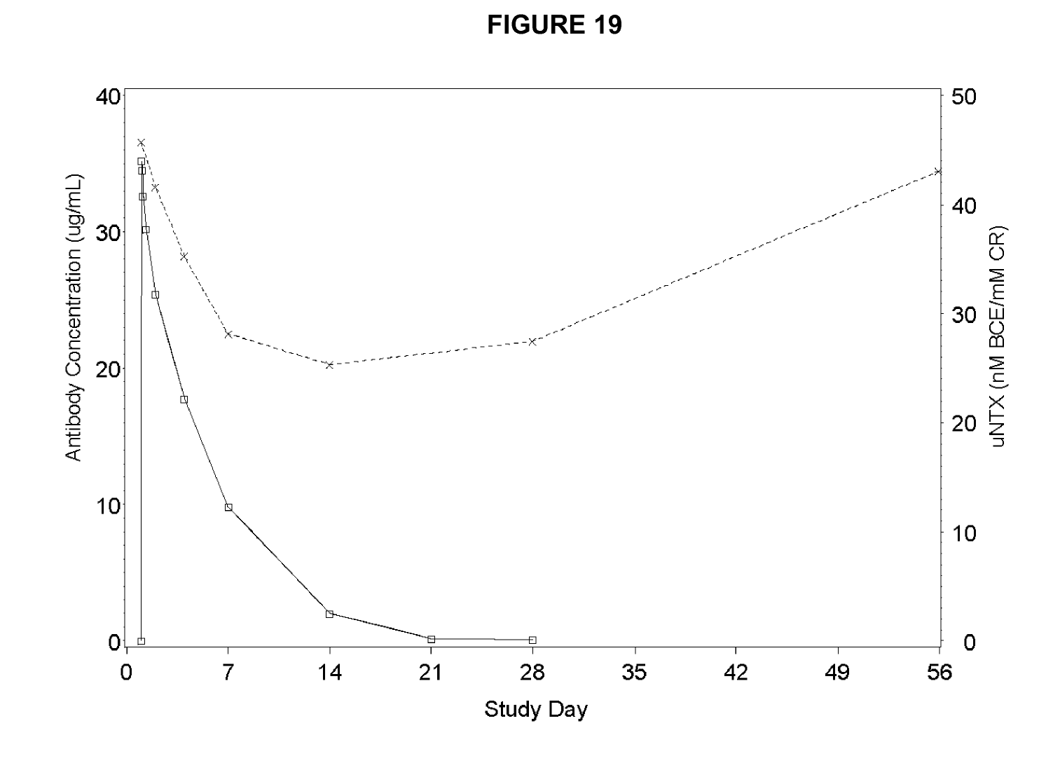

Mean uNTX-1 is presented in Table 13 by dose and Study Day and illustrated for the 100-mg dose in FIG. 19.

BSAP levels were not affected by treatment at doses up to and including 100 mg. At 300 mg, there was a trend toward increasing BSAP noted on Days 7 through 28 followed by a return to baseline by Day 56. However, the maximum absolute values observed in subjects receiving 300 mg were comparable to that observed in other treatment groups including placebo.

TABLE 13
Mean uNTX-1 (nM BCE/mM creatinine) Following
Administration of Single Intravenous Solution Doses to Healthy
Subjects

| | Study Day | | | | | | | |
|---|---|---|---|---|---|---|---|---|
| | 1 | 2 | 4 | 7 | 14 | 28 | 56 | 84 |
| Dose | 0 mg | | | | | | | |
| N | 12 | 12 | 12 | 12 | 12 | 10 | 5 | 2 |
| Mean | 51.73 | 54.12 | 46.48 | 51.46 | 51.73 | 40.58 | 38.08 | 0.21 |
| Median | 45.25 | 51.70 | 47.75 | 42.75 | 47.05 | 34.50 | 34.50 | 0.21 |
| STD | 24.89 | 24.37 | 22.12 | 23.44 | 22.89 | 18.18 | 12.21 | 0.01 |
| Dose | 3 mg | | | | | | | |
| N | 6 | 6 | 6 | 6 | 6 | | | |
| Mean | 59.02 | 50.65 | 49.50 | 52.58 | 45.57 | | | |
| Median | 58.35 | 51.00 | 44.75 | 52.25 | 44.90 | | | |
| STD | 23.79 | 17.03 | 12.09 | 14.09 | 10.62 | | | |
| Dose | 10 mg | | | | | | | |
| N | 6 | 6 | 6 | 6 | 6 | 6 | | |
| Mean | 44.65 | 37.50 | 32.50 | 36.72 | 38.02 | 44.55 | | |
| Median | 31.85 | 33.25 | 27.80 | 31.75 | 27.40 | 46.05 | | |
| STD | 31.60 | 25.40 | 21.25 | 17.76 | 24.56 | 20.90 | | |
| Dose | 30 mg | | | | | | | |
| N | 6 | 6 | 6 | 6 | 6 | 6 | | |
| Mean | 49.38 | 41.12 | 38.58 | 31.78 | 37.83 | 38.50 | | |
| Median | 49.70 | 46.55 | 39.95 | 32.10 | 36.75 | 39.95 | | |
| STD | 18.55 | 16.59 | 13.63 | 13.18 | 12.67 | 13.49 | | |
| Dose | 100 mg | | | | | | | |
| N | 12 | 12 | 12 | 12 | 12 | 12 | 12 | |
| Mean | 46.01 | 43.67 | 38.59 | 29.04 | 27.83 | 31.95 | 43.60 | |
| Median | 45.65 | 41.55 | 35.20 | 28.15 | 25.30 | 27.45 | 43.05 | |
| STD | 13.79 | 15.97 | 22.99 | 8.61 | 15.64 | 13.80 | 17.53 | |
| Dose | 300 mg | | | | | | | |
| N | 6 | 6 | 6 | 6 | 6 | 6 | 5 | 6 |
| Mean | 43.00 | 32.98 | 31.13 | 24.08 | 17.42 | 17.17 | 30.26 | 0.24 |
| Median | 41.35 | 32.70 | 30.00 | 22.50 | 17.40 | 13.90 | 23.80 | 0.24 |
| STD | 17.11 | 10.16 | 11.82 | 7.49 | 2.76 | 5.91 | 20.20 | 0.03 |

### Discussion

Antibody 8.10.3F was generally well tolerated following administration of a single intravenous dose of 3-300 mg to healthy adult volunteers. Treatment with antibody 8.10.3F caused rapid changes in pharmacodynamic markers that were reversible following clearance of the drug. The primary biomarker for mechanism was the number of circulating CD14⁺CD16⁺ monocytes, which had been linked to efficacy in preclinical arthritis models. All doses of antibody 8.10.3F caused a rapid decline in CD14⁺CD16⁺ monocytes with a nadir ranging from approximately 60 to 80% suppression on Day 4. Increasing doses maintained suppression of CD14⁺CD16⁺ monocytes for increasing duration. Absolute numbers of CD14⁺CD16⁺ monocytes returned to baseline within 14 days for the 3 and 10 mg doses, within 28 days for the 30 mg dose, and within 56 days for the 100 and 300 mg dose in this study.

Based upon preclinical models using a surrogate antibody, doses predicted to provide therapeutic benefit are those that maintain suppression of CD14⁺CD16⁺ monocytes at levels ≦50% of baseline values. In this Study, doses ≧30 mg were able to suppress circulating CD14⁺CD16⁺ monocytes below 50% of baseline for at least 14 days. The 30 mg dose had median CD14⁺CD16⁺ monocyte values of 25%, 46%, and 120% of baseline on Study Days 7, 14, and 28, respectively. The 100 mg dose had median CD14⁺CD16⁺ monocyte values of 19%, 26%, and 61% of baseline on Study Days 7, 14, and 28, respectively. CD14⁺CD16⁺ monocytes may decline further with repeated dosing.

Inhibition of M-CSF was predicted to inhibit osteoclast differentiation leading to inhibition of bone resorption. Treatment with antibody 8.10.3F was associated with dose and time dependent decrease in uNTX-1. The rate of decrease of uNTX-1 was slower than for CD14⁺CD16⁺ monocytes and recovery from suppression was more protracted. In the 30, 100, and 300 mg dose groups, uNTX-1 declined to a minimum of 64.4, 60.5, and 39.9% of baseline values, which occurred on Days 7, 14, and 28, respectively. On Day 28, mean uNTX-1 was 78.0, 69.4, and 39.3% for the same respective dose groups. uNTX-1 returned to baseline by approximately Day 56 for doses ≦100 mg. BSAP levels were not affected by treatment at doses up to and including 100 mg. At 300 mg, there was a trend toward increasing BSAP noted on Days 7 through 28 followed by a return to baseline by Day 56. However, the maximum absolute values observed in subjects receiving 300 mg were comparable to that observed in other treatment groups including placebo.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

SEQUENCES
Key:
Signal peptide: underlined lower case
CDRs 1, 2, 3: underlined UPPERCASE
Variable domain: UPPER CASE
Constant domain: lower case
Mutations from germline in bold
SEQ ID NO: 1
252 Heavy Chain [Gamma chain] nucleotide sequence
atggagttggggctgtgctggattttccttgttgctattataaaaggtgtccagtgtCAGGTGCAGCT
GGTGGAGTCTGGGGGAGGCTTGGTCAAGCCTGGAGGGTCCCTGAGA
CTCTCCTGTGCAGCCTCTGGATTCACCTTCAGTGACTACTACATGAGC
TGGATCCGCCAGGCTCCAGGGAAGGGGCTGGAGTGGATTTCATACAT
TAGTGGTAGTGGTAGTACCATATACTACGCAGACTCTGTGAAGGGCC
GATTCACCATCTCCAGGGACAACGCCAAGAACTCACTGTATCTGCAAA
TGAACAGCCTGAGAGCCGAGGACACGGCCGTGTATCACTGTGCGAGA
GCCCTGGGTGGGATGGACGTCTGGGGCCAAGGGACCACGGTCACCG
TCTCCTCAGCTtccaccaagggcccatccgtcttccccctggcgccctgctctagaagcacctcc
gagagcacagcggccctgggctgcctggtcaaggactacttccccgaaccggtgacggtgtcgtgga
actcaggcgctctgaccagcggcgtgcacaccttcccagctgtcctacagtcctcaggactctactccct
cagcagcgtggtgaccgtgccctccagcaacttcggcacccagacctacacctgcaacgtagatcac
aagcccagcaacaccaaggtggacaagacagttgagcgcaaatgttgtgtcgagtgcccaccgtgcc
cagcaccacctgtggcaggaccgtcagtcttcctcttccccccaaaacccaaggacaccctcatgatct
cccggacccctgaggtcacgtgcgtggtggtggacgtgagccacgaagaccccgaggtccagttcaa
ctggtacgtggacggcgtggaggtgcataatgccaagacaaagccacgggaggagcagttcaacag
cacgttccgtgtggtcagcgtcctcaccgttgtgcaccaggactggctgaacggcaaggagtacaagtg
caaggtctccaacaaaggcctcccagcccccatcgagaaaaccatctccaaaaccaaagggcagc
cccgagaaccacaggtgtacaccctgcccccatcccgggaggagatgaccaagaaccaggtcagc
ctgacctgcatggtcaaaggcttctaccccagcgacatcgccgtggagtgggagagcaatgggcagc
cggagaacaactacaagaccacacctcccatgctggactccgacggctccttcttcctctacagcaag
ctcaccgtggacaagagcaggtggcagcaggggaacgtcttctcatgctccgtgatgcatgaggctct
gcacaaccactacacgcagaagagcctctccctgtctccgggtaaa
SEQ ID NO: 2
252 Heavy Chain [Gamma chain] protein sequence
melglcwiflvaiikgvqcQVQLVESGGGLVKPGGSLRLSCAASGFTFSDYYMSW
IRQAPGKGLEWISYISGSGSTIYYADSVKGRFTISRDNAKNSLYLQMNSLR
AEDTAVYHCARALGGMDVWGQGTTVTVSSAstkgpsvfplapcsrstsestaalgcl
vkdyfpepvtvswnsgaltsgvhtfpavlqssglyslssvvtvpssnfgtqtytcnvdhkpsntkvdktve
rkccvecppcpappvagpsvflfppkpkdtlmisrtpevtcvvvdvshedpevqfnwyvdgvevhna
ktkpreeqfnstfrvvavitvvhqdwlngkeykckvsnkglpapiektisktkgqprepqvytippsree
mtknqvsltclvkgfypsdiavewesngqpennykttppmldsdgsfflyskltvdksrwqqgnvfscs
vmhealhnhytqkslslspgk
SEQ ID NO: 3
252 Light Chain [Kappa chain] nucleotide sequence
atgagggtccctgctcagctcctggggctcctgctactctggctccgaggtgccagatgtGACATCC
AGATGACCCAGTCTCCATCCTCCCTGTCTGCATCTGTAGGAGACAGA
GTCACCATCACTTGCCGGGCAAGTCAGAGCATTAGCGGCTTTTTAAAT
TGGTATCAGCAGAAACCAGGGAAAGCCCCTAAGCTCCTGATCTATGCT
ACATCCAGTTTGCAAAGTGGGGTCCCATTCAGGTTCAGTGGCAGTGG
ATCTGGGACAGATTTCACTCTCACCATCAGCAGTCTGCAACCTGAAGA
TTTTGCAACTTATTACTGTCAACAGAGTTACAGTGTCCCATTCACTTTC
GGCCCTGGGACCAAAGTGGATATCAAACGAactgtggctgcaccatctgtcttcatc
ttcccgccatctgatgagcagttgaaatctggaactgctagcgttgtgtgcctgctgaataacttctatccca
gagaggccaaagtacagtggaaggtggataacgccctccaatcgggtaactcccaggagagtgtca
cagagcaggacagcaaggacagcacctacagcctcagcagcaccctgacgctgagcaaagcaga
ctacgagaaacacaaagtctacgcctgcgaagtcacccatcagggcctgagctcgcccgtcacaaa
gagcttcaacaggggagagtgt
SEQ ID NO: 4
252 Light Chain [Kappa chain] protein sequence
mrvpaqllgllllwlrgarcDIQMTQSPSSLSASVGDRVTITCRASQSISGFLNWYQ
QKPGKAPKLLIYATSSLQSGVPFRFSGSGSGTDFTLTISSLQPEDFATYYC
QQSYSVPFTFGPGTKVDIKRtvaapsvfifppsdeqlksgtasvvcllnnfypreakvqwkv
dnalqsgnsqesvteqdskdstyslsstltlskadyekhkvyacevthqglsspvtksfnrgec
SEQ ID NO: 5
88 Heavy Chain [Gamma chain] nucleotide sequence
atggaatttgggctgtgctgggttttccttgttgctattttagaaggtgtccagtgtGAGGTGCAGCTG
GTGGAGTCTGGGGGAGGCTTGGTCCAGCCTGGGGGGTCCCTGAGAC
TCTCCTGTGCAGCCTCTGGATTCACCTTTAGTAGCTATTGGATGAGCT
GGGTCCGCCAGGCTCCAGGGAAGGGGCTGGAGTGGGTGGCCAACAT
AAAGCAAGATGGPAGTGAGAAATACTATGTGGACTCTGTGAAGGGCC
GATTCACCATCTCCAGAGACAACGCCAAGAACTCACTGTATCTGCAAA
TGAACAGCCTGAGAGCCGAGGACACGGCTGTGTATTACTGTGCTCCG
GGTATAGCAGCAGCTGGTAGGGCCTACTGGGGCCAGGGAACCCTGG
TCACCGTCTCCTCAGCTtccaccaagggcccatccgtcttccccctggcgccctgctctaga
agcacctccgagagcacagcggccctgggctgcctggtcaaggactacttccccgaaccggtgacgg
tgtcgtggaactcaggcgctctgaccagcggcgtgcacaccttcccagctgtcctacagtcctcaggact
ctactccctcagcagcgtggtgaccgtgccctccagcaacttcggcacccagacctacacctgcaacgt
agatcacaagcccagcaacaccaaggtggacaagacagttgagcgcaaatgttgtgtcgagtgccc
accgtgcccagcaccacctgtggcaggaccgtcagtcttcctcttccccccaaaacccaaggacaccc
tcatgatctcccggacccctgaggtcacgtgcgtggtggtggacgtgagccacgaagaccccgaggtc
cagttcaactggtacgtggacggcgtggaggtgcataatgccaagacaaagccacgggaggagcag
ttcaacagcacgttccgtgtggtcagcgtcctcaccgttgtgcaccaggactggctgaacggcaaggag
tacaagtgcaaggtctccaacaaaggcctcccagcccccatcgagaaaaccatctccaaaaccaaa
gggcagccccgagaaccacaggtgtacaccctgcccccatcccgggaggagatgaccaagaacca
ggtcagcctgacctgcctggtcaaaggcttctaccccagcgacatcgccgtggagtgggagagcaatg
ggcagccggagaacaactacaagaccacacctcccatgctggactccgacggctccttcttcctctac
agcaagctcaccgtggacaagagcaggtggcagcaggggaacgtcttctcatgctccgtgatgcatg
aggctctgcacaaccactacacgcagaagagcctctccctgtctccgggtaaa
SEQ ID NO: 6
88 Heavy Chain [Gamma chain] protein sequence
mefglcwvflvailegvqcEVQLVESGGGLVQPGGSLRLSCAASGFTFSSYWMS
WVRQAPGKGLEWVANIKQDGSEKYYVDSVKGRFTISRDNAKNSLYLQM
NSLRAEDTAVYYCAPGIAAAGRAYWGQGTLVTVSSAstkgpsvfplapcsrstse
staalgclvkdyfpepvtvswnsgaltsgvhtfpavlqssglyslssvvtvpssnfgtqtytcnvdhkpsnt
kvdktverkccvecppcpappvagpsvflfppkpkdtlmisrtpevtcvvvdvshedpevqfnwyvdg
vevhnaktkpreeqfnstfrvvsvltvvhqdwlngkeykckvsnkglpapiektisktkgqprepqvytlp
psreemtknqvsltclvkgfypsdiavewesngqpennykttppmldsdgsfflyskltvdksrwqqgn
vfscsvmhealhnhytqkslslspgk
SEQ ID NO: 7
88 Light Chain [Kappa chain] nucleotide sequence
atgagggtccctgctcagctcctggggctcctgctactctggctccgaggtgccagatgtGACATCC
AGATGACCCAGTCTCCATCCTCCCTGTCTGCATCTGTTGGAGACAGAG
TCACCATCACTTGCCGGCCAAGTCAGGACATTAGCAGTTATTTAAATT
GGTATCAGCAGAAACCAGGGAAAGCCCCTAAGCTCCTGATCTATGCT
GCATCCAGTTTGCAAAGTGGGGTCCCATTAAGGTTCAGTGGCAGTGG
ATCTGGGACAGATTTCACTCTCACCATCAGCAGTCTGCAACCTGAAGA
TTTTGCAACTTACTACTGTCAACAGAGTTACAGTACCCCATTCACTTTC
GGCCCTGGGACCAAAGTGGATATCAAACGAactgtggctgcaccatctgtcttcatc
ttcccgccatctgatgagcagttgaaatctggaactgctagcgttgtgtgcctgctgaataacttctatccca
gagaggccaaagtacagtggaaggtggataacgccctccaatcgggtaactcccaggagagtgtca
cagagcaggacagcaaggacagcacctacagcctcagcagcaccctgacgctgagcaaagcaga
ctacgagaaacacaaagtctacgcctgcgaagtcacccatcagggcctgagctcgcccgtcacaaa
gagcttcaacaggggagagtgt
SEQ ID NO: 8
88 Light Chain [Kappa chain] protein sequence
mrvpaqllgllllwlrgarcDIQMTQSPSSLSASVGDRVTITCRPSQDISSYLNWYQ
QKPGKAPKLLIYAASSLQSGVPLRFSGSGSGTDFTLTISSLQPEDFATYYC
QQSYSTPFTFGPGTKVDIKRtvaapsvfifppsdeqlksgtasvvcllnnfypreakvqwkv
dnalqsgnsqesvteqdskdstyslsstitlskadyekhkvyacevthqglsspvtksfnrgec
SEQ ID NO: 9
100 Heavy Chain [Gamma chain] nucleotide sequence
atggagtttgggctccgctggatttttcttgtggctattttaaaaggtgtccagtgtGAGGTGCAGCT
GTTGGAGTCTGGGGGAGGCTTGGTACAGCCTGGGGGGTCCCTGAGA
CTCTCCTGTGCAGCCTCTGGATTCACCTTTAGCAGCTATGCCATGAGC
TGGGTCCGCCAGGCTCCAGGGAAGGGGCTGGAATGGGTCTCAGCTA
TTAGTGGTCGTGGTGGTAGGACATACTTCGCAGACTCCGTGAAGGGC
CGGTTCACCATCTCCAGAGACAATTCCAAGAACACGCTGTATCTGCAA
ATGAACAGCCTGAGAGCCGAGGACACGGCCGTATATTTCTGTGCGGT
AGAAGGCTATAGTGGGCGCTACGGATTTTTTGACTACTGGGGCCAGG
GAACCCTAGTCACCGTCTCCTCAGCCtccaccaagggcccatcggtcttccccctgg
cgccctgctctagaagcacctccgagagcacagcggccctgggctgcctggtcaaggactacttcccc
gaaccggtgacggtgtcgtggaactcaggcgctctgaccagcggcgtgcacaccttcccagctgtcct
acagtcctcaggactctactccctcagcagcgtggtgaccgtgccctccagcaacttcggcacccaga
cctacacctgcaacgtagatcacaagcccagcaacaccaaggtggacaagacagttgagcgcaaat
gttgtgtcgagtgcccaccgtgcccagcaccacctgtggcaggaccgtcagtcttcctcttccccccaaa
acccaaggacacccttcatgatctcccggacccctgaggtcacgtgcgtggtggtggacgtgagccac
gaagaccccgaggtccagttcaactggtacgtggacggcgtggaggtgcataatgccaagacaaag
ccacgggaggagcagttcaacagcacgttccgtgtggtcagcgtcctcaccgttgtgcaccaggactg
gctgaacggcaaggagtacaagtgcaaggtctccaacaaaggcctcccagcccccatcgagaaaa
ccatctccaaaaccaaagggcagccccgagaaccacaggtgtacaccctgcccccatcccgggag
gagatgaccaagaaccaggtcagcctgacctgcctggtcaaaggcttctaccccagcgacatcgccg
tggagtgggagagcaatgggcagccggagaacaactacaagaccacacctcccatgctggactccg
acggctccttcttcctctacagcaagctcaccgtggacaagagcaggtggcagcaggggaacgtcttct
catgctccgtgatgcatgaggctctgcacaaCCactacacgcagaagagcctctccctgtctccgggta
aa
SEQ ID NO: 10
100 Heavy Chain [Gamma chain] protein sequence
mefglrwiflvailkgvqcEVQLLESGGGLVQPGGSLRLSCAASGFTFSSYAMSW
VRQAPGKGLEWVSAISGRGGRTYFADSVKGRFTISRDNSKNTLYLQMNS
LRAEDTAVYFCAVEGYSGRYGFFDYWGQGTLVTVSSAstkgpsvfplapcsrst
sestaalgclvkdyfpepvtvswnsgaltsgvhtfpavlqssglysissvvtvpssnfgtqtytcnvdhkp
sntkvdktverkccvecppcpappvagpsvflfppkpkdtlmisrtpevtcvvvdvshedpevqfnwy
vdgvevhnaktkpreeqfnstfrvvsvltvvhqdwlngkeykckvsnkglpapiektisktkgqprepqv
ytlppsreemtknqvsltclvkgfypsdiavewesngqpennykttppmldsdgsfflyskltvdksrwq
qgnvfscsvmhealhnhytqkslslspgk
SEQ ID NO: 11
100 Light Chain [Kappa chain] nucleotide sequence
atggaagccccagctcagcttctcttcctcctgctactctggctcccagataccactggaGAAATAGT
GATGACGCAGTCTCCAGCCACCCTGTCTGTGTCTCCAGGGGAAAGAG
CCACCCTCTCCTGCAGGGCCAGTCAGAGTGTTAGCAGCAACTTAGCC
TGGTACCAGCAGAAACCTGGCCAGGCTCCCAGGCTCCTCATCTATGG
TGCATCCACCAGGGCCAGTGGTATCCCAGACAGGATCAGTGGCAGTG
GGTCTGGAACAGAGTTCACTCTCATCATCAGCAGCCTGCAGTCTGAA
GATTTTGCAGTTTATTACTGTCAGCAGTCTAATAACTGGCCATTCACTT
TCGGCCCTGGGACCAAAGTGGATATCAAACGAactgtggctgcaccatctgtcttc
atcttcccgccatctgatgagcagttgaaatctggaactgctagcgttgtgtgcctgctgaataacttctatc
ccagagaggccaaagtacagtggaaggtggataacgccctccaatcgggtaactcccaggagagtg
tcacagagcaggacagcaaggacagcacctacagcctcagcagcaccctgacgctgagcaaagc
agactacgagaaacacaaagtctacgcctgcgaagtcacccatcagggcctgagctcgcccgtcac
aaagagcttcaacaggggagagtgt
SEQ ID NO: 12
100 Light Chain [Kappa chain] protein sequence
meapaqllfllllwlpdttgEIVMTQSPATLSVSPGERATLSCRASQSVSSNLAWYQ
QKPGQAPRLLIYGASTRASGIPDRISGSGSGTEFTLIISSLQSEDFAVYYC
QQSNNWPFTFGPGTKVDIKRtvaapsvfifppsdeqlksgtasvvcllnnfypreakvqwkv
dnalqsgnsqesvteqdskdstyslsstltlskadyekhkvyacevthqglsspvtksfnrgec
SEQ ID NO: 14
3.8.3 Heavy Chain [Gamma chain] protein sequence
mefgiswvflvaiikgvqcQVQLVESGGGLVKPGGSLRLSCAASGFTFSDYYMS
WIRQAPGKGLEWFSYISSSGSTIYYADSVKGRFTISRDNAKNSLSLQMNS
LRAEDTAVYYCARGLTGDYWGQGTLVTVSSAstkgpsvfplapcsrstsestaalgcl
vkdyfpepvtvswnsgaltsgvhtfpavlqssglyslssvvtvpssnfgtqtytcnvdhkpsntkvdktve
rkccverppcpappvagpsvflfppkpkdtlmisrtpevtcvvvdvshedpevqfnwyvdgvevhna
ktkpreeqfnstfrvvsvltvvhqdwlngkeykckvsnkglpapiektisktkgqprepqvytlppsree
mtknqvsltclvkgfypsdiavewesnggpennykttppmldsdgsfflyskltvdksrwqqgnviscs
vmhealhnhytqkslslspgk
SEQ ID NO: 16
3.8.3 Light Chain [Kappa chain] protein sequence
mdmrvpaqllgllllwfpgsrcDIQMTQSPSSVSASVGDRVTISCRASQDISGWLA
WYQQKPGKAPKLLISATSSLHSGVPSRFSGSGSGTDFTLTISSLQPEDFA
TYYCQQTNSFPFTFGPGTKVDIKRtvaapsvfifppsdeqlksgtasvvcllnnfypreakv
qwkvdnalqsgnsqesvteqdskdstyslsstitlskadyekhkvyacevthqgisspvtksfnrgec
SEQ ID NO: 18
2.7.3 Heavy Chain [Gamma chain] protein sequence
mefglswvflvallrgcqcQVQLVESGGGVVQPGRSLRLSCAASGFTFSSYGMH
WVRQAPGKGLEWVAFIWYDGSNKYYADSVKGRFTISRDNSKNTLYLQM
NSLRAEDTAVYYCARGYRVYFDYWGQGTLVTVSSAstkgpsvfplapcsrstses
taalgclvkdyfpepvtvswnsgaltsgvhtfpavlqssglyslssvvtvpsssigtktytcnvdhkpsntk
vdkrveskygppcpscpapeflggpsvflfppkpkdtlmisrtpevtcvvvdvsqedpevqfnwyvdg
vevhnaktkpreeqfnstyrvvsvltvlhqdwlngkeykckvsnkglpssiektiskakgqprepqvytl
ppsqeemtknqvsltclvkgfypsdiavewesngqpennykttppvldsdgsfflysrltvdksrwqeg
nvfscsvmhealhnhytqkslslspgk
SEQ ID NO: 20
2.7.3 Light Chain [Kappa chain] protein sequence
mdmrvpaqllgllllwfpgsrcDIQMTQSPSSVSASVGDRVTITCRASQDISSWLA
WYQRKPGKAPKLQIYAASSLESGVPSRFNGSGSGTDFTLSISSLQPEDFA
TYYCQQTNSFPLTFGGGTKVEIKRtvaapsvfifppsdeqlksgtasvvcllnnfypreakv
qwkvdnalqsgnsqesvteqdskdstyslsstltlskadyekhkvyacevthqgisspvtksfnrgec
SEQ ID NO: 22
1.120.1 Heavy Chain [Gamma chain] protein sequence
mewtwsflflvaaatgahsQVQLVQSGAEVKKPGASVKVSCKASGYTFTSYGIS
WVRQAPGQGLEWMGWISAYNGNTNYAQKLQDRVTMTTDTSTTTAYME
LRSLRSDDTAVYYCARRAYGANFFDYWGQGTLVTVSSAstkgpsvfplapcsr
stsestaalgclvkdyfpepvtvswnsgaltsgvhtfpavlqssglysissvvtvpssnfgtqtytcnvdhk
psntkvdktverkccvecppcpappvagpsvflfppkpkdtlmisrtpevtcvvvdvshedpevqfnw
yvdgvevhnaktkpreeqfnstfrvvsvltvvhqdwlngkeykckvsnkglpapiektisktkgqprepq
vytippsreemtknqvsltclvkgfypsdiavewesngqpennykttppmldsdgsfflyskltvdksrw
qqgnvfscsvmhealhnhytqkslslspgk
SEQ ID NO: 24
1.120.1 Light Chain [Kappa chain] protein sequence
mvlqtqvfislllwisgaygDIVMTQSPDSLAVSLGERATINCKSSQSILFFSNNKNY
LAWYRQKPGQPPNLLIYWASTRESGVPDRFSGSGSGTDFTLTISSLQAE
DVAVYYCQQYYSSPWTFGQGTKVEIKRtvaapsvfifppsdeqlksgtasvvcllnnfy
preakvqwkvdnalqsgnsqesvteqdskdstyslsstltlskadyekhkvyacevthqglsspvtksf
nrgec
SEQ ID NO: 25
9.14.4I Heavy Chain [Gamma Chain] nucleotide sequence
atggagtttgggctgagctgggttttccttgttgctattataaaaggtgtCCAGTGTCAGGTGCAG
CTGGTGGAGTCTGGGGGAGGCTTGGTCAAGCCTGGAGGGTCCCTGA
GACTCTCCTGTGCAGCCTCTGGATTCACCTTCAGTGACTACTATATGA
GCTGGATCCGCCAGGCTCCAGGGAAGGGACTGGAGTGGGTTTCATA
CATTAGTAGTAGTGGTAGTACCATATACTACGCAGACTCTGTGAAGGG
CCGATTCACCATCTCCAGGGACAACGCCAAGAACTCACTGTATCTGCA
AATGAACAGCCTGAGAGCCGAGGACACGGCCGTGTATTACTGTGCGA
GAGGCCTAACTGGGGACTACTGGGGCCAGGGAACCCTGGTCACCGT
CTCCTCAGCTtccaccaagggcccatccgtcttccccctggcgccctgctctagaagcacctccg
agagcacagcggccctgggctgcctggtcaaggactacttccccgaaccggtgacggtgtcgtggaa
ctcaggcgctctgaccagcggcgtgcacaccttcccagctgtcctacagtcctcaggactctactccctc
agcagcgtggtgaccgtgccctccagcaacttcggcacccagacctacacctgcaacgtagatcaca
agcccagcaacaccaaggtggacaagacagttgagcgcaaatgttgtgtcgagtgcccaccgtgccc
agcaccacctgtggcaggaccgtcagtcttcctcttccccccaaaacccaaggacaccctcatgatctc
ccggacccctgaggtcacgtgcgtggtggtggacgtgagccacgaagaccccgaggtccagttcaac
tggtacgtggacggcgtggaggtgcataatgccaagacaaagccacgggaggagcagttcaacagc
acgttccgtgtggtcagcgtcctcaccgttgtgcaccaggactggctgaacggcaaggagtacaagtgc
aaggtctccaacaaaggcctcccagcccccatcgagaaaaccatctccaaaaccaaagggcagcc
ccgagaaccacaggtgtacaccctgcccccatcccgggaggagatgaccaagaaccaggtcagcct
gacctgcctggtcaaaggcttctaccccagcgacatcgccgtggagtgggagagcaatgggcagccg
gagaacaactacaagaccacacctcccatgctggactccgacggctccttcttcctctacagcaagctc
accgtggacaagagcaggtggcagcaggggaacgtcttctcatgctccgtgatgcatgaggctctgca
caaccactacacgcagaagagcctctccctgtctccgggtaaa
SEQ ID NO: 26
914.4I Heavy Chain [Gamma Chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARGLTGDYWGQGTLVYVSSAstkgpsvfplapcsrstsestaalgcl
vkdyfpepvtvswnsgaltsgvhtfpavlqssglyslssvvtvpssnfgtqtytcnvdnkpsntkvdktve
rkccvecppcpappvagpsvflfppkpkdtlmisrtpevtcvvvdvshedpevqfnwyvdgvevhna
ktkpreeqfnstfrvvsvltvvhqdwlngkeykckvsnkglpapiektisktkgqprepqvytlppsree
mtknqvsltclvkgfypsdiavewesngqpennykttppmldsdgsfflyskltvdksrwqqgnvfscs
vmhealhnhytqkslslspgk
SEQ ID NO: 27
9.14.4, 9.14.4I, 9.14.4-Ser and 9.14.4-G1 Light Chain
[Kappa Chain] nucleotide sequence
atggacatgagggtccccgctcagctcctggggctcctgctactctggctccgaggtgccagatgTGA
CATCCAGATGACCCAGTCTCCATCCTCCCTGTCTGCATCTGTCGGAGA
CAGAGTCACCATCACTTGCCGGCCAAGTCAGATCATTAGCAGTTTATT
AAATTGGTATCAGCAGAAACCAGGGAAAGCCCCTAAGCTCCTGATCCA
TGCTGCATCCAGTTTGCAAAGTGGGGTCCCATCAAGGTTCAGTGGCA
GTGGATCTGGGACAGATTTCACTCTCACCATCAGTAGTCTGCAACCTG
AAGATTTTGCAACTTACTACTGTCAACAGAGTTACAGTACCCCATTCAC
TTTCGGCCCTGGGACCAAAGTGGATATCAAACGAactgtggctgcaccatctgtc
ttcatcttcccgccatctgatgagcagttgaaatctggaactgcctctgttgtgtgcctgctgaataacttctat
cccagagaggccaaagtacagtggaaggtggataacgccctccaatcgggtaactcccaggagagt
gtcacagagcaggacagcaaggacagcacctacagcctcagcagcaccctgacgctgagcaaag
cagactacgagaaacacaaagtctacgcctgcgaagtcacccatcagggcctgagctcgcccgtcac
aaagagcttcaacaggggagagtgt
SEQ ID NO: 28
9.14.4, 9.14.4I, 9.14.4-Ser and 9.14.4-G1 Light Chain
[Kappa Chain] protein sequence
mdmrvpaqllgllllwlrgarcDIQMTQSPSSLSASVGDRVTITCRPSQIISSLLNWY
QQKPGKAPKLLIHAASSLQSGVPSRFSGSGSGTDFTLTISSLQPEDFATY
YCQQSYSTPFTFGPGTKVDIKRtvaapsvfifppsdeqlksgtasvvcllnnfypreakvqw
kvdnalqsgnsqesvteqdskdstyslsstltlskadyekhkvyacevthqglsspvtksfnrgec
SEQ ID NO: 37
9.14.4 Heavy Chain [Gamma Chain] nucleotide sequence
atggagtttgggctgagctgggttttccttgttgctattataaaaggtgtCCAGTGTCAGGTGCAG
CTGGTGGAGTCTGGGGGAGGCTTGGTCAAGCCTGGAGGGTCCCTGA
GACTCTCCTGTGCAGCCTCTGGATTCACCTTCAGTGACTACTATATGA
GCTGGATCCGCCAGGCTCCAGGGAAGGGACTGGAGTGGGTTTCATA
CATTAGTAGTAGTGGTAGTACCATATACTACGCAGACTCTGTGAAGGG
CCGATTCACCATCTCCAGGGACAACGCCAAGAACTCACTGTATCTGCA
AATGAACAGCCTGAGAGCCGAGGACACGGCCGTGTATTACTGTGCGA
GAGGCCTAACTGGGGACTACTGGGGCCAGGGAACCCTGGTCACCGT
CTCCTCAGCTtccaccaagggcccatccgtcttccccctggcgccctgctctagaagcacctccg
agagcacagcggccctgggctgcctggtcaaggactacttccccgaaccggtgacggtgtcgtggaa
ctcaggcgctctgaccagcggcgtgcacaccttcccagctgtcctacagtcctcaggactctactccctc
agcagcgtggtgaccgtgccctccagcagcttgggcacgaagacctacacctgcaacgtagatcaca
agcccagcaacaccaaggtggacaagagagttgagtccaaatatggtcccccatgcccatcatgccc
agcacctgagttcctggggggaccatcagtcttcctgttccccccaaaacccaaggacactctcatgatc
tcccggacccctgaggtcacgtgcgtggtggtggacgtgagccaggaagaccccgaggtccagttca
actggtacgtggatggcgtggaggtgcataatgccaagacaaagccgcgggaggagcagttcaaca
gcacgtaccgtgtggtcagcgtcctcaccgtcctgcaccaggactggctgaacggcaaggagtacaa
gtgcaaggtctccaacaaaggcctcccgtcctccatcgagaaaaccatctccaaagccaaagggca
gccccgagagccacaggtgtacaccctgcccccatcccaggaggagatgaccaagaaccaggtca
gcctgacctgcctggtcaaaggcttctaccccagcgacatcgccgtggagtgggagagcaatgggca
gccggagaacaactacaagaccacgcctcccgtgctggactccgacggctccttcttcctctacagca
ggctaaccgtggacaagagcaggtggcaggaggggaatgtcttctcatgctccgtgatgcatgaggct
ctgcacaaccactacacacagaagagcctctccctgtctccgggtaaa
SEQ ID NO: 38
9.14.4 Heavy Chain [Gamma Chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARGLTGDYWGQGTLVTVSSAstkgpsvfplapcsrstsestaalgcl
vkdyfpepvtvswnsgaltsgvhtfpavlqssglysissvvtvpsssigtktytcnvdhkpsntkvdkrve
skygppcpsccapeflggpsvflfppkpkdtlmisrtpevtcvvvdvsqedpevqfnwyvdgvevhn
aktkpreeqfnstyrvvsvltvlhqdwlngkeykckvsnkglpssiektiskakgqprepqvytlppsqe
emtknqvsltclvkgfypsdiavewesngqpennykttppvldsdgsfflysrltvdksrwqegnvfscs
vmhealhnhytqkslslspgk
SEQ ID NO: 54
9.14.4C-Ser Heavy Chain [Gamma chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARGLTGDYWGQGTLVTVSSAstkgpsvfplapcsrstsestaalgcl
vkdyfpepvtvswnsgaltsgvhtfpavlqssglyslssvvtvpssslgtktytcnvdhkpsntkvdkrve
skygppcppcpapeflggpsvflfppkpkdtlmisrtpevtcvvvdvsqedpevqfnwyvdgvevhn
aktkpreeqfnstyrvvsvltvlhqdwlngkeykckvsnkglpssiektiskakgqprepqvytlppsqe
emtknqvsltclvkgfypsdiavewesngqpennykttppvldsdgsfflysrltvdksrwqegnvfscs
vmhealhnhytqkslslspgk
SEQ ID NO: 56
9.14.4C-Ser, 9.14.4-CG2 and 9.14.4-CG4 Light Chain
[Kappa chain] protein sequence
mdmrvpaqllgllllwlrgarcDIQMTQSPSSLSASVGDRVTITCRPSQIISSLLNWY
QQKPGKAPKLLIYAASSLQSGVPSRFSGSGSGTDFTLTISSLQPEDFATY
YCQQSYSTPFTFGPGTKVDIKRtvaapsvfifppsdeqlksgtasvvcllnnfypreakvqw
kydnalqsgnsqesvteqdskdstyslsstltlskadyekhkvyacevthqgisspvtksfnrgec
SEQ ID NO: 74
9.14.4-CG2 Heavy Chain [Gamma chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARGLTGDYWGQGTLVTVSSAstkgpsvfplapesrstsestaalgcl
vkdyfpepvtvswnsgaltsgyhtfpavlqssglysissvvtvpssnfgtqtytcnvdhkpsntkvdktve
rkccvecppcpappvagpsvflfppkpkdtlmisrtpevtcvvvdvshedpevqfnwyvdgvevhna
ktkpreeqfnstfrvvsvltvvhqdwlngkeykckvsnkglpapiektisktkgqprepqvytlppsree
mtknqvsltclvkgfypsdiavewesngqpennykttppmldsdgsfflyskltvdksrwqqgnvfscs
vmhealhnhytqkslslspgk
SEQ ID NO: 78
9.14.4-CG4 Heavy Chain [Gamma chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARGLTGDYWGQGTLVTVSSAstkgpsvfplapcsrstsestaalgcl
vkdyfpepvtvswnsgaltsgvhtfpavlqssglyslssvvtvpssslgtktytcnvdhkpsntkvdkrve
skygppcpscpapeflggpsvflfppkpkdtlmisrtpevtcvvvdvsqedpevqfnwyydgvevhn
aktkpreeqfnstyrvvsvltvlhqdwlngkeykckvsnkglpssiektiskakgqprepqvytlppsqe
emtknqvsltclvkgfypsdiavewesngqpennykttppvldsdgsfflysrltvdksrwqegnvfscs
vmhealhnhytqkslslspgk
SEQ ID NO: 82
9.14.4-Ser Heavy Chain [Gamma chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARGLTGDYWGQGTLVTVSSAstkgpsvfplapcsrstsestaalgcl
vkdyfpepvtvswnsgaltsgvhtfpavlqssglyslssvvtvpssslgtktytcnvdhkpsntkvdkrve
skygppcppcpapeflggpsvflfppkpkdtlmisrtpevtcvvvdvsqedpevqfnwyvdgvevhn
aktkpreeqfnstyrvvsvltvlhqdwlngkeykckvsnkglpssiektiskakgqprepqvytlppsqe
emtknqvsltclvkgfypsdiavewesngqpennykttppvldsdgsfflysrltvdksrwqegnvfscs
vmhealhnhytqkslslspgk
SEQ ID NO: 101
9.14.4G1 Heavy chain (gamma chain) nucieotide sequence
atggagtttgggctgagctgggttttccttgttgctattataaaaggtgtccagtgtCAGGTGCAGCT
GGTGGAGTCTGGGGGAGGCTTGGTCAAGCCTGGAGGGTCCCTGAGA
CTCTCCTGTGCAGCCTCTGGATTCACCTTCAGTGACTACTATATGAGC
TGGATCCGCCAGGCTCCAGGGAAGGGACTGGAGTGGGTTTCATACAT
TAGTAGTAGTGGTAGTACCATATACTACGCAGACTCTGTGAAGGGCCG
ATTCACCATCTCCAGGGACAACGCCAAGAACTCACTGTATCTGCAAAT
GAACAGCCTGAGAGCCGAGGACACGGCCGTGTATTACTGTGCGAGA
GGCCTAACTGGGGACTACTGGGGCCAGGGAACCCTGGTCACCGTCT
CCTCAGCTtccaccaagggcccatcggtcttccccctggcaccctcctccaagagcacctctggg
ggcacagcggccctgggctgcctggtcaaggactacttccccgaaccggtgacggtgtcgtggaactc
aggcgccctgaccagcggcgtgcacaccttcccggctgtcctacagtcctcaggactctactccctcag
cagcgtggtgaccgtgccctccagcagcttgggcacccagacctacatctgcaacgtgaatcacaagc
ccagcaacaccaaggtggacaagaaagttgagcccaaatcttgtgacaaaactcacacatgcccac
cgtgcccagcacctgaactcctggggggaccgtcagtcttcctcttccccccaaaacccaaggacacc
ctcatgatctcccggacccctgaggtcacatgcgtggtggtggacgtgagccacgaagaccctgaggt
caagttcaactggtacgtggacggcgtggaggtgcataatgccaagacaaagccgcgggaggagca
gtacaacagcacgtaccgtgtggtcagcgtcctcaccgtcctgcaccaggactggctgaatggcaagg
agtacaagtgcaaggtctccaacaaagccctcccagcccccatcgagaaaaccatctccaaagcca
aagggcagccccgagaaccacaggtgtacaccctgcccccatcccgggatgagctgaccaagaac
caggtcagcctgacctgcctggtcaaaggcttctatcccagcgacatcgccgtggagtgggagagcaa
tgggcagccggagcacaactacaagaccacgcctcccgtgctggactccgacggctccttcttcctcta
cagcaagctcaccgtggacaagagcaggtggcagcaggggaacgtcttctcatgctccgtgatgcat
gaggctctgcacaaccactacacgcagaagagcctctccctgtctccgggtaaatag
SEQ ID NO: 102
9.14.4G1 Heavy chain (gamma chain) protein sequence
mefglswvflvaiikqvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARGLTGDYWGQGTLVTVSSAstkgpsvfplapsskstsggtaalgc
lvkdyfpepvtvswnsgaltsgvhtfpavlqssglyslssvvtvpssslgtqtyicnvnhkpsntkvdkkv
epkscdkthtcppcpapellggpsvflfppkpkdtlmisrtpevtcvvvdvshedpevkfnwyvdgvev
nnaktkpreeqynstyrvvsvltvlhqdwlngkeykckvsnkalpapiektiskakgqprepqvytlpps
rdeltknqvsltclvkgfypsdiavewesngqpennykttppvldsdgsfflyskltvdksrwqqgnvfsc
svmheaihnhytqkslslspgk
SEQ ID NO: 29
8.10.3 and 8.10.3F Heavy Chain [Gamma chain]
nucleotide sequence
atggagttggggctgtgctgggttttccttgttgctattttagaaggtgtccagtgtGAGGTGCAGCT
GGTGGAGTCTGGGGGAGGCTTGGTACAGCCTGGGGGGTCCCTGAGA
CTCTCCTGTGCAGCCTCTGGATTCACCTTCAGTAGTTTTAGTATGACC
TGGGTCCGCCAGGCTCCAGGAAAGGGGCTGGAGTGGGTTTCATACAT
TAGTAGTAGAAGTAGTACCATATCCTACGCAGACTCTGTGAAGGGCCG
ATTCACCATCTCCAGAGACAATGCCAAGAACTCACTGTATCTGCAAAT
GAACAGCCTGAGAGACGAGGACACGGCTGTGTATTACTGTGCGAGAG
ATCCTCTTCTAGCGGGAGCTACCTTCTTTGACTACTGGGGCCAGGGA
ACCCTGGTCACCGTCTCCTCAGCCtccaccaagggcccatcggtcttccccctggcgc
cctgctccaggagcacctccgagagcacagcggccctgggctgcctggtcaaggactacttccccga
accggtgacggtgtcgtggaactcaggcgctctgaccagcggcgtgcacaccttcccagctgtcctaca
gtcctcaggactctactccctcagcagcgtggtgaccgtgccctccagcaacttcggcacccagaccta
cacctgcaacgtagatcacaagcccagcaacaccaaggtggacaagacagttgagcgcaaatgttg
tgtcgagtgcccaccgtgcccagcaccacctgtggcaggaccgtcagtcttcctcttccccccaaaacc
caaggacaccctcatgatctcccggacccctgaggtcacgtgcgtggtggtggacgtgagccacgaa
gaccccgaggtccagttcaactggtacgtggacggcgtggaggtgcataatgccaagacaaagcca
cgggaggagcagttcaacagcacgttccgtgtggtcagcgtcatcaccgttgtgcaccaggactggctg
aacggcaaggagtacaagtgcaaggtctccaacaaaggcctcccagcccccatcgagaaaaccat
ctccaaaaccaaagggcagccccgagaaccacaggtgtacaccctgcccccatcccgggaggaga
tgaccaagaaccaggtcagcctgacctgcctggtcaaaggcttctaccccagcgacatcgccgtgga
gtgggagagcaatgggcagccggagaacaactacaagaccacacctcccatgctggactccgacg
gctccacttcctctacagcaagctcaccgtggacaagagcaggtggcagcaggggaacgtcttctcat
gctccgtgatgcatgaggctctgcacaaccactacacgcagaagagcctctccctgtctccgggtaaa
SEQ ID NO: 30
8.10.3 and 8.10 3F Heavy Chain [Gamma chain] protein sequence
melglcwvflvailegvqcEVQLVESGGGLVQPGGSLRLSCAASGFTFSSFSMT
WVRQAPGKGLEWVSYISSRSSTISYADSVKGRFTISRDNAKNSLYLQMN
SLRDEDTAVYYCARDPLLAGATFFDYWGQGTLVTVSSAstkgpsvfplapcsrs
tsestaalgclvkdyfpepvtvswnsgaltsgvhtfpavlqssglyslssvvtvpssnfgtqtytcnvdhkp
sntkvdktverkccvecppcpappvagpsvflfppkpkdtlmisrtpevtcvvvdvshedpevqfnwy
vdgvevhnaktkpreeqfnstfrvvsvltvvhqdwlngkeykckvsnkglpapiektisktkgqprepqv
ytlppsreemtknqvsltclvkgfypsdiavewesngqpennykttppmldsdgsfflyskltvdksrwq
qgnvfscsvmhealhnhytqkslslspgk
SEQ ID NO: 31
8.10.3FG1 and 8.10.3F Light Chain [Kappa chain] nucleotide sequence
atggaaaccccagcgcagcttctcttcctcctgctactctggctcccagataccaccggaGAATTTG
TGTTGACGCAGTCTCCAGGCACCCTGTCTTTGTCTCCAGGGGAAAGA
GCCACCCTCTCCTGCAGGGCCAGTCAGAGTGTTAGCAGCAGTTACTT
AGCCTGGTACCAGCAGAAACCTGGCCAGGCTCCCAGGCTCCTCATCT
ATGGTGCATCCAGCAGGGCCACTGGCATCCCAGACAGGTTCAGTGGC
AGTGGGTCTGGGACAGACTTCACTCTCACCATCAGCAGACTGGAGCC
TGAAGATTTTGCAGTGTATTACTGTCAGCAGTATGGTAGCTCACCTCT
CACTTTCGGCGGAGGGACCAAGGTGGAGATCAAACGAactgtggctgcacc
atctgtcttcatcttcccgccatctgatgagcagttgaaatctggaactgcctctgttgtgtgcctgctgaata
acttctatcccagagaggccaaagtacagtggaaggtggataacgccctccaatcgggtaactcccag
gagagtgtcacagagcaggacagcaaggacagcacctacagcctcagcagcaccctgacgctgag
caaagcagactacgagaaacacaaagtctacgcctgcgaagtcacccatcagggcctgagctcgcc
cgtcacaaagagcttcaacaggggagagtgt
SEQ ID NO: 32
8.10.3FG1 and 8.10.3F Light Chain [Kappa chain] protein sequence
metpaqllfllllwlpdttgEFVLTQSPGTLSLSPGERATLSCRASQSVSSSYLAWY
QQKPGQAPRLLIYGASSRATGIPDRFSGSGSGTDFTLTISRLEPEDFAVYY
CQQYGSSPLTFGGGTKVEIKRtvaapsvfifppsdeqlksgtasvvcllnnfypreakvqwk
vdnalqsgnsqesvteqdskdstyslsstltlskadyekhkvyacevthqglsspvtksfnrgec
SEQ ID NO: 43
8.10.3 and 8.10.3-Ser Light Chain [Kappa chain] nucleotide sequence
atggaaaccccagcgcagcttctcttcctcctgctactctggctcccagataccaccggaGAATTTG
TGTTGACGCAGTCTCCAGGCACCCTGTCTTTGTCTCCAGGGGAAAGA
GCCACCCTCTCCTGCAGGGCCAGTCAGAGTGTTAGCAGCAGTTACTT
AGCCTGGTACCAGCAGAAACCTGGCCAGGCTCCCAGGCTCCTCATCT
ATGGTGCATCCAGCAGGGCCACTGGCATCCCAGACAGGTTCAGTGGC
AGTGGGTCTGGGACAGACTTCACTCTCACCATCAGCAGACTGGAGCC
TGAAGATTTTGTAGTGTATTACTGTCAGCAGTATGGTAGCTCACCTCTC
ACTTTCGGCGGAGGGACCAAGGTGGAGATCAAACGAactgtggctgcaccat
ctgtcttcatcttcccgccatctgatgagcagttgaaatctggaactgcctctgttgtgtgcctgctgaataac
ttctatcccagagaggccaaagtacagtggaaggtggataacgccctccaatcgggtaactcccagg
agagtgtcacagagcaggacagcaaggacagcacctacagcctcagcagcaccctgacgctgagc
aaagcagactacgagaaacacaaagtctacgcctgcgaagtcacccatcagggcctgagctcgccc
gtcacaaagagcttcaacaggggagagtgt
SEQ ID NO: 44
8.10.3 and 8.10.3-Ser Light Chain [Kappa chain] protein sequence
metpaqllfllllwlpdttgEFVLTQSPGTLSLSPGERATLSCRASQSVSSSYLAWY
QQKPGQAPRLLIYGASSRATGIPDRFSGSGSGTDFTLTISRLEPEDFVVYY
CQQYGSSPLTFGGGTKVEIKRtvaapsvfifppsdeqlksgtasvvcllnnfypreakvqwk
vdnalqsgnsqesvteqdskdstyslsstltlskadyekhkvyacevthqgisspvtksfnrgec
SEQ ID NO: 58
8.10.3C-Ser Heavy Chain [Gamma chain] protein sequence
melglcwvflvailegvqcEVQLVESGGGLVQPGGSLRLSCAASGFTFSSFSMT
WVRQAPGKGLEWVSYISSRSSTISYADSVKGRFTISRDNAKNSLYLQMN
SLRDEDTAVYYCARDPLLAGATFFDYWGQGTLVTVSSAstkgpsvfplapcsrs
tsestaalgclvkdyfpepvtvswnsgaltsgvhtfpavlqssglysissvvtvpssslgtktytcnvdhkp
sntkvdkrveskygppcppcpapeflggpsvflfppkpkdtlmisrtpevtcvvvdvsqedpevqfnw
yvdgvevhnaktkpreeqfnstyrvvsvltvlhqdwlngkeykckvsnkglpssiektiskakgqprep
qvytlppsqeemtknqvsltclvkgfypsdiavewesngdpennykttppvldsdgsfflysrltvdksr
wqegnvfscsvmhealhnhytqkslslspgk
SEQ ID NO: 60
8.10.3-CG2, 8.10.3-CG4 and 8.10.3C-Ser Light Chain [kappa chain]
protein sequence
metpagllfllllwlcdttgEIVLTQSPGTLSLSPGERATLSCRASQSVSSSYLAWYQ
QKPGQAPRLLIYGASSRATGIPDRFSGSGSGTDFTLTISRLEPEDFAVYYC
QQYGSSPLTFGGGTKVEIKRtvaapsvfifppsdeqlksgtasvvcllhnfypreakvqwkv
dnalqsgnsqesvteqdskdstyslsstltlskadyekhkvyacevthqglsspvtksfnrgec
SEQ ID NO: 62
8.10.3-CG2 Heavy Chain [Gamma chain] protein sequence
melglcwvflvailegvqcEVQLVESGGGLVQPGGSLRLSCAASGFTFSSFSMT
WVRQAPGKGLEWVSYISSRSSTISYADSVKGRFTISRDNAKNSLYLQMN
SLRDEDTAVYYCARDPLLAGATFFDYWGQGTLVTVSSAstkgpsvfplapcsrs
tsestaalgclvkdyfpepvtvswnsgaltsgvhtfpavlqssglyslssvvtvpssnfgtqtytcnvdhkp
sntkvdktverkccvecppcpappvagpsvflfppkpkdtlmisrtpevtcvvvdvshedpevqfnwy
vdgvevhnaktkpreeqfnstfrvvsvltvvhqdwlngkeykckvsnkglpapiektisktkgqprepqv
ytlppsreemtknqvsltclvlkgfypsdiavewesngqpennykttppmidsdgsfflyskltvdksrwq
qgnvfscsvmhealhnhytqkslslspgk
SEQ ID NO: 90
8.10.3-Ser Heavy Chain [Gamma chain] protein sequence
melglcwvflvailegvqcEVQLVESGGGLVQPGGSLRLSCAASGFTFSSFSMT
WVRQAPGKGLEWVASYISSRSSTISYADSVKGRFTISRDNAKNSLYLQMN
SLRDEDTAVYYCARDPLLAGATFFDYWGQGTLVTVSSAstkgpsvfplapcsrs
tsestaalgclvkdyfpepvtvswnsgaltsgvhtfpavlqssglyslssvvtvpssslgtktytcnvdhkp
sntkvdkrveskygppcppcpapeflggpsvflfppkpkdtlmisrtpevtcvvvdvsqedpevqfnw
yvdgvevhnaktkpreeqfnstyrvvsvltylhqdwlngkeykckvsnkglpssiektiskakgqprep
qvytlppsqeemntknqvsltclvkgfypsdiavewesngqpennykttppvldsdgsfflysrltvdksr
wqegnvfscsvmhealhnhytqkslslspgk
SEQ ID NO: 94
8.10.3-CG4 Heavy Chain [Gamma chain] protein sequence
melglcwvflvailegvqcEVQLVESGGGLVQPGGSLRLSCAASGFTFSSFSMT
WVRQAPGKGLEWVSYISSRSSTISYADSVKGRFTISRDNAKNSLYLQMN
SLRDEDTAVYYCARDPLLAGATFFDYWGQGTLVTVSSAstkgpsvfplapcsrs
tsestaalgclvkdyfpepvtvswnsgaltsgvhtfpavlqssglyslssvvtvpssslgtktytcnvdhkp
sntkvdkrveskygppcpscpapeflggpsvflfppkpkdtlmisrtpevtcvvvdvsqedpevqfnwy
vdgvevhnaktkpreeqfnstyrvvsvltvlhqdwlngkeykckvsnkglpssiektiskakgqprepq
vytlppsqeemtknqvsltclvkgfypsdiavewesnggpennykttppvldsdgsfflysrltvdksrw
qegnvfscsvmhealhnhytqkslslspgk
SEQ ID NO: 97
8.90.3FG1 Heavy Chain nucleotide sequence
atggagttggggctgagctgggttttccttgttgctattataaaaggtgtccagtgtGAGGTGCAGCT
GGTGGAGTCTGGGGGAGGCTTGGTACAGCCTGGGGGGTCCCTGAGA
CTCTCCTGTGCAGCCTCTGGATTCACCTTCAGTAGTTTTAGTATGACC
TGGGTCCGCCAGGCTCCAGGAAAGGGGCTGGAGTGGGTTTCATACAT
TAGTAGTAGAAGTAGTACCATATCCTACGCAGACTCTGTGAAGGGCCG
ATTCACCATCTCCAGAGACAATGCCAAGAACTCACTGTATCTGCAAAT
GAACAGCCTGAGAGACGAGGACACGGCTGTGTATTACTGTGCGAGAG
ATCCTCTTCTAGCGGGAGCTACCTTCTTTGACTACTGGGGCCAGGGA
ACCCTGGTCACCGTCTCCTCAGCCtccaccaagggcccatcggtcttccccctggcac
cctcctccaagagcacctctgggggcacagcggccctgggctgcctggtcaaggactacttccccgaa
ccggtgacggtgtcgtggaactcaggcgccctgaccagcggcgtgcacaccttcccggctgtcctaca
gtcctcaggactctactccctcagcagcgtggtgaccgtgccctccagcagcttgggcacccagaccta
catctgcaacgtgaatcacaagcccagcaacaccaaggtggacaagaaagttgagcccaaatcttgt
gacaaaactcacacatgcccaccgtgcccagcacctgaactcctggggggaccgtcagtcttcctcttc
cccccaaaacccaaggacaccctcatgatctcccggacccctgaggtcacatgcgtggtggtggacgt
gagccacgaagaccctgaggtcaagttcaactggtacgtggacggcgtggaggtgcataatgccaag
acaaagccgcgggaggagcagtacaacagcacgtaccgtgtggtcagcgtcctcaccgtcctgcac
caggactggctgaatggcaaggagtacaagtgcaaggtctccaacaaagccctcccagcccccatc
gagaaaaccatctccaaagccaaagggcagccccgagaaccacaggtgtacaccctgcccccatc
ccgggatgagctgaccaagaaccaggtcagcctgacctgcctggtcaaaggcttctatcccagcgac
atcgccgtggagtgggagagcaatgggcagccggagaacaactacaagaccacgcctcccgtgctg
gactccgacggctccttcttcctctacagcaagctcaccgtggacaagagcaggtggcagcagggga
acgtcttctcatgctccgtgatgcatgaggctctgcacaaccactacacgcagaagagcctctccctgtct
ccgggtaaatag
SEQ ID NO: 98
8.10.3FG1 Heavy chain (gamma chain) protein sequence
melglcwvflvailegvqcEVQLVESGGGLVQPGGSLRLSCAASGFTFSSFSMT
WVRQAPGKGLEWVSYISSRSSTISYADSVKGRFTISRDNAKNSLYLQMN
SLRDEDTAVYYCARDPLLAGATFFDYWGQGTLVTVSSAstkgpsvfplapssk
stsggtaalgclvkdyfpepvtvswnsgaltsgvhtfpavlqssglyslssvvtvpssslgtqtyicnvnhk
psntkvdkkvepkscdkthtcppcpapellggpsvflfppkpkdtlmisrtpevtcvvvdvshedpevkf
nwyvdgvevhnaktkpreeqynstyrvvsvltvlhqdwlngkeykckvsnkalpapiektiskakggp
repqvytlppsrdeltknqvsltclvkgfypsdiavewesngqpennykttppvldsdgsfflyskltvdks
rwqqgnvfscsvmhealhnhytqkslslspgk
SEQ ID NO: 33
9.7.2IF Heavy Chain [Gamma chain] nucleotide sequence
atggagtttgggctgagctgggttttccttgttgctattataaaaggtgtccagtgtcAGGTGCAGCT
GGTGGAGTCTGGGGGAGGCTTGGTCAAGCCTGGAGGGTCCCTGAGA
CTCTCCTGTGCAGCCTCTGGATTCACCTTCAGTGACTACTACATGAGC
TGGATCCGCCAGGCTCCAGGGAAGGGGCTGGAGTGGGTTTCATACAT
TAGTAGTAGTGGTAGTACCATATACTACGCAGACTCTGTGAAGGGCCG
ATTCACCATCTCCAGGGACAACGCCAAGAATTCACTGTATCTGCAAAT
GAACAGCCTGAGAGCCGAGGACACGGCCGTGTATTACTGTGCGAGG
CGTATAGGAGGTATGGACGTCTGGGGCCAAGGGACCACGGTCACCG
TCTCCTCAGCTtccaccaagggcccatccgtcttccccctggcgccctgctctagaagcacctcc
gagagcacagcggccctgggctgcctggtcaaggactacttccccgaaccggtgacggtgtcgtgga
actcaggcgctctgaccagcggcgtgcacaccttcccagctgtcctacagtcctcaggactctactccct
cagcagcgtggtgaccgtgccctccagcaacttcggcacccagacctacacctgcaacgtagatcac
aagcccagcaacaccaaggtggacaagacagttgagcgcaaatgttgtgtcgagtgcccaccgtgcc
cagcaccacctgtggcaggaccgtcagtcttcctcttccccccaaaacccaaggacaccctcatgatct
cccggacccctgaggtcacgtgcgtggtggtggacgtgagccacgaagaccccgaggtccagttcaa
ctggtacgtggacggcgtggaggtgcataatgccaagacaaagccacgggaggagcagttcaacag
cacgttccgtgtggtcagcgtcctcaccgttgtgcaccaggactggctgaacggcaaggagtacaagtg
caaggtctccaacaaaggcctcccagcccccatcgagaaaaccatctccaaaaccaaagggcagc
cccgagaaccacaggtgtacaccctgcccccatcccgggaggagatgaccaagaaccaggtcagc
ctgacctgcctggtcaaaggcttctaccccagcgacatcgccgtggagtgggagagcaatgggcagc
cggagaacaactacaagaccacacctcccatgctggactccgacggctccttcttectctacagcaag
ctcaccgtggacaagagcaggtggcagcaggggaacgtcttctcatgctccgtgatgcatgaggctct
gcacaaccactacacgcagaagagcctctccctgtctccgggtaaa
SEQ ID NO: 34
9.7.2IF Heavy Chain [Gamma Chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARRIGGMDVWGQGTTVTVSSAstkgpsvfplapcsrstsestaalg
clvkdyfpepvtvswnsgaltsgvhtfpavlqssglyslssvvtvpssnfgtqtytcnvdhkpsntkvdkt
verkccvecppcpappvagpsvflfppkpkdtlmisrtpevtcvvvdvshedpevqfnwyvdgvevh
naktkpreeqfnstfrvvsvltvvhqdwlngkeykckvsnkglpapiektisktkgqprepqvytlppsre
emtknqvsltclvkgfypsdiavewesngqpennykttppmldsdgsfflyskltvdksrwqqgnvfsc
svmhealhnhytqkslslspgk
SEQ ID NO: 35
9.7.2IF Light Chain [Kappa chain] nucleotide sequence
atggacatgagggtccccgctcagctcctggggctcctgctactctggctccgaggtgccagatgtGA
CATCCAGATGACCCAGTCTCCATCCTCCCTGTCTGCATCTGTAGGAGA
CAGAGTCACCATCACTTGCCGGGCAAGTCAGAGCATTAGCGGCTTTTT
AATTTGGTATCAGCAGAGACCAGGGAAAGCCCCTAAGCTCCTGATCTA
TGCTACATCCAGTTTACAAAGTGGGGTCCCATCAAGGTTCAGTGGCAG
TGGATCTGGGACAGATTTCACTCTCACCATCAGCAGTCTGCAACCTGA
AGATTTTGCAACTTACTACTGTCAACAGAGTTACAGTACCCCATTCACT
TTCGGCCCTGGGACCAAAGTGGATATCAAACGAactgtggctgcaccatctgtctt
catcttcccgccatctgatgagcagttgaaatctggaactgcctctgttgtgtgcctgctgaataacttctatc
ccagagaggccaaagtacagtggaaggtggataacgccctccaatcgggtaactcccaggagagtg
tcacagagcaggacagcaaggacagcacctacagcctcagcagcaccctgacgctgagcaaagc
agactacgagaaacacaaagtetacgcctgcgaagtcacccatcagggcctgagctcgcccgtcac
aaagagcttcaacaggggagagtgt
SEQ ID NO: 36
9.7.2IF Light Chain [Kappa chain] protein sequence
mdmrvpaqllqllllwlrgarcDIQMTQSPSSLSASVGDRVTITCRASQSISGFLIWY
QQRPGKAPKLLIYATSSLQSGVPSRFSGSGSGTDFTLTISSLQPEDFATY
YCQQSYSTPFTFGPGTKVDIKRtvaapsvfifppsdeqlksgtasvvcllnnfypreakvqw
kvdnalqsgnsqesvteqdskdstyslsstltlskadyekhkvyacevthqglsspvtksfnrgec
SEQ ID NO: 45
9.7.2 Heavy Chain [Gamma chain] nucleotide sequence
atggagtttgggctgagctgggttttccttgttgctattataaaaggtgtccagtgtcAGGTGCAGCT
GGTGGAGTCTGGGGGAGGCTTGGTCAAGCCTGGAGGGTCCCTGAGA
CTCTCCTGTGCAGCCTCTGGATTCACCTTCAGTGACTACTACATGAGC
TGGATCCGCCAGGCTCCAGGGAAGGGGCTGGAGTGGGTTTCATACAT
TAGTAGTAGTGGTAGTACCATATACTACGCAGACTCTGTGAAGGGCCG
ATTCACCATCTCCAGGGACAACGCCAAGAATTCACTGTATCTGCAAAT
GAACAGCCTGAGAGCCGAGGACACGGCCGTGTATTACTGTGCGAGG
CGTATAGGAGGTATGGACGTCTGGGGCCAAGGGACCACGGTCACCG
TCTCCTCAGCTtccaccaagggcccatccgtcttccccctggcgccctgctctagaagcacctcc
gagagcacagcggccctgggctgcctggtcaaggactacttccccgaaccggtgacggtgtcgtgga
actcaggcgctctgaccagcggcgtgcacaccttcccagctgtcctacagtcctcaggactctactccct
cagcagcgtggtgaccgtgccctccagcagcttgggcacgaagacctacacctgcaacgtagatcac
aagcccagcaacaccaaggtggacaagagagttgagtccaaatatggtcccccatgcccatcatgcc
cagcacctgagttcctggggggaccatcagtcttcctgttccccccaaaacccaaggacactctcatgat
ctcccggacccctgaggtcacgtgcgtggtggtggacgtgagccaggaagaccccgaggtccagttc
aactggtacgtggatggcgtggaggtgcataatgccaagacaaagccgcgggaggagcagttcaac
agcacgtaccgtgtggtcagcgtcctcaccgtcctgcaccaggactggctgaacggcaaggagtaca
agtgcaaggtctccaacaaaggcctcccgtcctccatcgagaaaaccatctccaaagccaaagggc
agccccgagagccacaggtgtacaccctgcccccatcccaggaggagatgaccaagaaccaggtc
agcctgacctgcctggtcaaaggcttctaccccagcgacatcgccgtggagtgggagagcaatgggc
agccggagaacaactacaagaccacgcctcccgtgctggactccgacggctccttcttcctctacagc
aggctaaccgtggacaagagcaggtggcaggaggggaatgtcttctcatgctccgtgatgcatgaggc
tctgcacaaccactacacacagaagagcctctccctgtctccgggtaaa
SEQ ID NO: 46
9.7.2 Heavy Chain [Gamma Chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARRIGGMDVWGQGTIVTVSSAstkgpsvfplapcsrstsestaalg
clvkdyfpepvtvswnsgaltsgvhtfpavlqssglyslssvvtvpsssigtktytcnvdhkpsntkvdkrv
eskygppcpscpapeflggpsvflfppkpkdtlmisrtpevtcvvvdvsqedpevqfnwyydgvevh
naktkpreeqfnstyrvvsvltvlhqdwlngkeykckvsnkglpssiektiskakgqprepqvytlppsq
eemtknqvsltclvkgfypsdiavewesngqpennykttppvldsdgsfflysrltvdksrwqegnvfsc
svmhealhnhytqkslslspgk
SEQ ID NO: 47
9.7.2 and 9.7.2-Ser Light Chain [Kappa chain] nucleotide sequence
atggacatgagggtccccgctcagctcctggggctcctgctactctggctccgaggtgccagatgtGA
CATCCAGATGACCCAGTCTCCATCCTCCCTGTCTGCATCTGTAGGAGA
CAGAGTCACCATCACTTGCCGGGCAAGTCAGAGCATTAGCGGCTTTTT
AATTTGGTATCAGCAGAGACCAGGGAAAGCCCCTAAGCTCCTGATCTA
TGCTACATCCAGTTTACAAAGTGGGGTCCCATTAAGGTTCAGTGGCAG
TGAATCTGGGACAGATTTCACTCTCACCATCAGCAGTCTGCAACCTGA
AGATTTTGCAACTTACTACTGTCAACAGAGTTACAGTACCCCATTCACT
TTCGGCCCTGGGACCAAAGTGGATATCAAACGAactgtggctgcaccatctgtctt
catcttcccgccatctgatgagcagttgaaatctggaactgcctctgttgtgtgcctgctgaataacttctatc
ccagagaggccaaagtacagtggaaggtggataacgccctccaatcgggtaactcccaggagagtg
tcacagagcaggacagcaaggacagcacctacagcctcagcagcaccctgacgctgagcaaagc
agactacgagaaacacaaagtctacgcctgcgaagtcacccatcagggcctgagctcgcccgtcac
aaagagcttcaacaggggagagtgt
SEQ ID NO: 48
9.7.2 and 9.7.2-Ser Light Chain [Kappa chain] protein sequence
mdmrvpaqlgllllwlrgarcDIQMTQSPSSLSASVGDRVTITCRASQSISGFLIWY
QQRPGKAPKLLIYATSSLQSGVPLRFSGSESGTDFTLTISSLQPEDFATYY
CQQSYSTPFTFGPGTKVDIKRtvaapsvflfppsdeqlksgtasvvcllnnfypreakvqwk
vdnalqsgnsqesvteqdskdstyslsstltlskadyekhkvyaceythqgisspytksfnrgec
SEQ ID NO: 50
9.7.2C-Ser Heavy Chain [Gamma chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCAIRIGGMDVWGQGTTVTVSSAstkgpsvfplapcsrstsestaalgc
lvkdyfpepvtvswnsgaltsgvhtfpavlqssglyslssvvtvpssslgtktytcnvdhkpsntkvdkrve
skygppcppcpapeflggpsvflfppkpkdtlmisrtpevtcvvvdvsqedpevqfnwyvdgvevhn
aktkpreeqfnstyrvvsvltvlhqdwlngkeykckvsnkglpssiektiskakgqprepqvytlppsqe
emtknqvsltclvkgfypsdiavewesngqpennykttppvldsdgsfflysrltvdksrwqegnvfscs
vmhealhnhytqkslslspgk
SEQ ID NO: 52
9.7.2C-Ser, 9.7.2-CG2 and 9.7.2-CG4 Light Chain [Kappa chain]
protein sequence
mdmrvpagllgllllwlrgarcDIQMTQSPSSLSASVGDRVTITCRASQSISGFLIWY
QQKPGKAPKLLIYATSSLQSGVPSRFSGSGSGTDFTLTISSLQPEDFATYY
CQQSYSTPFTFGPGTKVDIKRtvaapsvflfppsdeqlksgtasvvcllnnfypreakvqwk
vdnalqsgnsqesvteqdskdstyslsstltlskadyekhkvyacevthqglsspvtksfnrgec
SEQ ID NO: 66
9.7.2-CG2 Heavy Chain [Gamma chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCAIRIGGMDVWGQGTIVTVSSAstkgpsvfplapcsrstsestaalgc
lvkdyfpepvtvswnsgaltsgvhtfpavlqssglyslssvvtvpssnfgtqtytcnvdhkpsntkvdktv
erkccvecppcpappvagpsvflfppkpkdtlmisrtpevtcvvvdvshedpevqfnwyvdgvevhn
aktkpreeqfnstfrvvsvltvvhqdwlngkeykckvsnkgipapiektisktkgqprepqvytlppsree
mtknqvsltclvkgfypsdiavewesngqpennykttppmldsdgsfflyskltvdksrwqqgnvfscs
vmhealhnhytqkslslspgk
SEQ ID NO: 70
9.7.2-CG4 Heavy Chain [Gamma chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARIGGMDVWGQGTTVTVSSAstkgpsvfplapcsrstsestaalgcl
vkdyfpepvtvswnsgaltsgvhtfpavlqssglyslssvvtvpssslgtktytcnvdhkpsntkvdkrve
skygppcpscpapeflggpsvflfppkpkdtlmisrtpevtcvvvdvsqedpevqfnwyvdgvevhn
aktkpreeqfnstyrvvsvltvlhqdwlngkeykckvsnkglpssiektiskakgqprepqvytlppsqe
emtknqvsltclvkgfypsdiavewesngqpennykttppvldsdgsfflysrltvdksrwqegnvfscs
vmhealhnhytqkslslspgk
SEQ ID NO: 86
9.7.2-Ser Heavy Chain [Gamma chain] protein sequence
mefglswvflvaiikgvqcQVQLVESGGGLVKPGGSLRLSCAASGFTFSDYYMS
WIRQAPGKGLEWVSYISSSGSTIYYADSVKGRFTISRDNAKNSLYLQMNS
LRAEDTAVYYCARRIGGMDVWGQGTTVTVSSAstkgpsvfplapcsrstsestaalg
clvkdyfpepvtvswnsgaltsgvhtfpavlqssglyslssvvtvpssslgtktytcnvdhkpsntkvdkrv
eskygppcppcpapeflggpsvflfppkpkdtlmisrtpevtcvvvdvsqedpevqfnwyvdgvevh
naktkpreeqfnstyrvvsvltvlhqdwlngkeykckvsnkglpssiektiskakgqprepqvytlppsq
eemtknqvsltclvkgfypsdiavewesngqpennykttppvldsdgsfflysrltvdksrwqegnvfsc
svmhealhnhytqkslslspgk

1. A method for treating lupus comprising administering to a subject in need thereof a therapeutically effective amount of a human monoclonal antibody or an antigen-binding portion thereof that specifically binds to M-CSF.

2. The method according to claim 1, wherein said antibody or antigen-binding portion possesses at least one of the following properties:
a) binds to human secreted isoforms of M-CSF and membrane bound isoforms of M-CSF;
b) has a selectivity for M-CSF that is at least 100 times greater than its selectivity for GM-CSF or G-CSF;
c) binds to M-CSF with a K_{D} of 1.0×10⁻⁷ M or less;
d) has an off rate (k_{off}) for M-CSF of 2.0×10⁻⁴ s⁻¹ or smaller; or
e) binds human M-CSF in the presence of human c-fms.

3. The method according to claim 2, wherein said antibody or antigen-binding portion blocks binding to c-fms and binds M-CSF with a K_{D} of 1.0×10⁻⁷ M or less.

4. The method according to claim 1, wherein the antibody or antigen-binding portion has at least one property selected from the group consisting of:
a) cross-competes for binding to M-CSF with an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1;
b) competes for binding to M-CSF with an antibody selected from the group consisting of: 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1;
c) binds to the same epitope of M-CSF as an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1;
d) binds to M-CSF with substantially the same K_{D} as an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1; and
e) binds to M-CSF with substantially the same off rate as an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1.

5. The method according to claim 1, wherein the antibody or antigen-binding portion is selected from the group consisting of:
a) an antibody comprising the heavy chain amino acid sequence set forth in SEQ ID NO: 2 and the light chain amino acid sequence set forth in SEQ ID NO: 4, without the signal sequences;
b) an antibody comprising the heavy chain amino acid sequence set forth in SEQ ID NO: 6 and the light chain amino acid sequence set forth in SEQ ID NO: 8, without the signal sequences;
c) an antibody comprising the heavy chain amino acid sequence set forth in SEQ ID NO: 10 and the light chain amino acid sequence set forth in SEQ ID NO: 12, without the signal sequences;
d) an antibody comprising the heavy chain amino acid sequence set forth in SEQ ID NO: 14 and the light chain amino acid sequence set forth in SEQ ID NO: 16, without the signal sequences;
e) an antibody comprising the heavy chain amino acid sequence set forth in SEQ ID NO: 18 and the light chain amino acid sequence set forth in SEQ ID NO: 20, without the signal sequences;
f) an antibody comprising the heavy chain amino acid sequence set forth in SEQ ID NO: 22 and the light chain amino acid sequence set forth in SEQ ID NO: 24, without the signal sequences;
g) an antibody comprising the heavy chain amino acid sequence set forth in SEQ ID NO: 26 and the light chain amino acid sequence set forth in SEQ ID NO: 28, without the signal sequences;
h) an antibody comprising the heavy chain amino acid sequence set forth in SEQ ID NO: 38 and the light chain amino acid sequence set forth in SEQ ID NO: 28, without the signal sequences;
i) an antibody comprising the heavy chain amino acid sequence set forth in SEQ ID NO: 54 and the light chain amino acid sequence set forth in SEQ ID NO: 56, without the signal sequences;
j) an antibody comprising the heavy chain amino acid sequence set forth in SEQ ID NO: 74 and the light chain amino acid sequence set forth in SEQ ID NO: 56, without the signal sequences;
k) an antibody comprising the heavy chain amino acid sequence set forth in SEQ ID NO: 78 and the light chain amino acid sequence set forth in SEQ ID NO: 56, without the signal sequences;
l) an antibody comprising the heavy chain amino acid sequence set forth in SEQ ID NO: 82 and the light chain amino acid sequence set forth in SEQ ID NO: 28, without the signal sequences;
m) an antibody comprising the heavy chain amino acid sequence set forth in SEQ ID NO: 102 and the light chain amino acid sequence set forth in SEQ ID NO: 28, without the signal sequences;
n) an antibody comprising the heavy chain amino acid sequence set forth in SEQ ID NO: 30 and the light chain amino acid sequence set forth in SEQ ID NO: 32, without the signal sequences;
o) an antibody comprising the heavy chain amino acid sequence set forth in SEQ ID NO: 30 and the light chain amino acid sequence set forth in SEQ ID NO: 44, without the signal sequences;
p) an antibody comprising the heavy chain amino acid sequence set forth in SEQ ID NO: 58 and the light chain amino acid sequence set forth in SEQ ID NO: 60, without the signal sequences;
q) an antibody comprising the heavy chain amino acid sequence set forth in SEQ ID NO: 62 and the light chain amino acid sequence set forth in SEQ ID NO: 60, without the signal sequences;
r) an antibody comprising the heavy chain amino acid sequence set forth in SEQ ID NO: 90 and the light chain amino acid sequence set forth in SEQ ID NO: 44, without the signal sequences;
s) an antibody comprising the heavy chain amino acid sequence set forth in SEQ ID NO: 94 and the light chain amino acid sequence set forth in SEQ ID NO: 60, without the signal sequences;
t) an antibody comprising the heavy chain amino acid sequence set forth in SEQ ID NO: 98 and the light chain amino acid sequence set forth in SEQ ID NO: 32, without the signal sequences;
u) an antibody comprising the heavy chain amino acid sequence set forth in SEQ ID NO: 34 and the light chain amino acid sequence set forth in SEQ ID NO: 36, without the signal sequences;
v) an antibody comprising the heavy chain amino acid sequence set forth in SEQ ID NO: 46 and the light chain amino acid sequence set forth in SEQ ID NO: 48, without the signal sequences;
w) an antibody comprising the heavy chain amino acid sequence set forth in SEQ ID NO: 50 and the light chain amino acid sequence set forth in SEQ ID NO: 52, without the signal sequences;
x) an antibody comprising the heavy chain amino acid sequence set forth in SEQ ID NO: 66 and the light chain amino acid sequence set forth in SEQ ID NO: 52, without the signal sequences;
y) an antibody comprising the heavy chain amino acid sequence set forth in SEQ ID NO: 70 and the light chain amino acid sequence set forth in SEQ ID NO: 52, without the signal sequences; and
z) an antibody comprising the heavy chain amino acid sequence set forth in SEQ ID NO: 86 and the light chain amino acid sequence set forth in SEQ ID NO: 48, without the signal sequences.

6. The method according to claim 1, wherein said antibody or antigen-binding portion comprises:
a) a heavy chain CDR1, CDR2 and CDR3 independently selected from the heavy chain of an antibody selected from the group consisting of: monoclonal antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1; or
b) a light chain CDR1, CDR2 and CDR3 independently selected from the light chain of an antibody selected from the group consisting of: monoclonal antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1.

7. The method according to claim 1, wherein:
a) the antibody or antigen binding portion comprises the heavy chain CDR1, CDR2 and CDR3 of an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1;
b) the antibody or antigen binding portion comprises the heavy chain CDR1, CDR2 and CDR3 of an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1;
c) the antibody comprises a heavy chain of (a) and a light chain of (b); or
d) the antibody or antigen binding portion comprises a heavy chain of (a) and a light chain of (b), wherein the heavy chain and light chain CDR amino acid sequences are selected from the same antibody.

8. The method according to claim 1, wherein the antibody or antigen-binding portion comprises:
a) a heavy chain comprising the amino acid sequence from the beginning of the CDR1 through the end of the CDR3 of the heavy chain of an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1;
b) a light chain comprising the amino acid sequence from the beginning of the CDR1 through the end of the CDR3 of an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1;
c) the heavy chain of (a) and the light chain of (b); or
d) the heavy chain of (a) and the light chain of (b) wherein the heavy chain and light chain sequences are selected from the same antibody.

9. The method according to claim 1, wherein said monoclonal antibody or antigen-binding portion comprises:
a) the heavy chain variable domain (VH) amino acid sequence, without a signal sequence, of an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1;
b) the light chain variable domain (VL) amino acid sequence, without a signal sequence, of an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1;
c) the VH amino acid sequence of (a) and the VL amino acid sequence of (b); or
d) the VH amino acid sequence of (a) and the VL amino acid sequence of (b), wherein the VH and VL are from the same antibody.

10. The method according to claim 1, wherein said antibody or antigen-binding portion comprises one or more of an FR1, FR2, FR3 or FR4 amino acid sequence of an antibody selected from the group consisting of: antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 and 9.14.4G1.

11. The method according to claim 1, wherein the C-terminal lysine of the heavy chain of said antibody or antigen-binding portion is not present.

12. The method according to claim 1, wherein the antibody comprises:
a) a heavy chain amino acid sequence that is at least 90% identical to the heavy chain amino acid sequence of monoclonal antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, without the signal sequence;
b) a light chain amino acid sequence that is at least 90% identical to the light chain amino acid sequence of monoclonal antibody 252, 88, 100, 3.8.3, 2.7.3, 1.120.1, 9.14.4I, 8.10.3F, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1, without the signal sequence; or
c) the heavy chain amino acid sequence of (a) and the light chain amino acid sequence of (b).

13. The method according to claim 1, wherein the antibody or antigen binding portion comprises:
a) a heavy chain amino acid sequence that is at least 90% identical to the heavy chain amino acid sequence of monoclonal antibody 8.10.3F without the signal sequence;
b) a light chain amino acid sequence that is at least 90% identical to the light chain amino acid sequence of monoclonal antibody 8.10.3F;
c) the heavy chain amino acid sequence of (a) and the light chain amino acid sequence of (b); or
d) a heavy chain amino acid sequence and a light chain amino acid sequence that together are at least 90% identical to the heavy chain amino acid sequence and the light chain amino acid sequence of antibody 8.10.3F.

14. The method according to claim 13, wherein the heavy chain amino acid sequence of the antibody or antigen-binding portion is at least 95% identical to the heavy chain amino acid sequence of monoclonal antibody 8.10.3F without the signal sequence.

15. The method according to claim 13, wherein the light chain amino acid sequence of the antibody or antigen-binding portion is at least 95% identical to the light chain amino acid sequence of monoclonal antibody 8.10.3F without the signal sequence.

16. The method according to claim 13, wherein both the heavy chain amino acid sequence and light chain amino acid sequence of the antibody or antigen-binding portion together are at least 95% identical to the heavy chain amino acid sequence and light chain amino acid sequence of monoclonal antibody 8.10.3F without the signal sequence.

17. The method according to claim 13, wherein the heavy chain amino acid sequence of the antibody or antigen-binding portion is at least 97% identical to the heavy chain amino acid sequence of monoclonal antibody 8.10.3F without the signal sequence.

18. The method according to claim 13, wherein the light chain amino acid sequence of the antibody or antigen-binding portion is at least 97% identical to the light chain amino acid sequence of monoclonal antibody 8.10.3F without the signal sequence.

19. The method according to claim 13, wherein both the heavy chain amino acid sequence and light chain amino acid sequence of the antibody or antigen-binding portion together are at least 97% identical to the heavy chain amino acid sequence and light chain amino acid sequence of monoclonal antibody 8.10.3F without the signal sequence.

20. The method according to claim 1 wherein the antibody is monoclonal anti-M-CSF antibody 8.10.3F.

21. The method according to claim 1, wherein the antibody or antigen-binding portion comprises the heavy chain CDR1, CDR2, CDR3 and the light chain CDR1, CDR2, CDR3 of antibody 8.10.3F.

22. The method according to claim 1, wherein the antibody or antigen-binding portion comprises the variable heavy chain and variable light chain portion of antibody 8.10.3F.

23. The method according to claim 1, wherein the lupus is systemic lupus erythematosus (SLE).

24. The method according to claim 1, wherein the lupus is lupus nephritis.

25. The method according to claim 1, wherein the lupus is cutaneous lupus.

26. The method according to claim 1, wherein efficacy of the anti-M-CSF antibody in treating lupus is determined by examining patients for changes in conditions selected from symptoms, biomarkers, histological samples, and physiological conditions

27. The method according to claim 26, wherein the patients are examined for changes in conditions selected from skin lesions, proteinuria, lymphadenopathy, serum M-CSF levels, anti-dsDNA antibody levels, CD14+CD16+ monocyte population, osteocyte markers, and kidney pathology

28. The method according to claim 27, wherein kidney pathology is determined by examining conditions selected from macrophage infiltration, inflammatory infiltrates, proteinaceous casts, size of glomerular tufts, glomerular IgG deposits, and C3 deposits.

29. The method according to claim 26, wherein patients may be examined for changes in one or more biomarkers selected from the genes in Table 1C, erythrocyte sedimentation rate (ESR), C-reactive protein (CRP), complement (C3/C4), Ig levels (IgA, IgM, IgG), antinuclear antibodies (ANA), extractable nuclear antigen (ENA), and anti-dsDNA antibodies.

30-37. (canceled)
